SEQUENCE LISTING (1 sequence)

SEQ ID NO: 1 - protein, 1237 aa, Homo sapiens
GRSSKAKKPPGENDFDTIKLISNGAYGAVYLVRHRDTRQRFAMKKINKQNLILRNQIQQAFVERDILTFAENPFVVGMFCSFETRRHLCMVMEYVEGGDCATLLKNIGALPVEMARMYFAETVLALEYLHNYGIVHRDLKPDNLLITSMGHIKLTDFGLSKMGLMSLTTNLYEGHIEKDAREFLDKQVCGTPEYIAPEVILRQGYGKPVDWWAMGIILYEFLVGCVPFFGDTPEELFGQVISDDILWPEGDEALPTEAQLLISSLLQTNPLVRLGAGGAFEVKQHSFFRDLDWTGLLRQKAEFIPHLESEDDTSYFDTRSDRYHHVNSYDEDDTTEEEPVEIRQFSSCSPRFSKVYSSMEQLSQHEPKTPVAAAGSSKREPSTKGPEEKVAGKREGLGGLTLREKTWRGGSPEIKRFSASEASFLEGEASPPLGARRRFSALLEPSRFSAPQEDEDEARLRRPPRPSSDPAGSLDARAPKEETQGEGTSSAGDSEASPRATNDLVLRRARHQQMSGDVAVEKRPSRTGGKVIKSASATALSAGPMRGCSATALGGGRSRYREMSIFLVLISDHGCVSVVDPHGSSPLASPMSPRSLSSNPSSRDSSPSRDYSPAVSGLRSPITIQRSGKKYGFTLRAIRVYMGDTDVYSVHHIVWHVEEGGPAQEAGLCAGDLITHVNGEPVHGMVHPEVVELILKSGNKVAVTTTPFENTSIRIGPARRSSYKAKMARRNKRPSAKEGQESKKRSSLFRKITKQSNLLHTSRSLSSLNRSLSSSDSLPGSPTHGLPARSPTHSYRSTPDSAYLGITSCTCAGTEQRGVAWLSSSPASSTPNSPASSASHHIRPSTLHGLSPKLHRQYRSARCKSAGNIPLSPLAHTPSPTQASPPPLPGHTVGSSHTTQSFPAKLHSSPPVVRPRPKSAEPPRSPLLKRVQSAEKLGASLSADKKGALRKHSLEVGHPDFRKDFHGELALHSLAESDGETPPVEGLGAPRQVAVRRLGRQESPLSLGADPLLPEGASRPPVSSKEKESPGGAEACTPPRATTPGGRTLERDVGCTRHQSVQTEDGTGGMARAVAKAALSPVQEHETGRRSSSGEAGTPLVPIVVEPARPGAKAVVPQPLGADSKGLQEPAPLAPSVPEAPRGRERWVLEVVEERTTLSGPRSKPASPKLSPEPQTPSLAPAKCSAPSSAVTPVPPASLLGSGTKPQVGLTSRCPAEAVPPAGLTKKGVSSPAPPGP

(19) United States
(12) Patent Application Publication
(10) Pub. No.: US 2005/0032096 A1
(43) Pub. Date: Feb. 10, 2005

(54) Methods for predicting drug sensitivity in patients afflicted with hypertension

(75) Inventor: Hakon Hakonarson, Reykjavik (IS)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: deCODE genetics ehf.

(21) Appl. No.: 10/863,906

(22) Filed: Jun. 8, 2004

Related U.S. Application Data
(60) Provisional application No. 60/477,087, filed on Jun. 9, 2003.

(51) Int. Cl. C12Q 1/68; A61K 31/455; A61K 31/401

(52) U.S. Cl. 435/6; 514/355; 514/423

(57) ABSTRACT

Methods are disclosed for predicting the efficacy of a drug for treating hypertension in a patient, including: obtaining a sample of cells from the patient; obtaining a gene expression profile from the sample in the absence and presence of in vitro modulation of the cells with specific mediators; and comparing the gene expression profile of the sample with a reference gene expression profile, wherein similarity between the sample expression profile and the reference expression profile predicts the efficacy of the drug for treating hypertension in the patient.

### RELATED APPLICATION

This application claims the benefit of U.S. Provisional Application No. 60/477,087, filed Jun. 9, 2003. The entire teachings of the above application are incorporated herein by reference.

### BACKGROUND OF THE INVENTION

The field of pharmacogenomics measures differences in the effect of medications that are caused by genetic variations. Such differences are manifested by differences in the therapeutic effects or adverse events of drugs. For most drugs, the genetic variations that potentially characterize drug-responsive patients from non-responders remain unknown.

Hypertension, or high blood pressure, is the most common chronic illness in America. The American Heart Association estimates that more than 62 million Americans over the age of six suffer from high blood pressure, and that only a minority of these people have their blood pressure under control. Left untreated, hypertension can lead to stroke, heart attack, kidney damage, congestive heart failure, and death. Uncontrolled mild-to-moderate hypertension will reduce the life expectancy of a typical 35-year-old person by 16 years. Even the mildest form of high blood pressure, “borderline hypertension,” can cut one's life span by a few years and impact negatively on the quality of life.

In most cases, the mainstay of treatment for hypertension is medication. Drug treatment can bring blood pressure down quickly, and although it does not cure the disease, it does prevent the serious and even life-threatening complications that can result if high blood pressure is left untreated. Methods of treatment involve, for example, the use of an angiotensin converting enzyme (“ACE”) inhibitor, a calcium channel blocker or an angiotensin II receptor antagonist. These drugs can be used alone or in combination with each other or other drugs.

However, even with the availability of drugs for treating hypertension, not all patients afflicted with hypertension respond to these drugs, and there are significant adverse side effects. For example, ACE inhibitors may cause swelling of the mouth, tongue or throat, which could cause extremely serious risk and requires immediate medical care; persistent dry cough; dizziness; light-headedness due to low blood pressure; and potential death or injury to an unborn child (if taken during pregnancy). Calcium Channel inhibitors may cause edema, headache, fatigue, increased heart rate and dizziness. Angiotensin II receptor antagonists may lead to cough, dizziness, upper respiratory infection, allergic reactions, back and leg pain, diarrhea, indigestion, insomnia, muscle cramps or pain, nasal congestion, rash, sinus problems and swelling of face, lips, throat, and tongue.

Due to the potentially serious side effects of drug treatments for hypertension and the cost and inconvenience of having to change therapies due to non-responsiveness, a need exists to identify patients that will respond to therapy (e.g., optimal drug(s)) versus patients who will not respond to therapy, so that alternative therapy can be pursued.

### SUMMARY OF THE INVENTION

The present invention relates to methods for determining a patient's responsiveness to treatment for hypertension.

In one embodiment, the invention is directed to a method for predicting the efficacy of a drug for treating hypertension in a human patient, comprising: obtaining a sample of cells from the patient; obtaining a gene expression profile from the sample in the absence and presence of in vitro modulation of the cells with specific mediators; and comparing the gene expression profile of the sample with a reference gene expression profile, wherein similarity between the sample expression profile and the reference expression profile predicts the efficacy of the drug for treating hypertension in the patient. In a particular embodiment, the expression profile comprises expression values for one or more genes listed in FIGS. 2-4. In another embodiment, the invention further includes exposing the sample to the drug for treating hypertension prior to obtaining the gene expression profile of the sample.

In one embodiment, the drug is selected from a class of drugs selected from the group consisting of: an angiotensin converting enzyme inhibitor, a calcium channel blocker and an angiotensin II receptor antagonist. In a particular embodiment, the sample of cells is derived from blood and can comprise peripheral blood mononuclear cells (PBM cells or PBMCs). In a particular embodiment, the gene expression profile of the sample is obtained using a hybridization assay to oligonucleotides contained in a microarray. In one embodiment, the hybridization probes are capable of hybridizing to polynucleotides corresponding to the informative genes and reagents for detecting hybridization. In one embodiment, the gene expression profile of the sample is obtained by detecting the protein products of the informative genes. In another embodiment, the antibodies are capable of specifically binding protein products of the informative genes and reagents for detecting antibody binding. In one embodiment, the reference expression profile is that of cells derived from patients that do not have hypertension. In a particular embodiment, the cells are treated with the drug candidate before the expression profile is obtained.

### BRIEF DESCRIPTION OF THE DRAWINGS

FIG. 1 is a table describing the microarray parameters (e.g., number of chips and data points; an Affymetrix Hu95GeneFL was used) for data obtained for Cozaar™ (“CAR”), Norvasc™ (“NAC”) and Ramace™ (“RME”).

FIG. 2 is a table describing the genes that are used to predict Ramace™ responsiveness with greater than 85% accuracy. “class 0”=non-responders and “class 1”=responders. Gene names are indicated as GenBank accession numbers.

FIG. 3 is a table describing the genes that are used to predict Norvasc™ responsiveness with 80% accuracy or greater.

FIG. 4 is a table describing sets of genes that are used to predict Cozaar™ responsiveness with 82% accuracy or greater. “Condition 1” is baseline; “Condition 2” is the drug; “Condition 3” is pre-treatment with the drug for 45 minutes followed by stimulant; “Condition 4” is stimulant alone; “Condition ¾” is the difference in gene expression between Conditions 3 and 4. Five genes are best used in Condition 1; 9 genes are best used in Condition ¾; and 12 genes are best used for Condition 2.

FIGS. 5A-D are summaries of gene expression profile data. FIG. 5A shows results for Norvasc™. FIG. 5B shows results for Ramace™ (before and after drug treatment). FIG. 5C shows results for Ramace™ (responder versus non-responder data). FIG. 5D shows results for Cozaar™.

FIGS. 6.1-6.144 depict the differential expression data for Ramace™ response.

FIGS. 7.1-7.170 show the differential expression data for Norvasc™ response.

FIGS. 8.1-8.69 show the differential expression data for Cozaar™ response.

FIGS. 9.1-9.13 show the results of data mining algorithms (see Exemplification) of differential expression data.

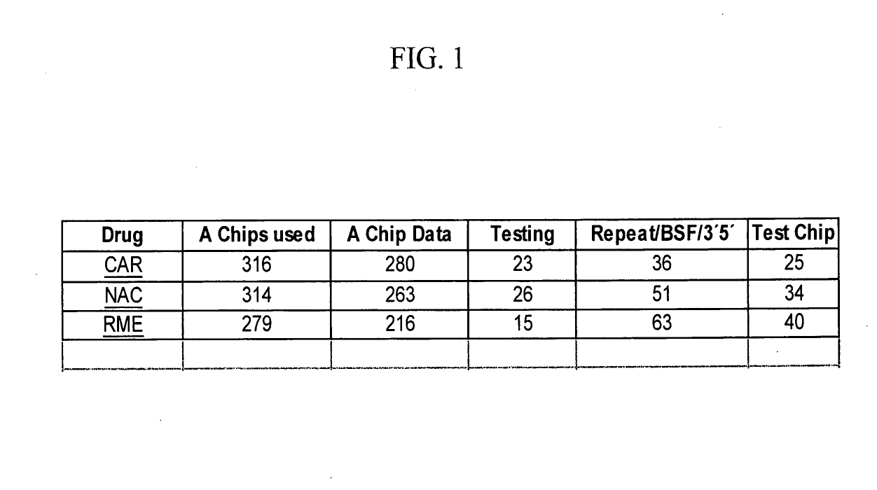

### DETAILED DESCRIPTION OF THE INVENTION

A description of preferred embodiments of the invention follows.

Hypertension is a major disease factor for coronary heart disease (CHD) as well as for heart failure, stroke, renal failure, and peripheral vascular disease. Around 50,000,000 Americans have hypertension, which was listed as a primary or contributing cause of death in about 202,000 of more that 2,000,000 U.S. deaths in 1996. Both high diastolic (DBP) and systolic (SBP) blood pressure have been shown to be related to higher events of stroke and myocardial infarction (fatal and nonfatal MI). The positive relationship between increased blood pressure and the risk of cardiovascular disease is well established and continues to be positive in terms of blood pressure levels and recurrent events (Flack, J. et al., 1995. Circulation, 92:2437-2445). There is also evidence that hypertensive patients have at least six times the risk of developing heart failure than normotensive patients and it has been estimated that each 5 mm Hg drop in DBP is associated with at least 25% decreased risk of end-stage renal disease (Klag, M. et al., 1996. N. Engl. J. Med., 334:13-18). Treatment methods are available for hypertension, however, due to significant side effects associated with these methods for treating hypertension, development of new drugs and screening methods for determining which patients will be responsive to treatment is important.

The present invention is directed to methods for predicting efficacy of drug treatment in patients afflicted with hypertension and to methods for screening drug candidates useful in treating hypertension. Current methods of treating hypertension involve, for example, the use of angiotensin converting enzyme inhibitors (“ACE” inhibitors), calcium channel inhibitors, and angiotensin-II inhibitors. As there are risks associated with methods for treating hypertension, identification of patients that will be responsive to treatment is important. Methods described herein are used to identify genes that are differentially expressed in responsive patients when compared to non-responsive patients, thereby allowing for a convenient determination of patients that are responsive to treatment. Described herein are methods for activating in cultured cells obtained from patient samples and methods for utilizing said cultured cells for drug screening and obtaining expression profiles.

ACE inhibitors, which include benazepril (Lotensin™), captopril (Capoten™), enalapril (Vasotec™), fosinopril (Monopril™), lisinopril (Prinivil™, Zestril™), moexipril (Univasc™), perindopril (Aceon™), quinapril (Accupril™), ramipril (Altace™, Ramace™), and trandolapril (Mavik™), block the production of angiotensin II, a chemical the body produces to raise blood pressure. Angiotensin's normal role is to maintain equilibrium when blood pressure drops. It acts directly on the arteries, tightening them up to raise the pressure. The ACE inhibitors can bring blood pressure down quickly but in very rare cases can cause kidney damage or a reduction in the number of white blood cells (leading to an increased susceptibility to infection).

Calcium channel blockers are the most widely prescribed drugs in the United States today. Like other drugs used for hypertension, they act by dilating the arteries and reducing resistance to the flow of blood. They have proved to be beneficial not only for high blood pressure, but also for angina and other problems of a weakened heart. Included in this group are drugs such as amlodipine (Norvasc™), bepridil (Vascor™), diltiazem (Cardizem™, Dilacor XR™, Tiazac™), felodipine (Plendil™), isradipine (DynaCirc™), nicardipine (Cardene™), nimodipine (Nimotop™), nisoldipine (Sular™), and verapamil (Calan™, Covera-HS™, Isoptin™, Verelan™). Some calcium channel blockers are now available combined with an ACE inhibitor in a single pill. Among these medications are brands named Lexxel™, Lotrel™, and Tarka™.

Angiotensin II receptor antagonists, work to lower blood pressure by blocking angiotensin from binding to receptor sites in the smooth muscles of the blood vessels. This blocking action stops the angiotensin from tightening the arteries and raising the blood pressure. The class of angiotensin II receptor antagonists includes, for example, valsartan (Diovan™), candesartan (Atacand™), eprosartan (Teveten™), irbesartan (Avapro™), losartan potassium (Cozaar™), olmesartan (Benicar™), and telmisartan (Micardis™). Most of these drugs are also available combined with a diuretic.

The present invention relates to methods for determining the treatment outcome of drugs used to treat hypertension. The methods rely on the identification of genes that are differentially expressed in samples obtained from patients and are associated with, for example, clinical responsiveness to the drug under study. The particular genes, herein referred to as “informative genes.” Informative genes are genes that are generally differentially expressed in different samples. For example, informative genes can be identified in cells that have been induced to mimic the disease condition (e.g., hypertension), or in tissue samples from patients diagnosed with hypertension, wherein the informative genes are differentially expressed in induced versus uninduced cells or in hypertensive versus non-hypertensive patients. Informative genes can be identified, for example, by determining the ratio of gene expression in induced versus uninduced cells and comparing the results between patients with variable drug sensitivity. Alternatively, informative genes can be identified based on the ratio of gene expression in disease versus normal tissue samples, or, in the case of informative genes used to identify drug responsiveness, informative genes can be identified by the ratio of gene expression in cells exposed to the drug versus cells not exposed to the drug, in subjects who qualify as responders versus non-responders to the drug. A ratio of 1.0 would indicate the gene is expressed at the same level in both samples. Ratios greater than one indicate increased expression over normal or uninduced cells, whereas ratios less than one indicate reduced expression relative to normal or uninduced cells.

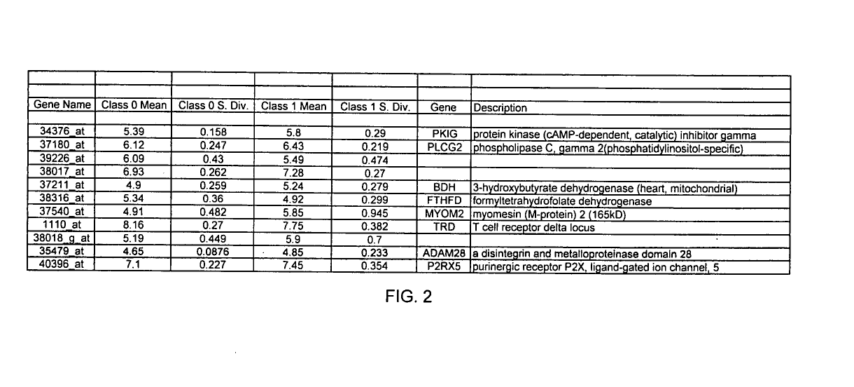

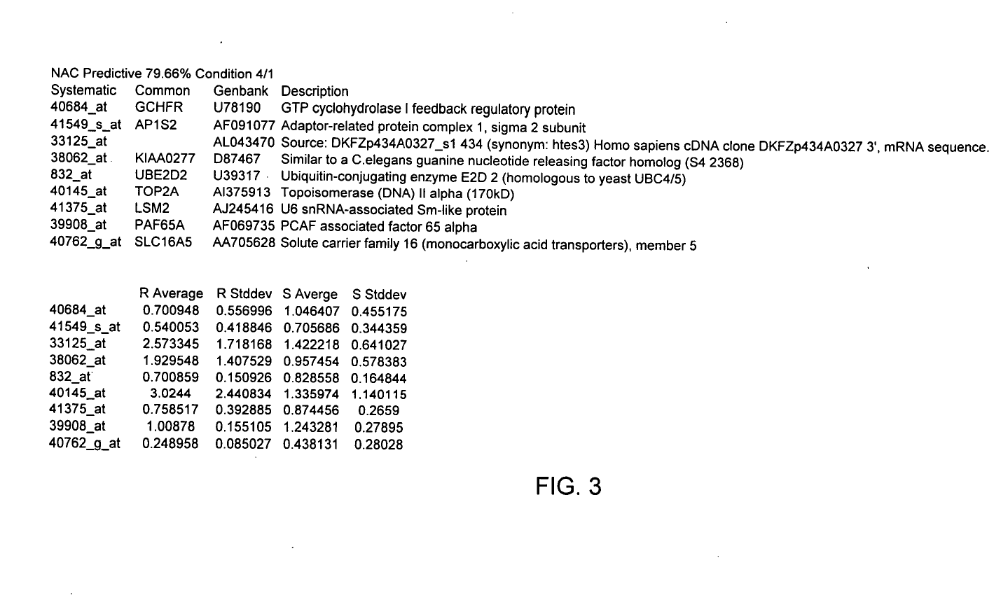

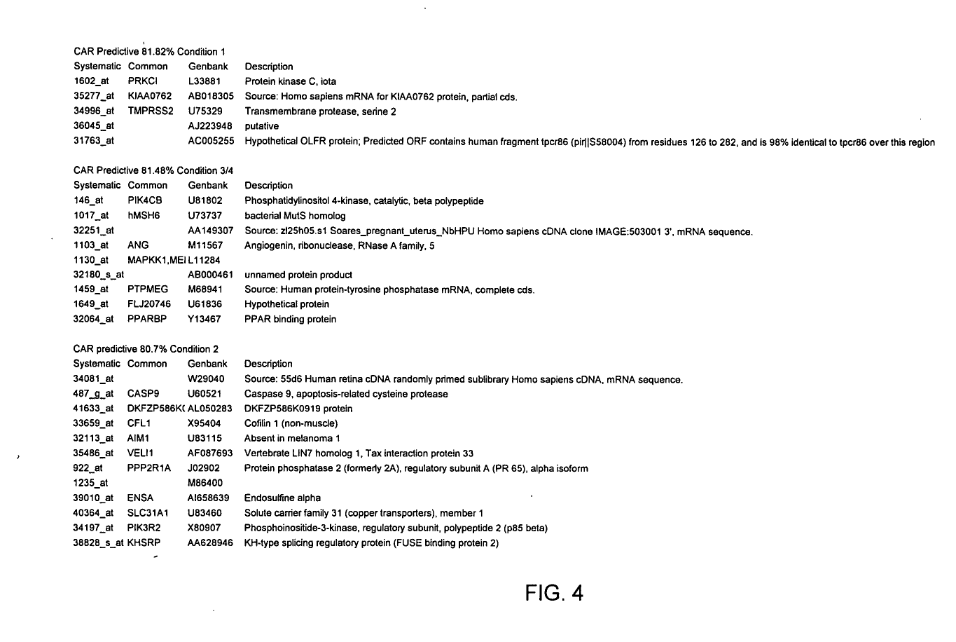

A subset or all informative genes can be assayed for gene expression in order to generate an “expression profile” for responsive versus non-responsive patients. As used herein, an “expression profile” refers to the level or amount of gene expression of one or more informative genes in a given sample of cells at one or more time points. A “reference” expression profile is a profile of a particular set of informative genes under particular conditions such that the expression profile is characteristic of a particular condition. For example, a reference expression profile that quantitatively describes the expression of the informative genes listed in FIGS. 2-4 can be used as a reference expression profile for drug treatment responsiveness. In one embodiment, expression profiles are comprised of the genes of FIG. 2 (including, e.g., subgroups of informative genes) to predict responsiveness to Ramace™. Eleven informative genes are listed in FIG. 2 that exhibit differential expression, under different conditions, and provide sufficient power to predict the responsiveness to the drug with high accuracy. In one embodiment, expression profiles are comprised of the genes of FIG. 3 (including, e.g., subgroups of informative genes) to predict responsiveness to Norvasc™. Nine informative genes are listed in FIG. 3 that exhibit differential expression, under different conditions, and provide sufficient power to predict the responsiveness to the drug with high accuracy. In one embodiment, expression profiles are comprised of the genes of FIG. 4 (including, e.g., subgroups for Condition 1, ¾ or 2 as indicated) to predict responsiveness to Cozaar™. Twenty-six informative genes are listed in FIG. 4 that exhibit differential expression, under different conditions, and provide sufficient power to predict the responsiveness to the drug with high accuracy. Other embodiments can include, for example, expression profiles containing about 5 informative genes, about 25 informative genes, about 100 informative genes, or any number of genes in the range of about 5 to about 400 informative genes. The informative genes that are used in expression profiles can be genes that exhibit increased expression over normal cells or decreased expression versus normal cells. The particular set of informative genes used to create an expression profile can be, for example, the genes that exhibit the greatest degree of differential expression, or they can be any set of genes that exhibit some degree of differential expression and provide sufficient power to accurately predict the responsiveness to the drug. The genes selected are those that have been determined to be differentially expressed in either a disease, drug-responsiveness, or drug-sensitive cell relative to a normal cell and confer power to predict the response to the drug. By comparing tissue samples from patients with these reference expression profiles, the patient's susceptibility to a particular disease, drug-responsiveness, or drug-resistance can be determined.

The generation of an expression profile requires both a method for quantitating the expression from informative genes and a determination of the informative genes to be screened. The present invention describes screening specific changes in individuals that affect the expression levels of gene products in cells. As used herein, “gene products” are transcription or translation products that are derived from a specific gene locus. The “gene locus” includes coding sequences as well as regulatory, flanking and intron sequences. Expression profiles are descriptive of the level of gene products that result from informative genes present in cells. Methods are currently available to one of skill in the art to quickly determine the expression level of several gene products from a sample of cells. For example, short oligonucleotides complementary to mRNA products of several thousand genes can be chemically attached to a solid support, e.g., a “gene chip,” to create a “microarray.” Specific examples of gene chips include Hu95GeneFL (Affymetrix, Santa Clara, Calif.), which was used in the Examples below, and the 6800 human DNA gene chip (Affymetrix, Santa Clara, Calif.). Such microarrays can be used to determine the relative amount of mRNA molecules that can hybridize to the microarrays (Affymetrix, Santa Clara, Calif.). This hybridization assay allows for a rapid determination of gene expression in a cell sample. Alternatively, methods are known to one of skill in the art for a variety of immunoassays to detect protein gene expression products. Such methods can rely, for example, on conjugated antibodies specific for gene products of particular informative genes.

Gene expression profiles can be used to identify informative genes based on clustering of gene expression profiles of individual genes, as determined by, for example, algorithms known in the art (Golub, T. et al., 1999., Science, 286:531-537). Such algorithms allow for the clustering of particular genes into groups that are indicative of particular classes (e.g., responders versus non-responders).

Informative genes can be identified and determined empirically, for example, in samples obtained from individuals identified through database screening to have a particular trait, e.g., drug sensitivity or drug resistance. In addition, informative genes identified in cultured cells can be verified by obtaining expression profiles from samples of known hypertension patients that are either responsive or non-responsive to a particular drug treatment. An example of a combination of obtaining samples from patients and searching particular databases for the genealogical and medical history of the individual from whom the sample was obtained is herein described for the genetically isolated population of Iceland.

The population of Iceland offers a unique opportunity to identify genetic elements associated with particular disorders. The unique opportunity is available due to at least three conditions: 1) the Icelandic population is genetically isolated; 2) detailed genealogical records are available; and 3) detailed medical records have been kept dating back to 1915. The identification of differentially expressed genes in responsive versus non-responsive patients would occur after an examination of a patient's genealogical past as well as the medical records of close relatives in addition to data obtained from samples derived from the individual.

An examination of genealogical and medical records identifies modem day individuals with a family history of exhibiting a particular trait. For example, individuals can be found that are afflicted with hypertension and that respond to a particular hypertension drug treatment, and an examination of a genealogical database might confirm that indeed the individual's close relatives exhibit the same traits, on average, more than the rest of the population. Thus, a tentative conclusion can be drawn that the individual in question likely has genetic determinants that could be used to identify responsive and non-responsive patients. Samples obtained from this individual, combined with samples obtained from other such individuals, are genotyped by any of the methods described above in order to identify informative genes that can subsequently be used to generate reference expression profiles.

Informative genes can also be identified ex vivo in cells derived from patient samples. For example, a tissue sample can be obtained from a patient and cells derived from this sample can be cultured in vitro. The cells can be cultured in the presence or absence of activators or other mediators such as, for example, angiotensin I, angiotensin II or Ca²⁺. As used herein, “mediator” refers to a molecular signal for a particular event. Cytokines are an example of a class of mediators that are low molecular weight, pharmacologically active proteins that are secreted by one cell for the purpose of altering either its own functions (autocrine effect) or those of adjacent cells (paracrine effect). In some instances, cytokines enter the circulation and have one or more of their effects systemically. Expression profiles of informative genes can be obtained from sample-derived cells in the presence and/or absence of cytokines or other mediators, and these profiles can be compared to reference expression profiles to determine sensitivity or resistance to drug treatment. Additionally, cells can be cultured in the presence or absence of the drug itself prior to obtaining the expression profile.

Once informative genes have been identified, polymorphic variants of informative genes can be determined and used in methods for detecting disorders in patient samples based on which polymorphic variant is present in the sample (e.g., through hybridization assays or immune detection assays using antibodies specific for gene products of particular polymorphic variants).

Alternatively, the approach described above can be used to verify the utility of informative genes identified in cultured cells. Once identified, informative genes can be verified as to their predictive ability in more genetically diverse populations, thus ensuring the utility of the predictive power of these informative genes in populations in addition to the genetically isolated population of, e.g., Iceland.

The “genetic isolation” of the Icelandic population implies a low degree of allelic variation among individuals. This circumstance reduces the background in screening for differences in a population. In “genetically diverse” populations, many differences appear between individuals that might contribute to the same trait. For example, an examination of individuals responsive for hypertension drug treatment might produce a finite yet large number of genetic differences with respect to non-responsive individuals. However, in a genetically diverse population, a great majority of these genetic differences are “artifactual” or background “noise signals” detected because of the diversity of the population. For a genetically isolated population, fewer differences would be expected to be found between the two groups, providing a higher probability that the differences that are discovered are likely to be directly related to the trait in question, in this case, responsiveness to hypertension drug treatment. Once determined in a genetically isolated environment, the utility of informative genes and expression profiles based on those informative genes can be verified for more general use in a genetically diverse population.

The invention will be further described with reference to the following non-limiting examples. The teachings of all the patents, patent applications and all other publications and websites cited herein are incorporated by reference in their entirety.

### EXEMPLIFICATION

Many classes of medications are available for the treatment of hypertension, including, for example, diuretics, α-blockers, β-blockers, ACE inhibitors, angiotensin II receptor antagonists (AIIAs), calcium channel blockers, and nitrates. Each class includes several to numerous distinct drugs. Because every drug is chemically unique, each is potentially vulnerable to the effects of a polymorphism in a particular patient, especially a polymorphism in the ligand-binding domain that confers drug resistance. The initial observation that certain drugs vary in their efficacy among ethnic groups (such as the diminished effectiveness of β-blockers and ACE inhibitors as monotherapy for hypertension in African American patients compared with Caucasian patients), has been refined by the identification of polymorphisms of α1A-adrenergic receptors, β2-adrenergic receptors, and ACE inhibitors that affect both short-term drug efficacy (e.g., blood pressure) and long-term efficacy (e.g., progression of diabetic nephropathy in patients with insulin-dependent diabetes mellitus). Despite the many therapeutic options, only 27% of patients with hypertension achieve adequate control of blood pressure in accordance with current recommendations from the Sixth Report of the Joint National Committee on Prevention, Detection, Evaluation, and Treatment of High Blood Pressure (1997. Arch. Intern. Med., 157:2413-2446). Therefore, hypertension management through judicious use of pharmacogenomics represents a prime opportunity for individualization of pharmacotherapy to enhance patient care.

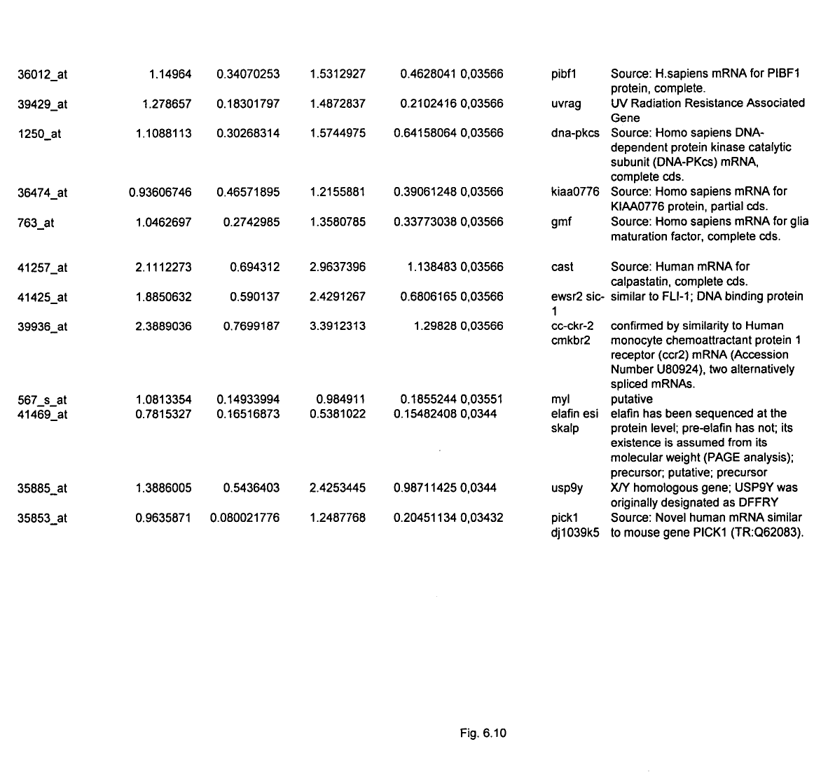

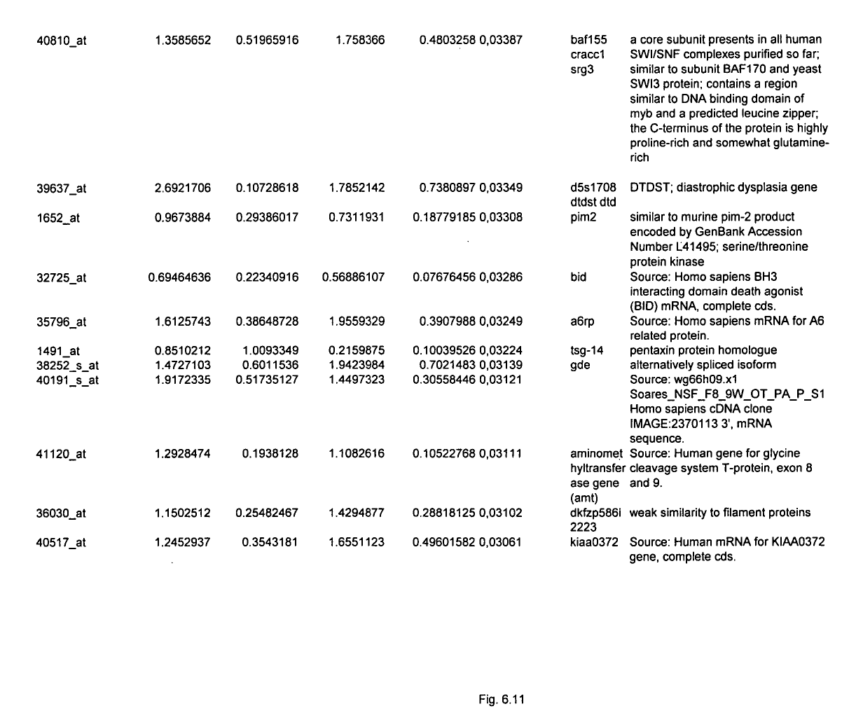

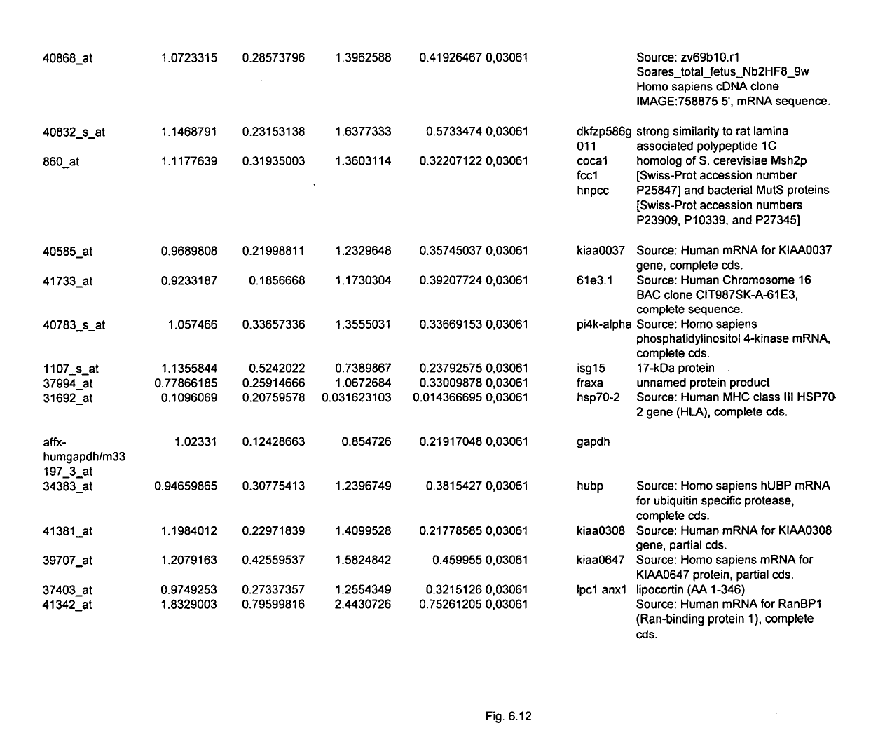

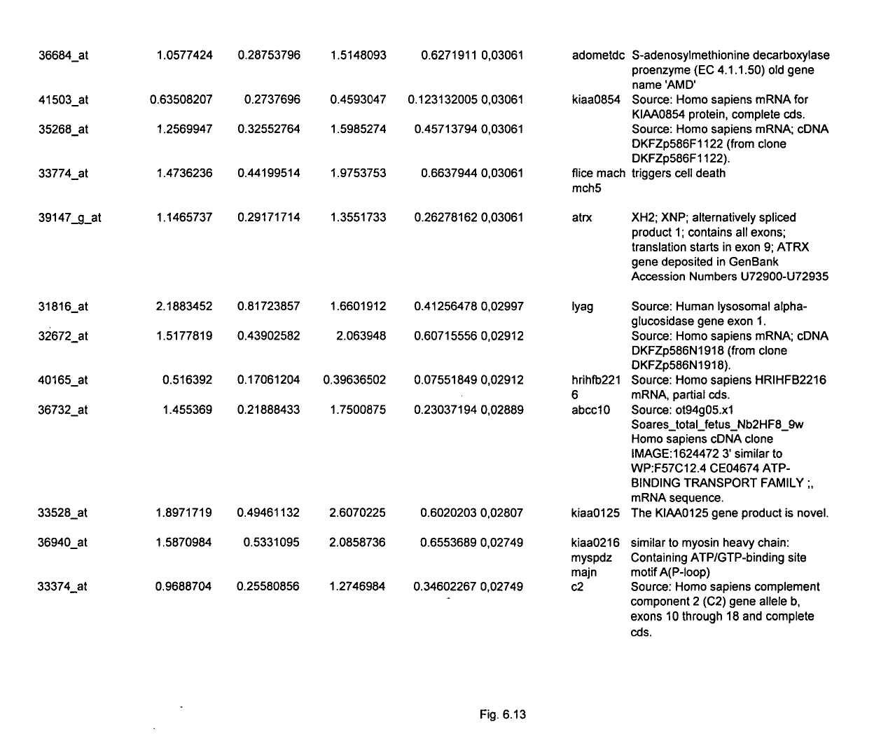

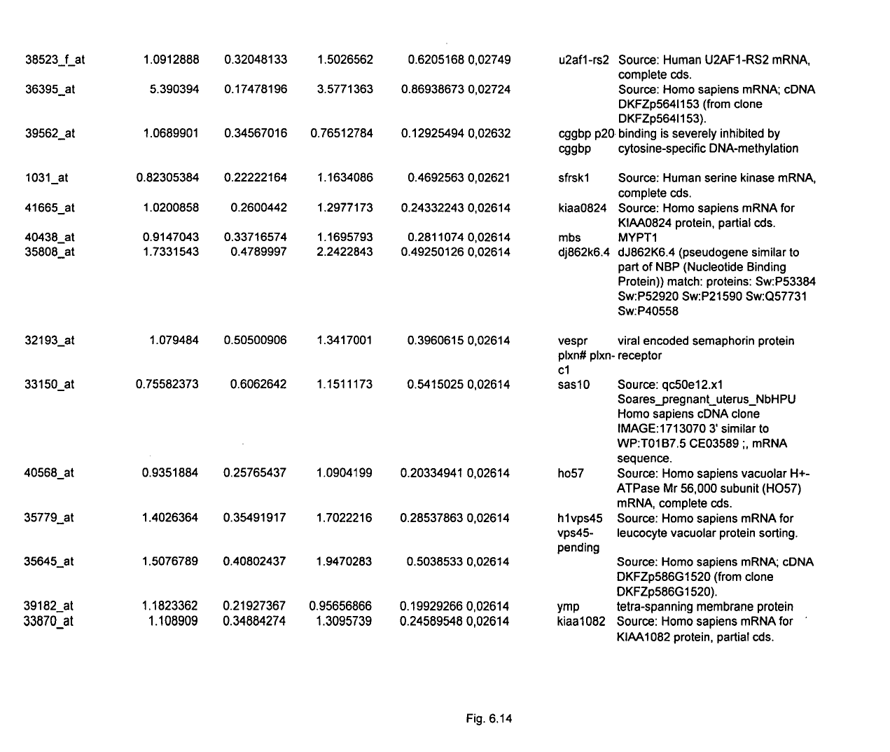

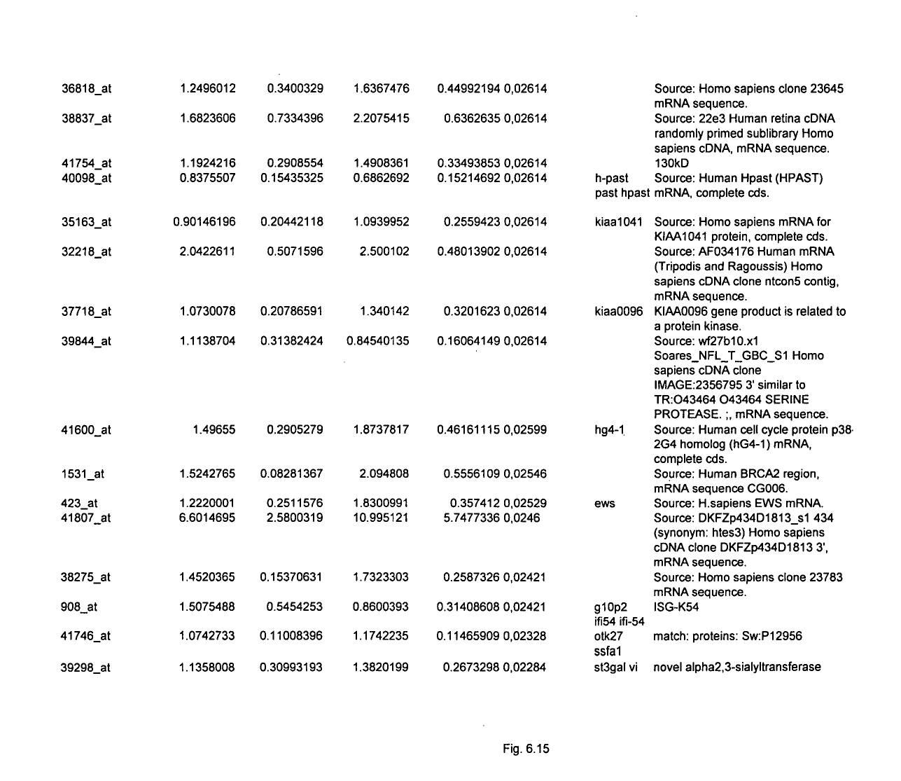

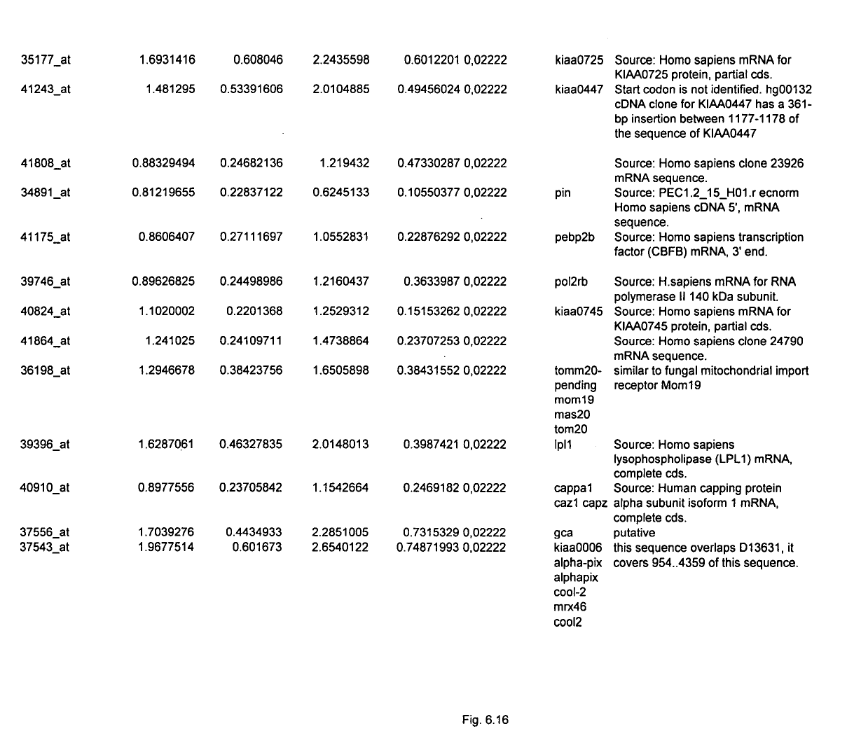

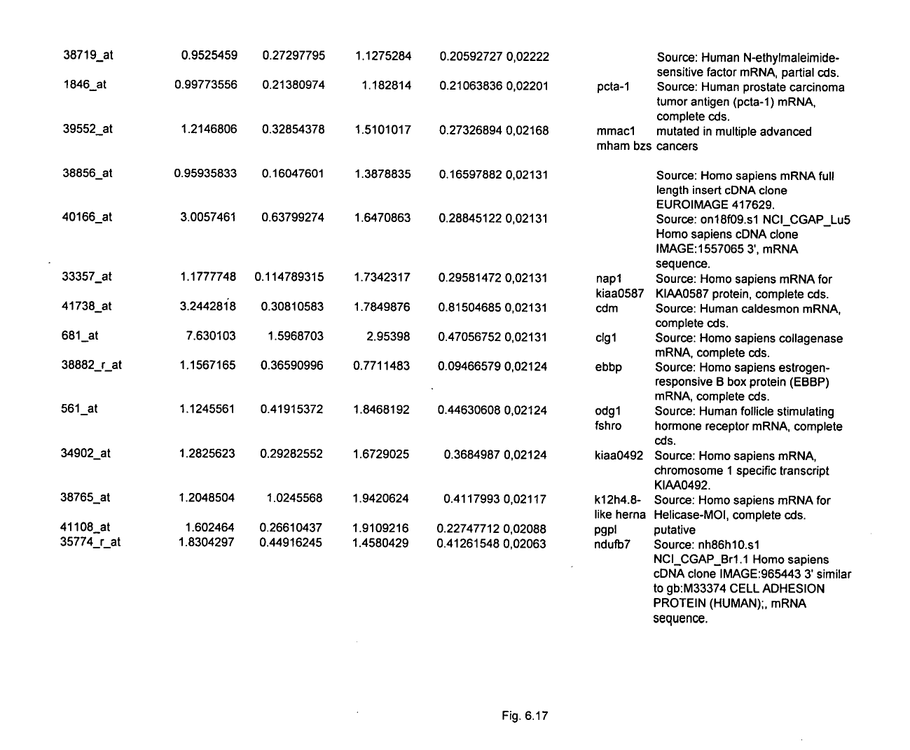

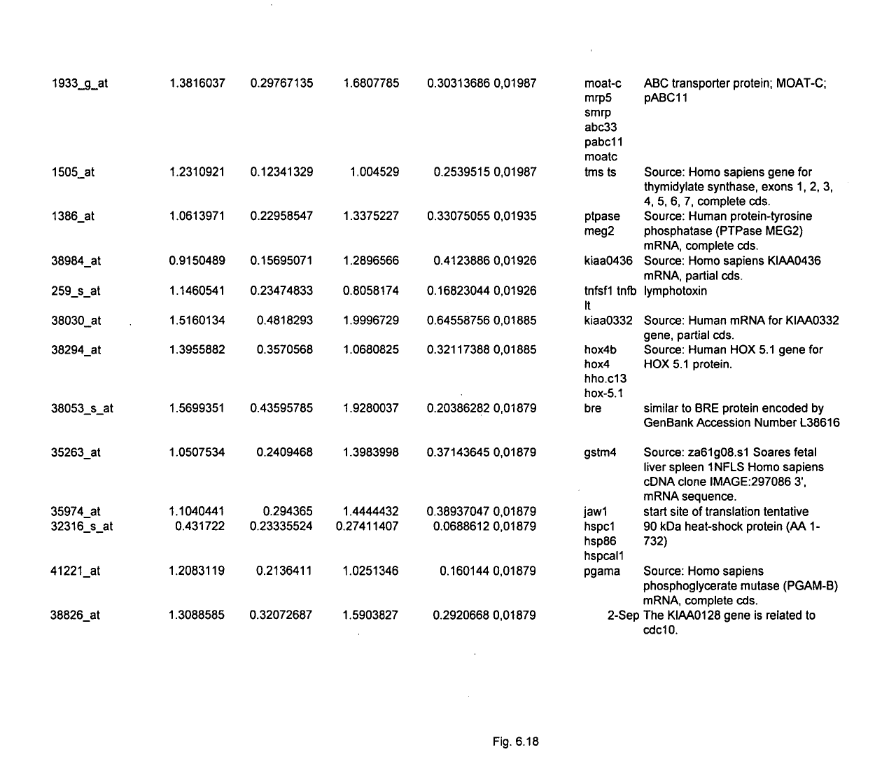

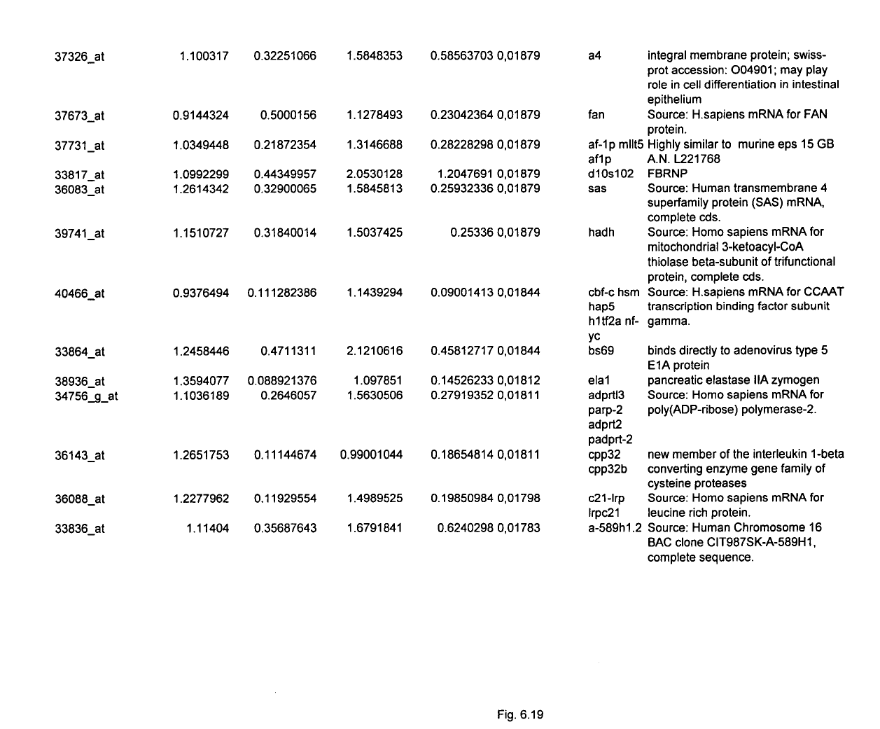

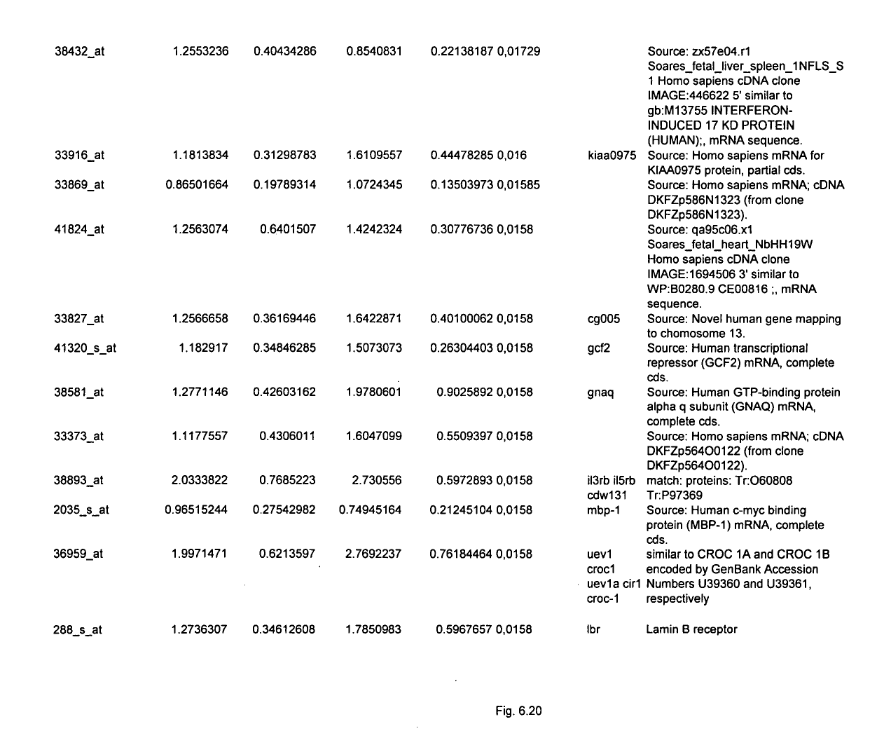

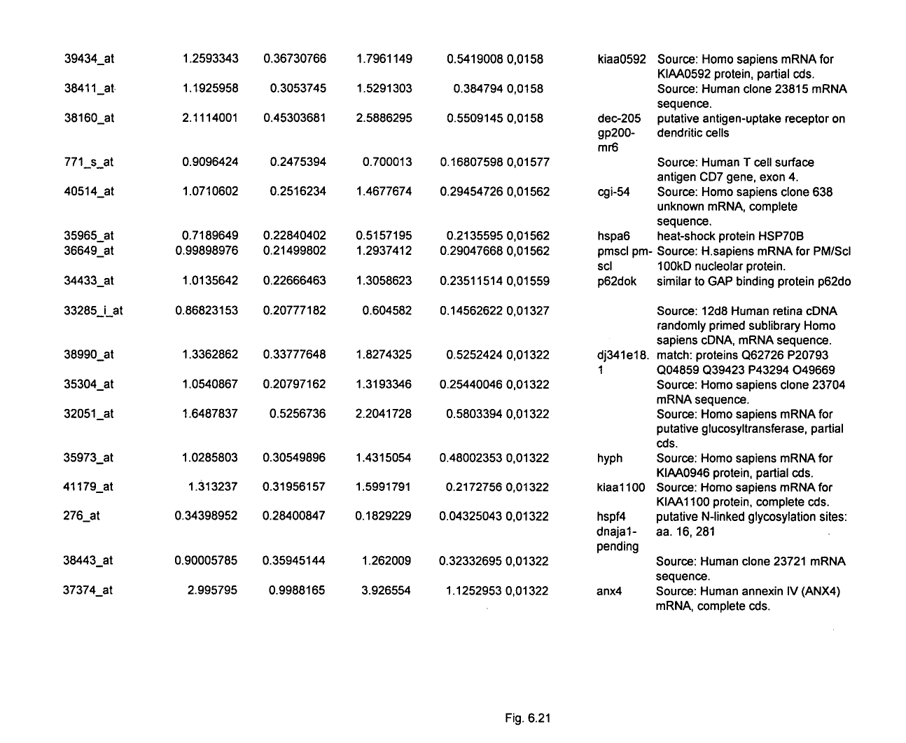

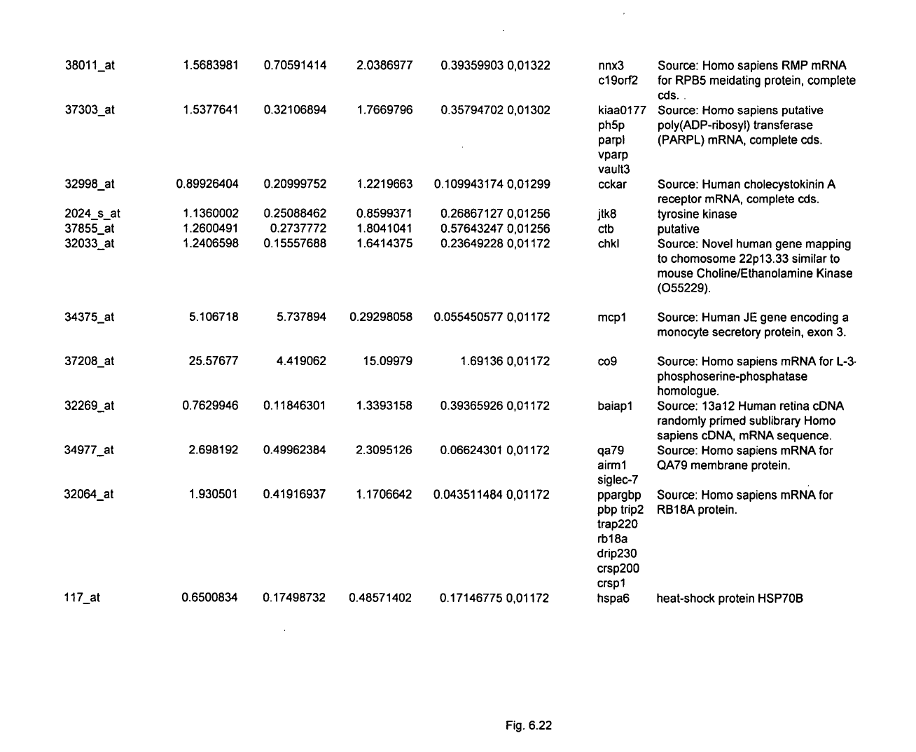

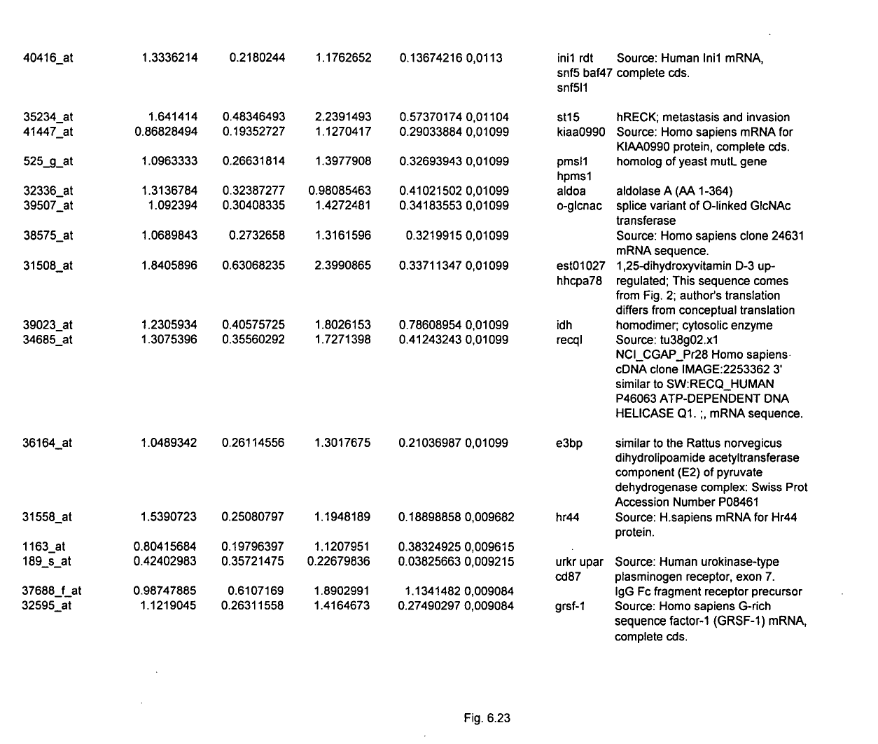

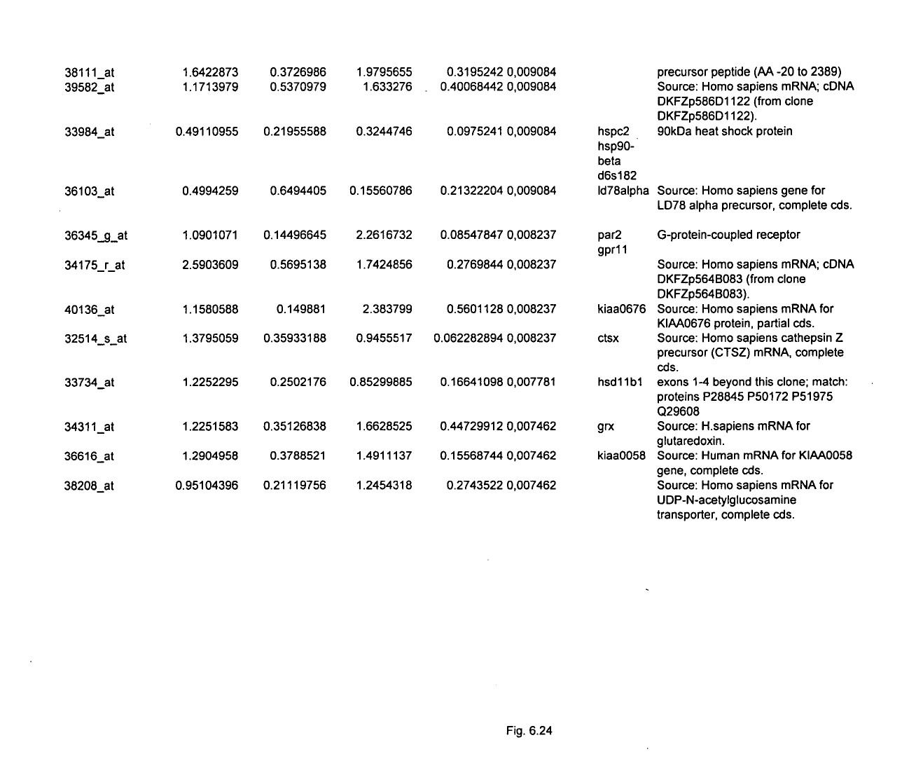

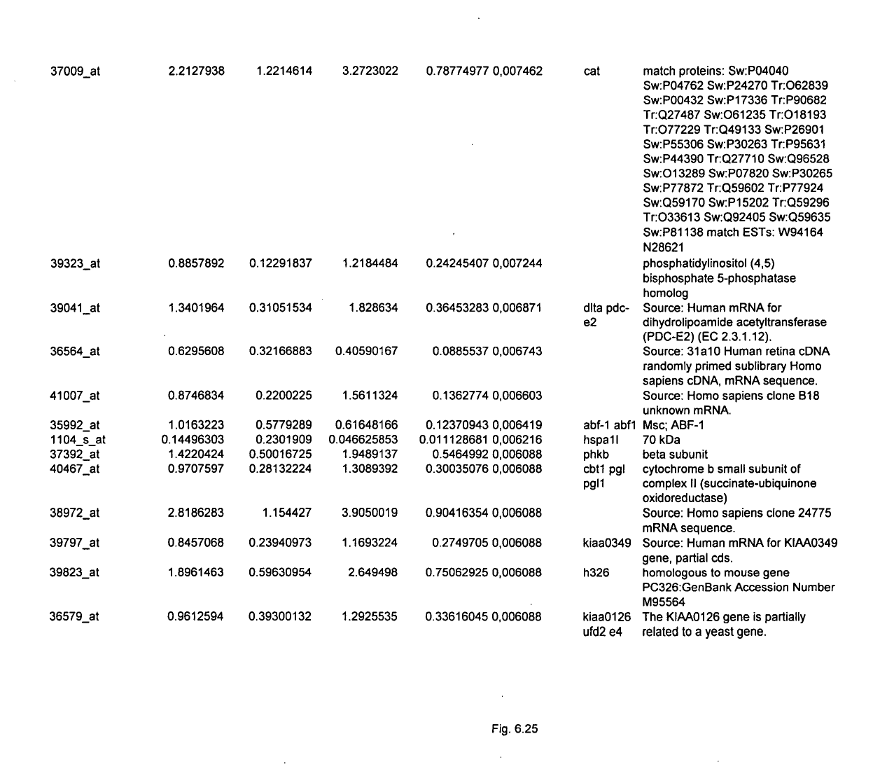

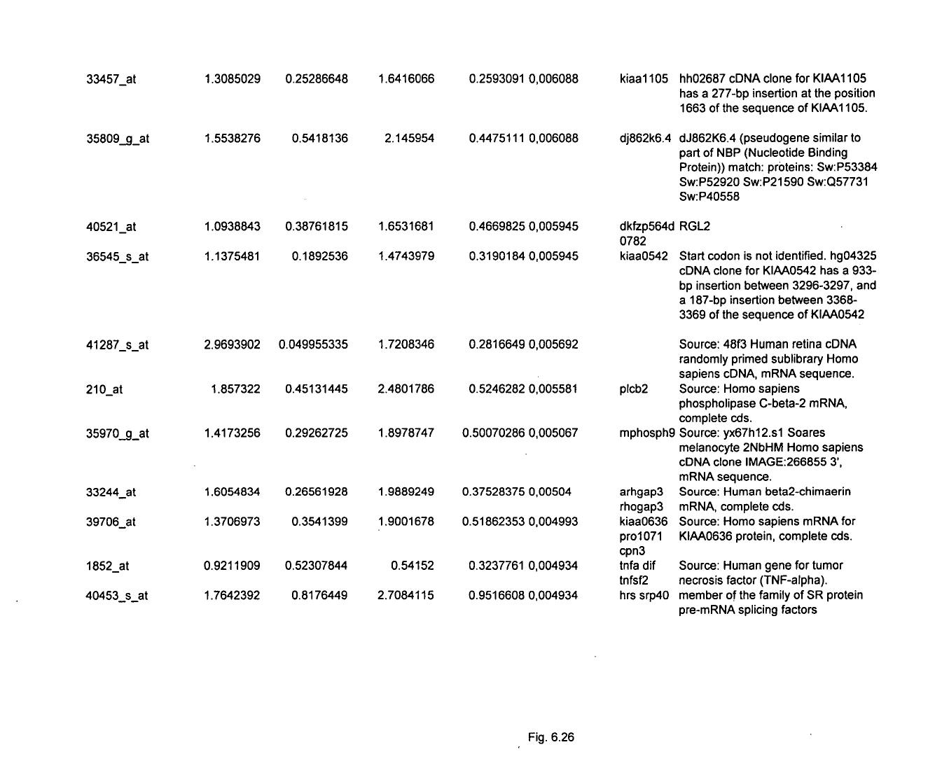

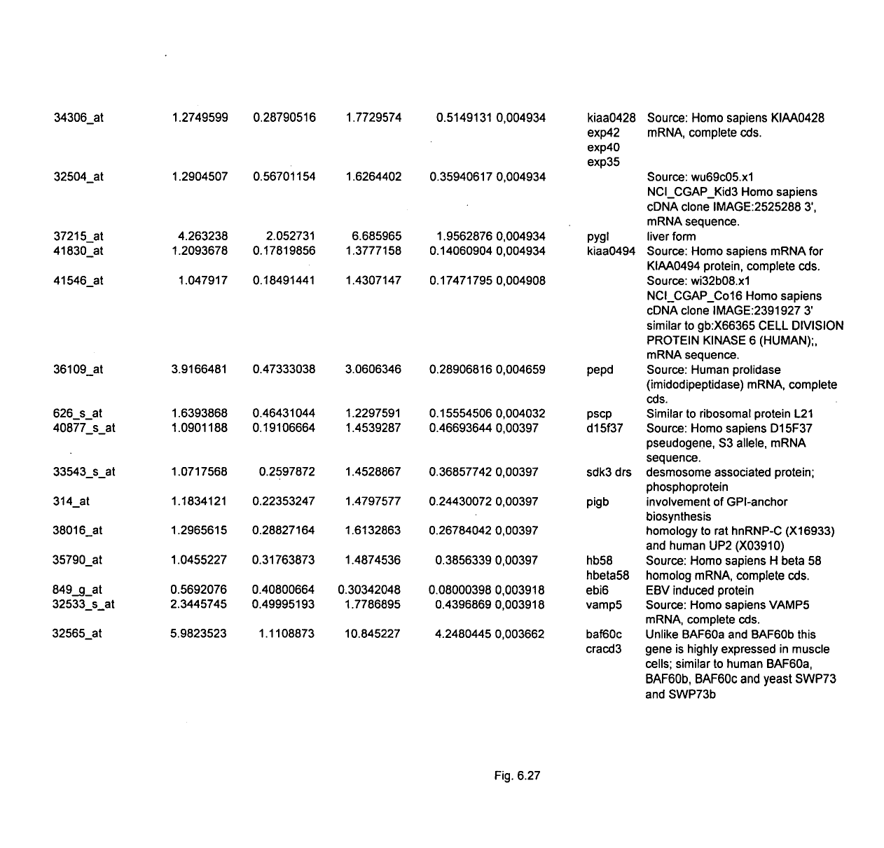

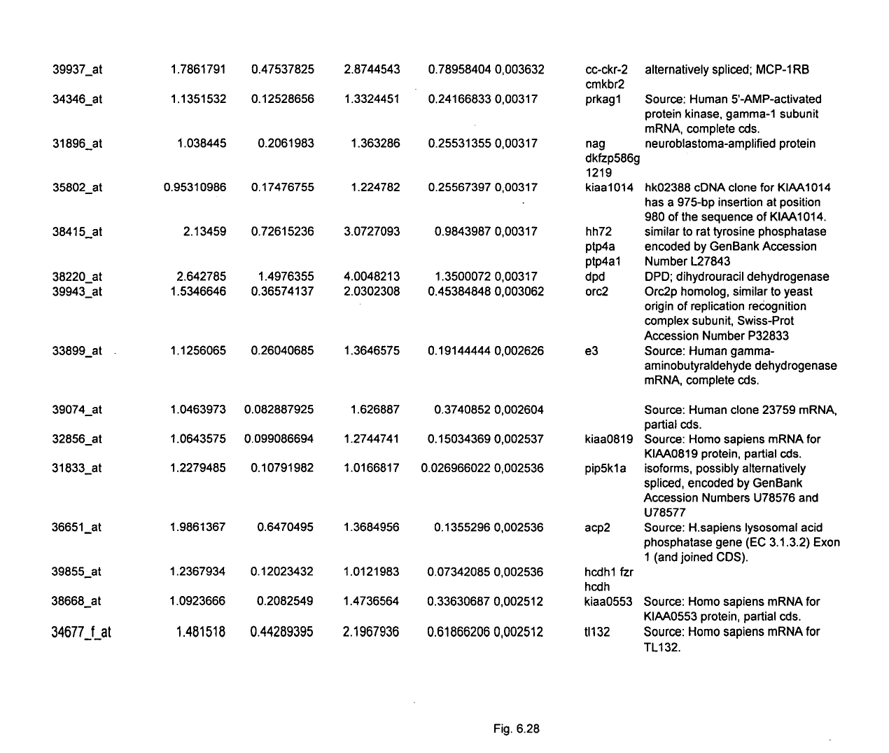

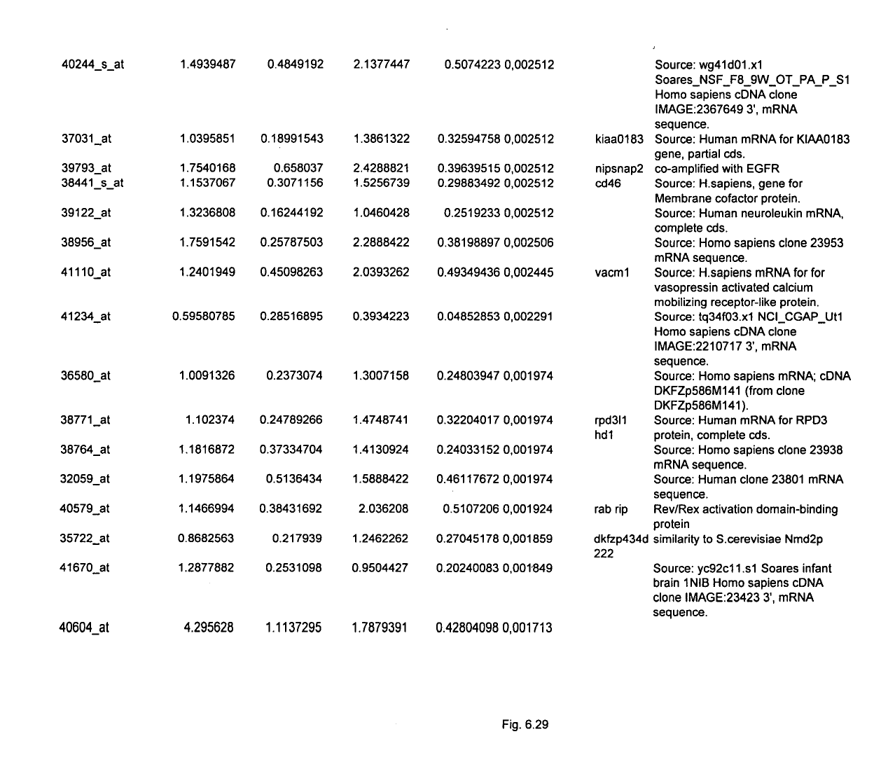

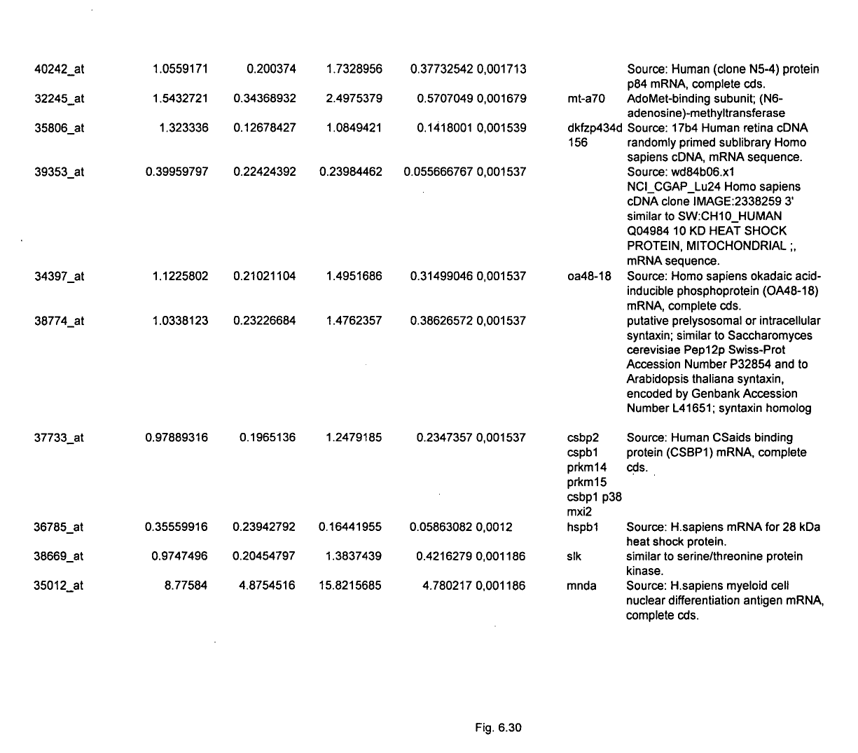

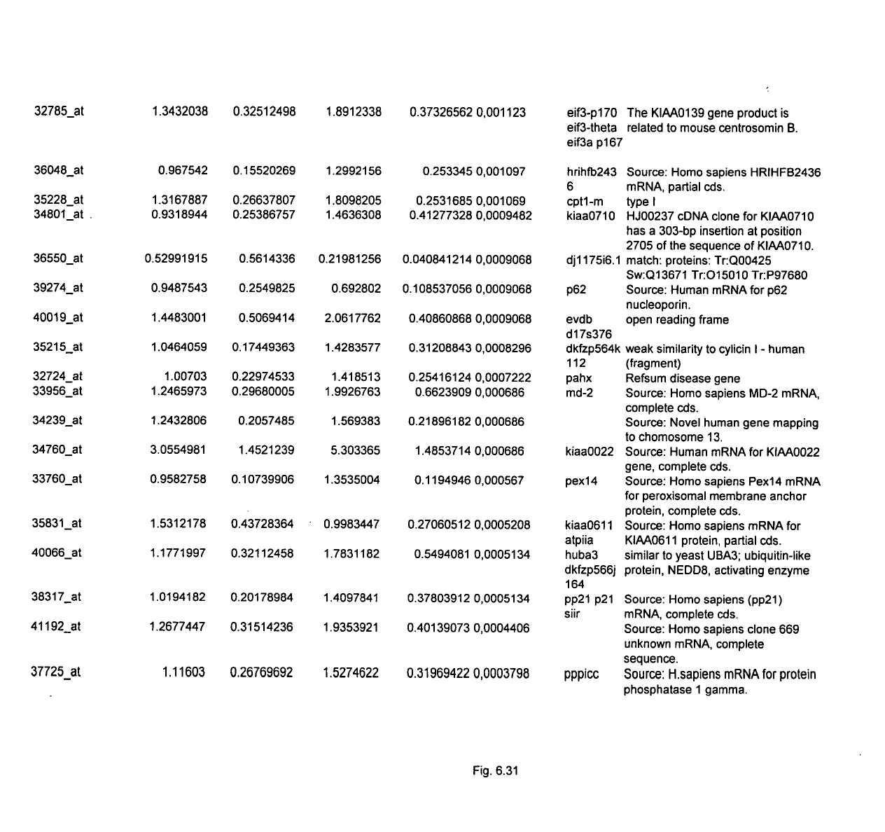

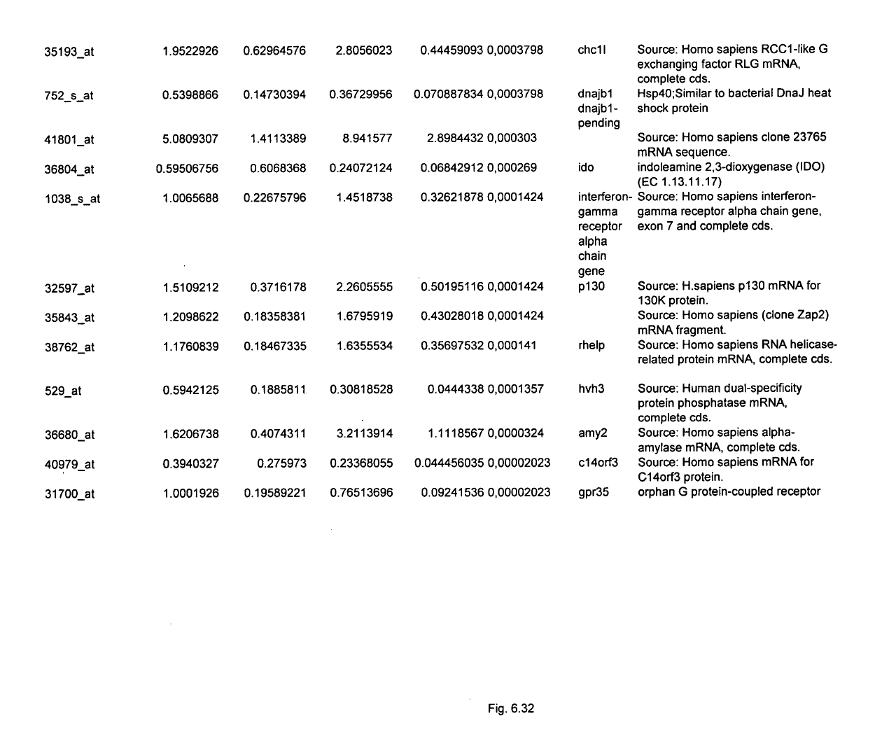

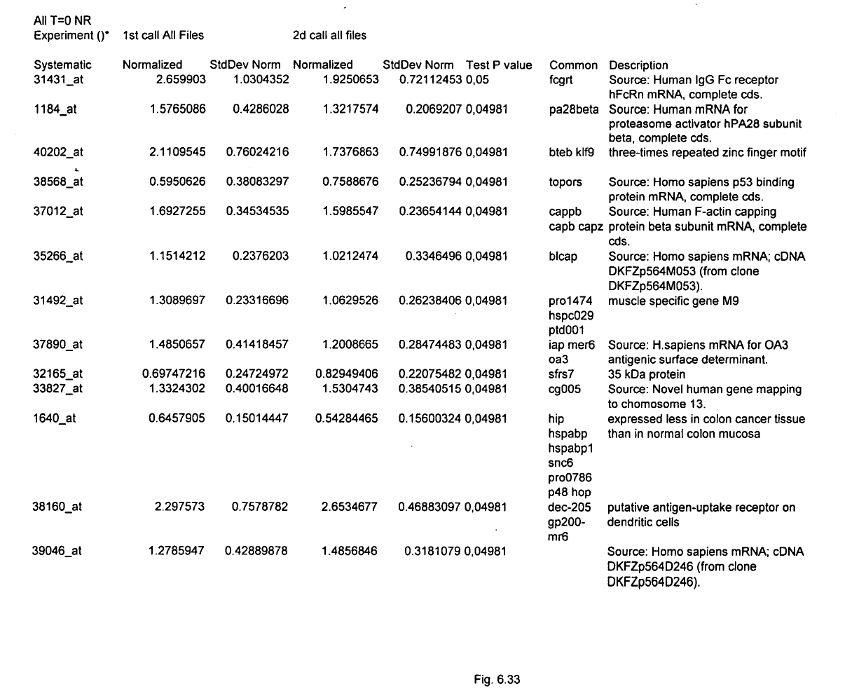

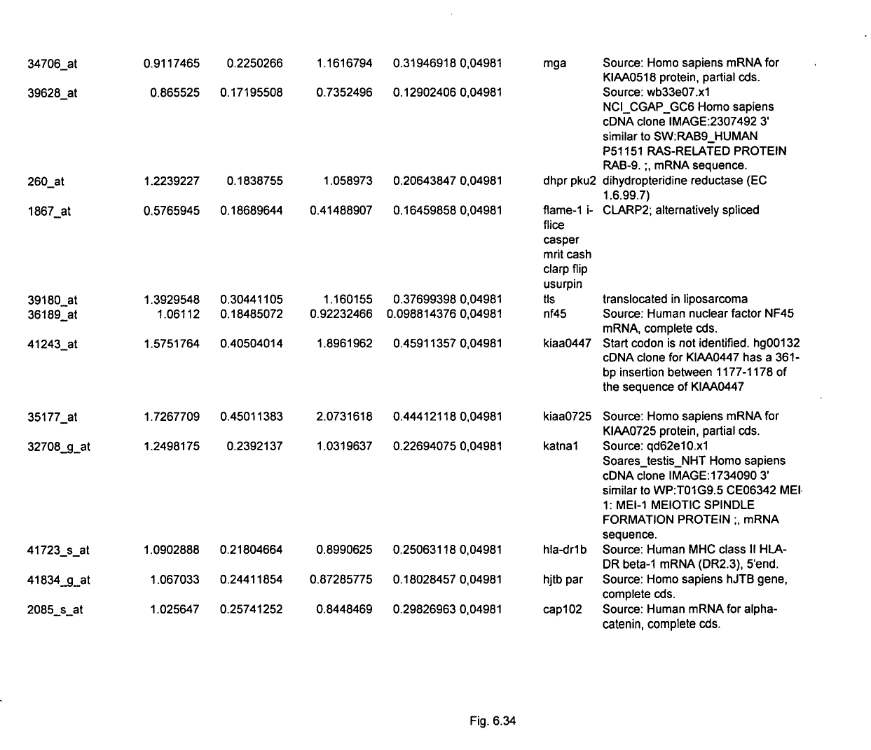

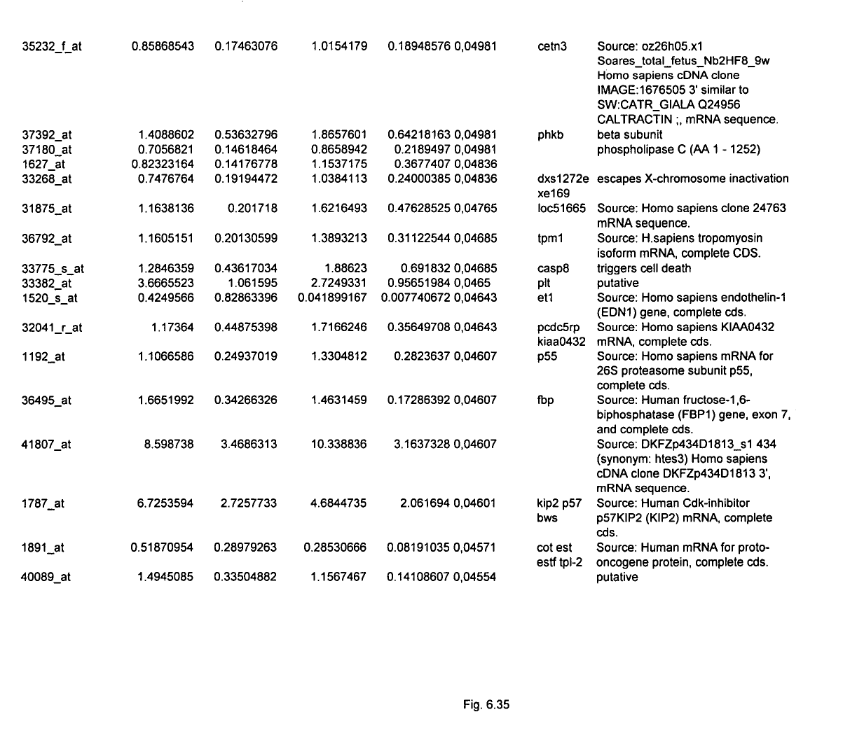

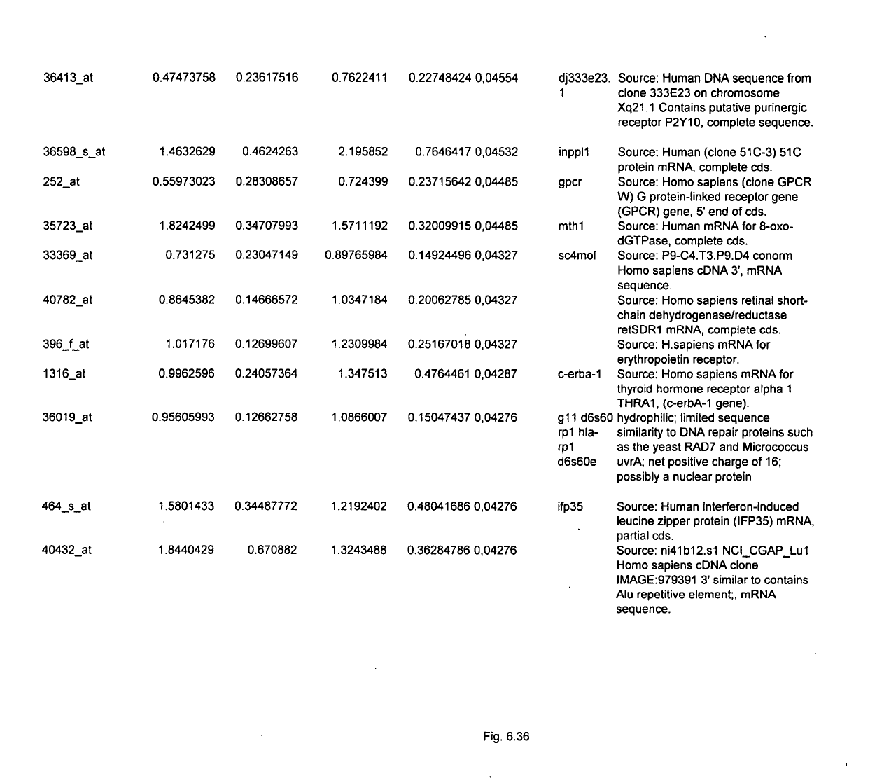

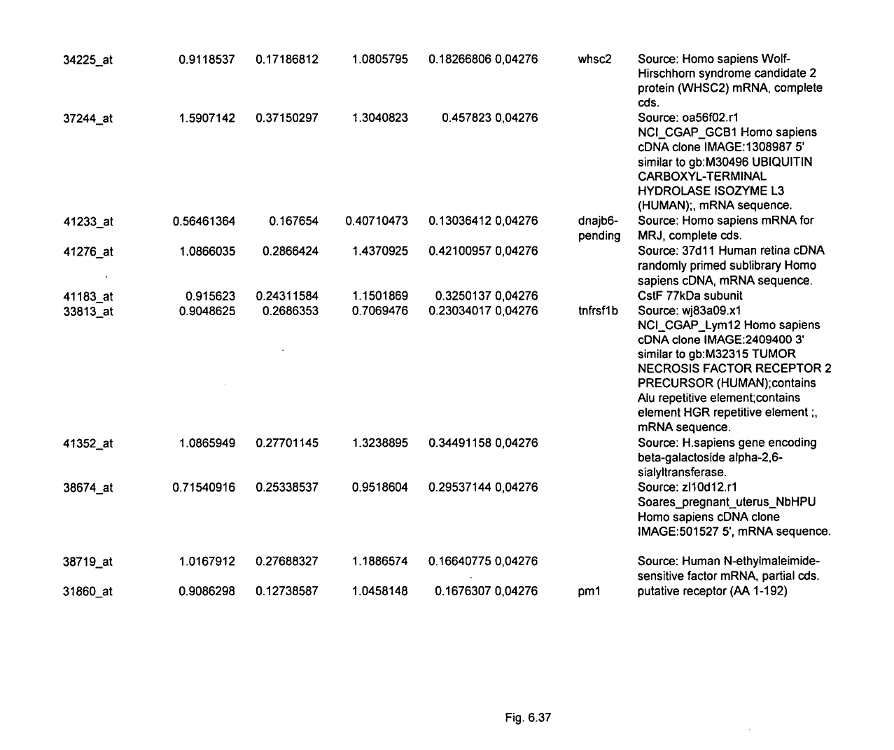

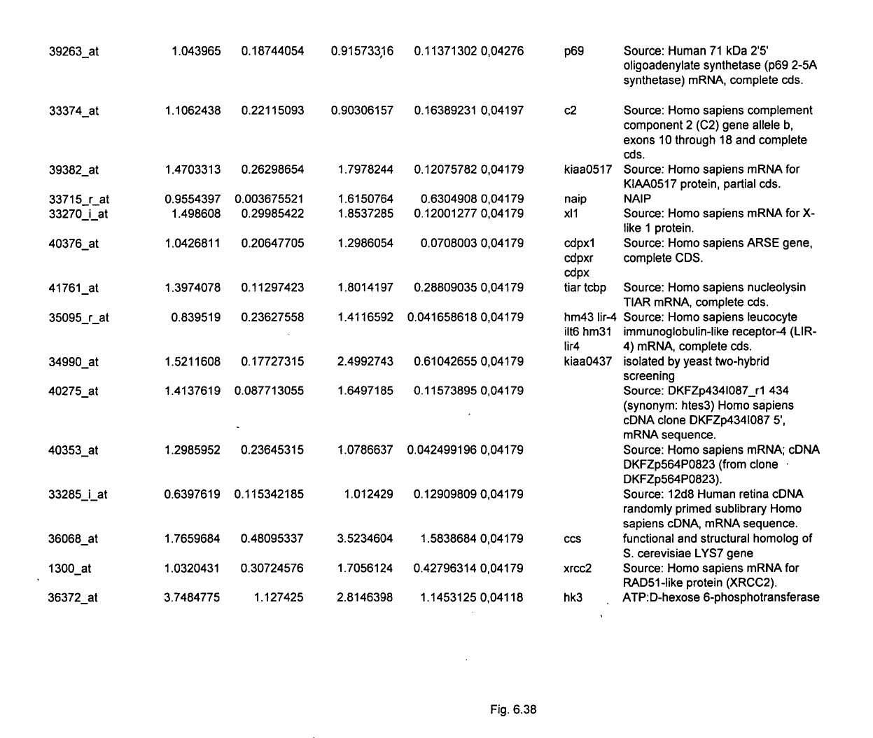

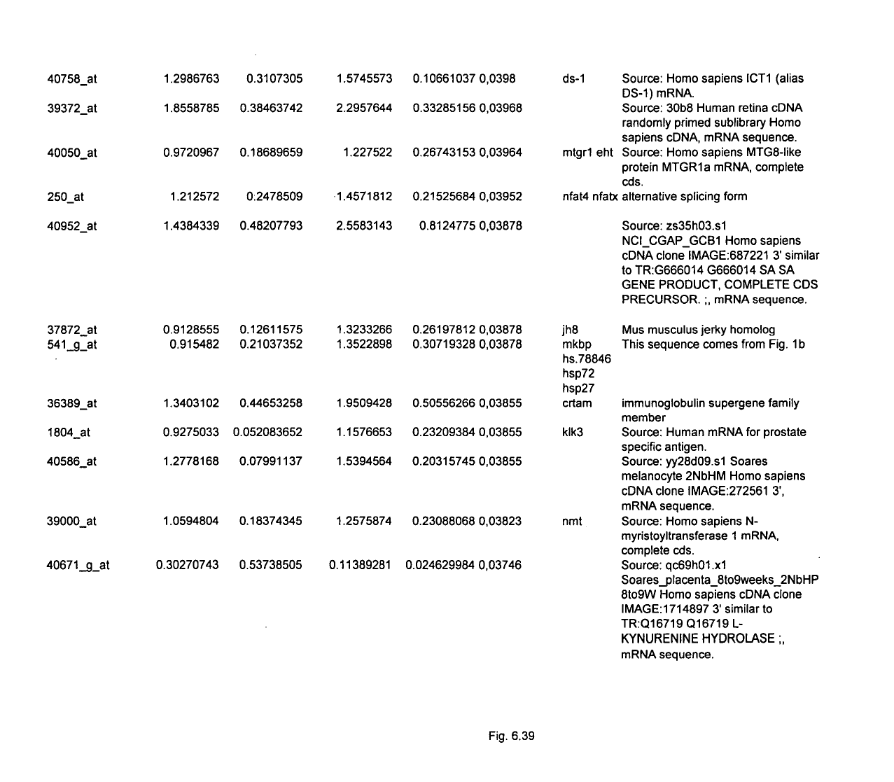

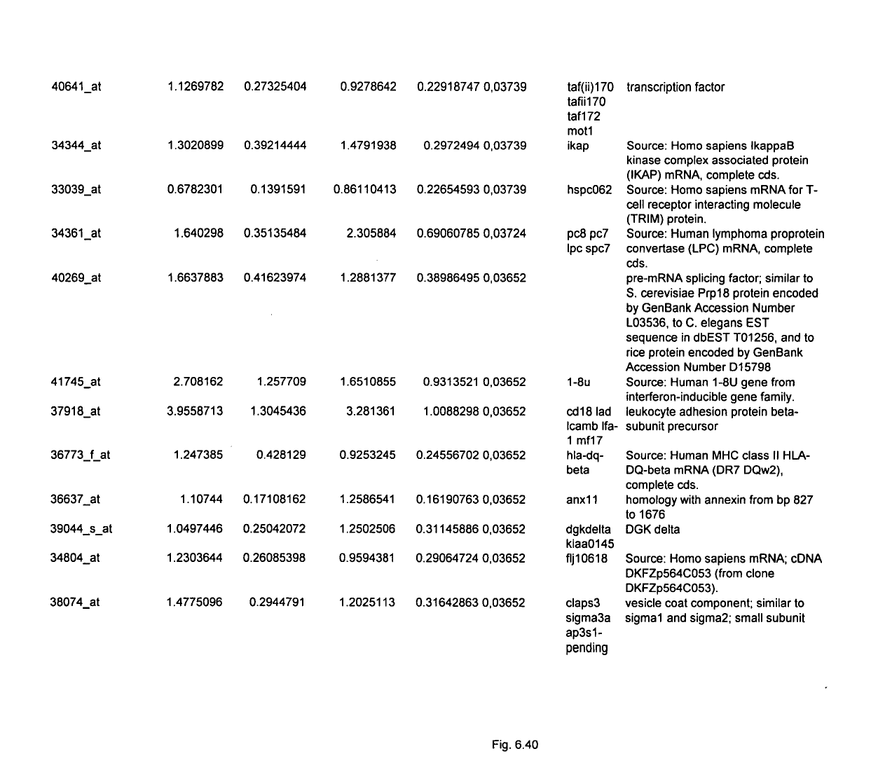

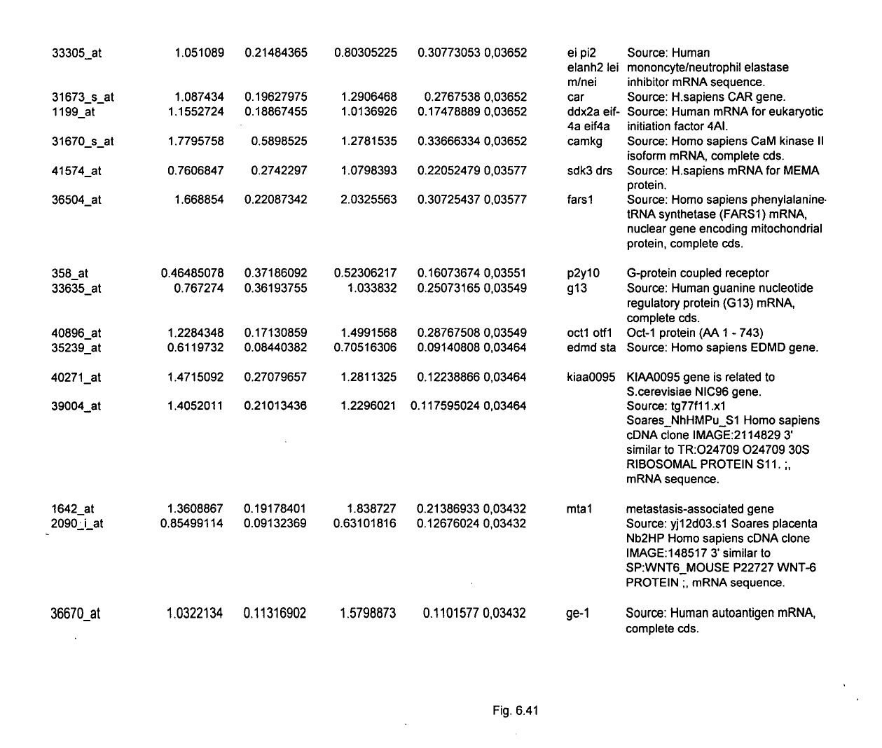

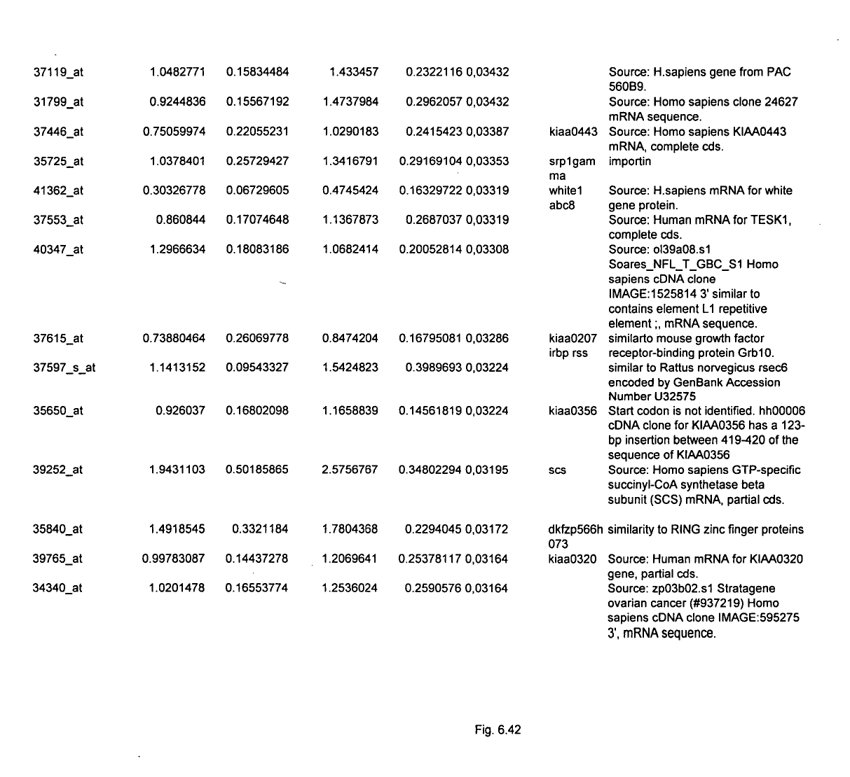

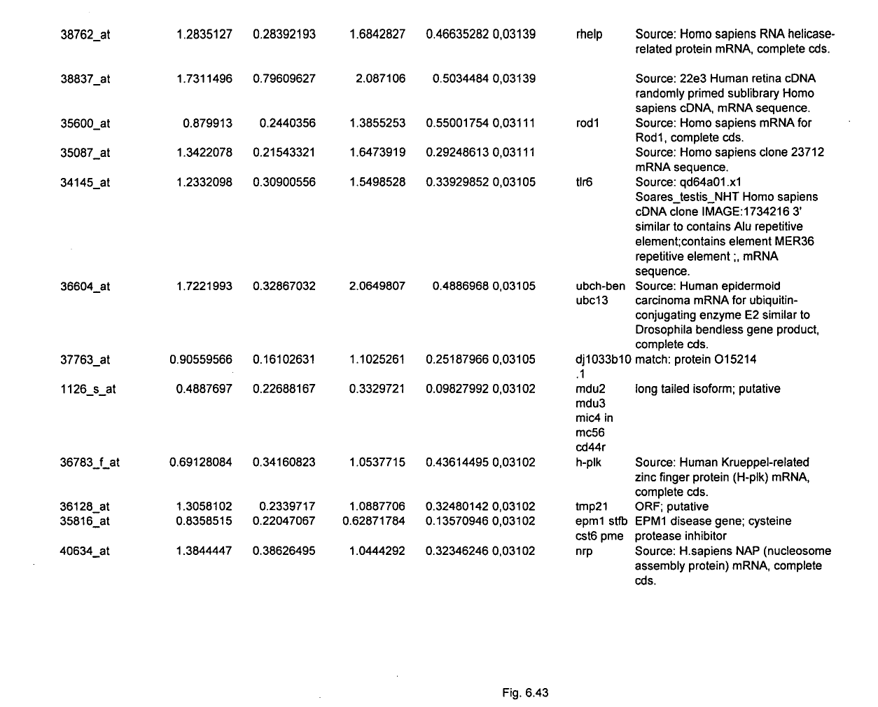

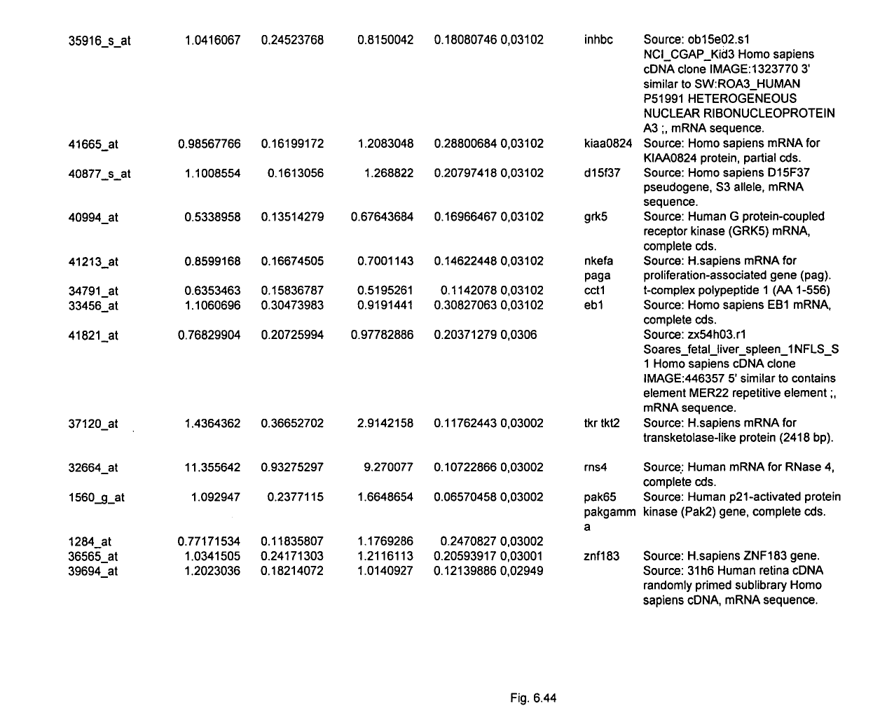

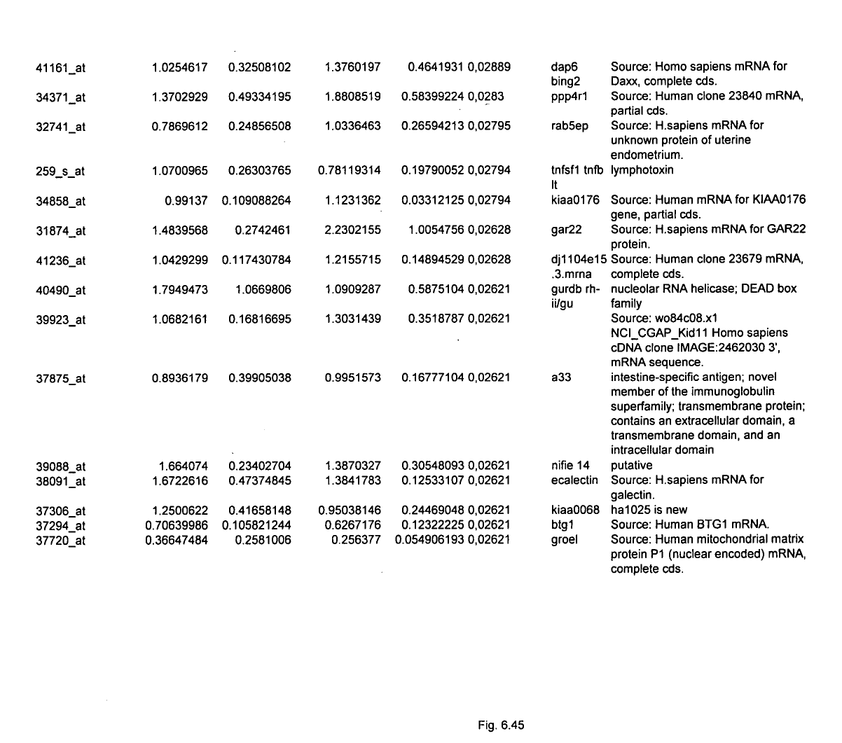

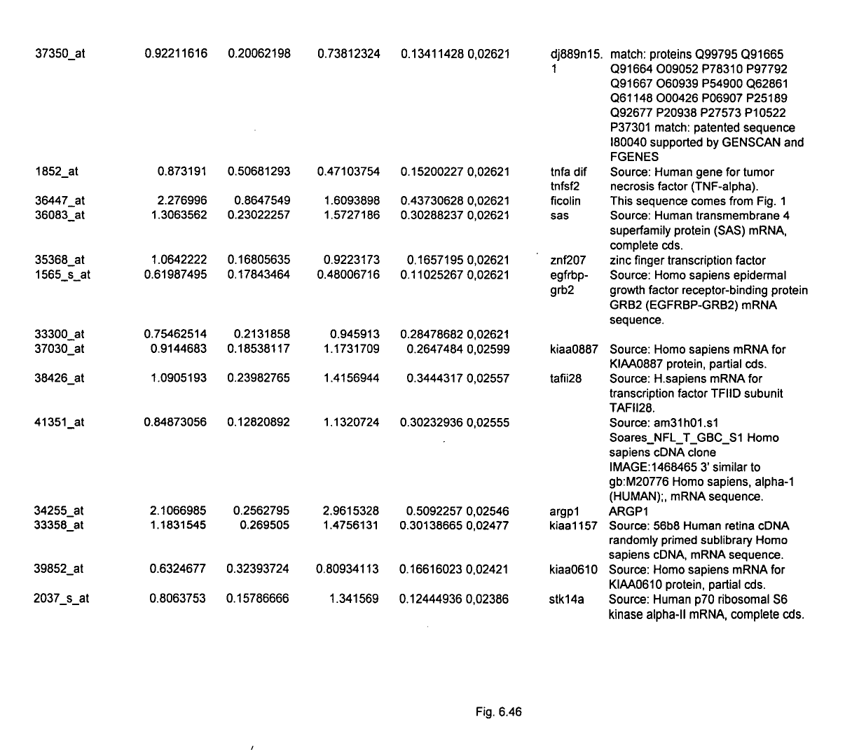

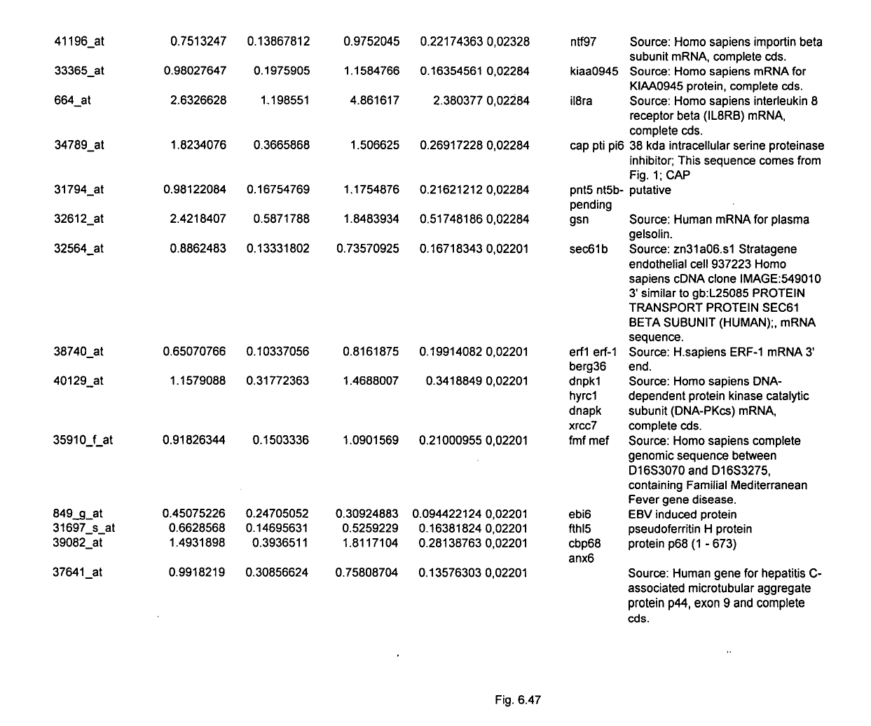

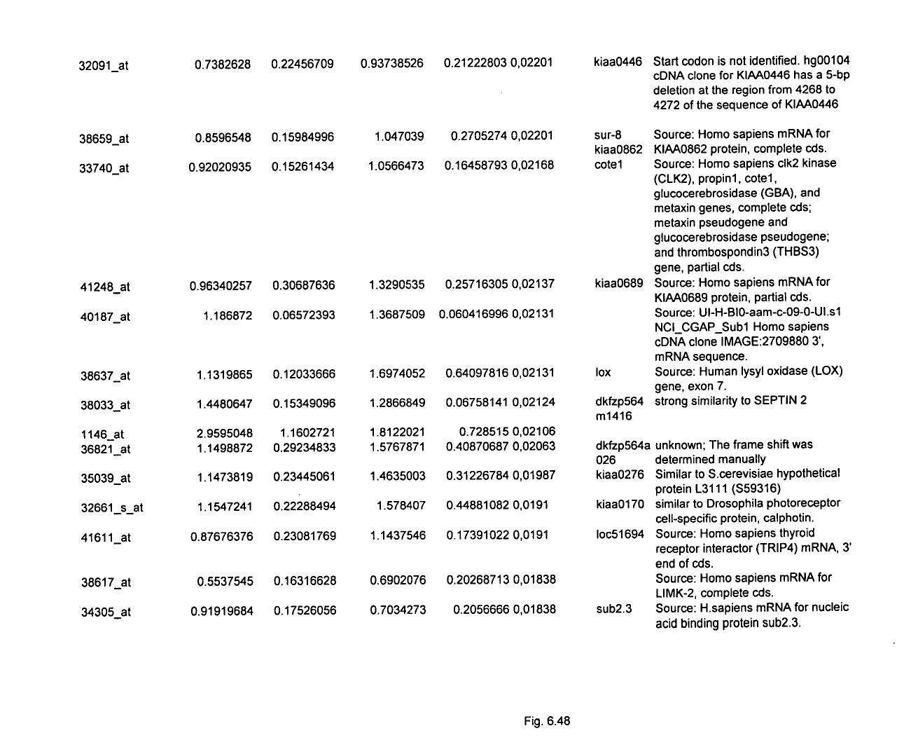

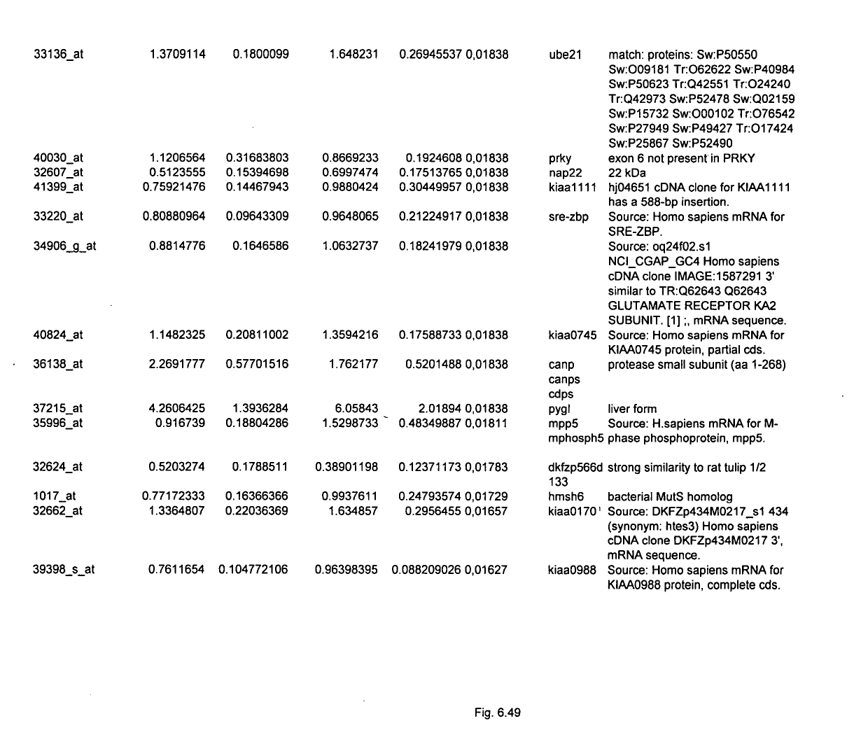

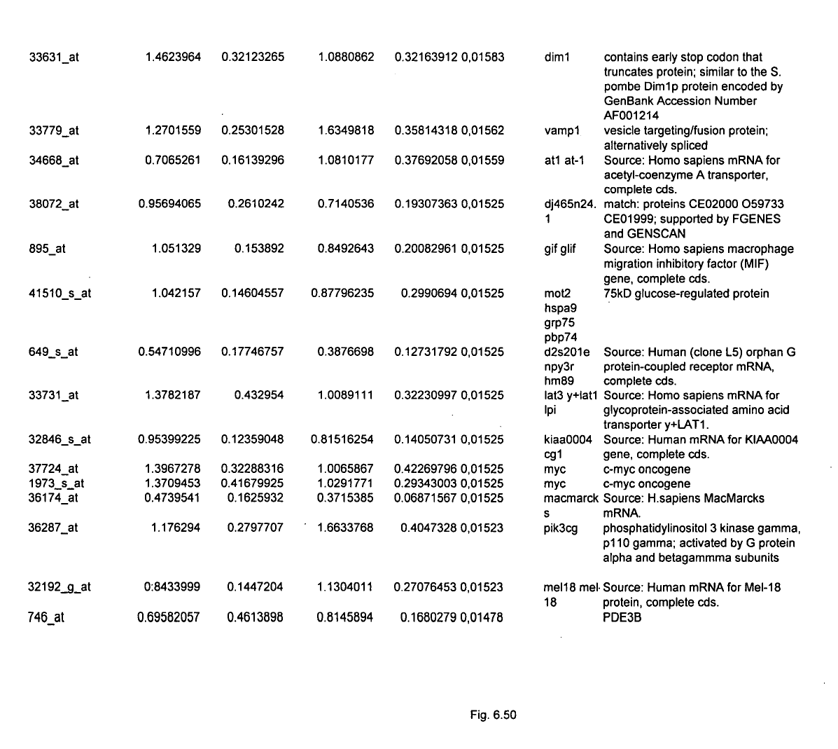

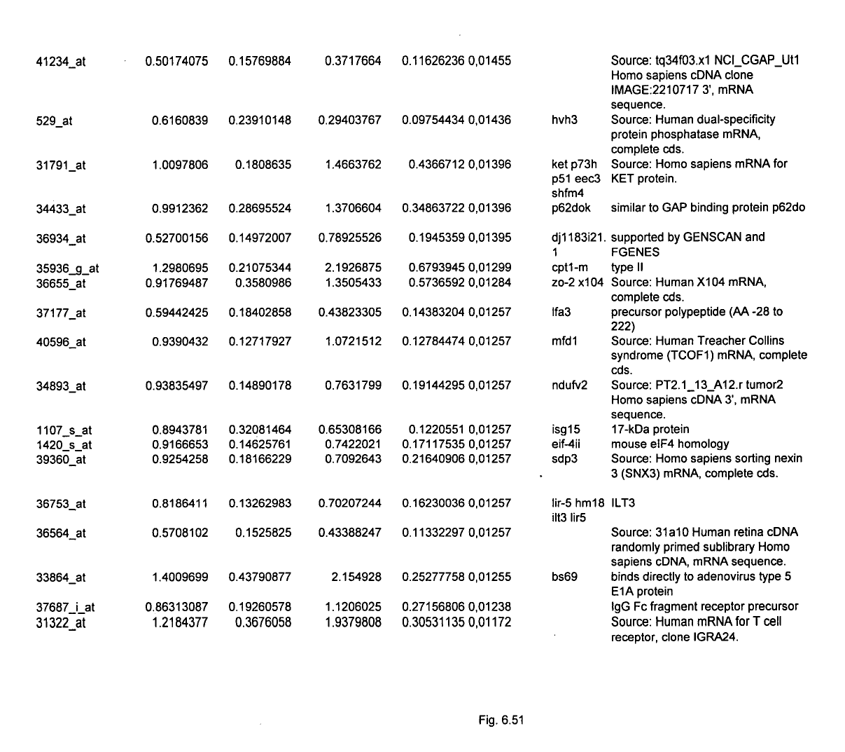

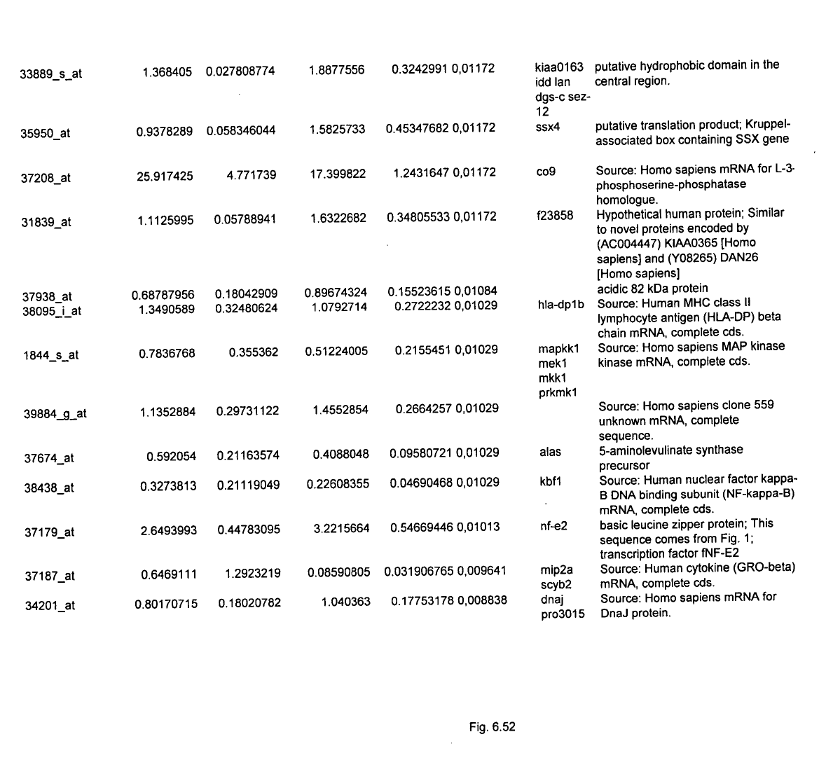

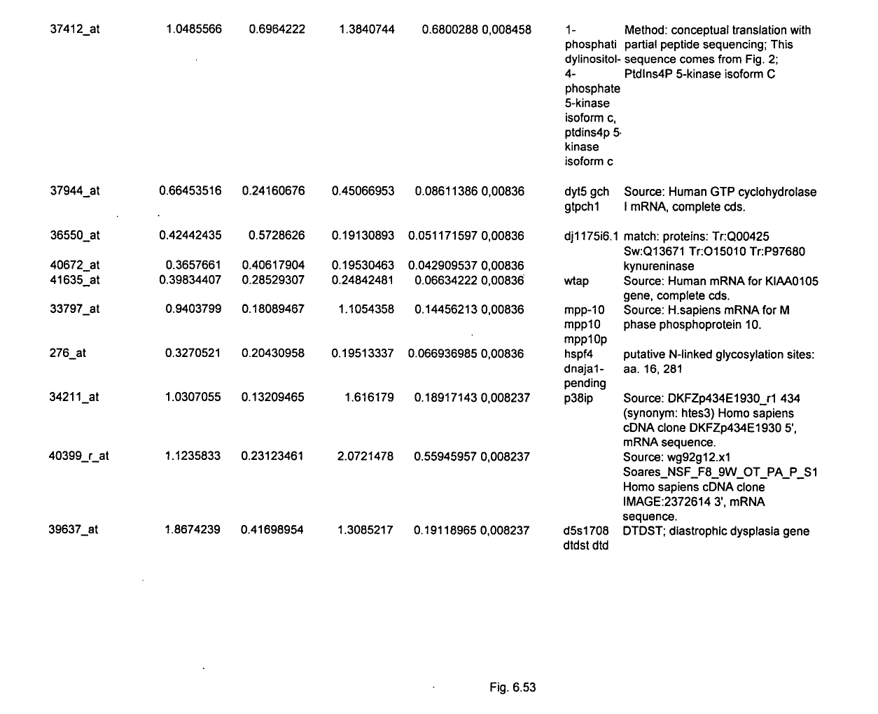

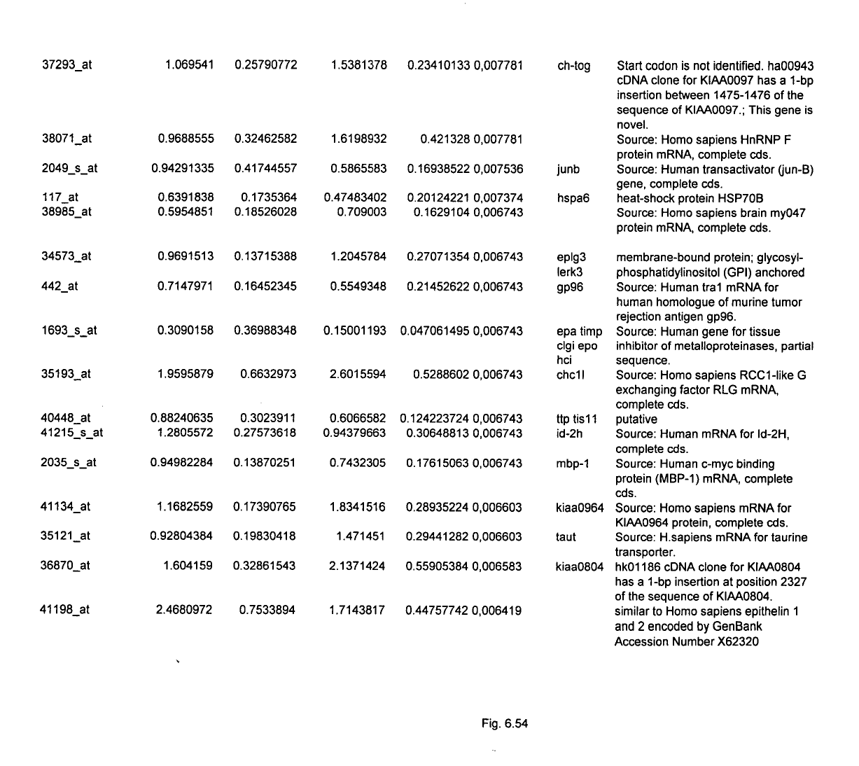

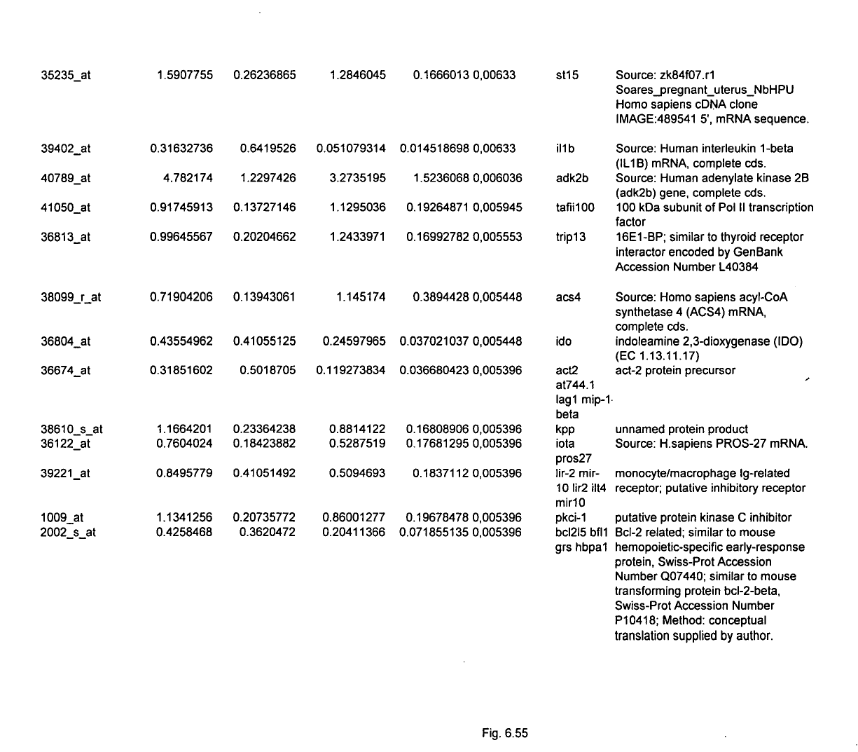

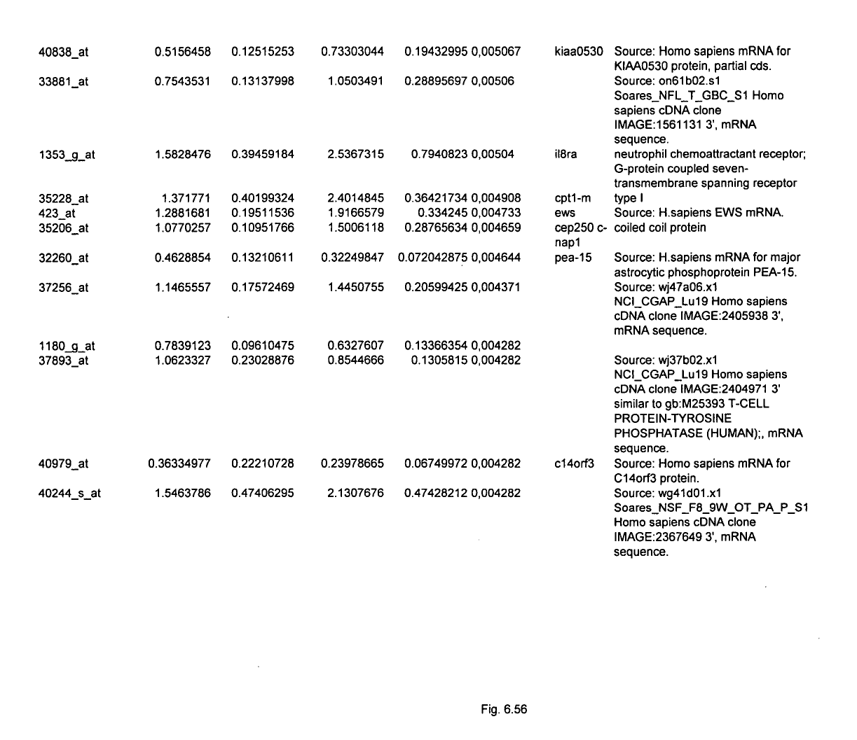

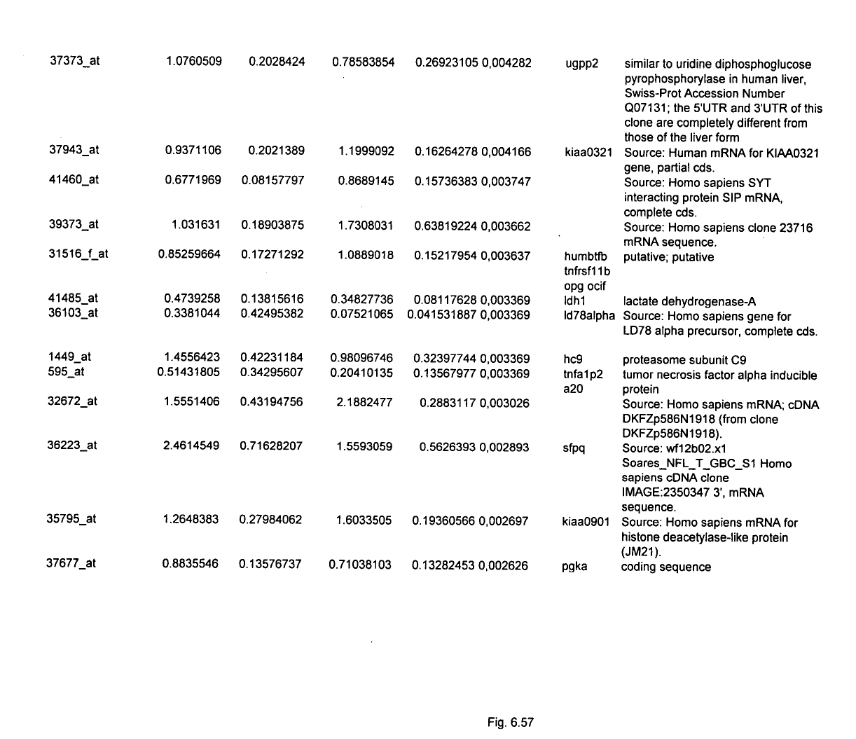

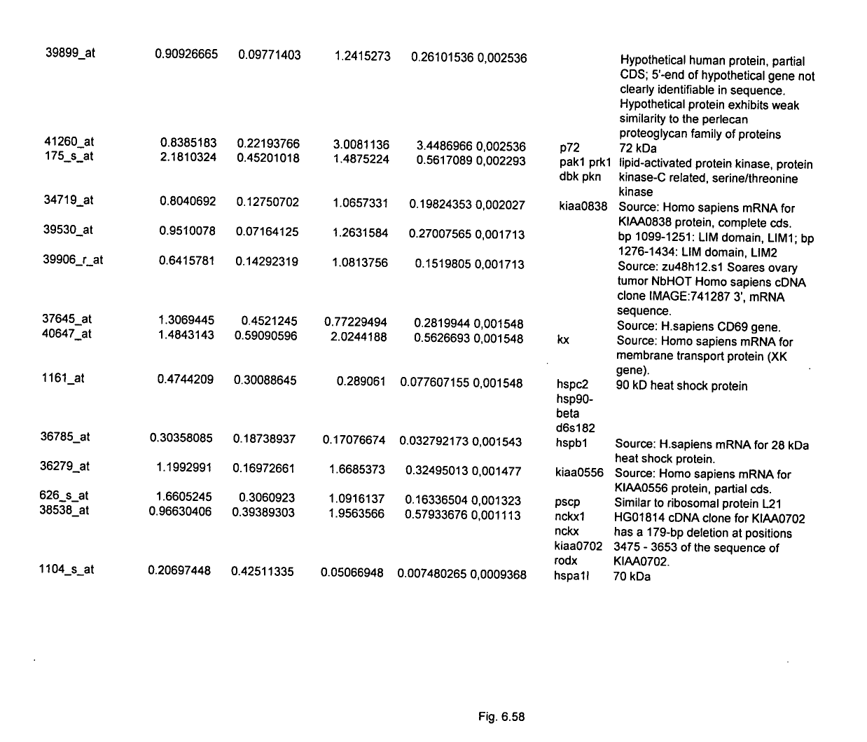

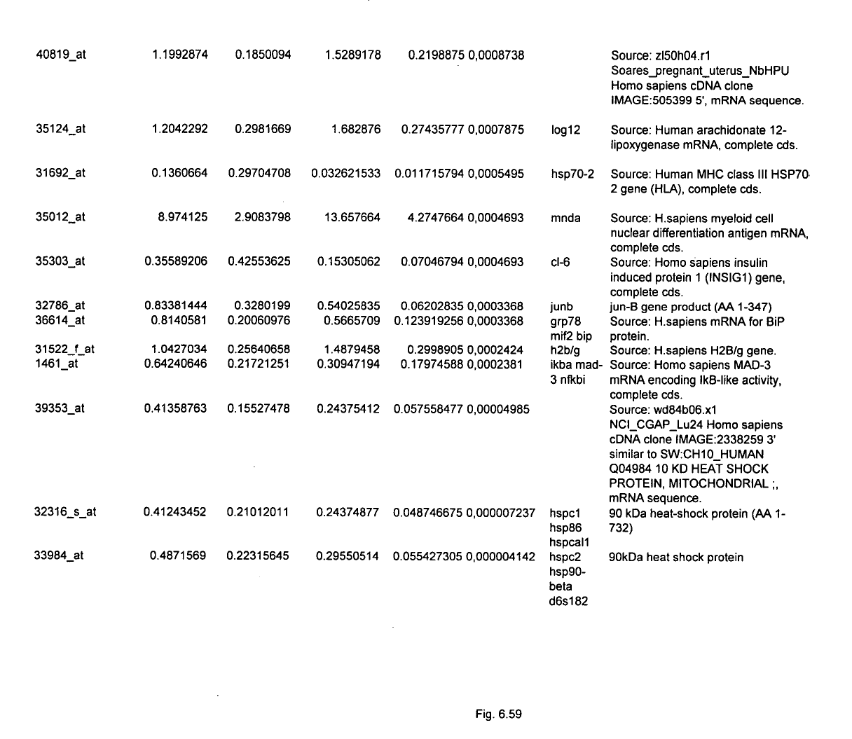

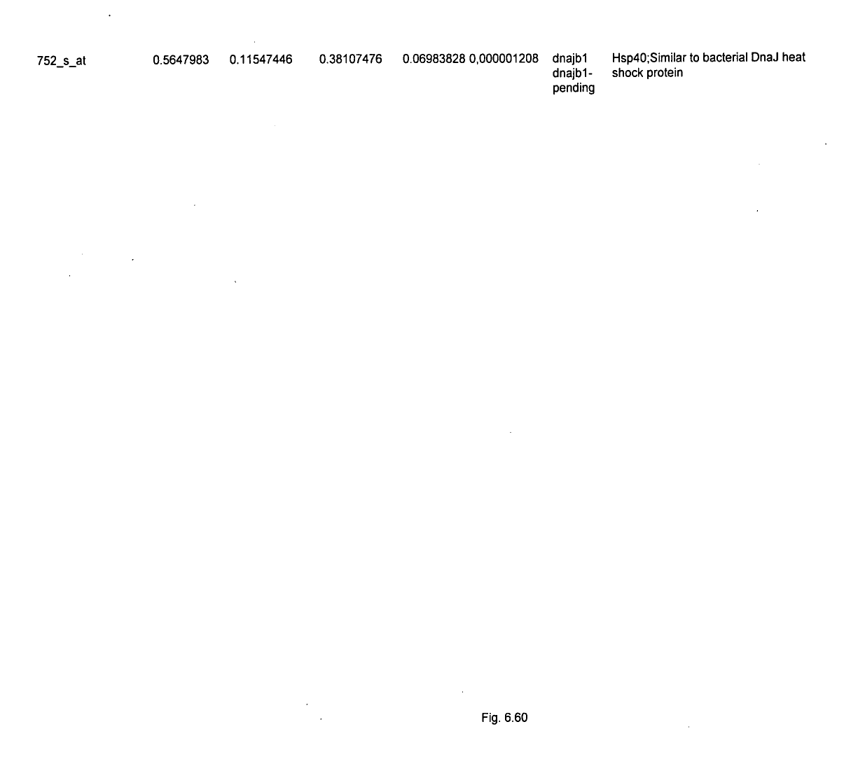

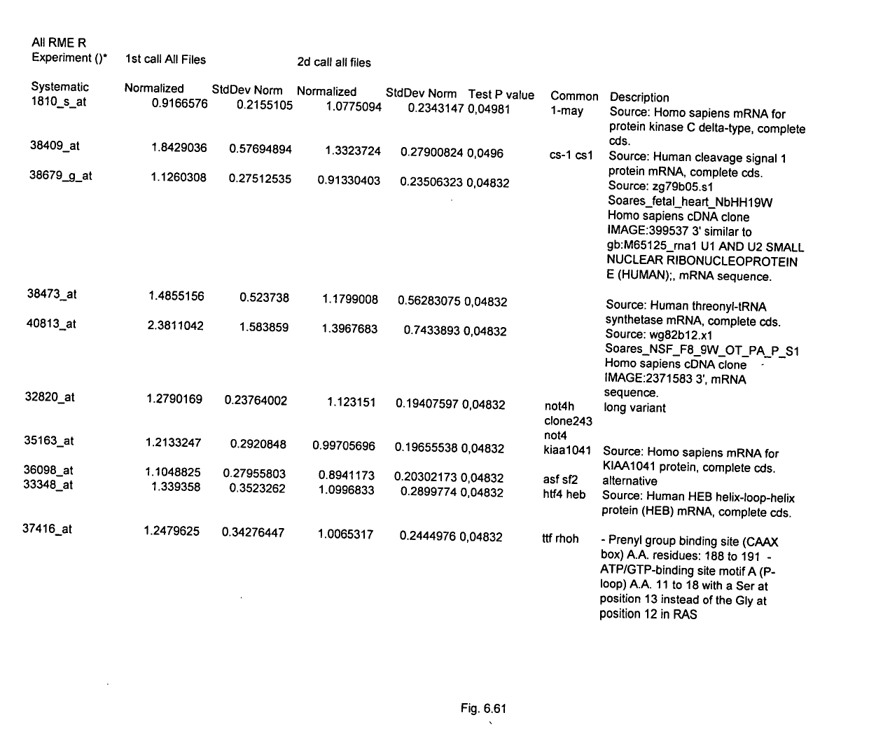

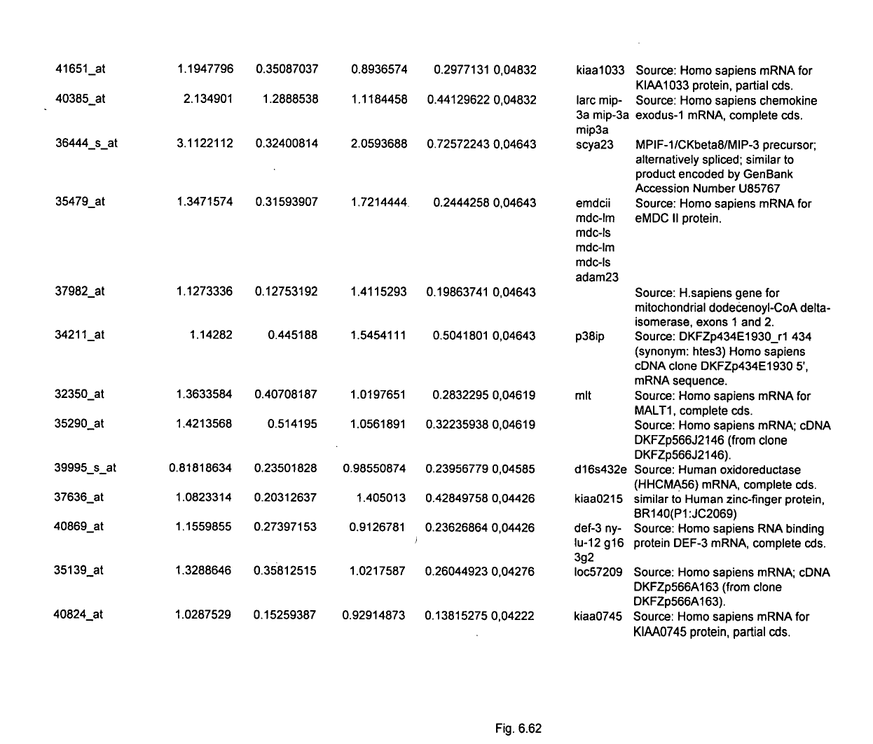

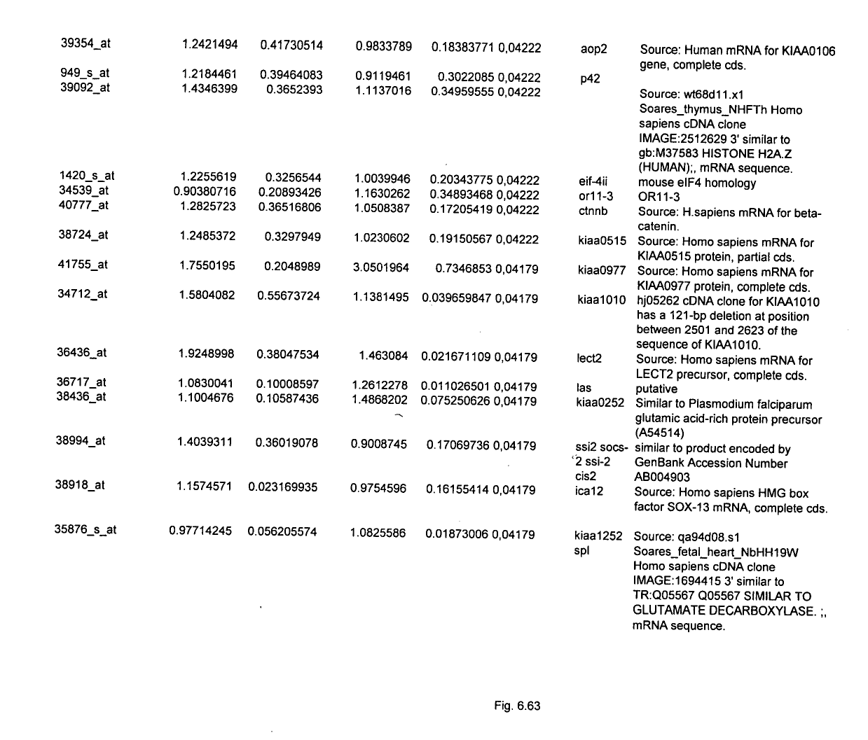

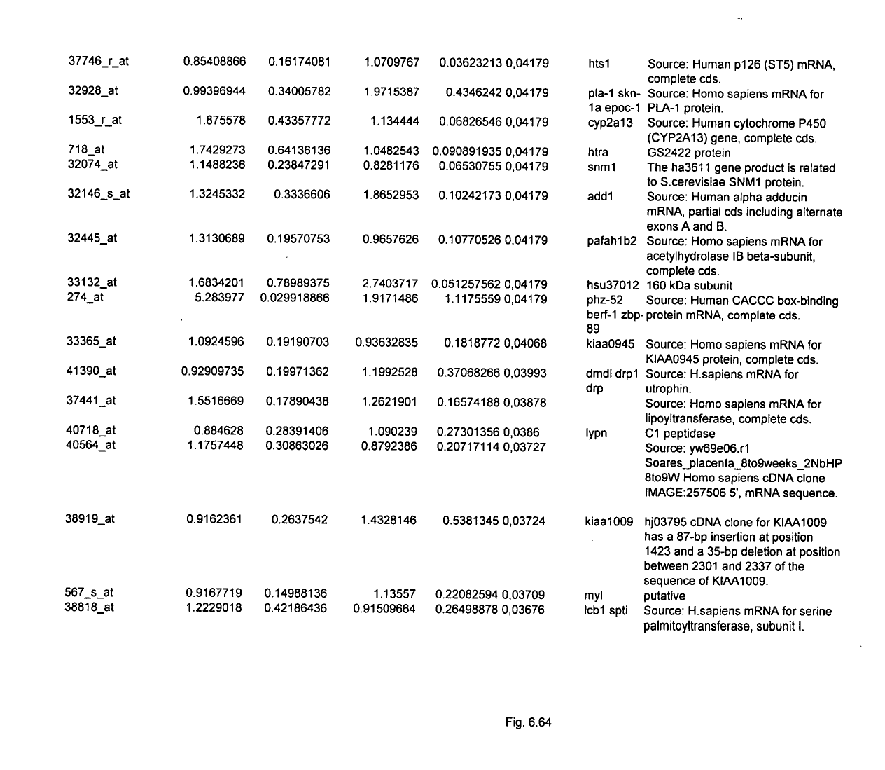

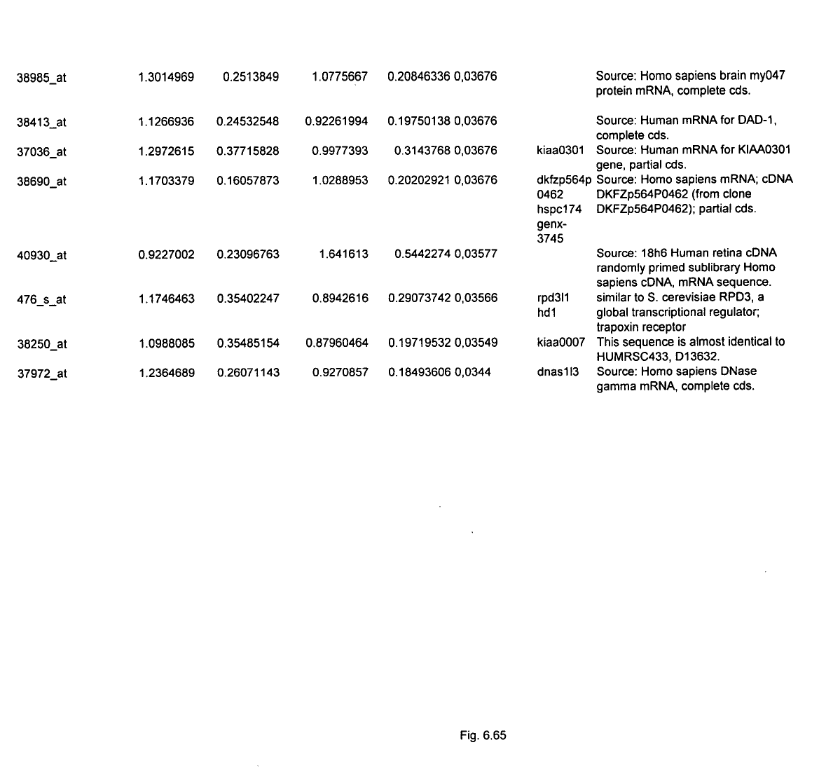

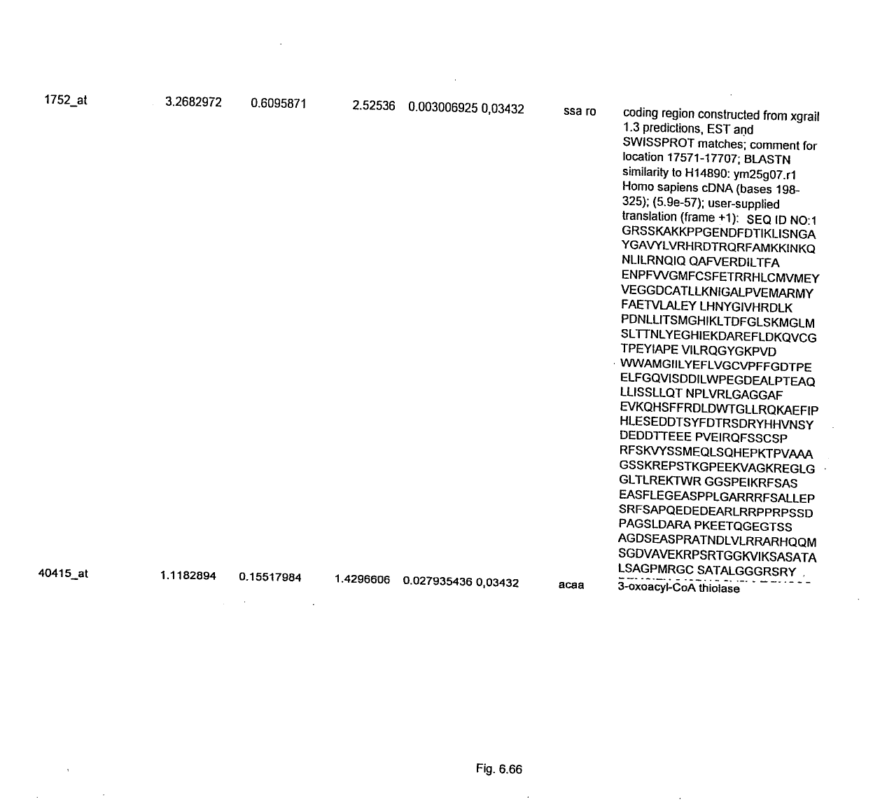

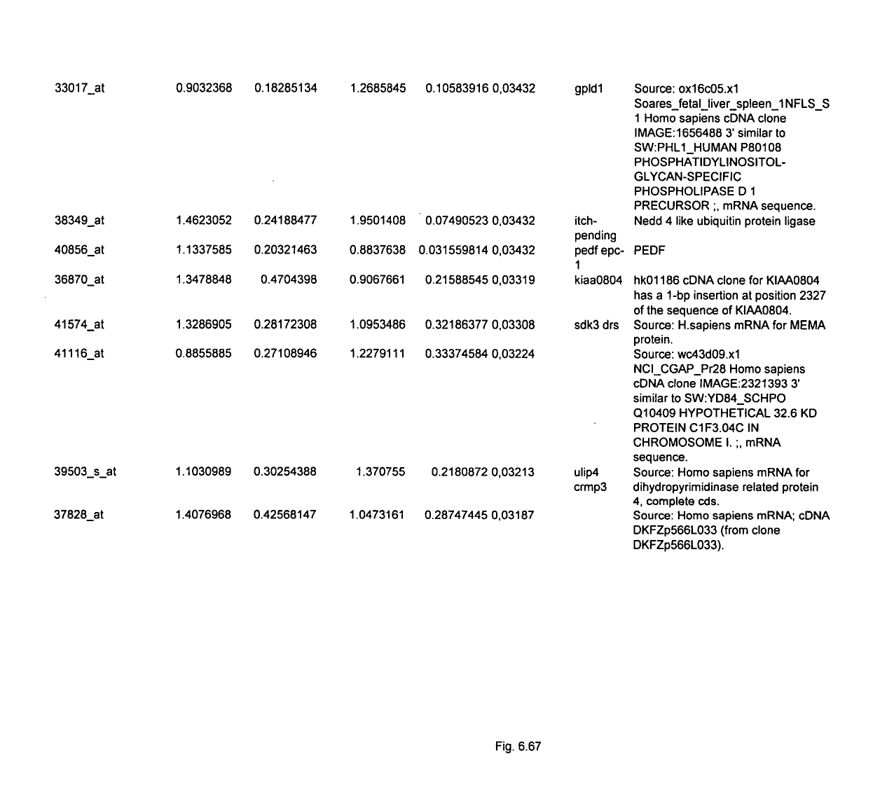

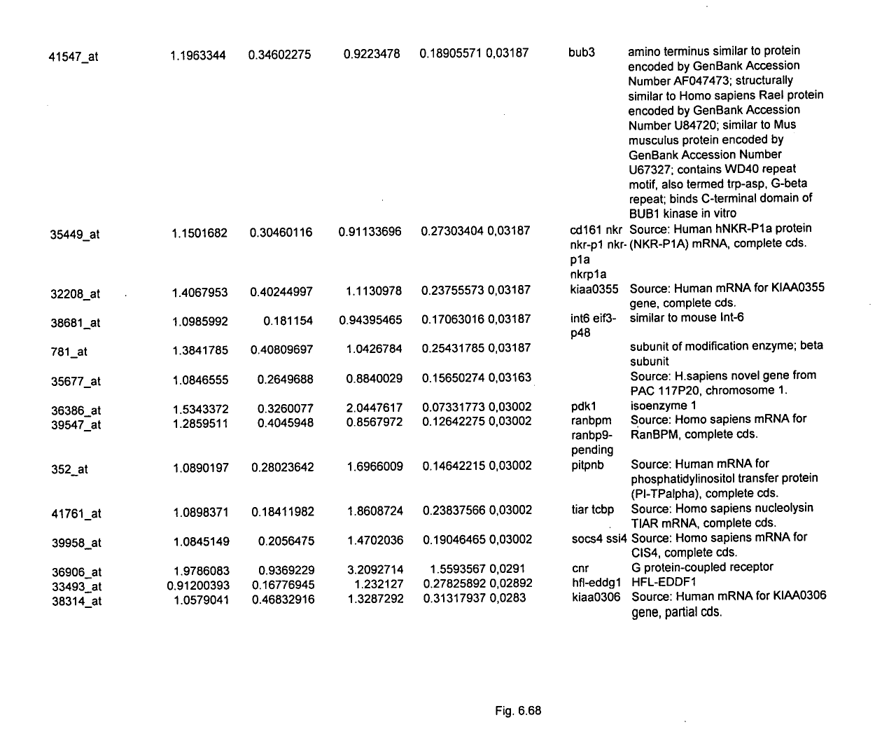

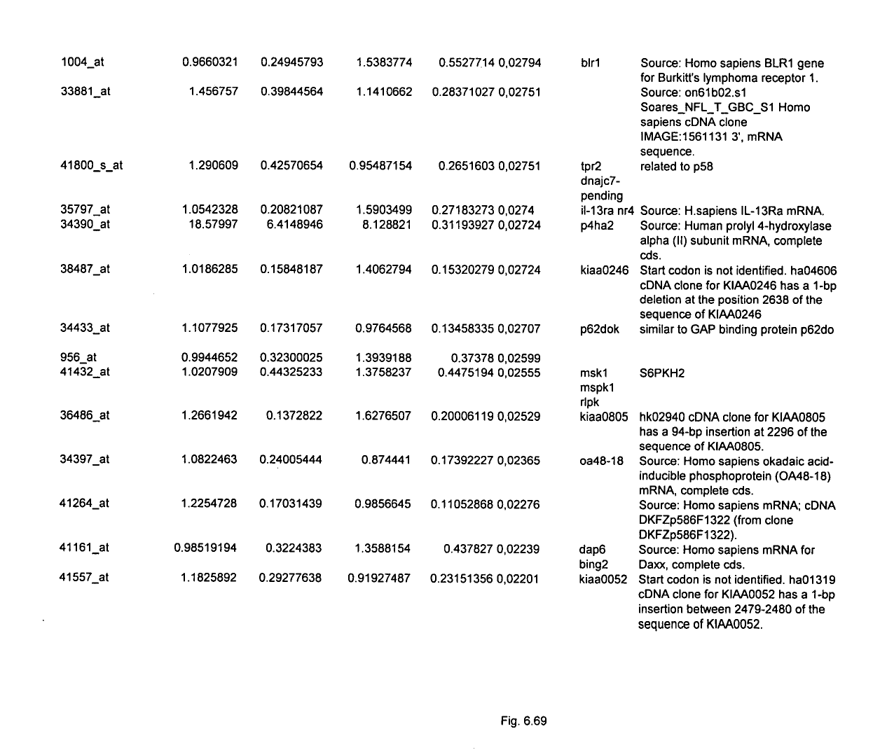

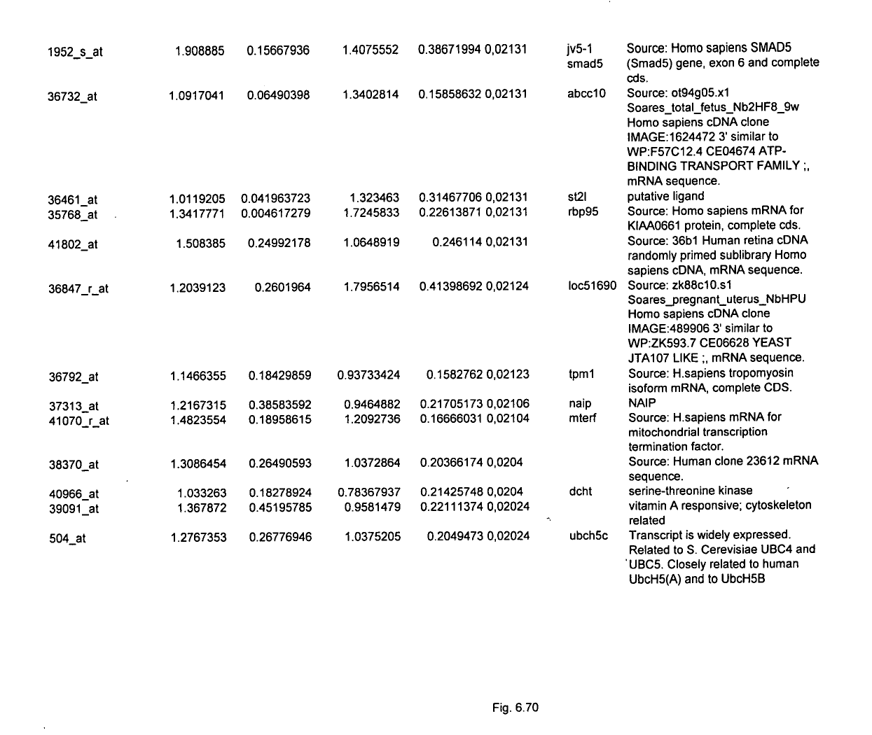

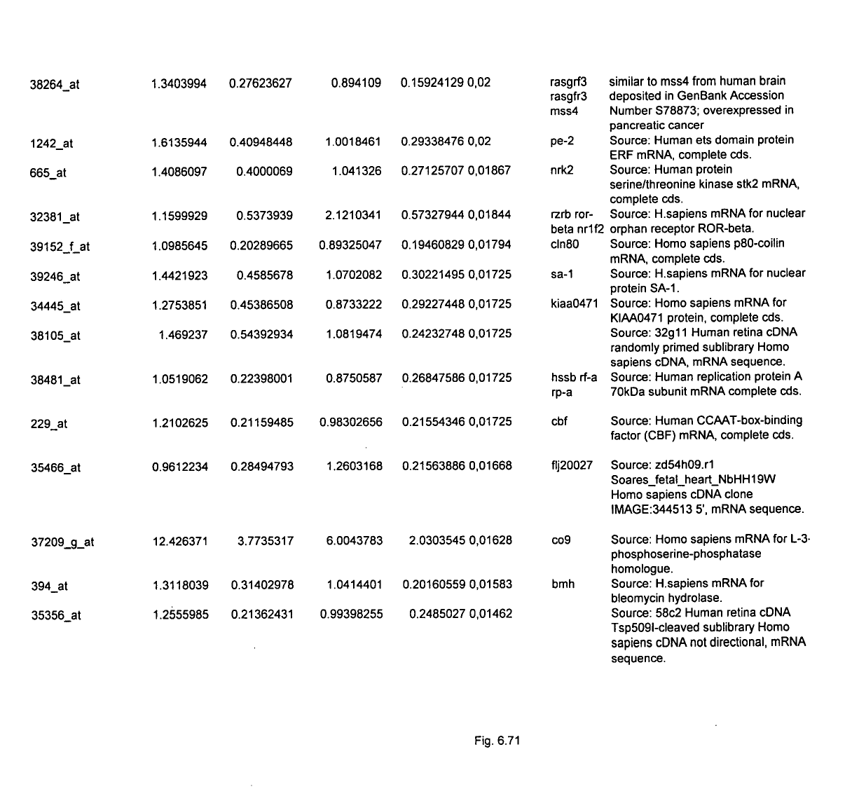

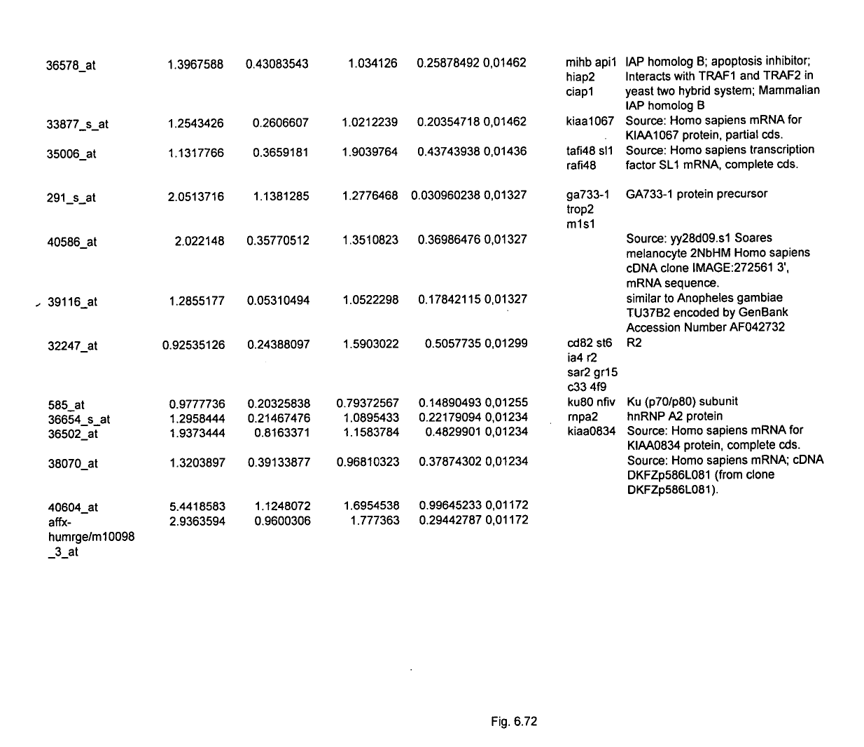

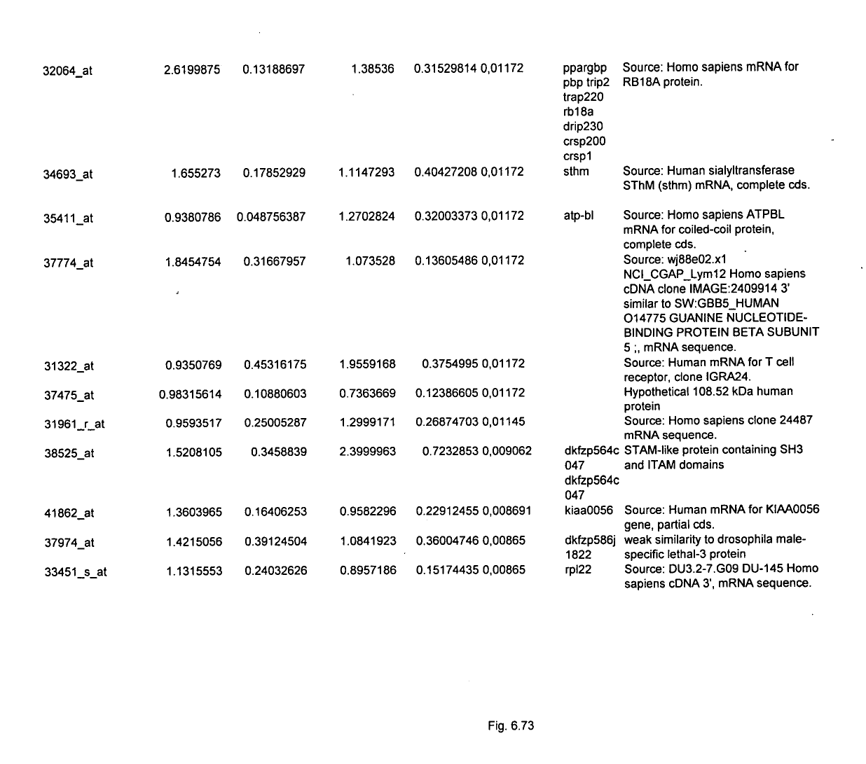

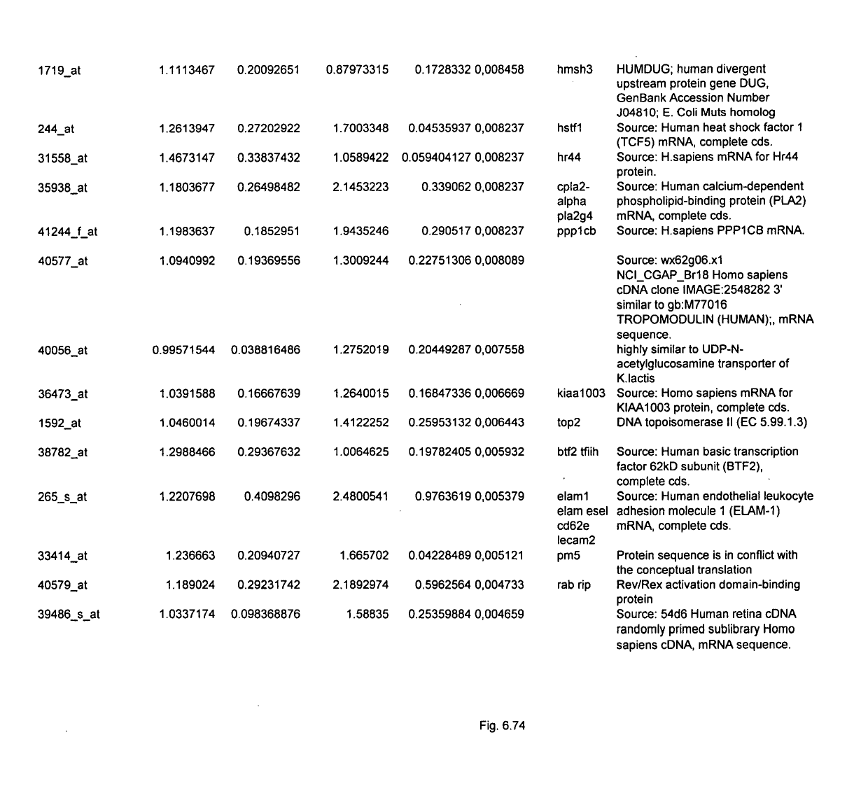

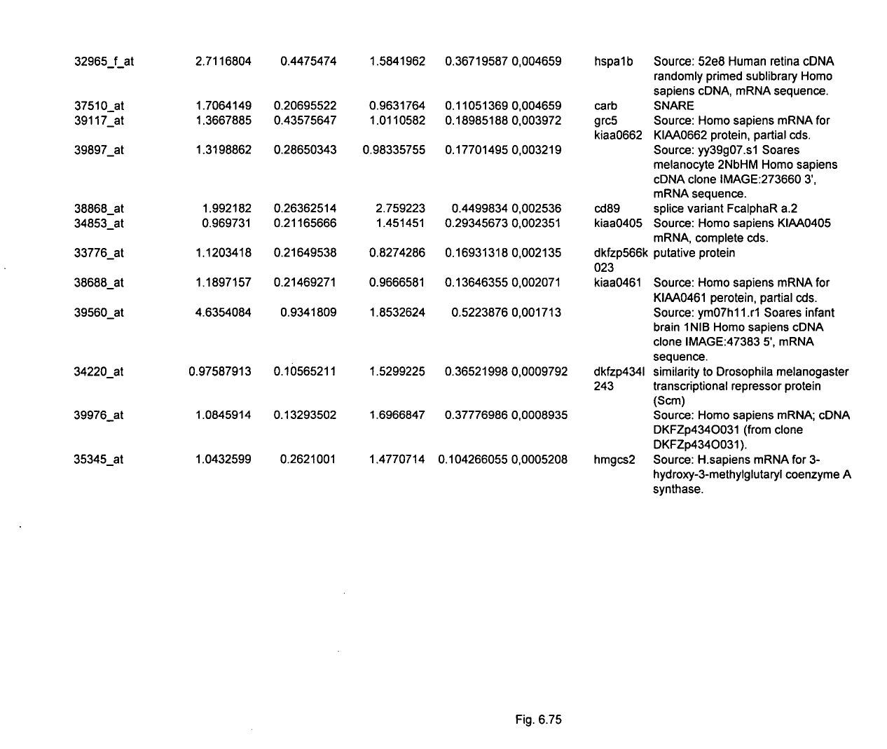

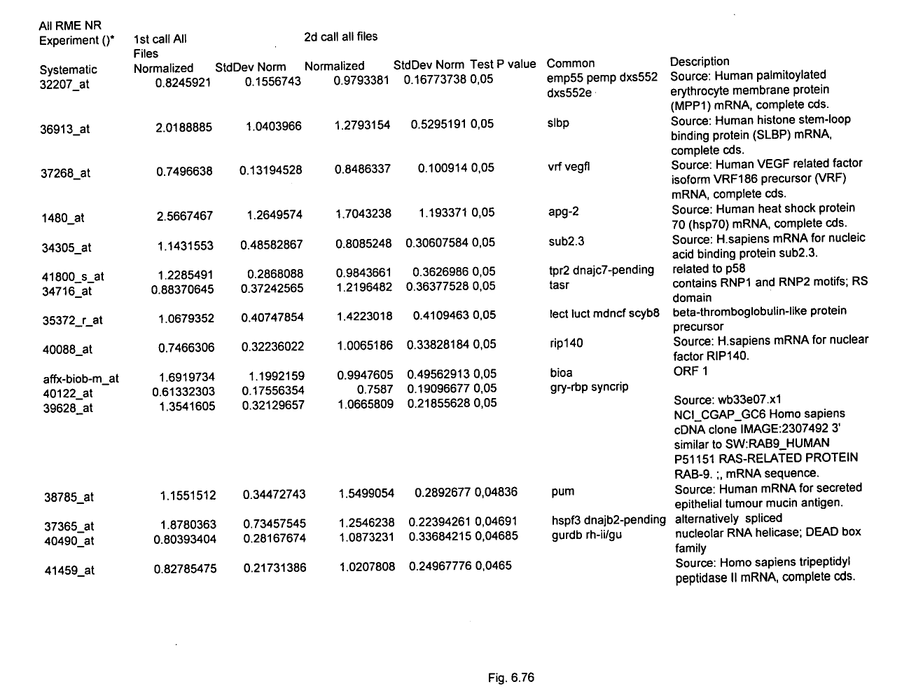

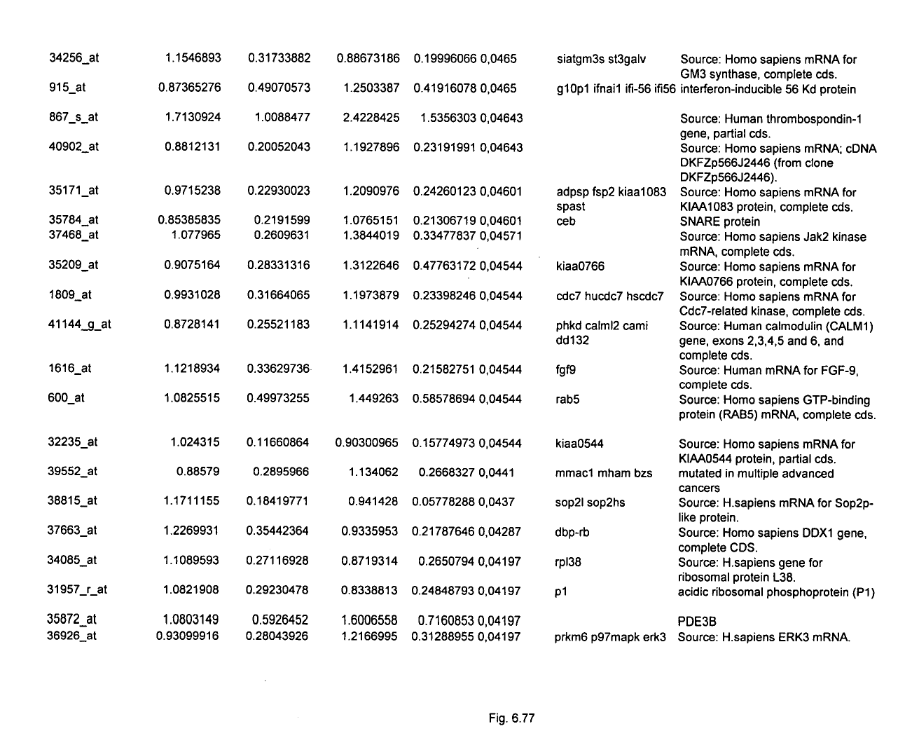

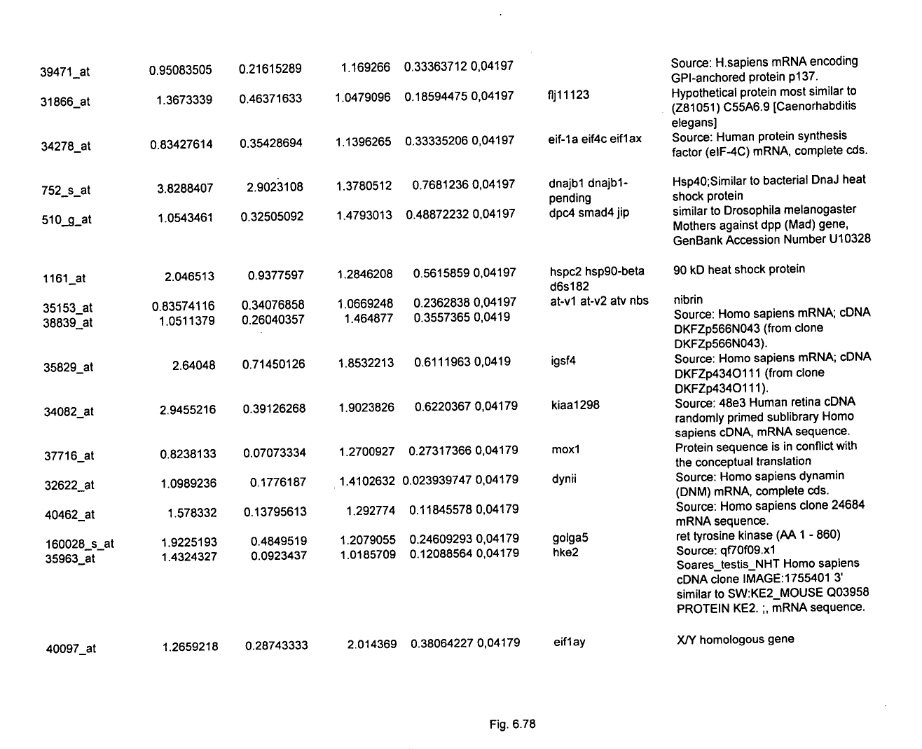

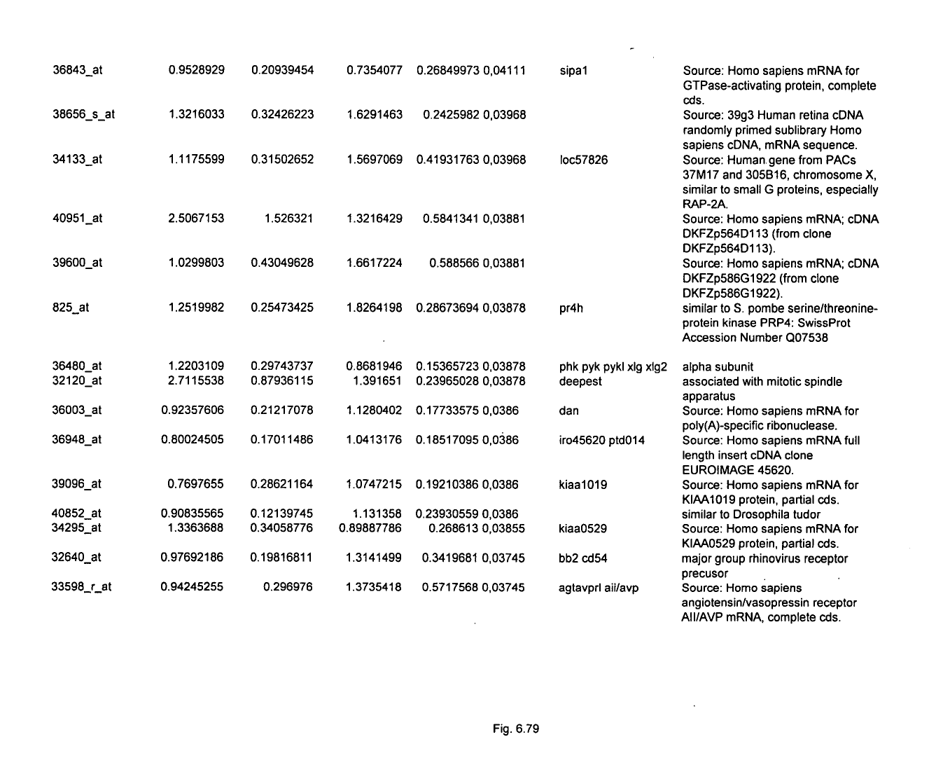

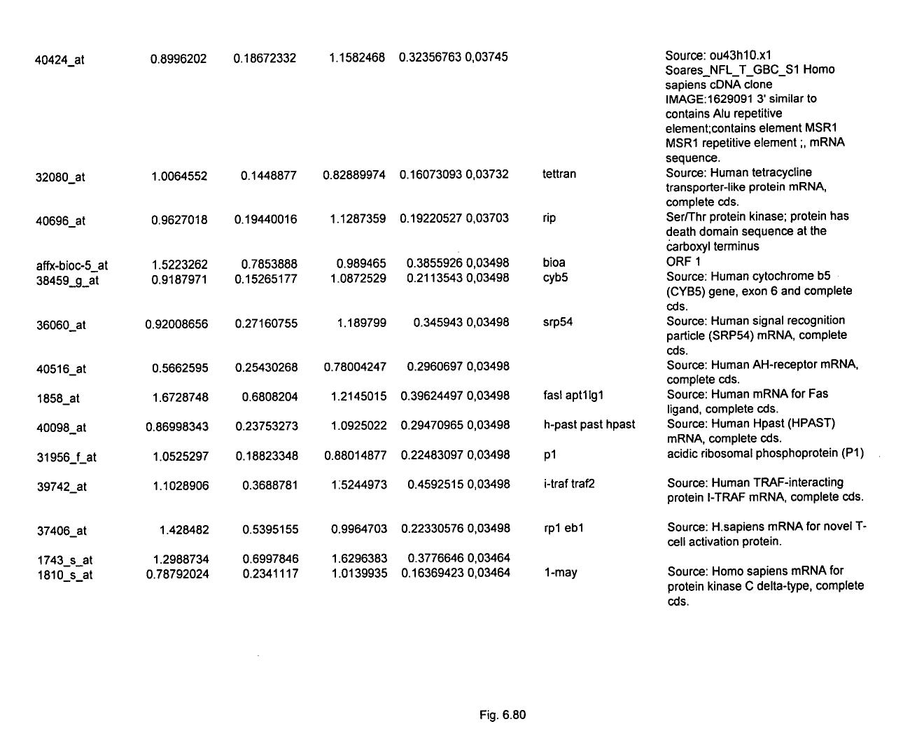

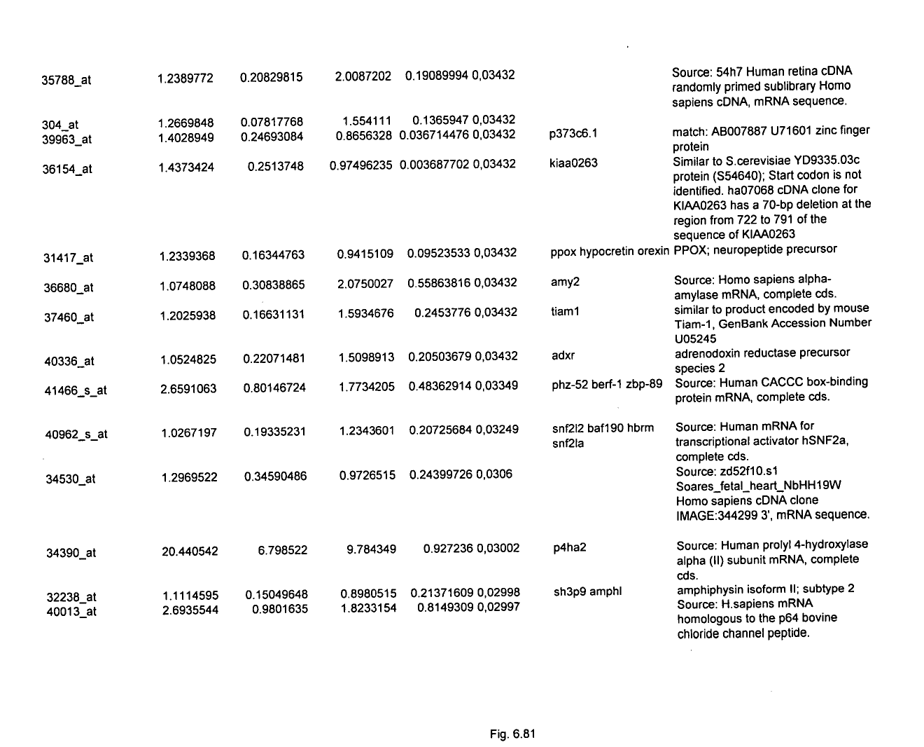

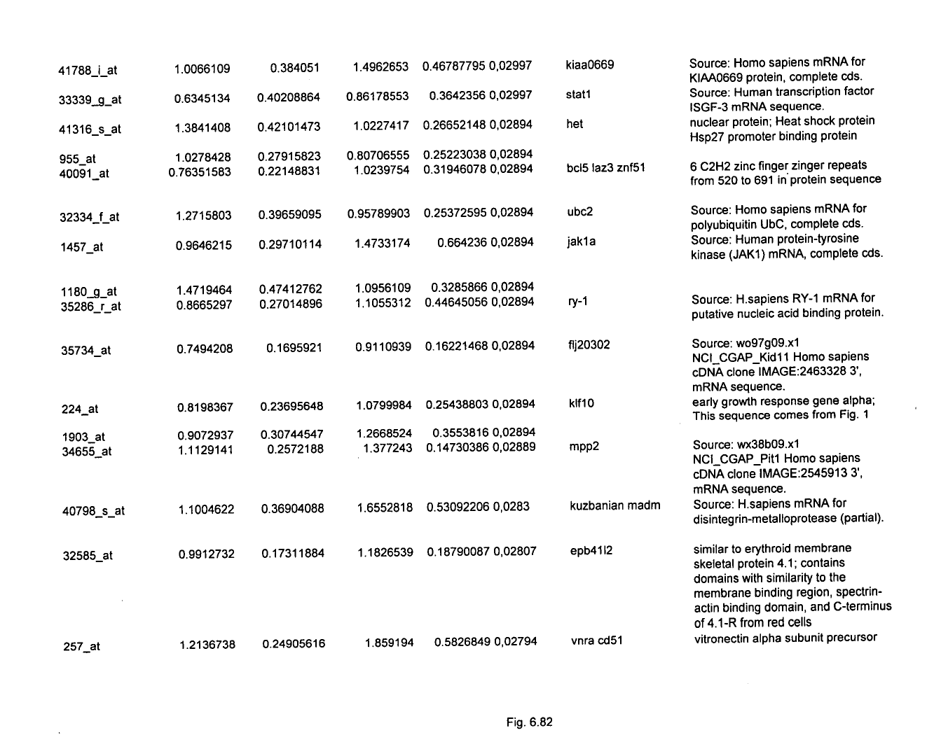

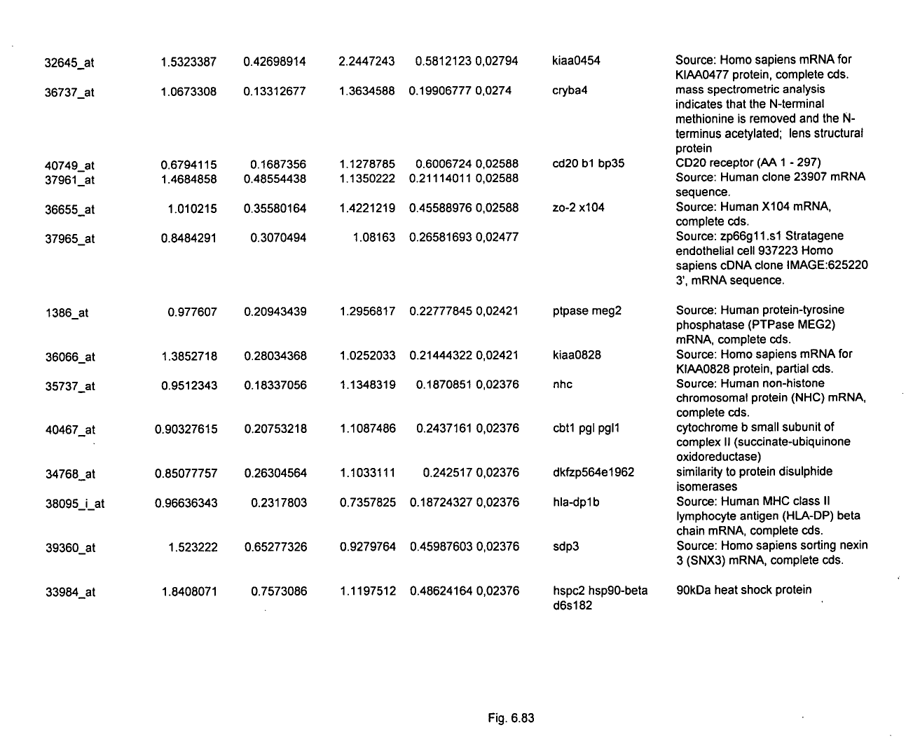

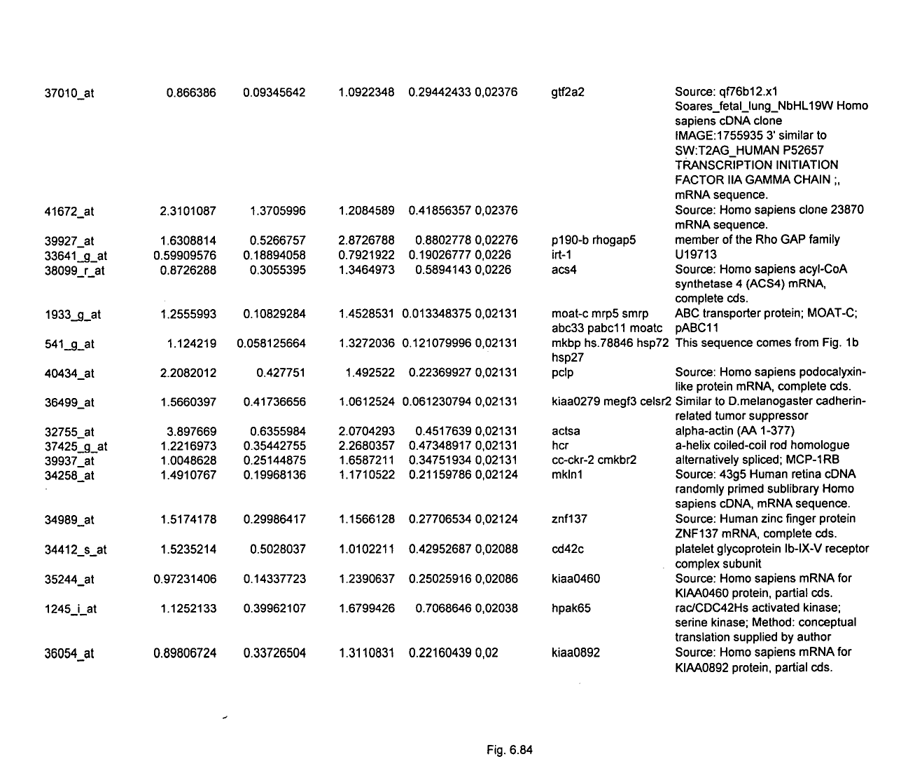

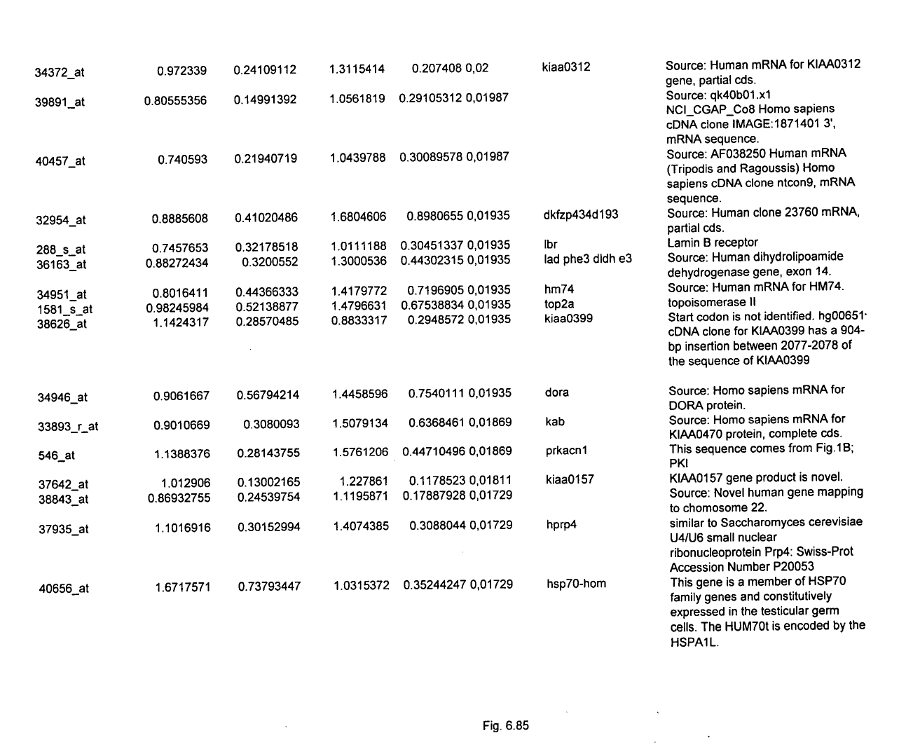

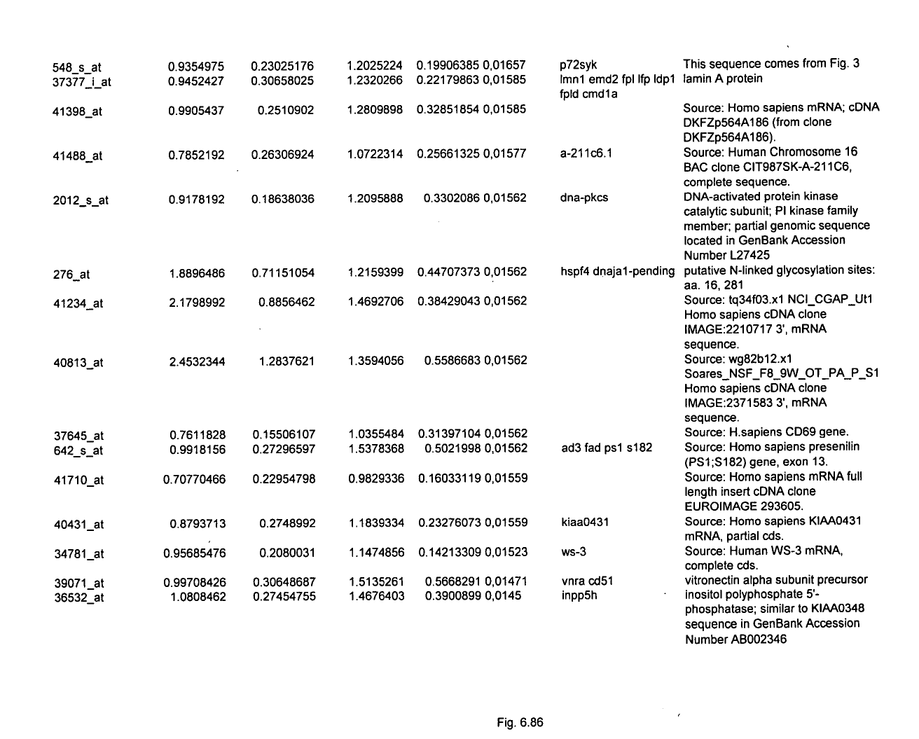

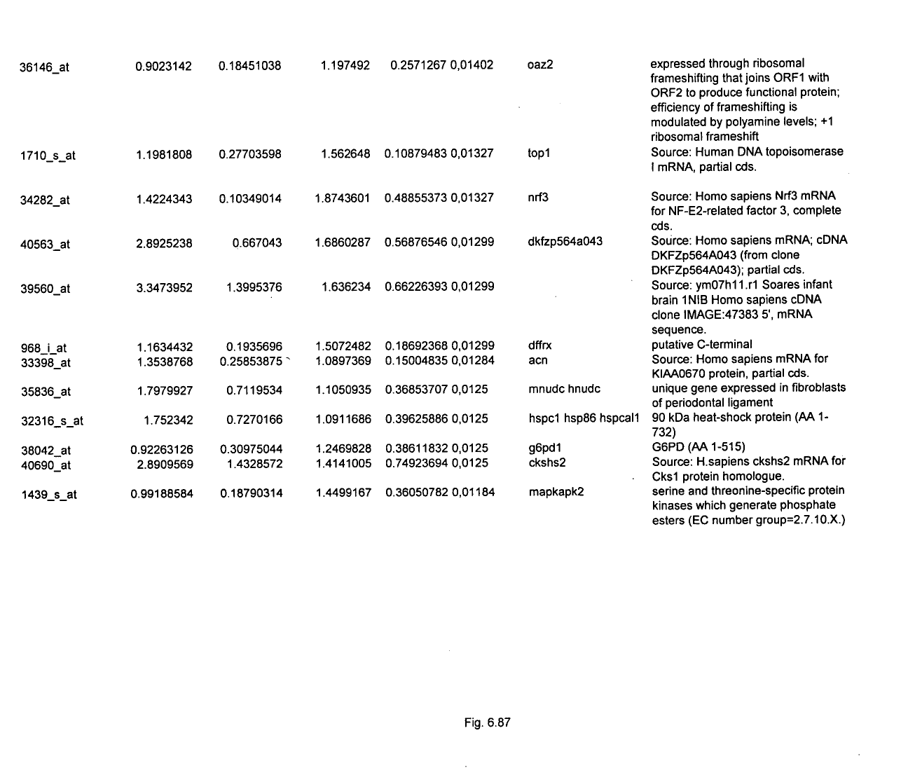

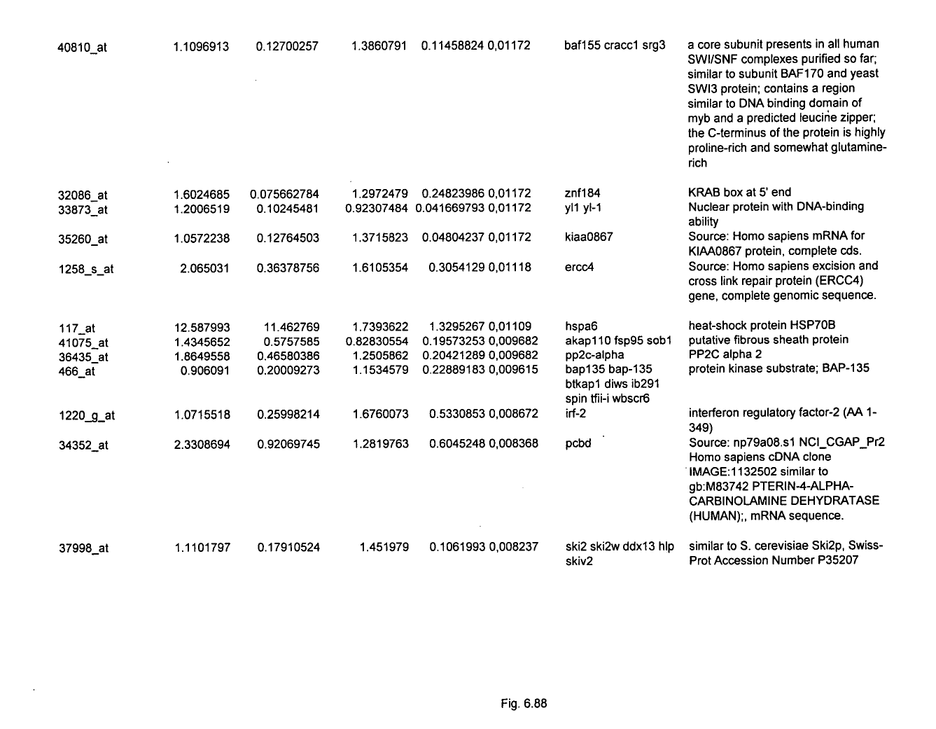

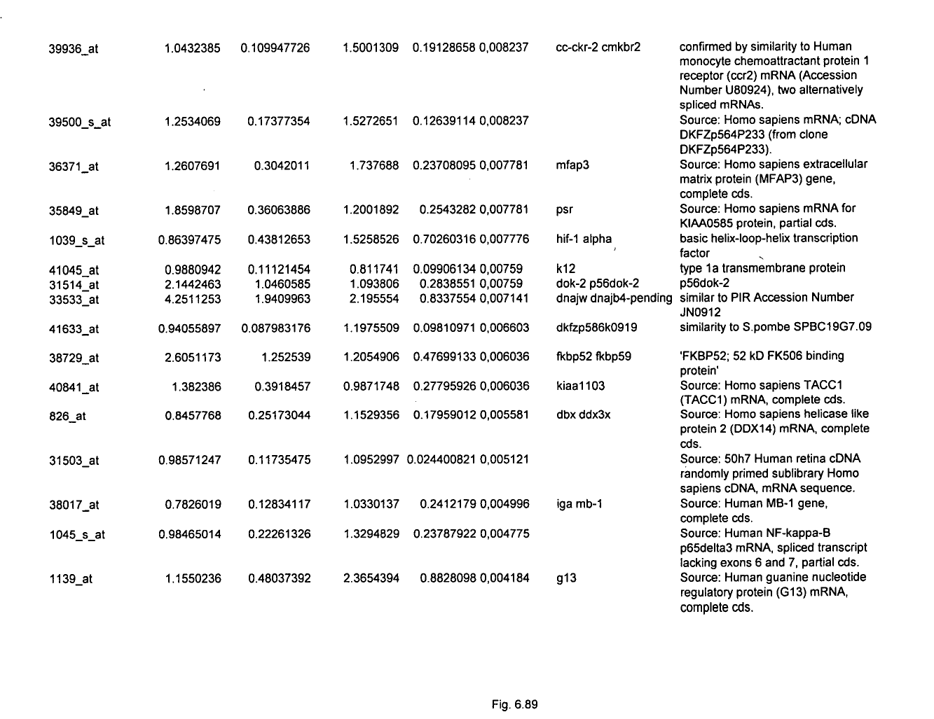

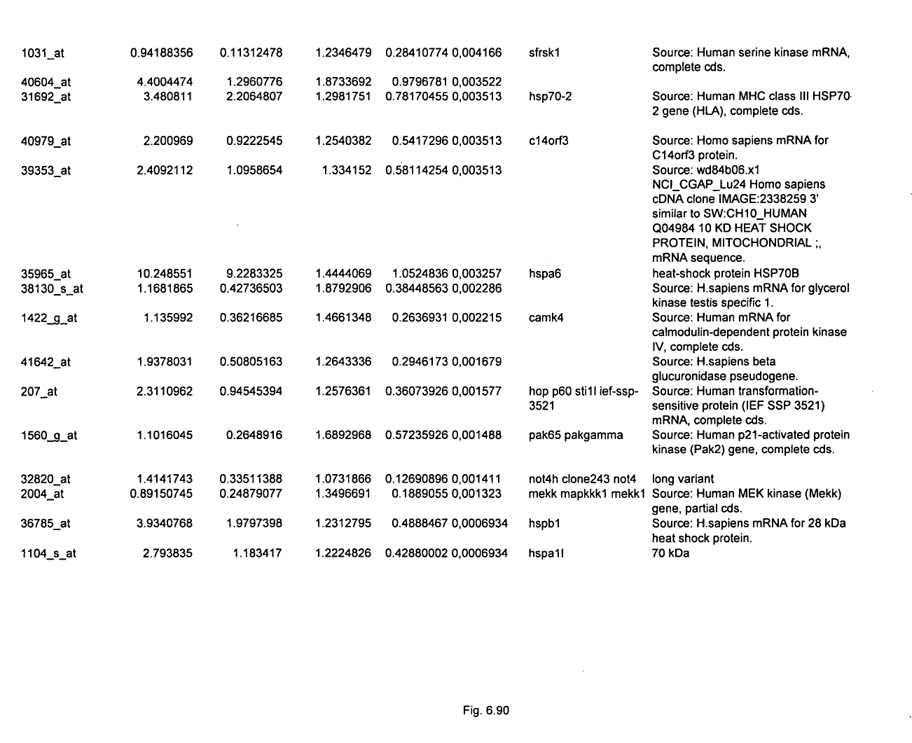

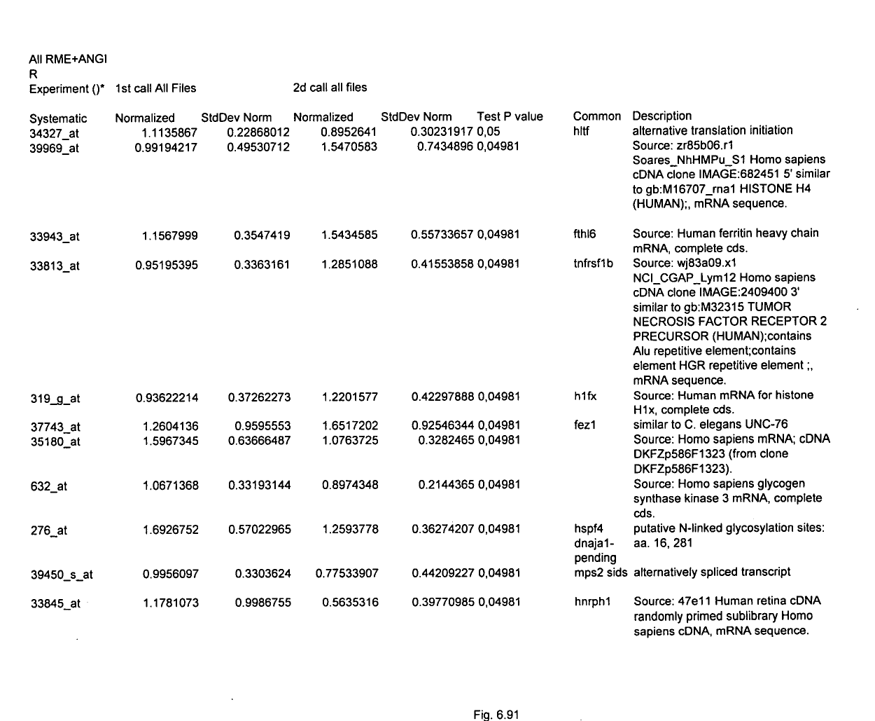

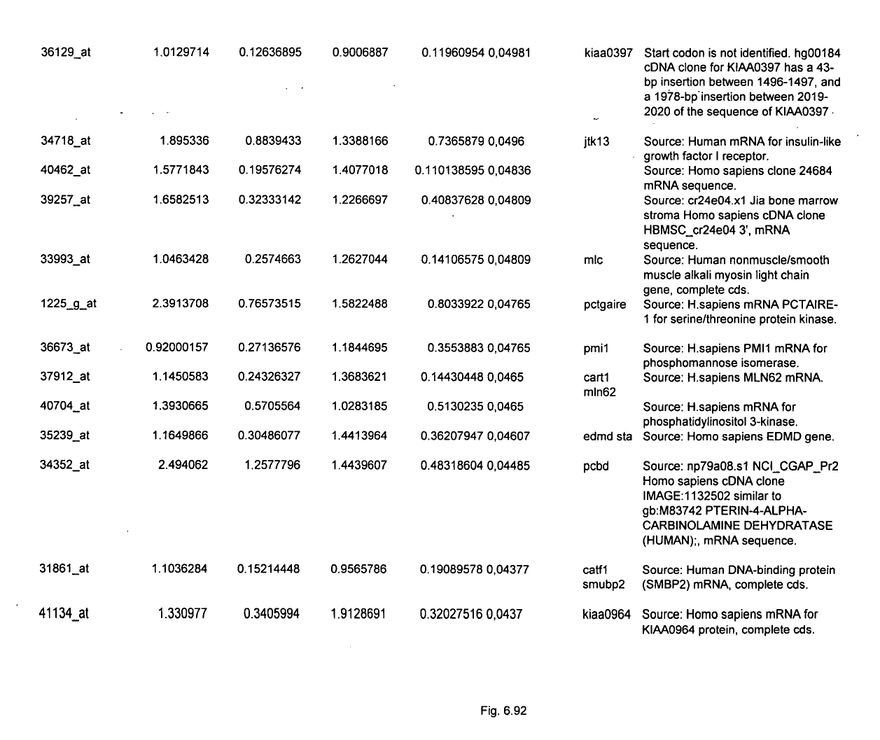

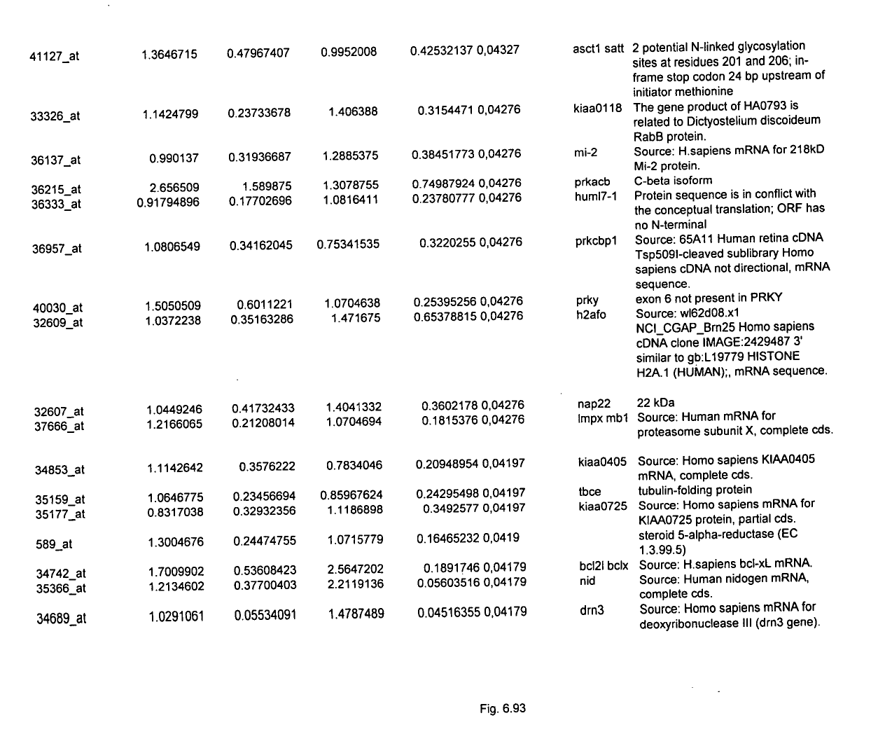

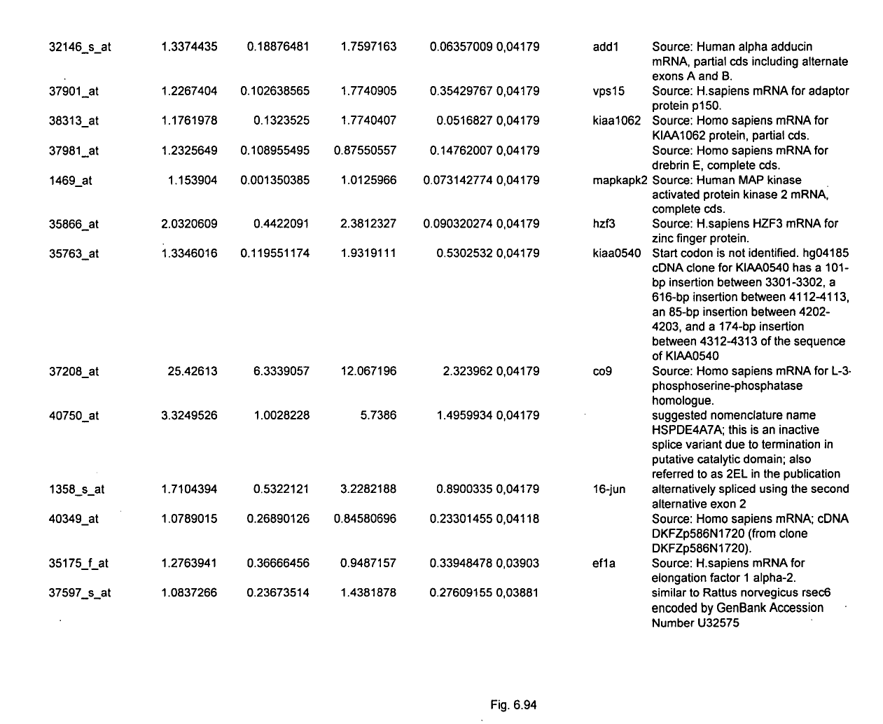

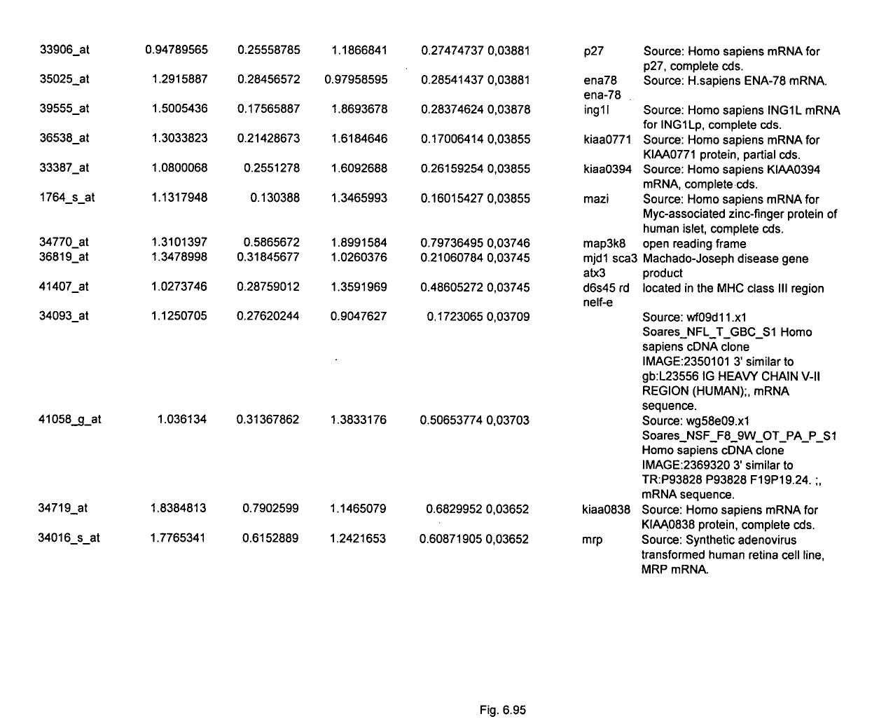

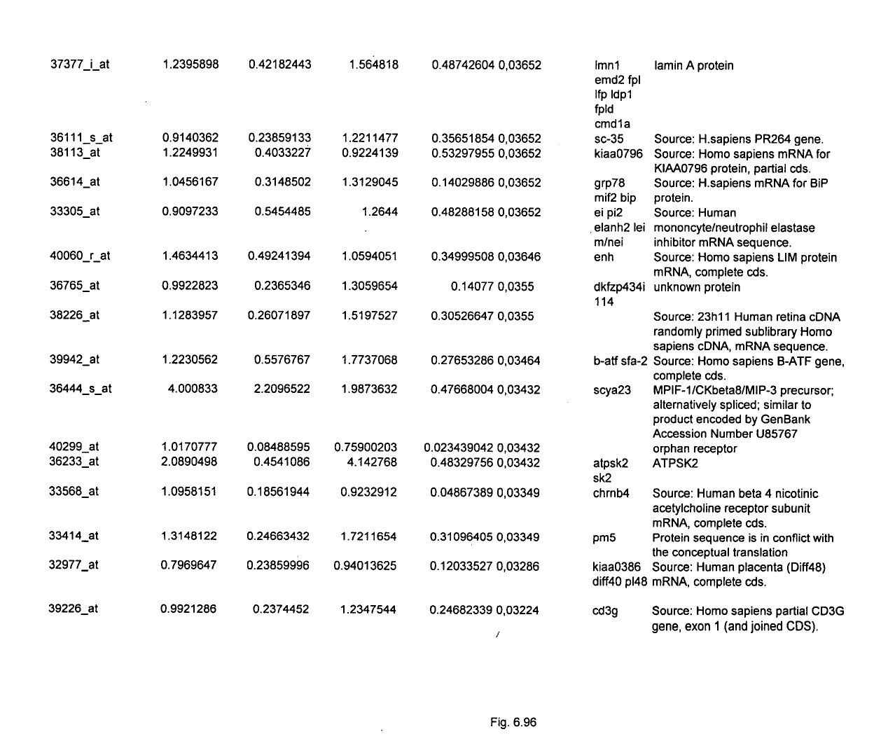

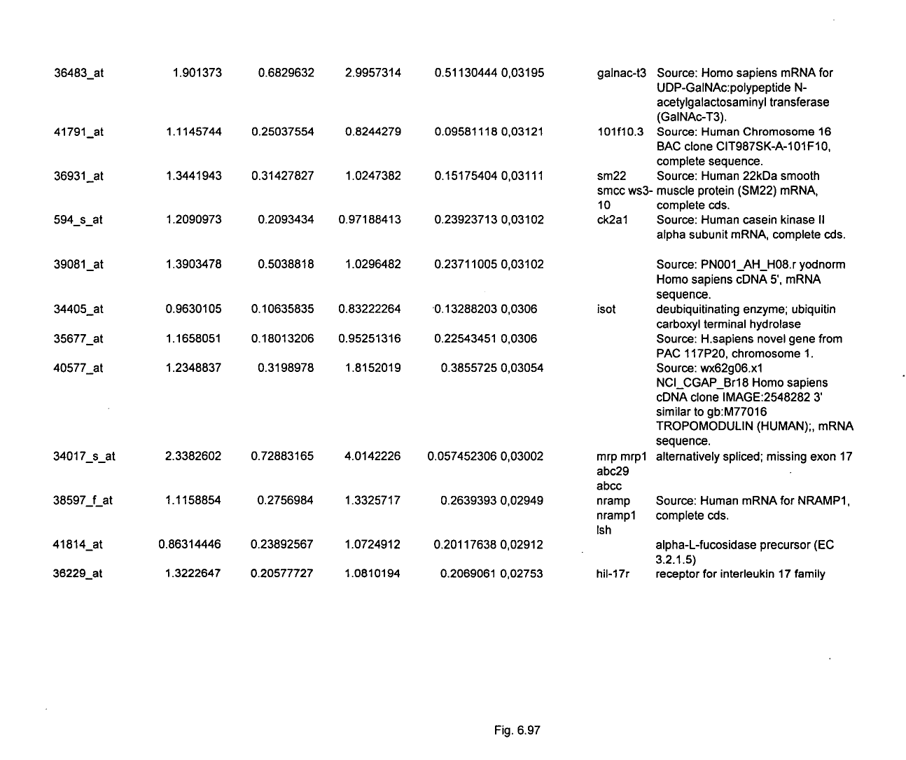

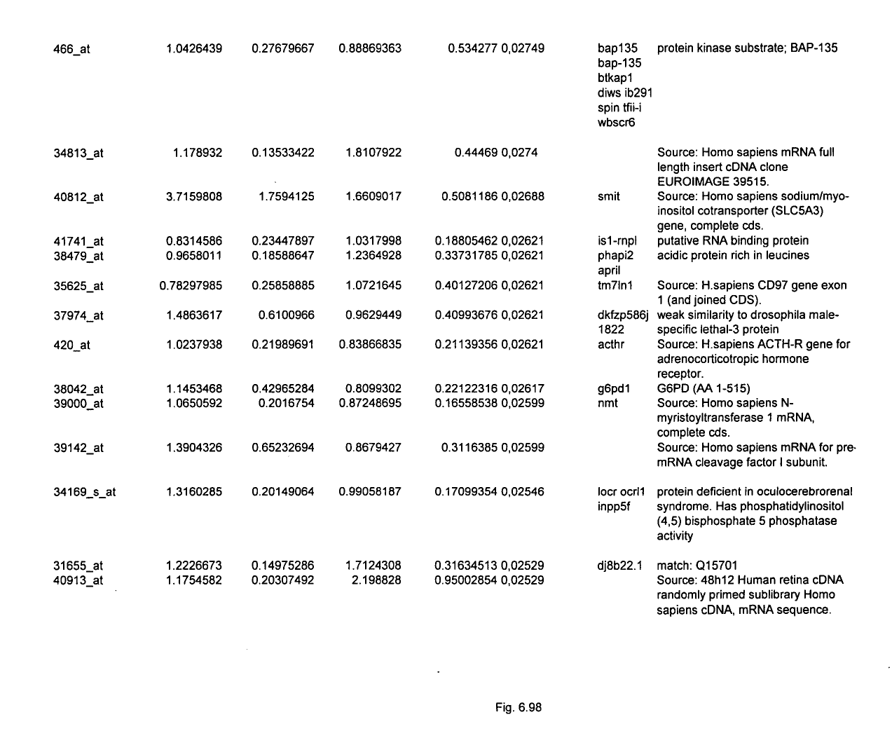

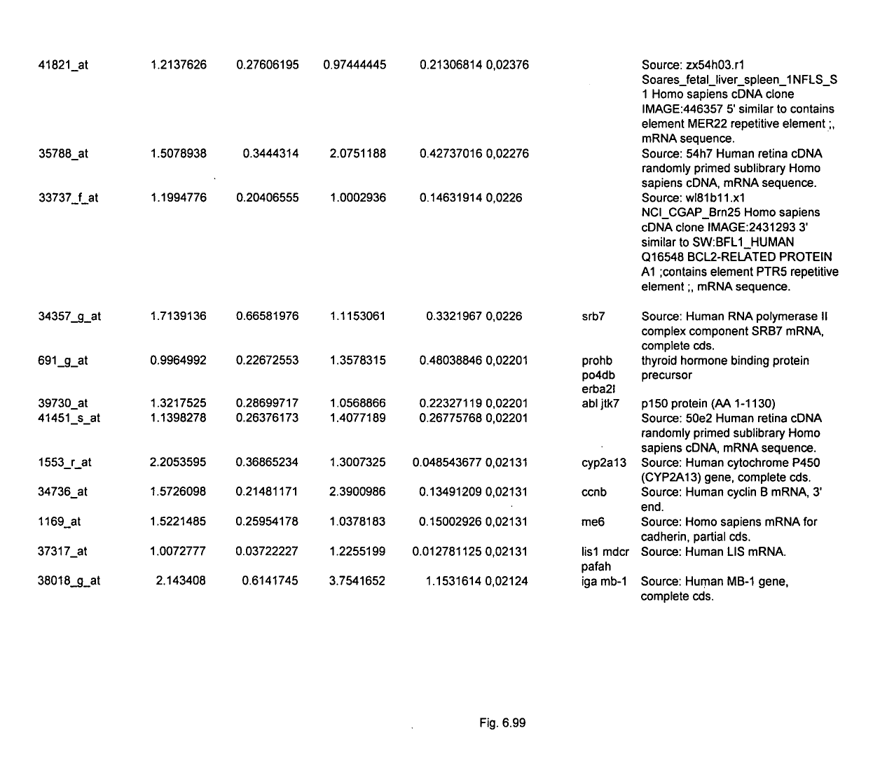

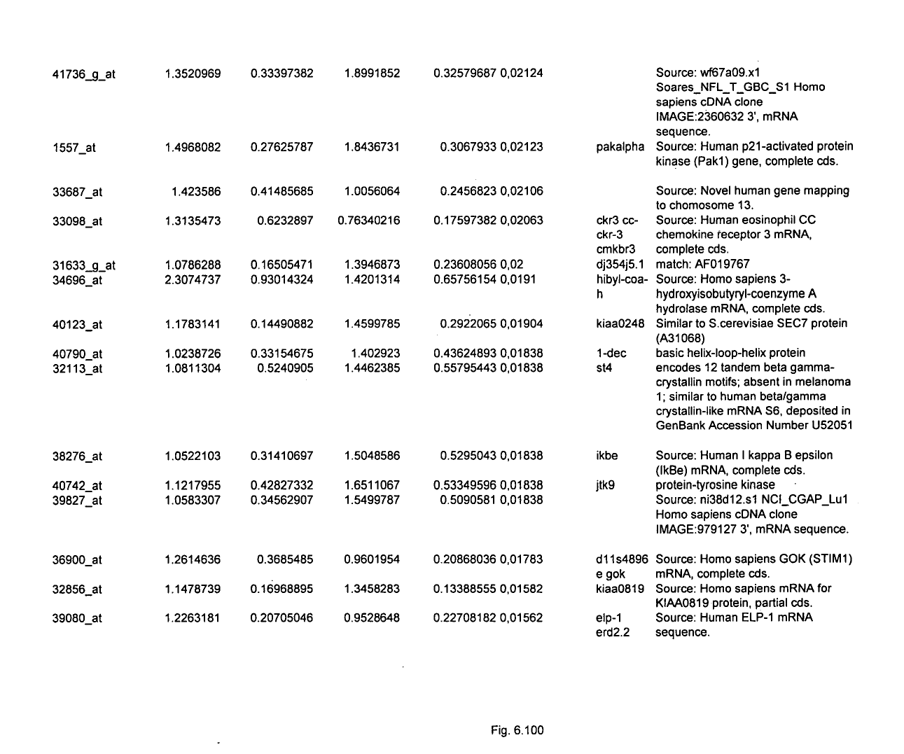

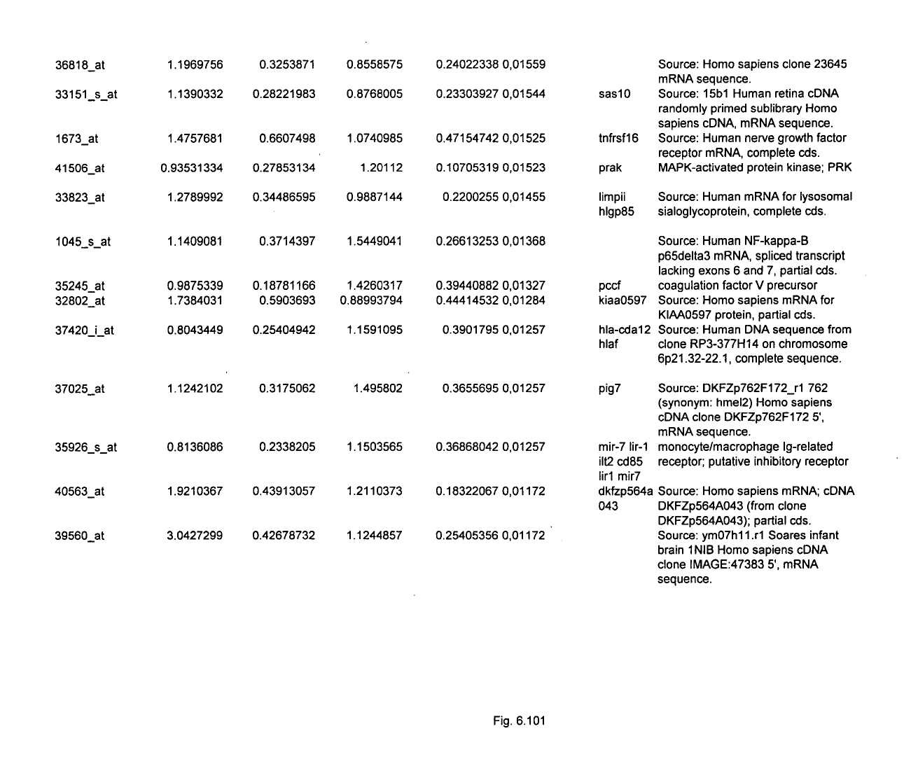

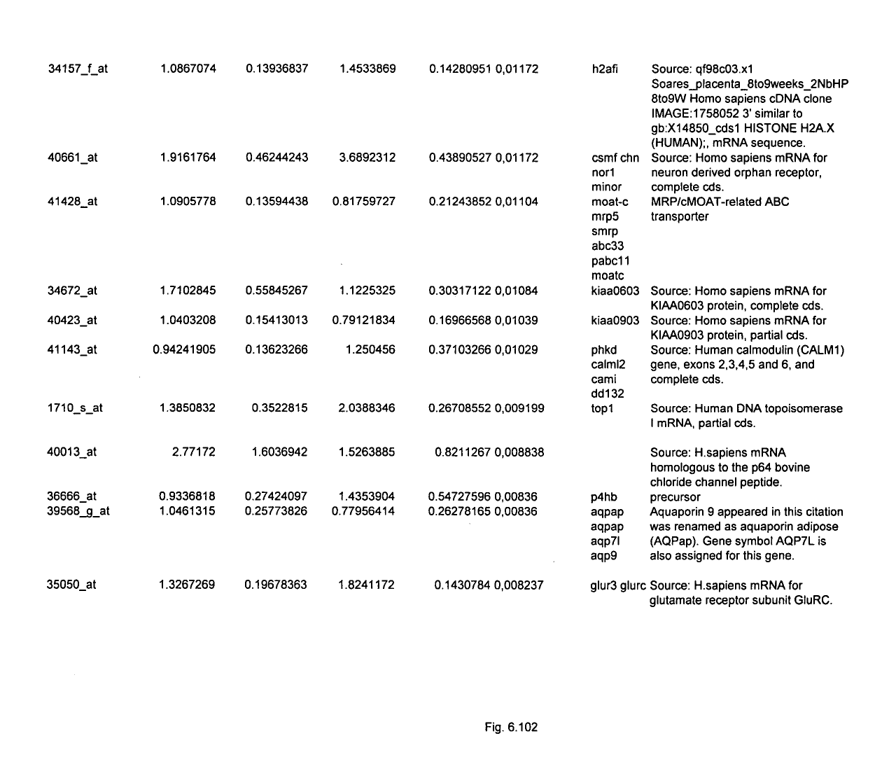

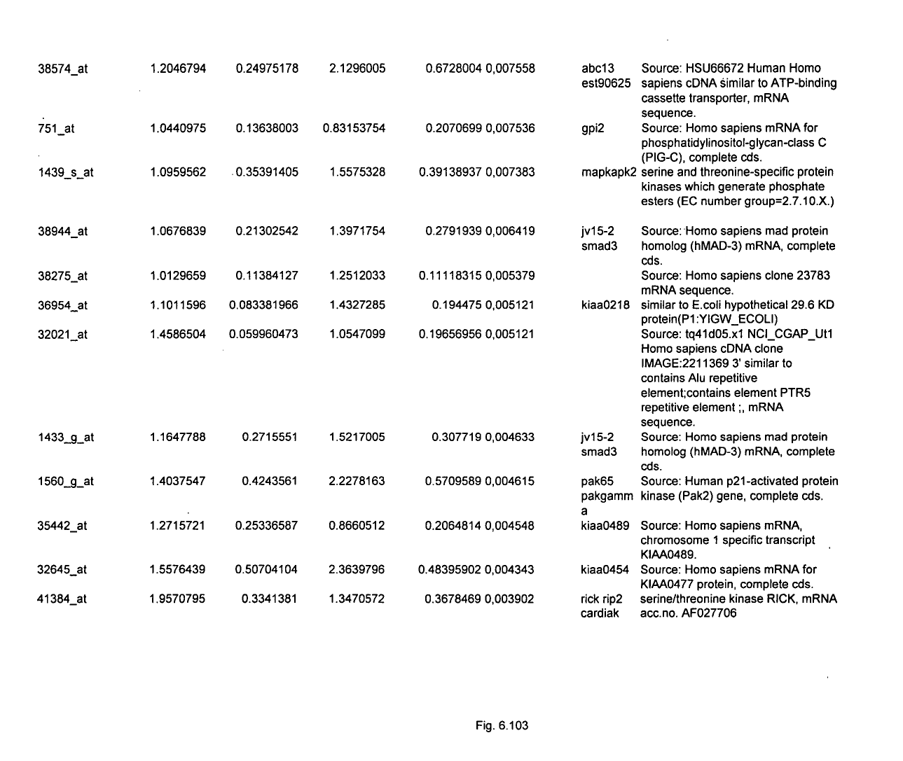

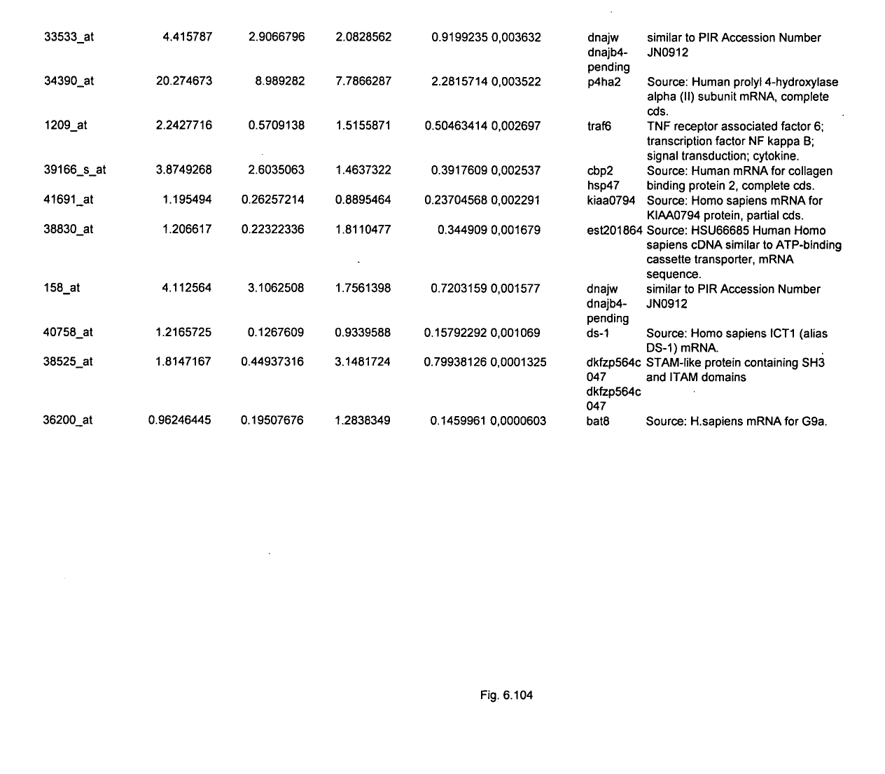

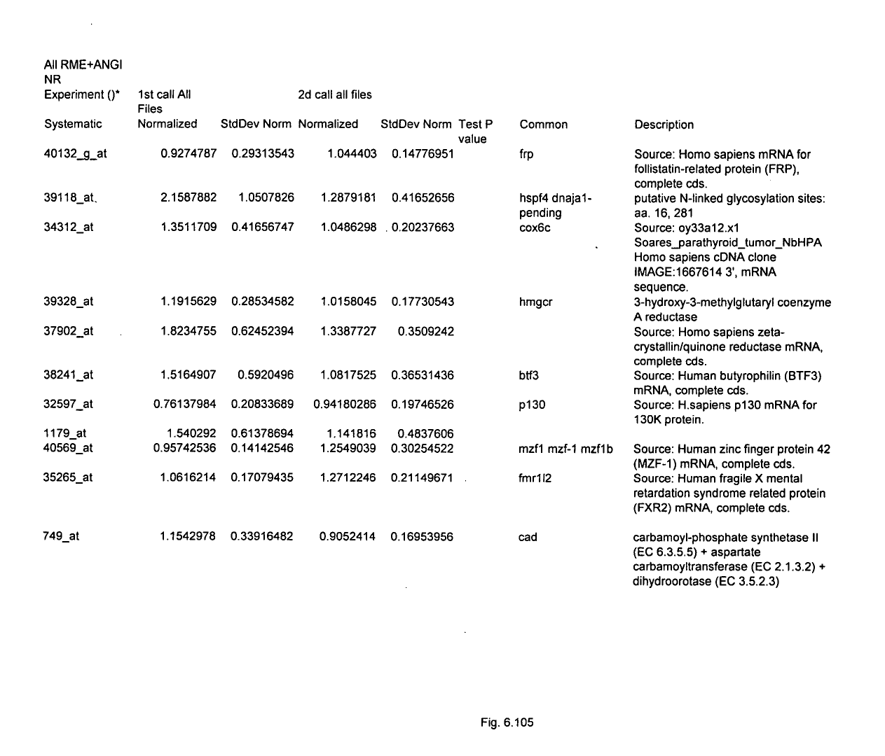

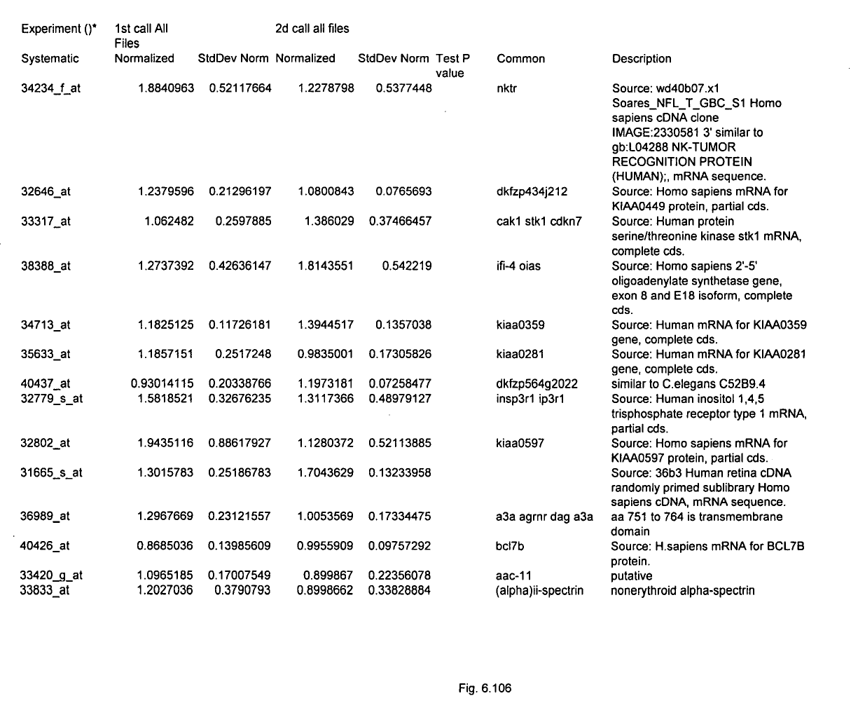

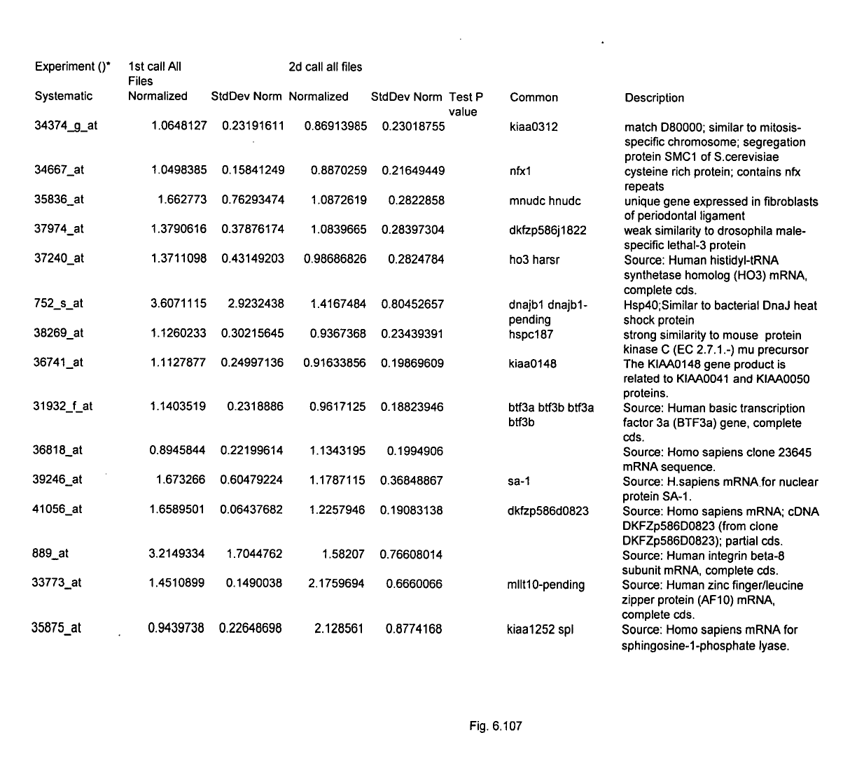

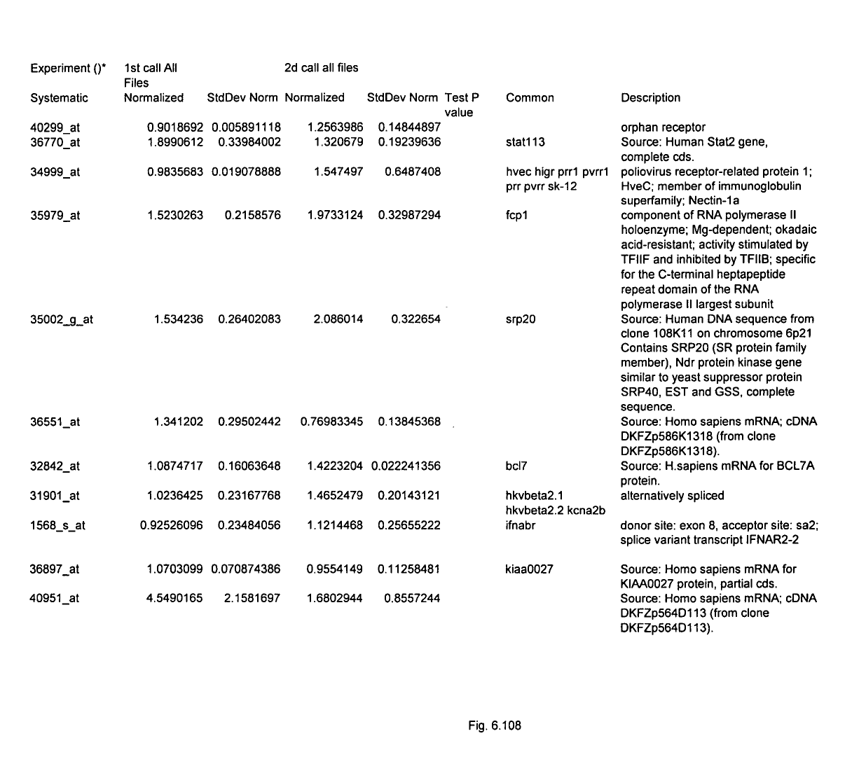

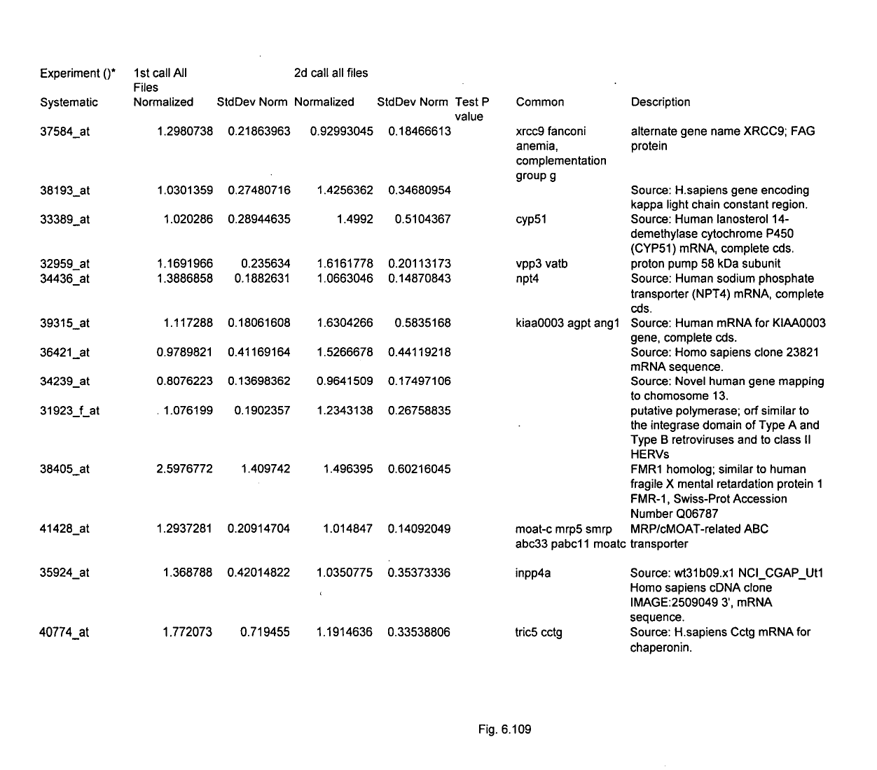

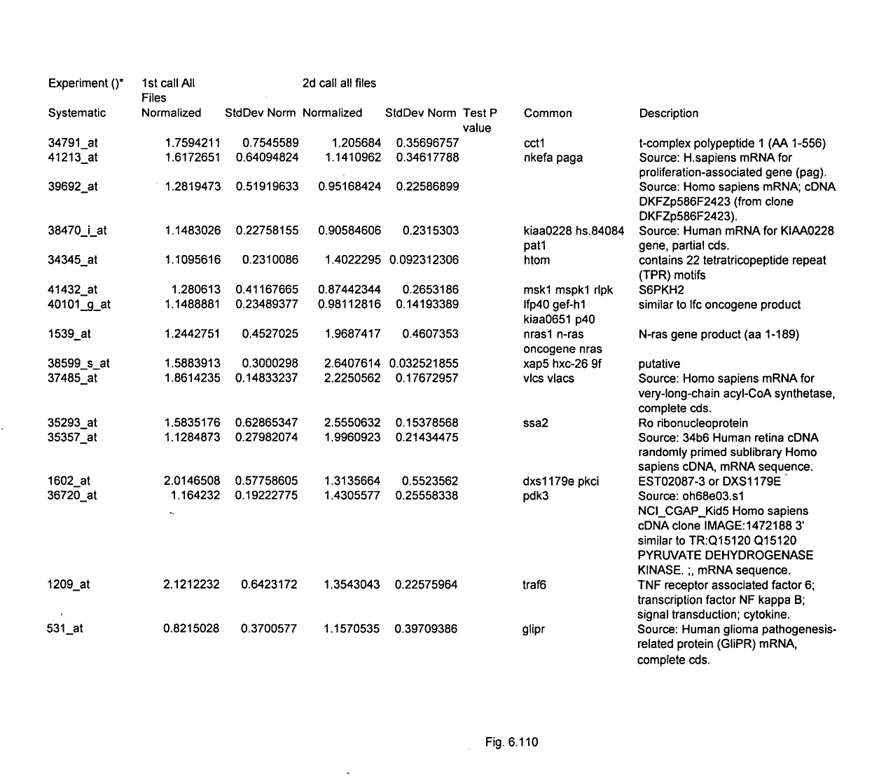

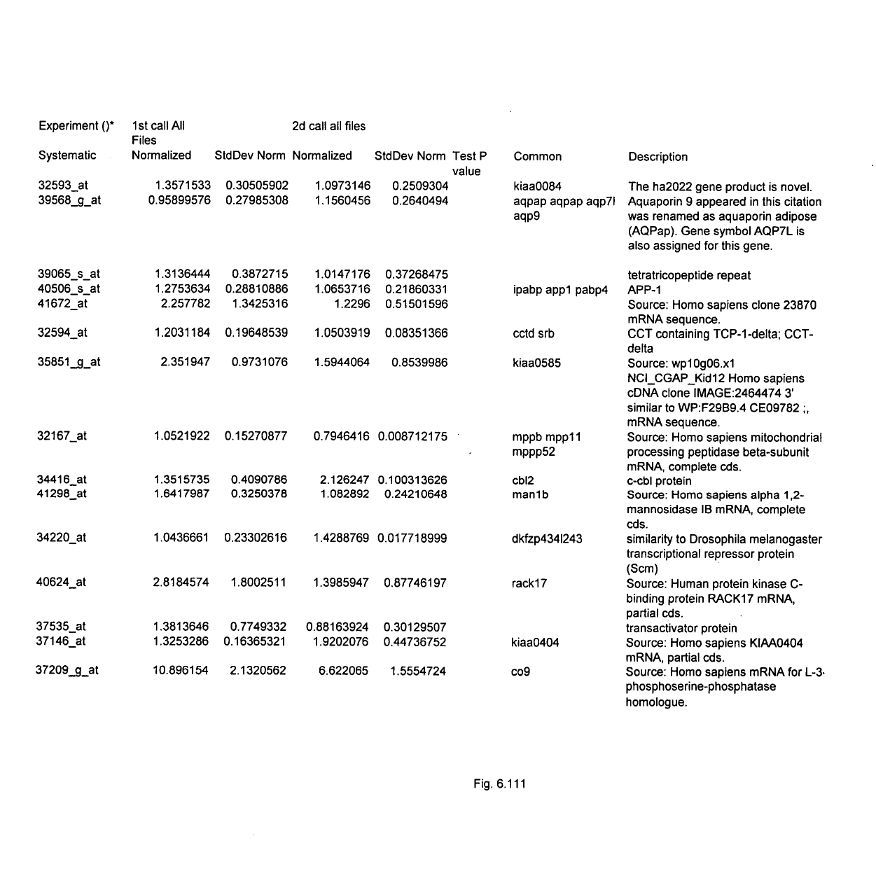

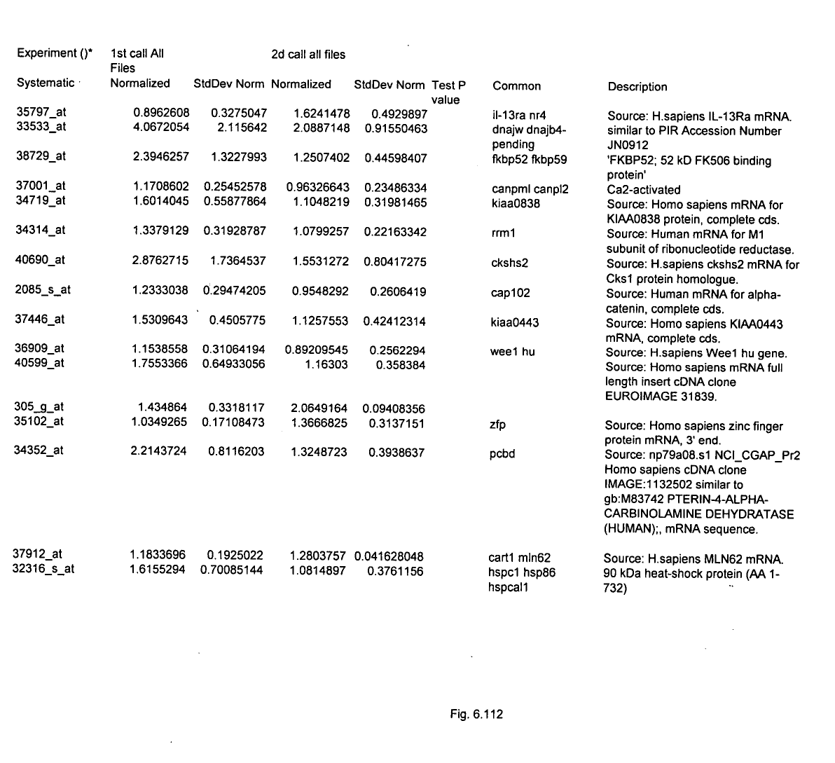

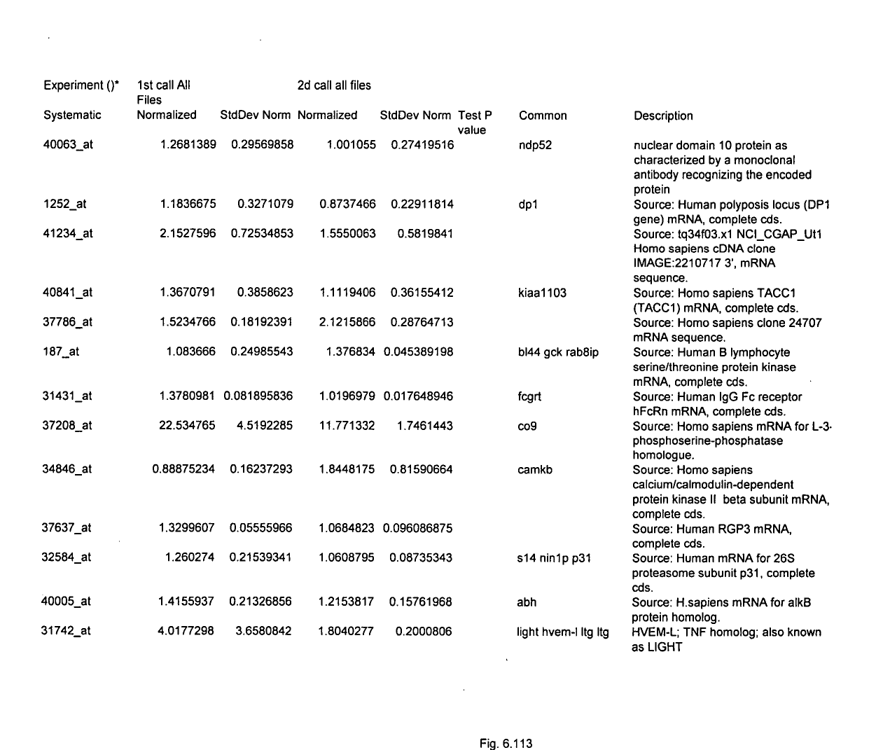

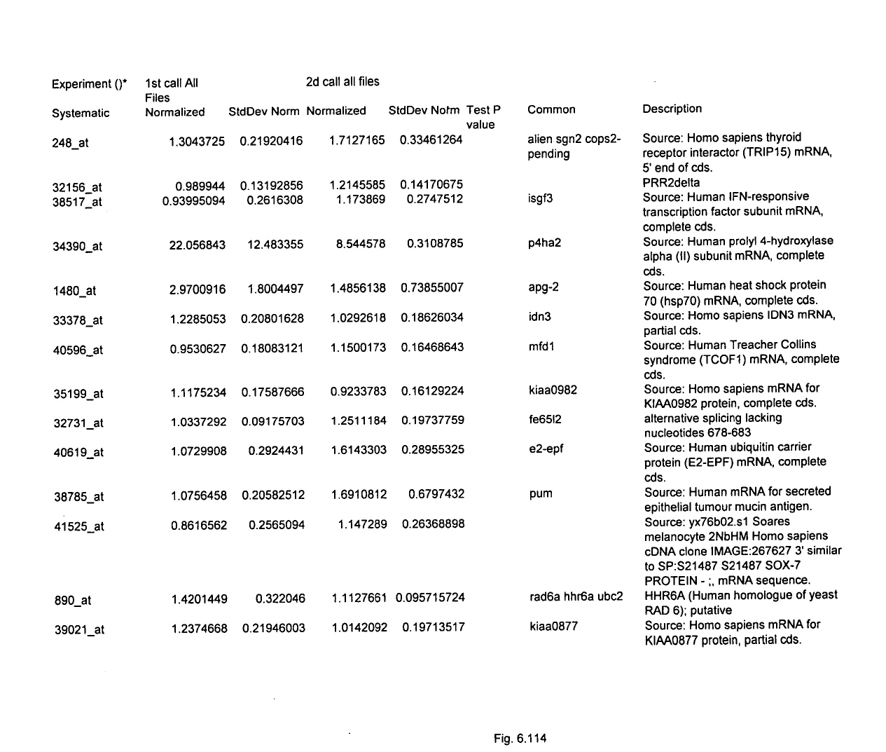

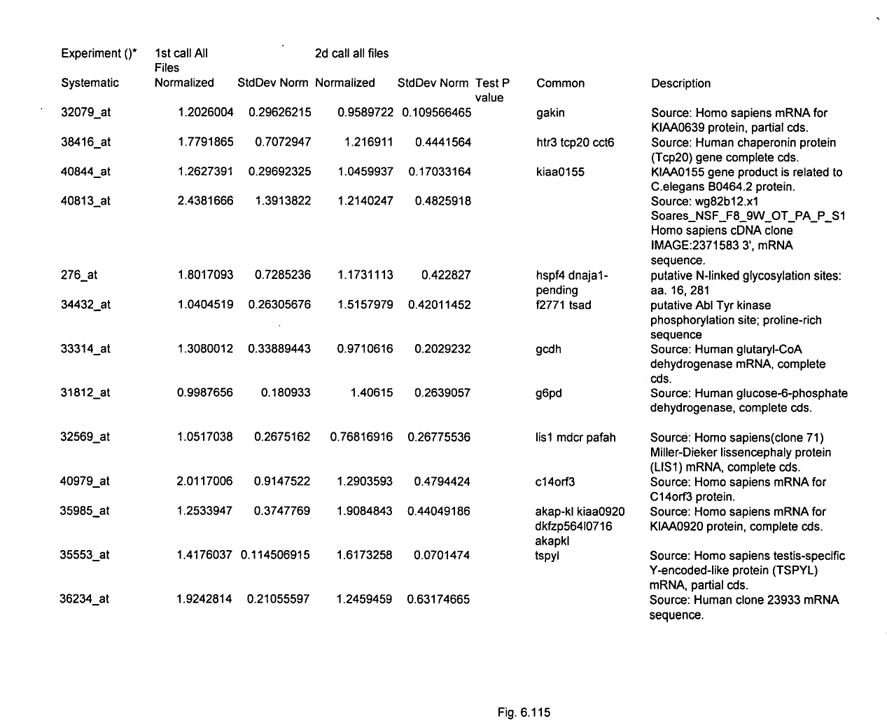

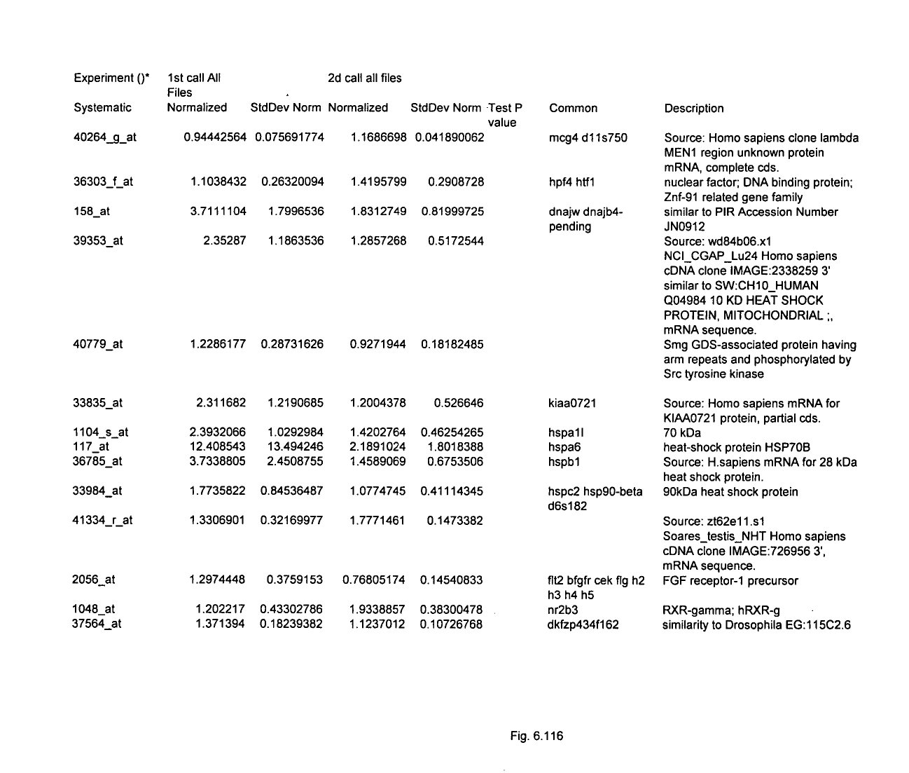

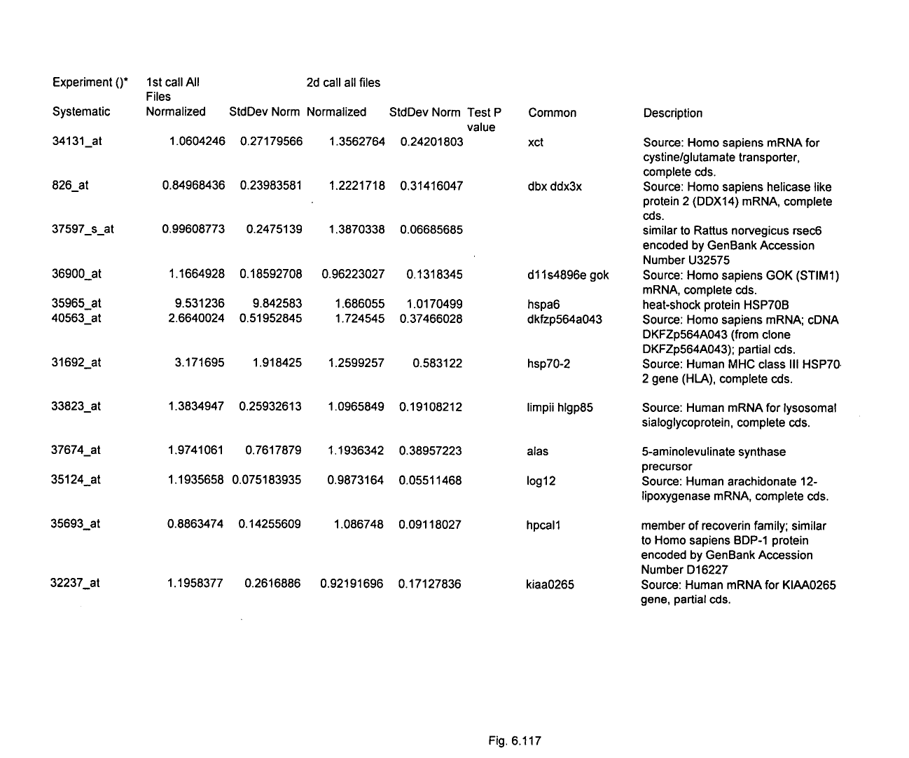

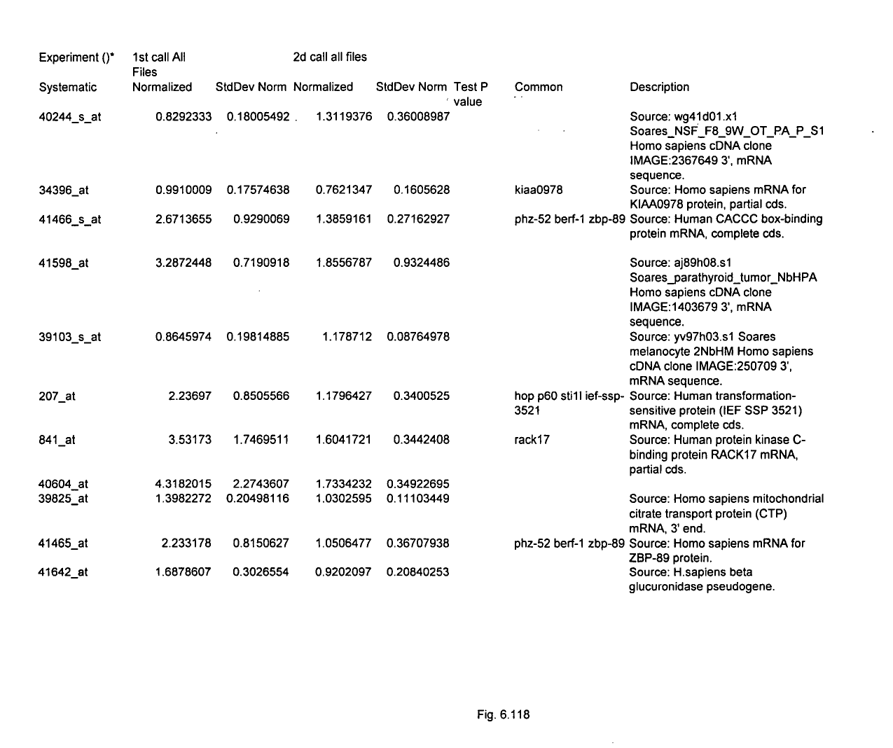

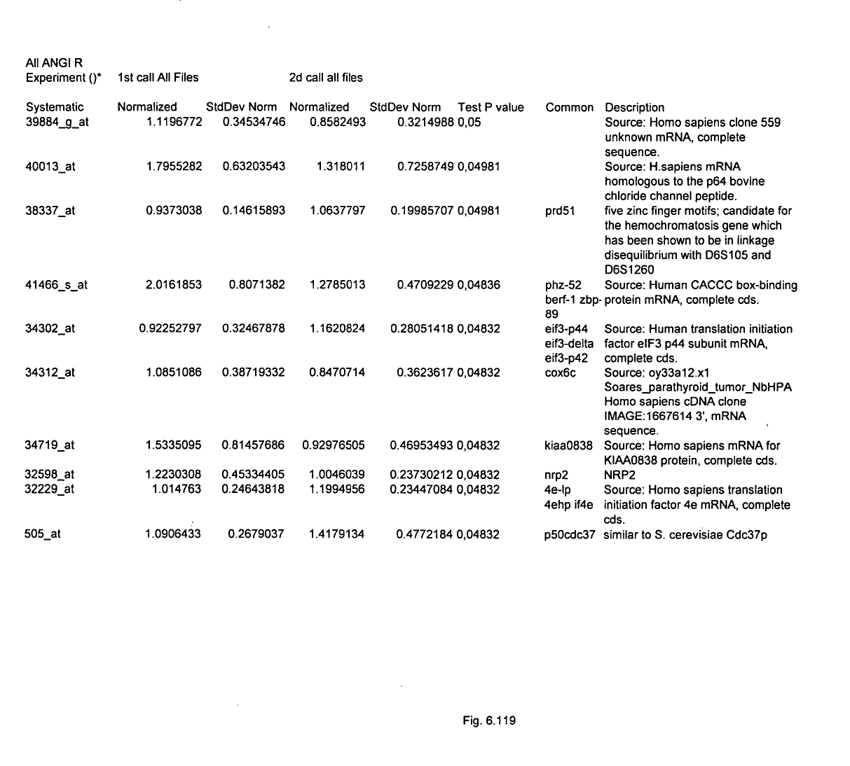

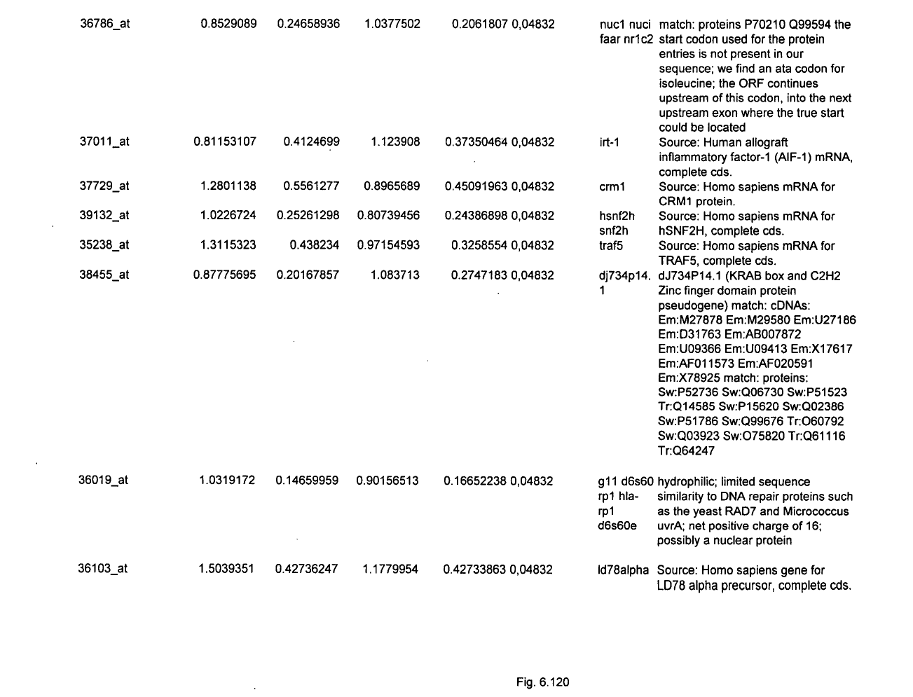

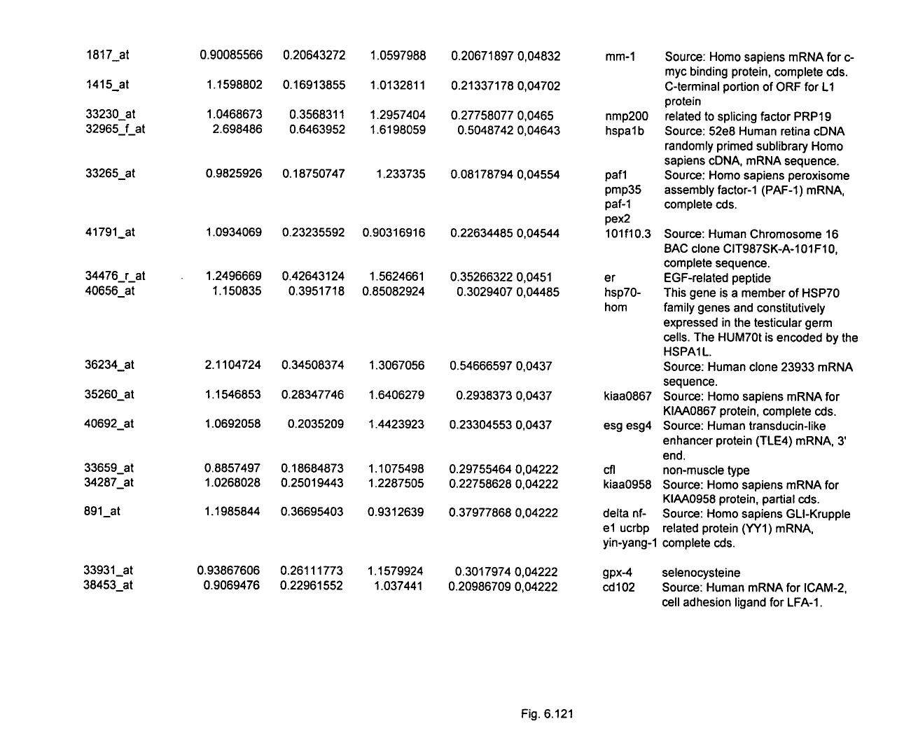

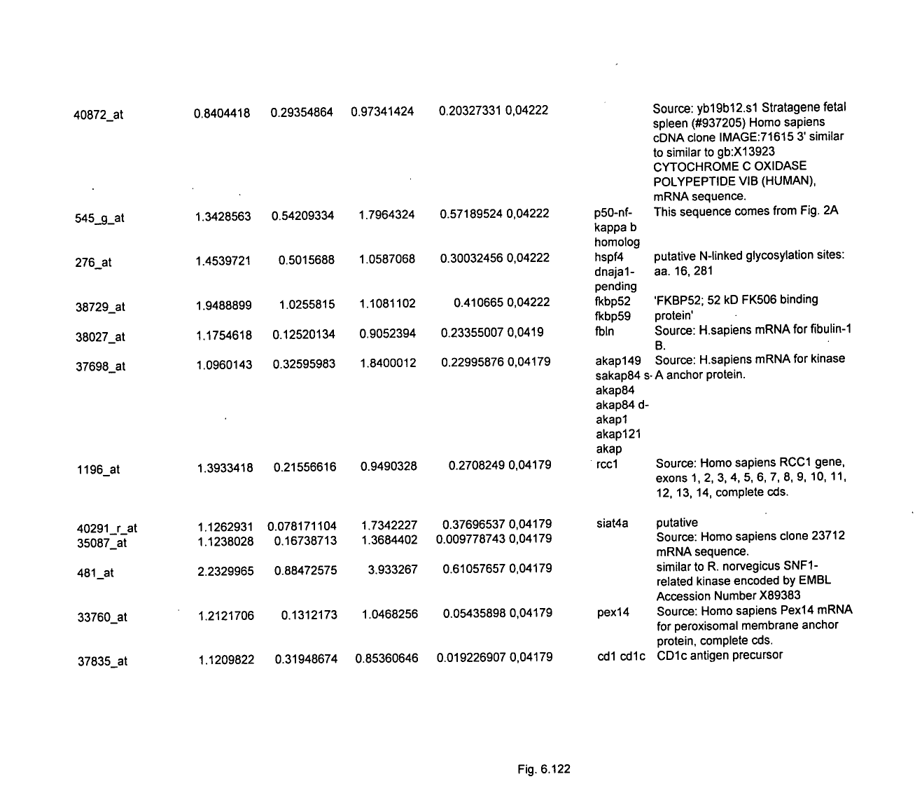

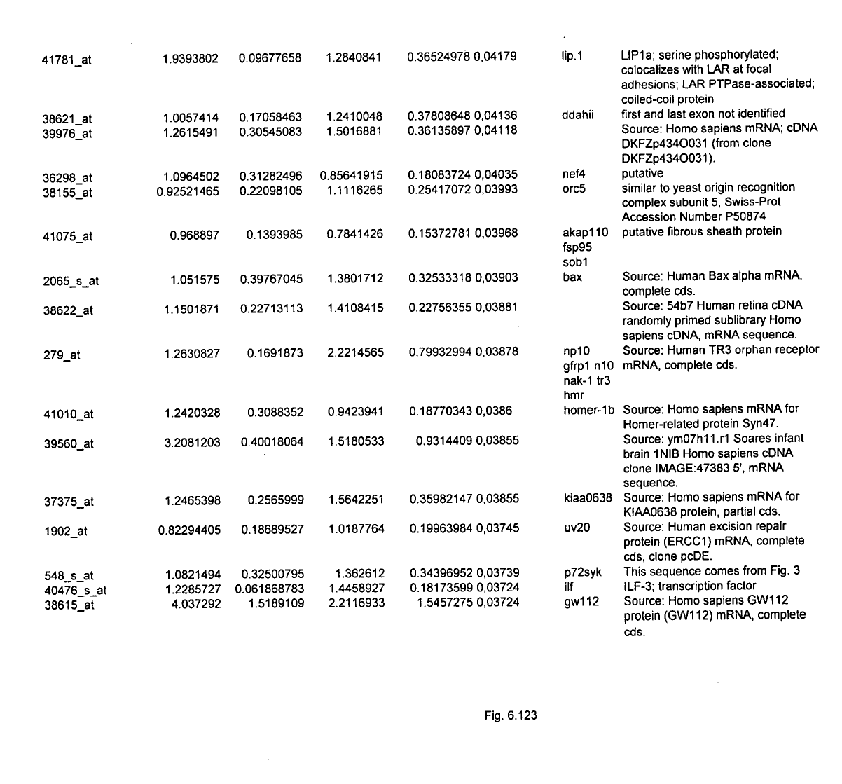

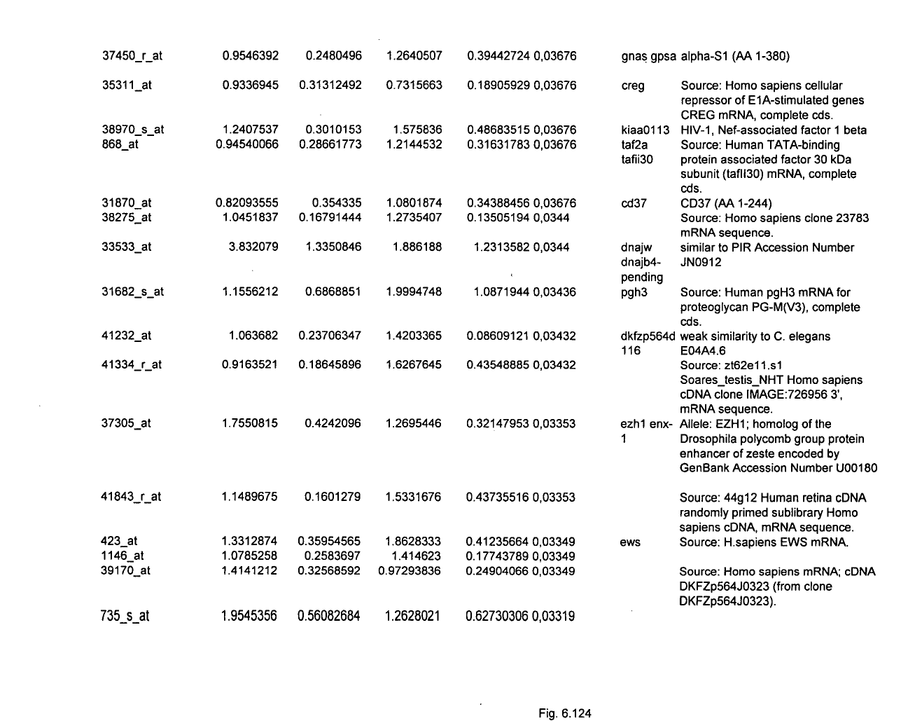

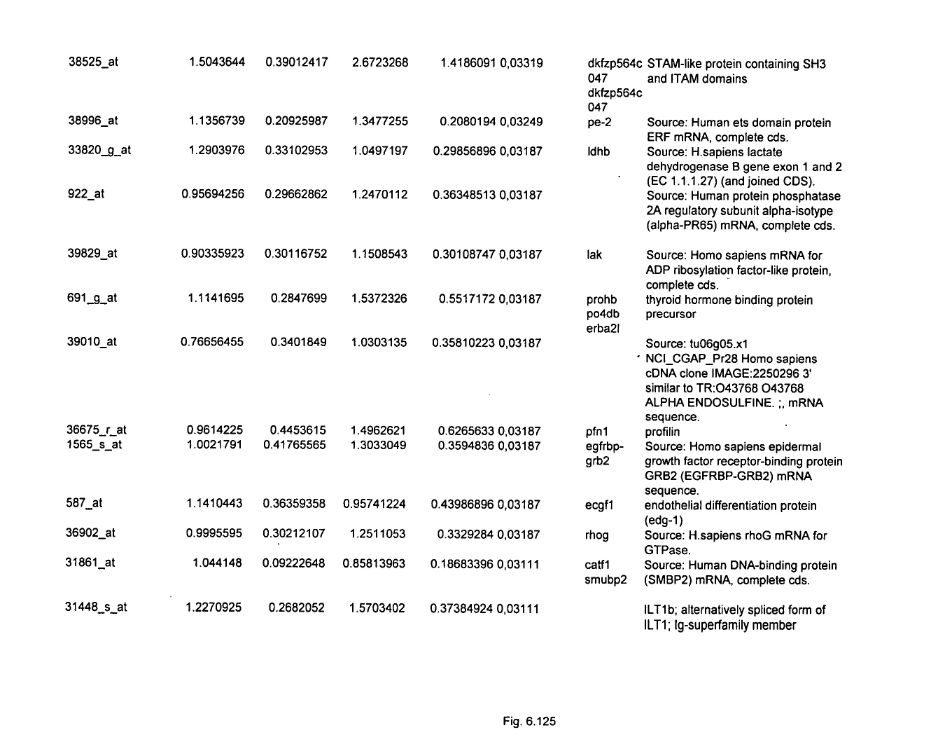

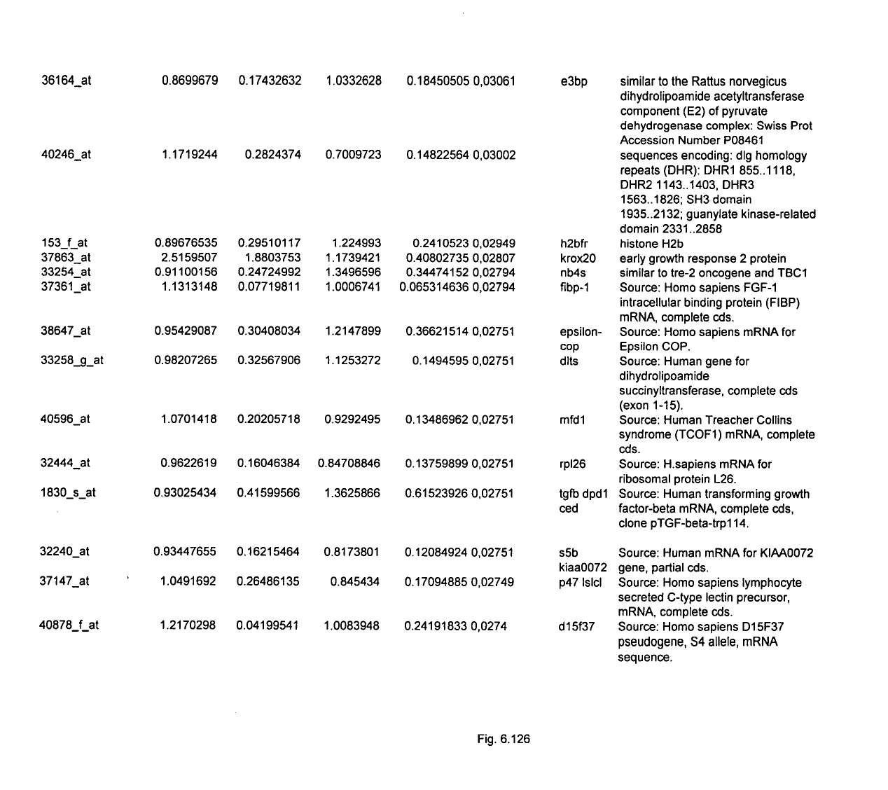

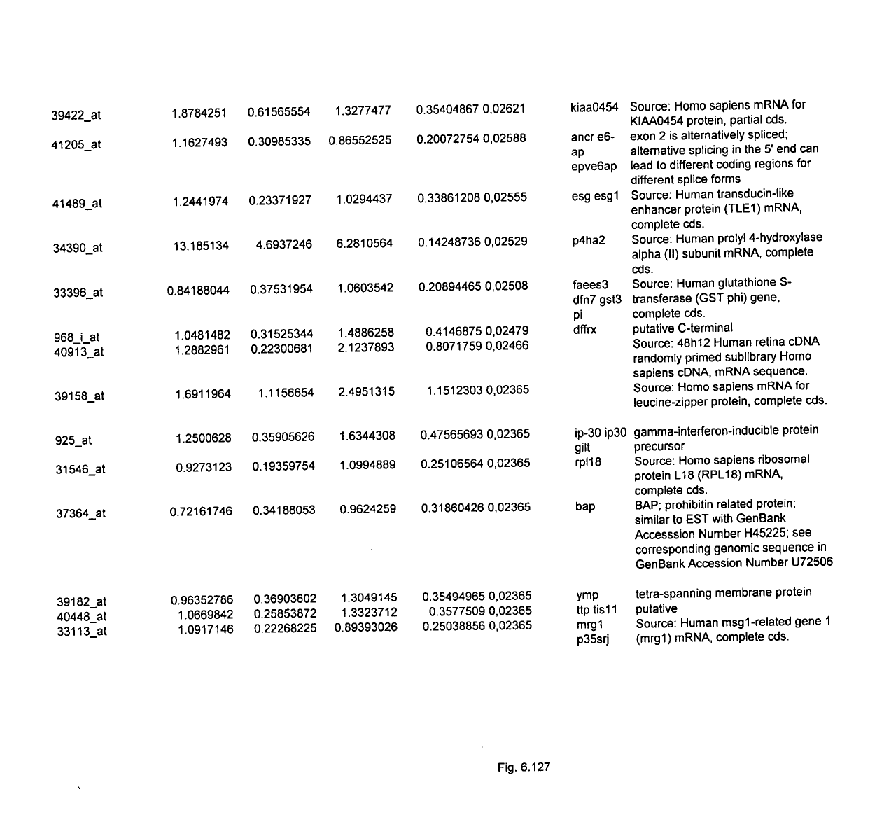

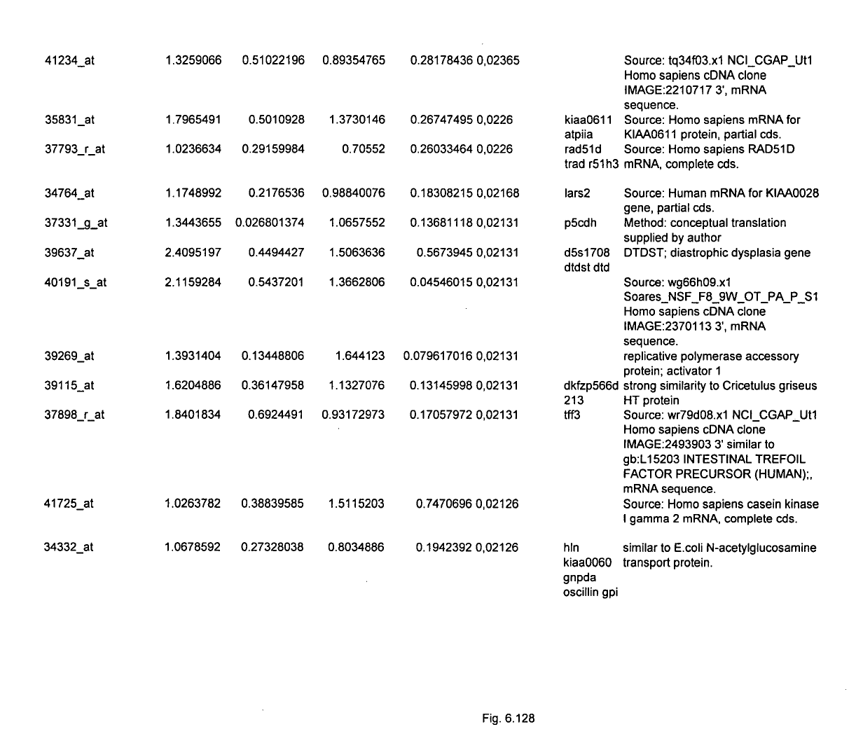

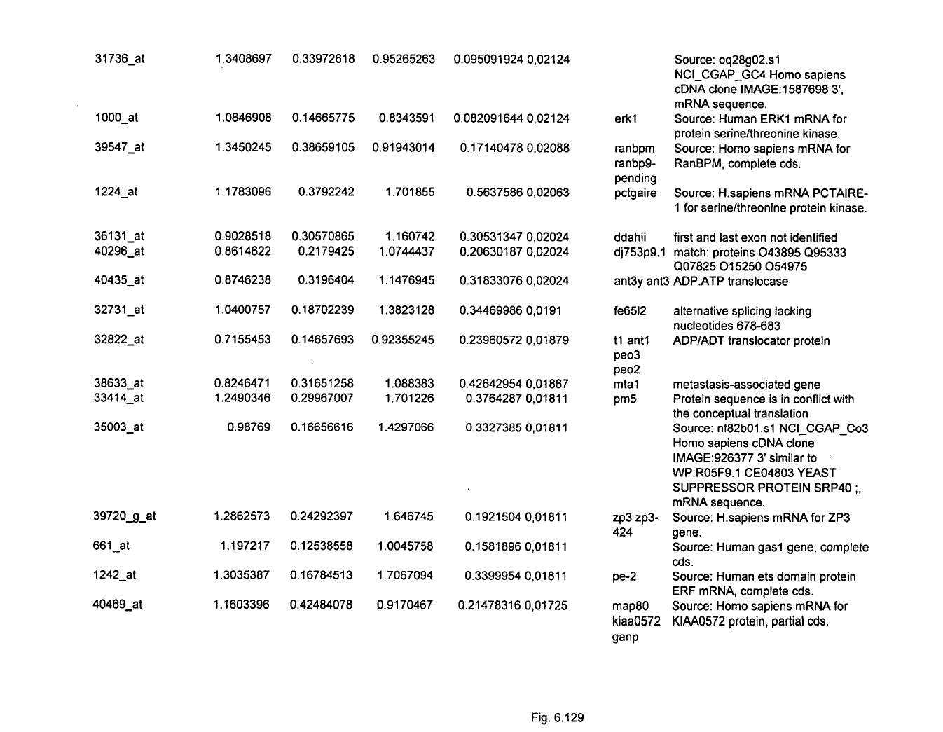

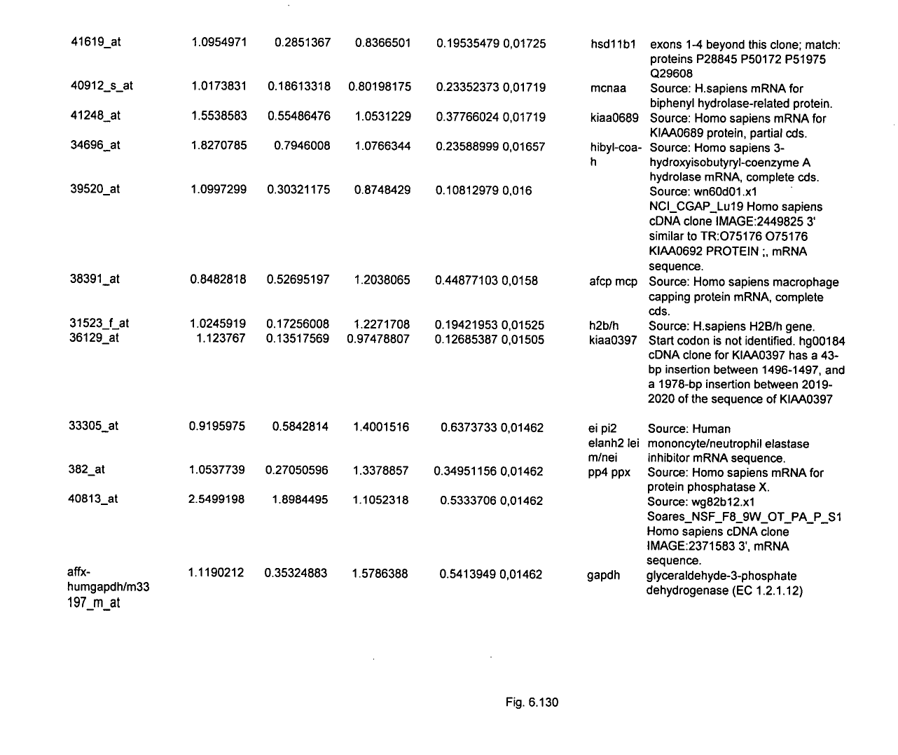

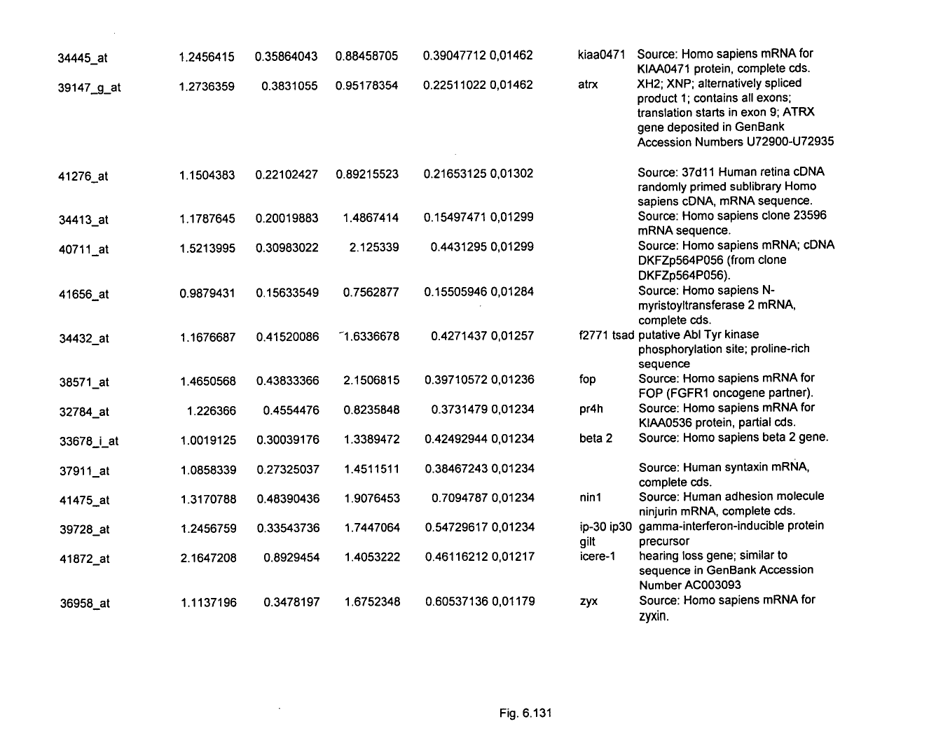

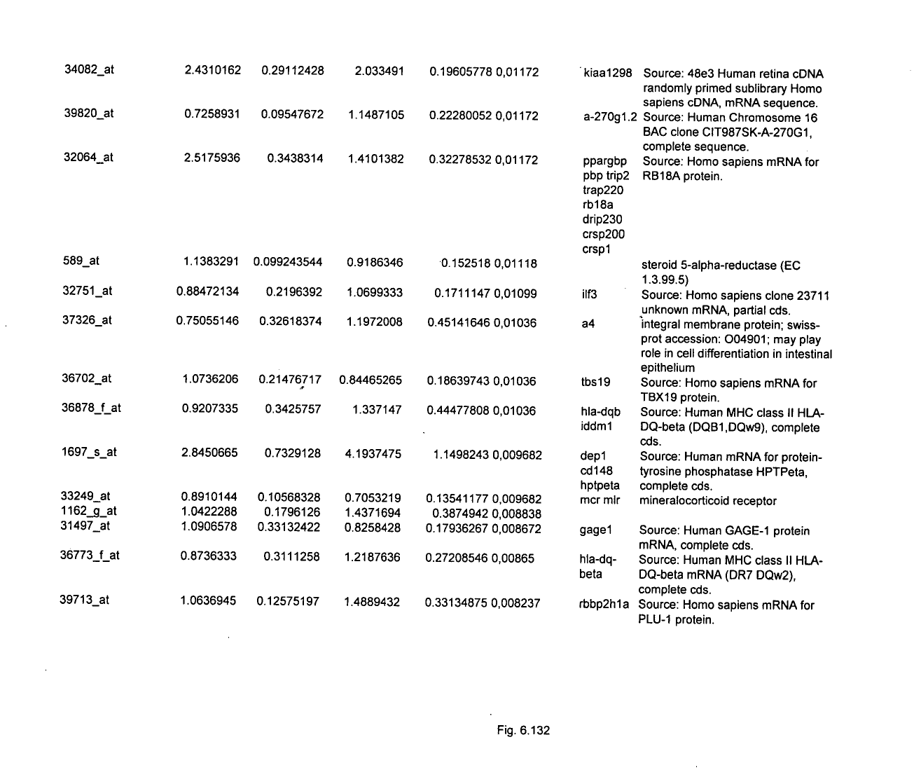

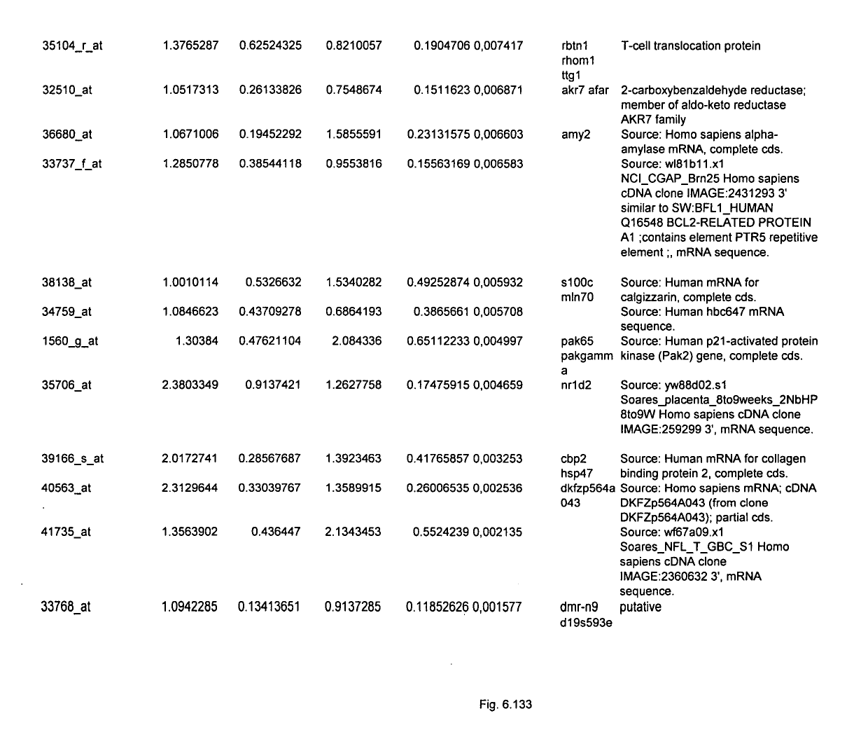

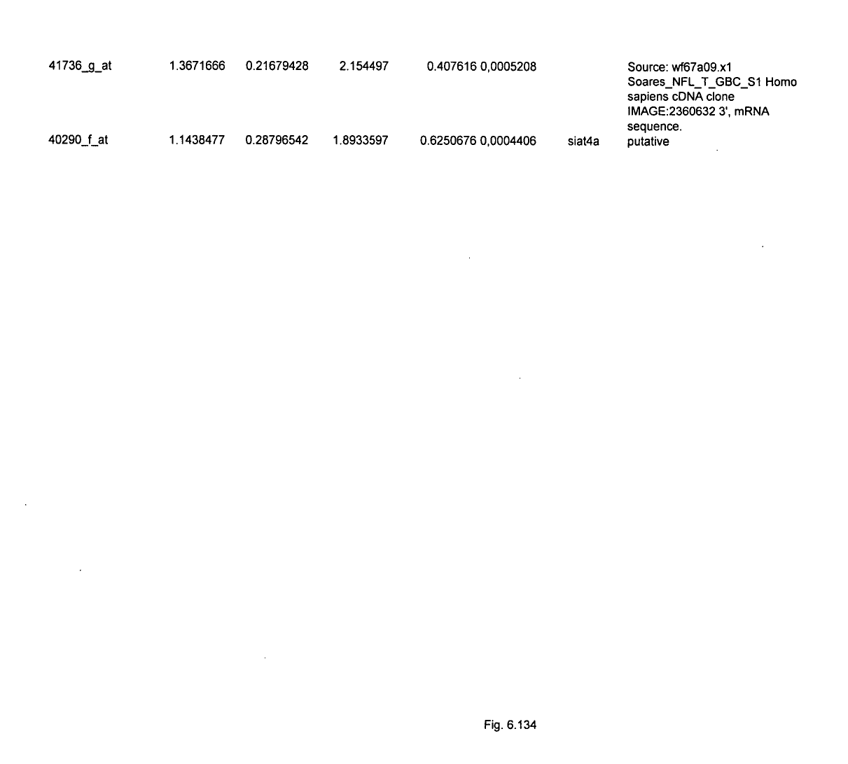

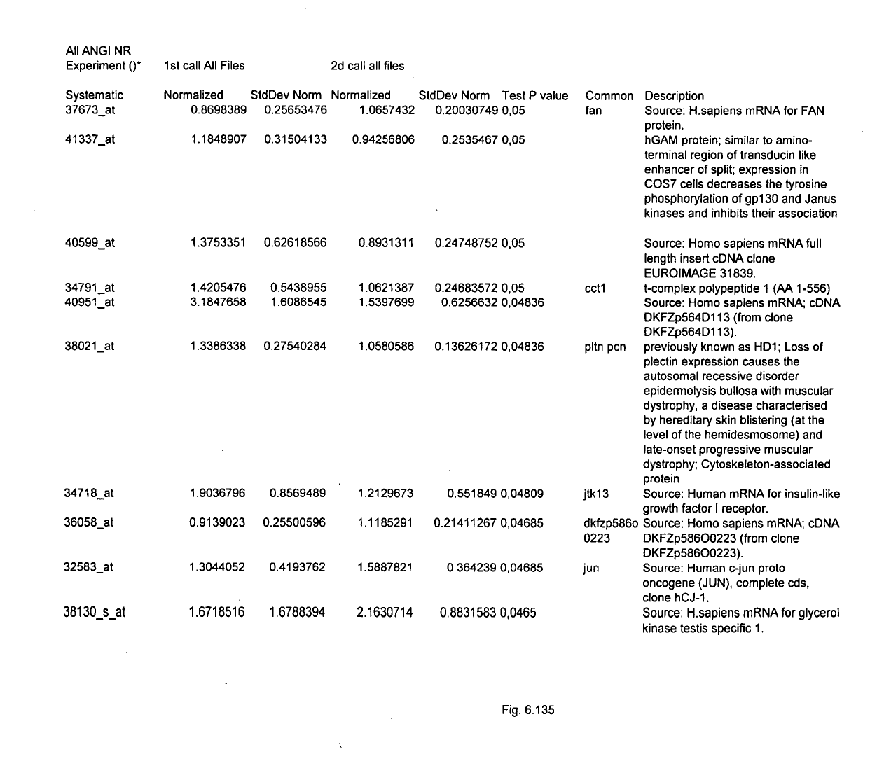

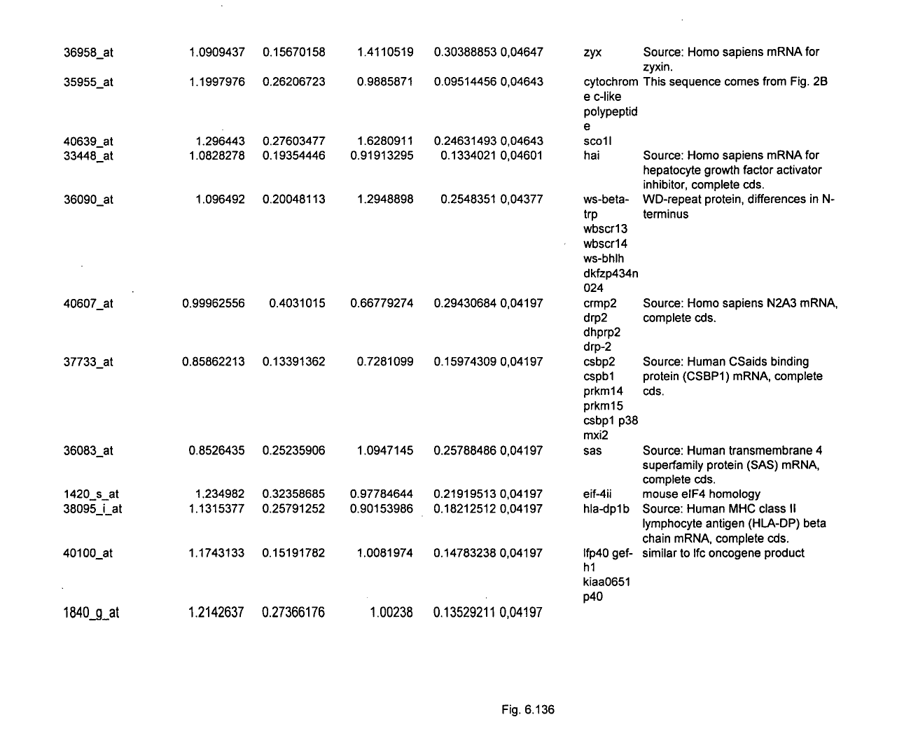

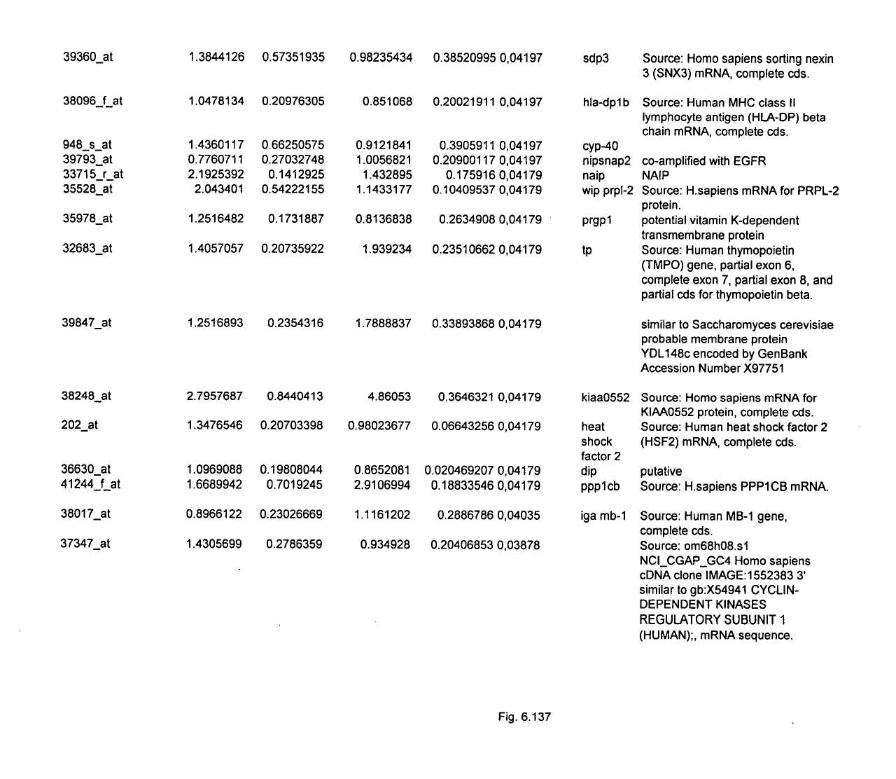

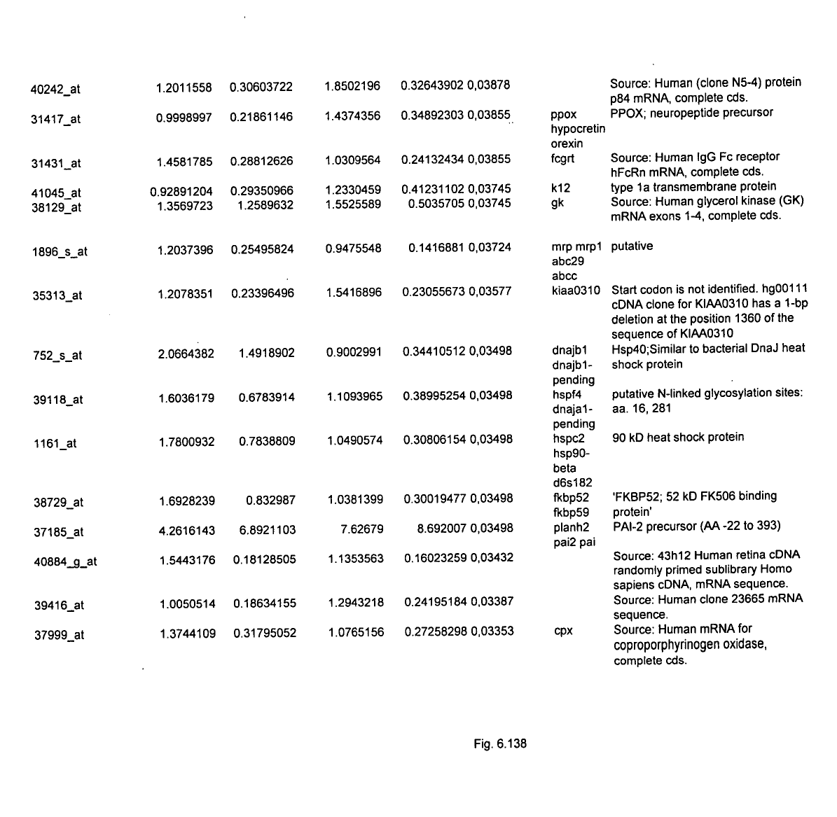

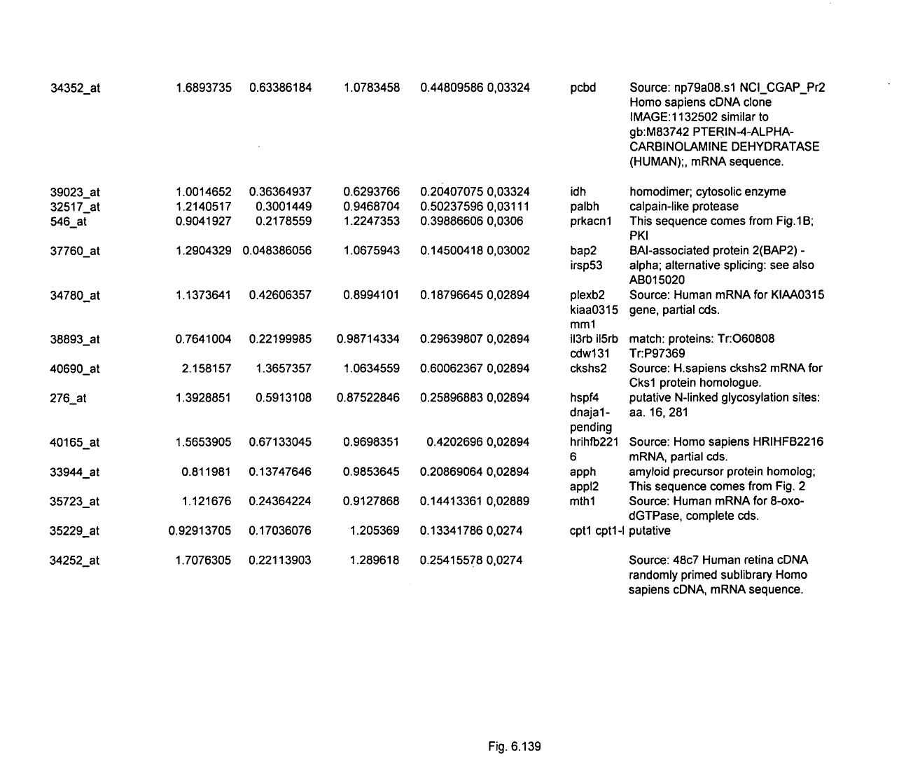

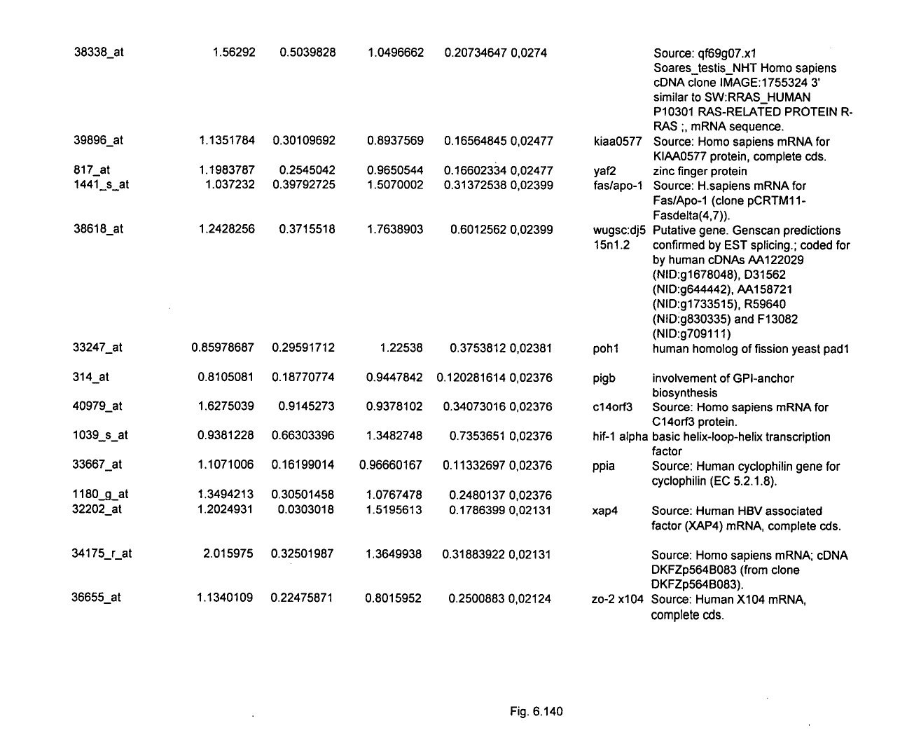

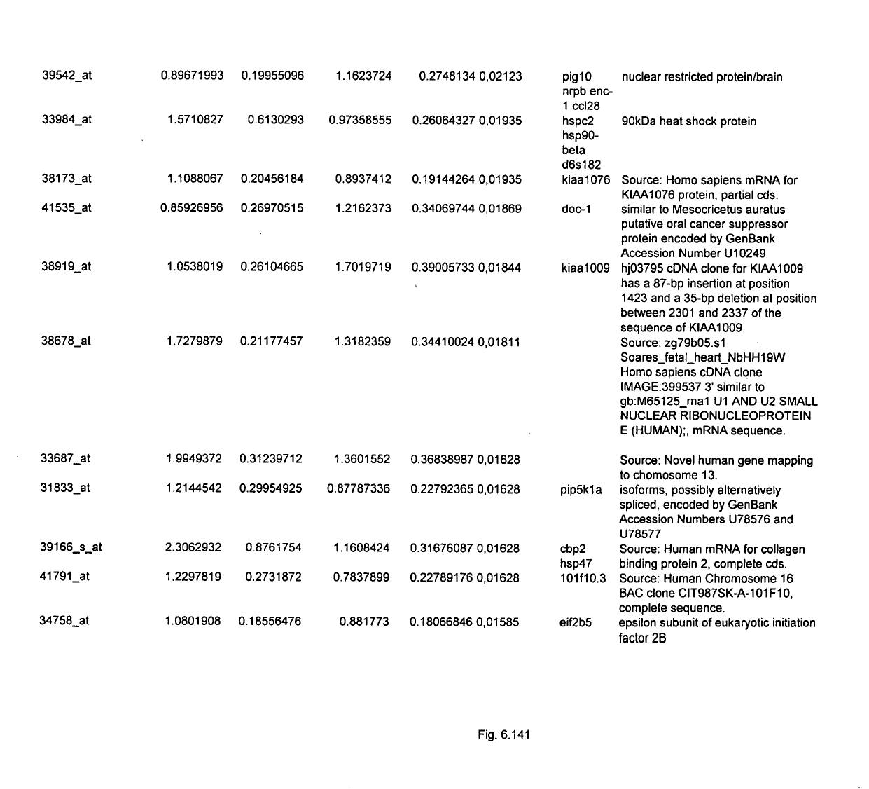

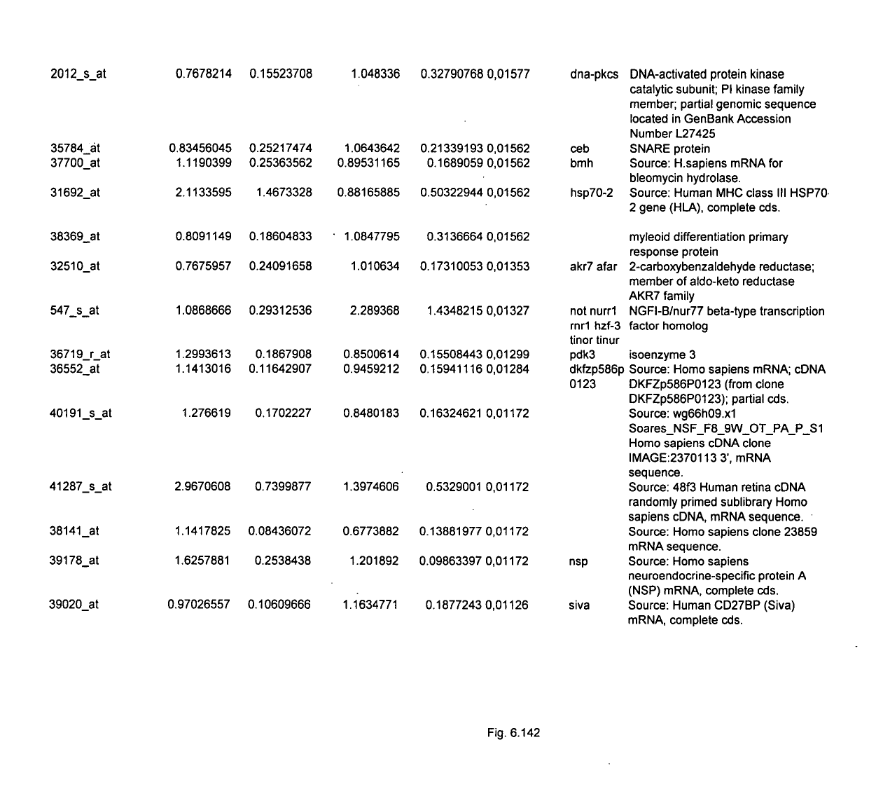

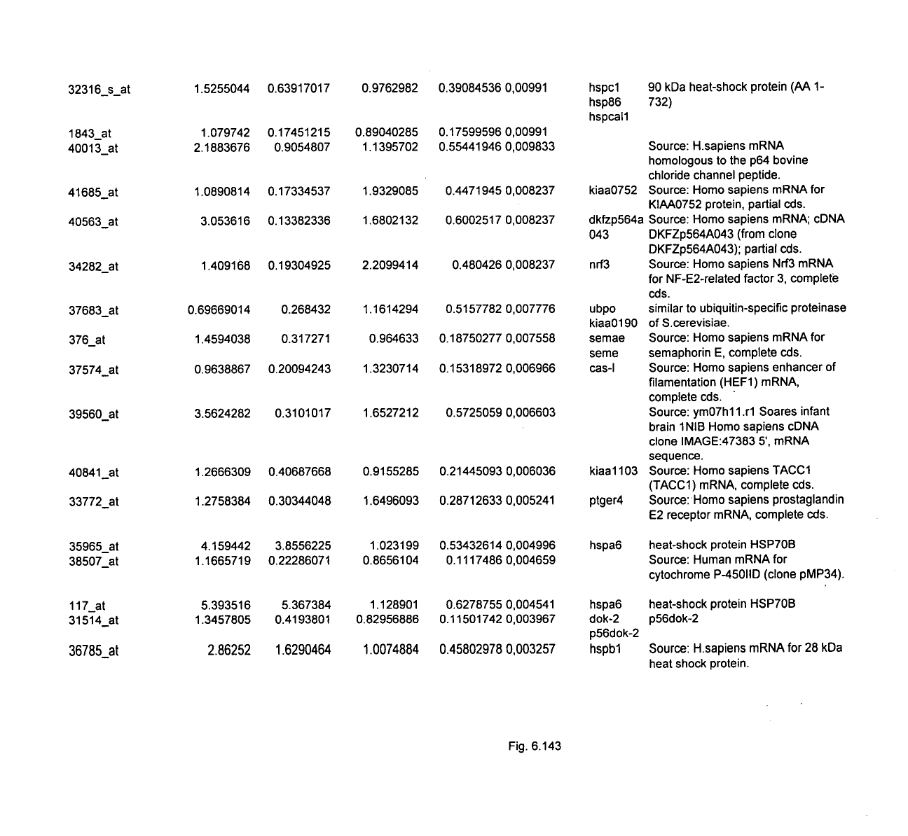

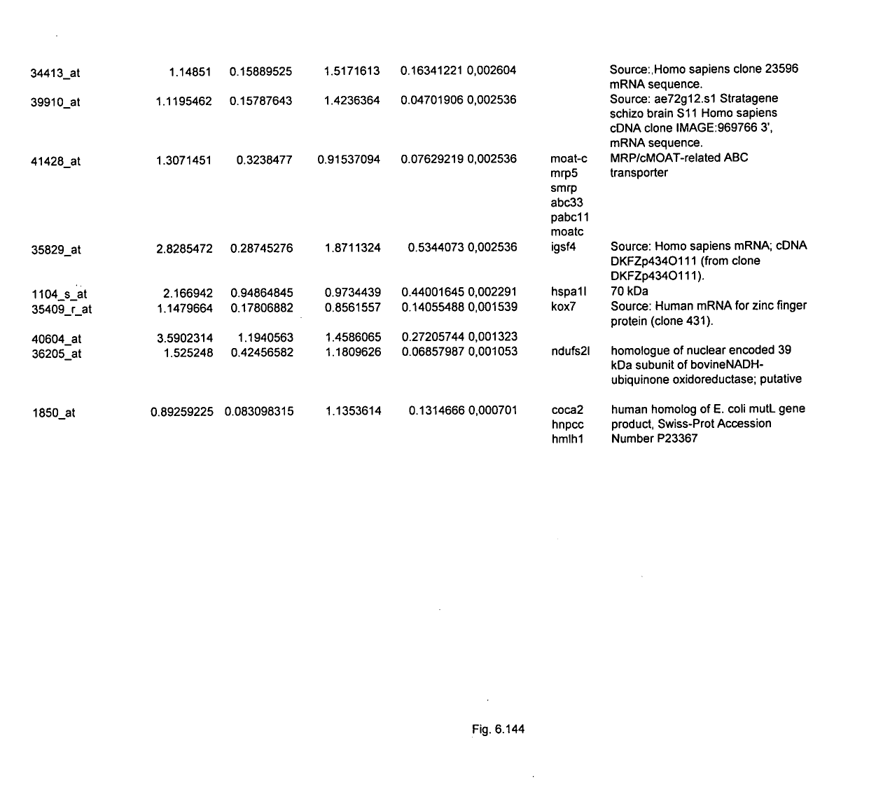

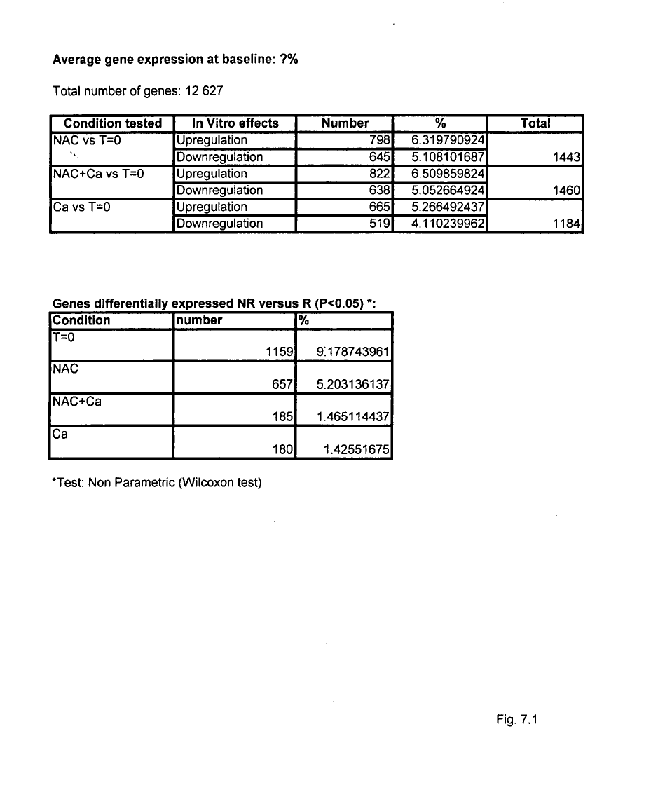

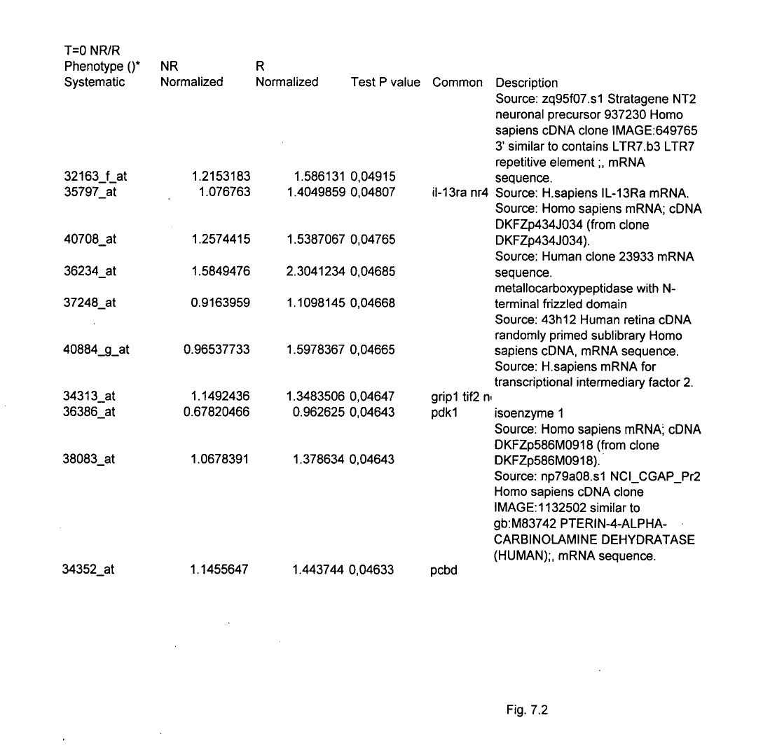

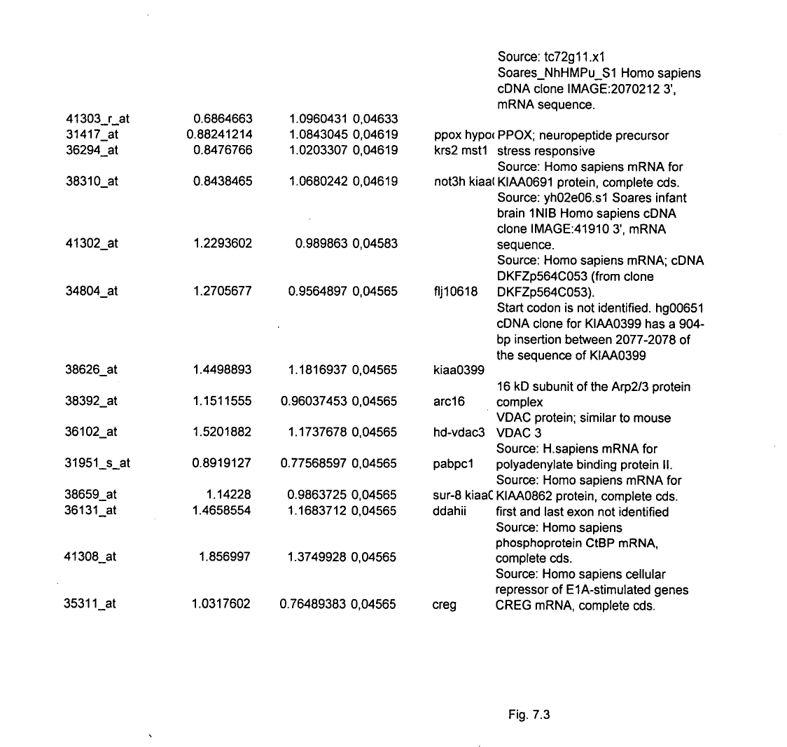

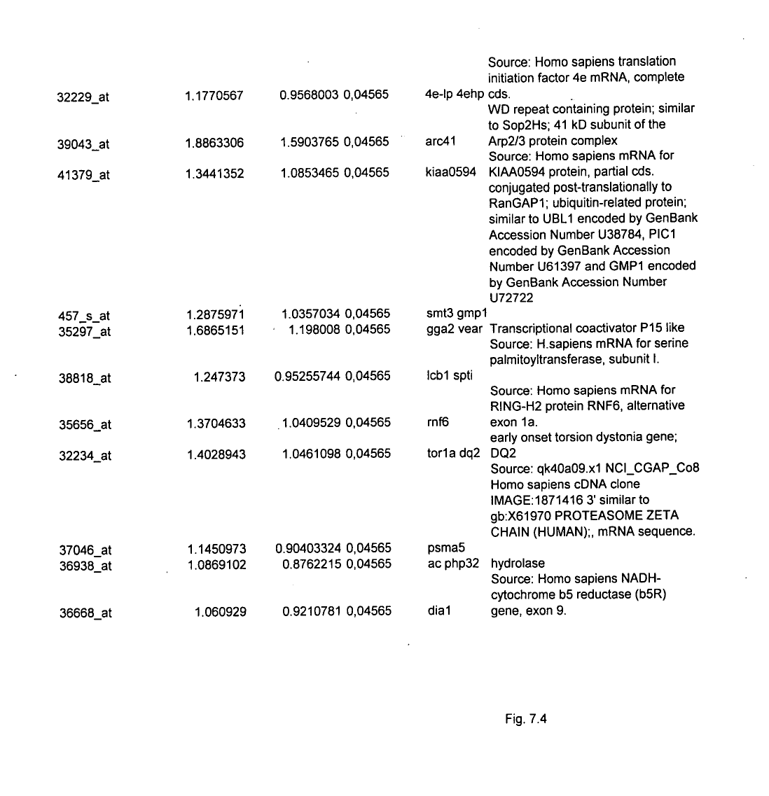

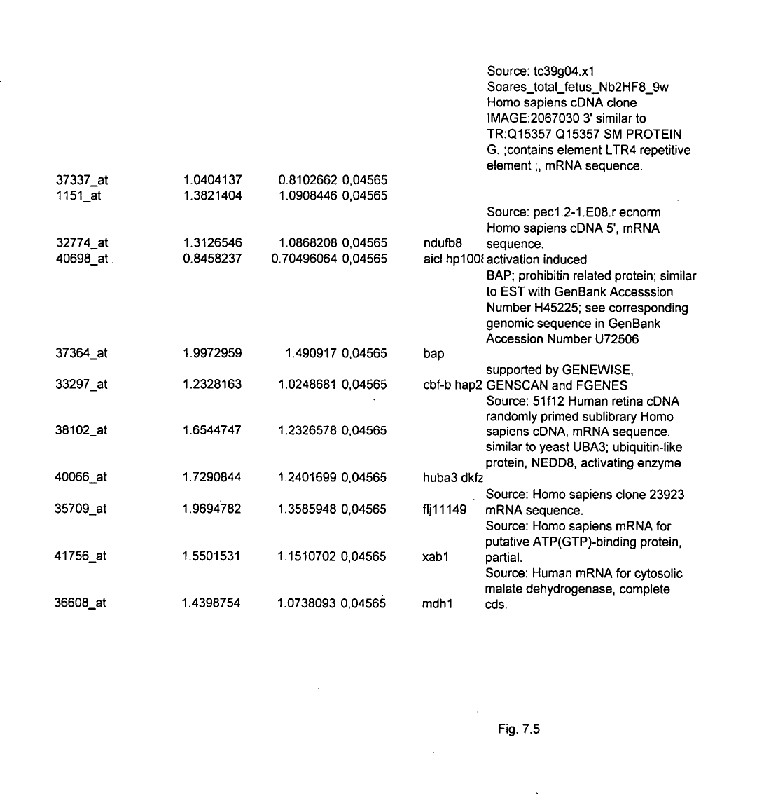

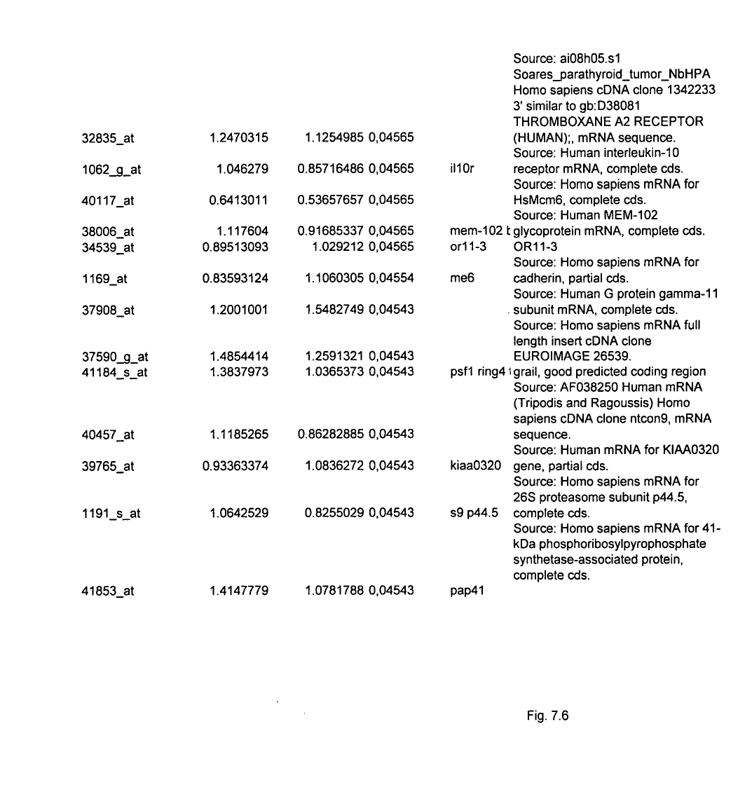

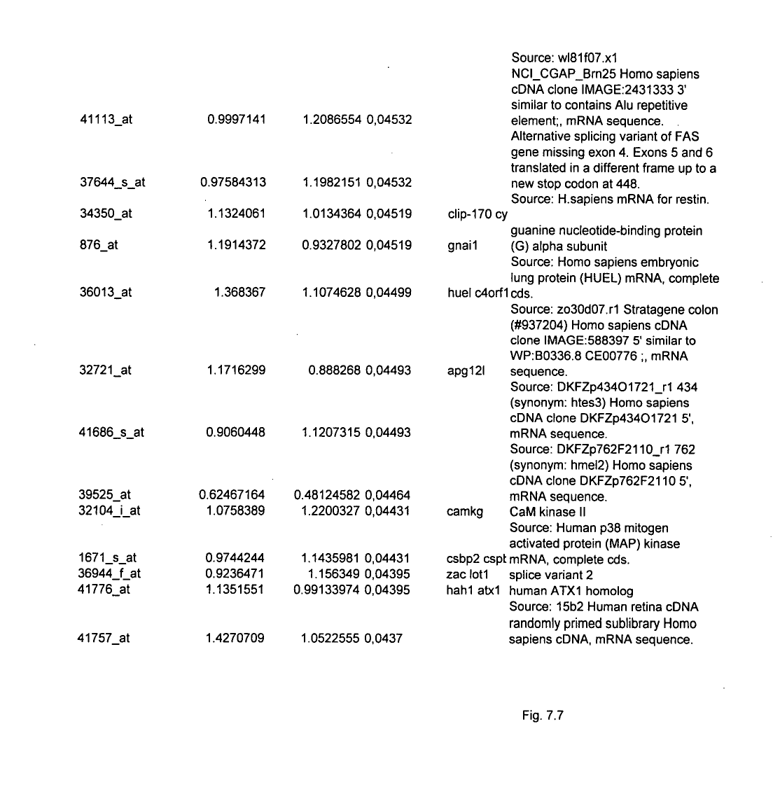

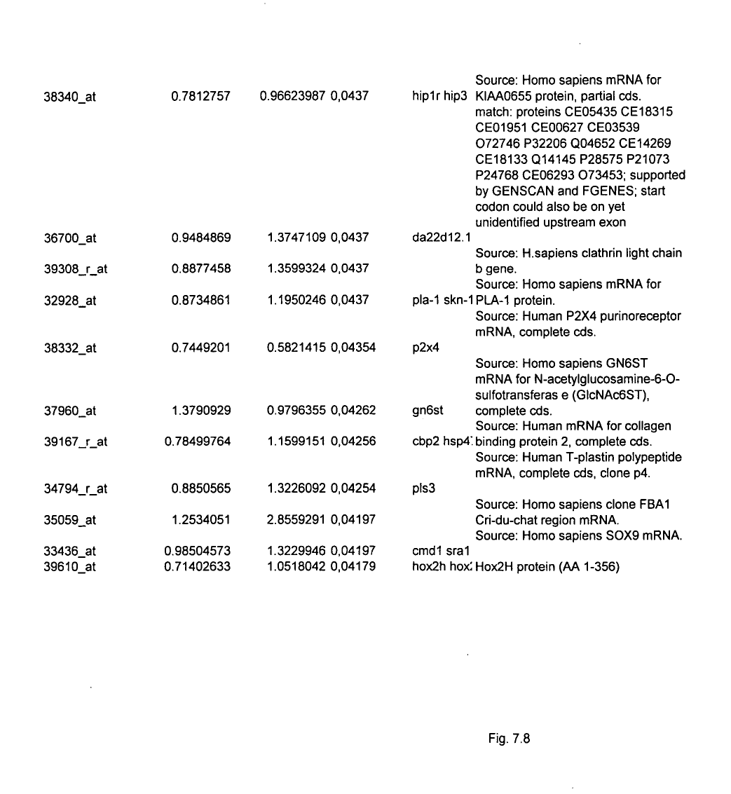

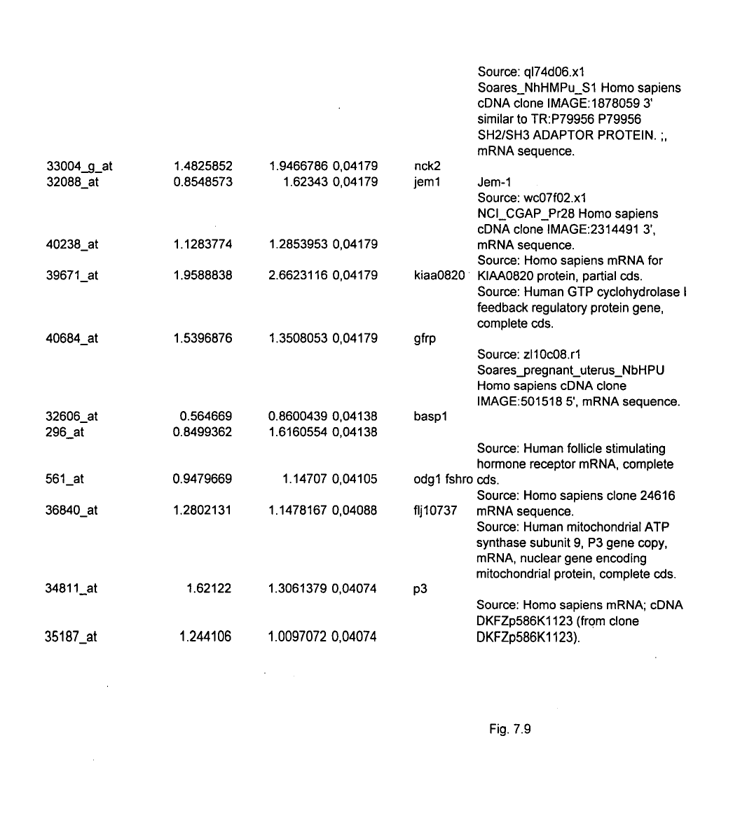

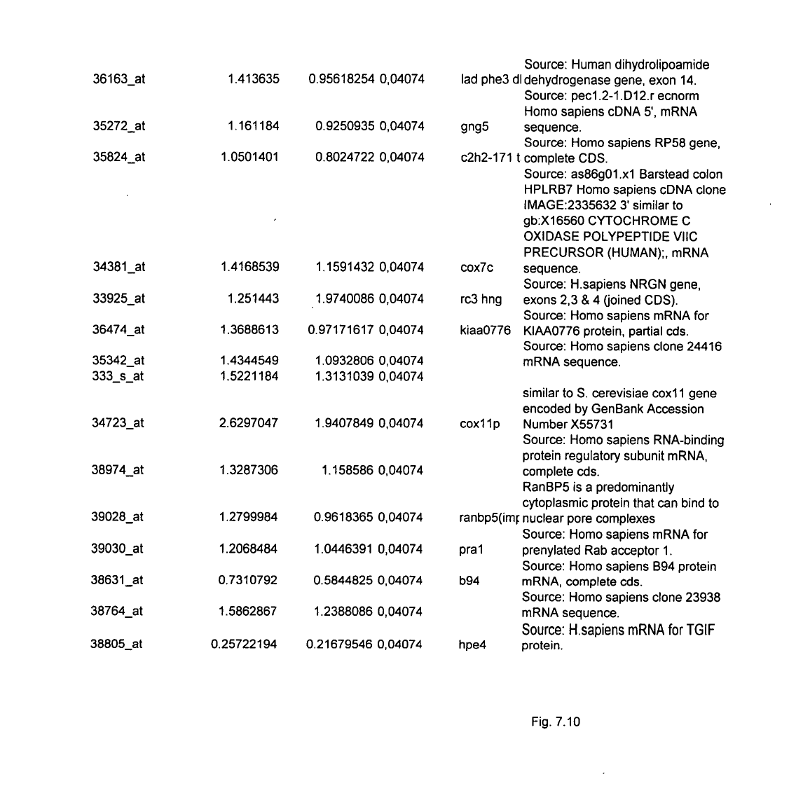

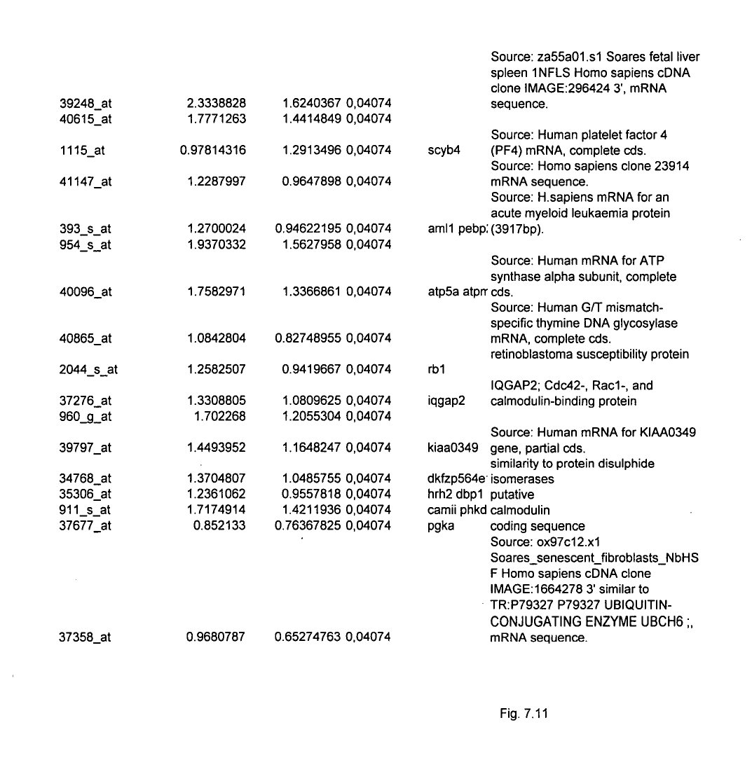

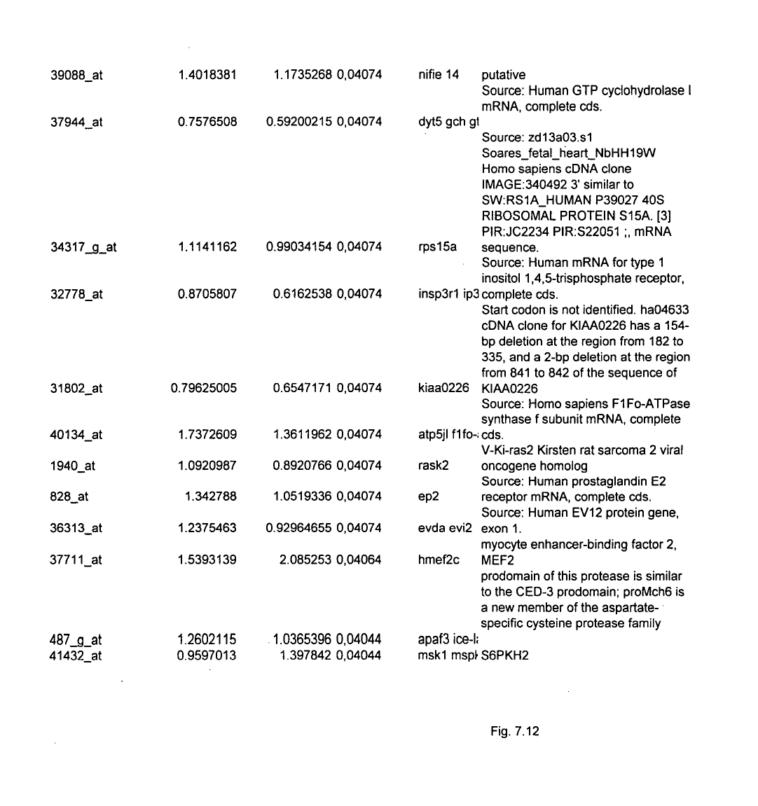

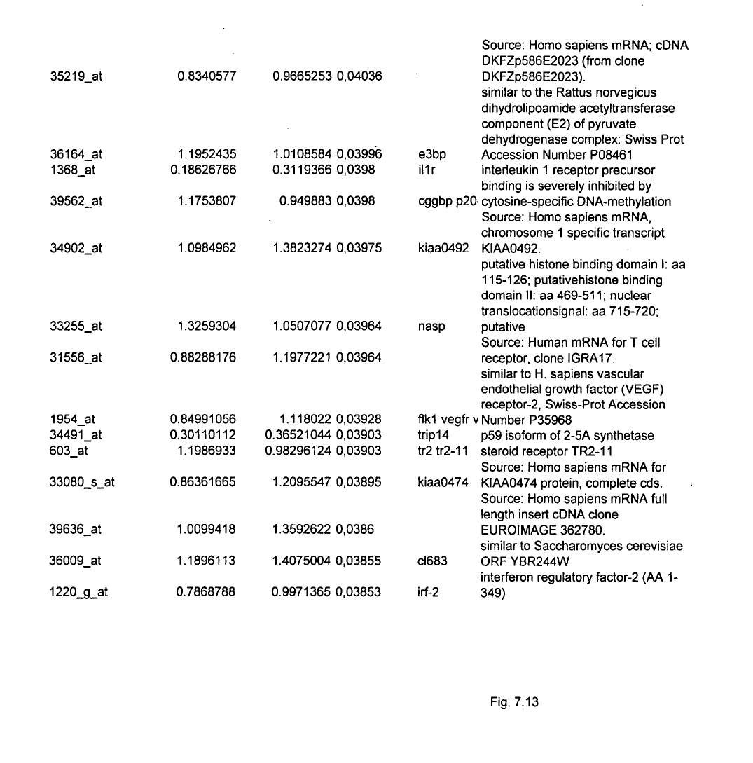

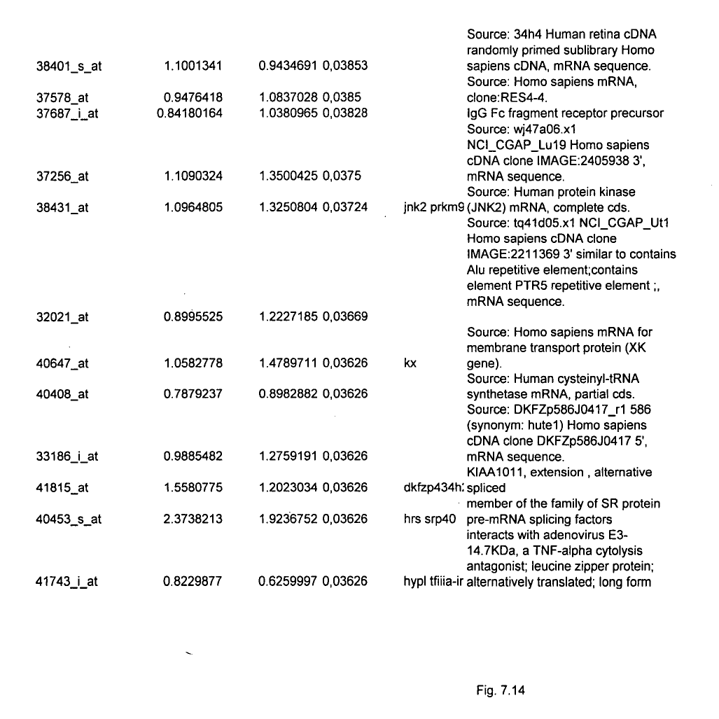

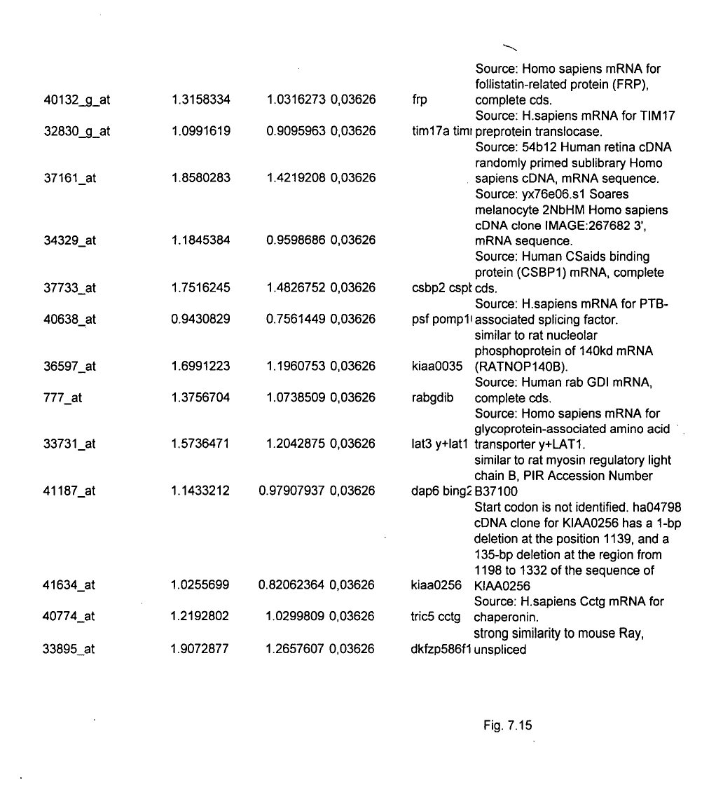

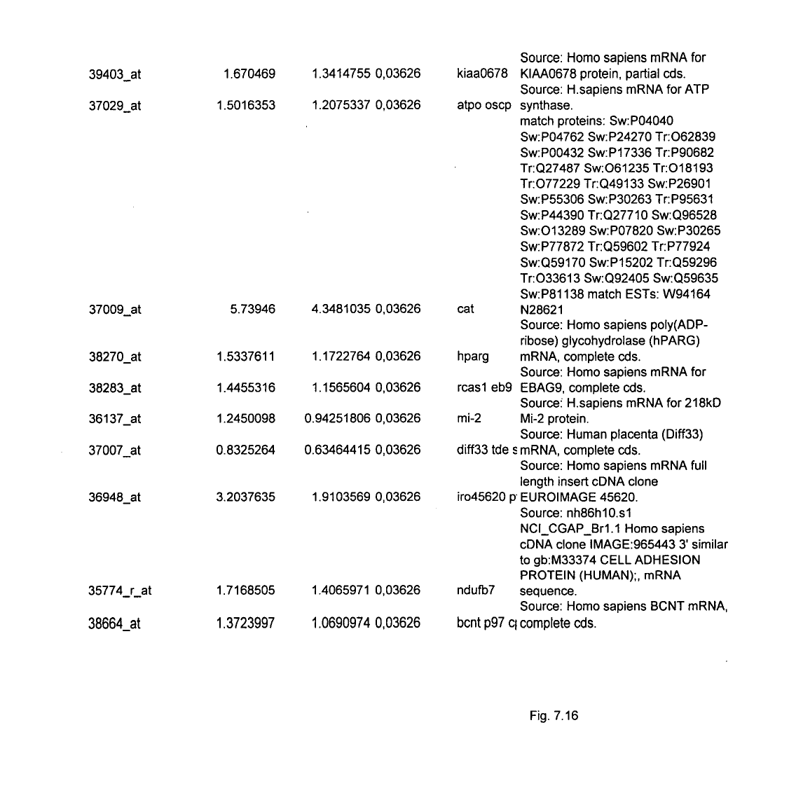

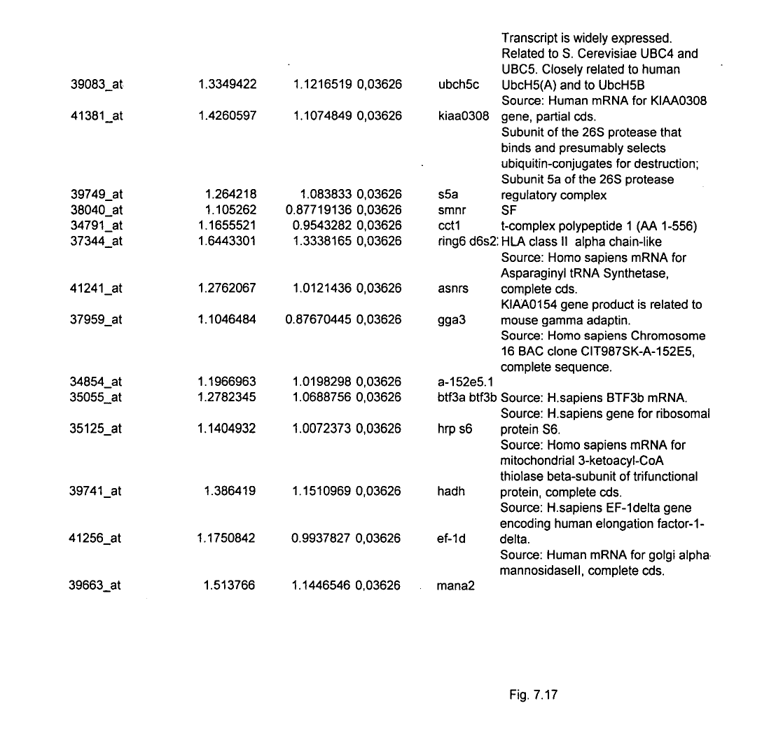

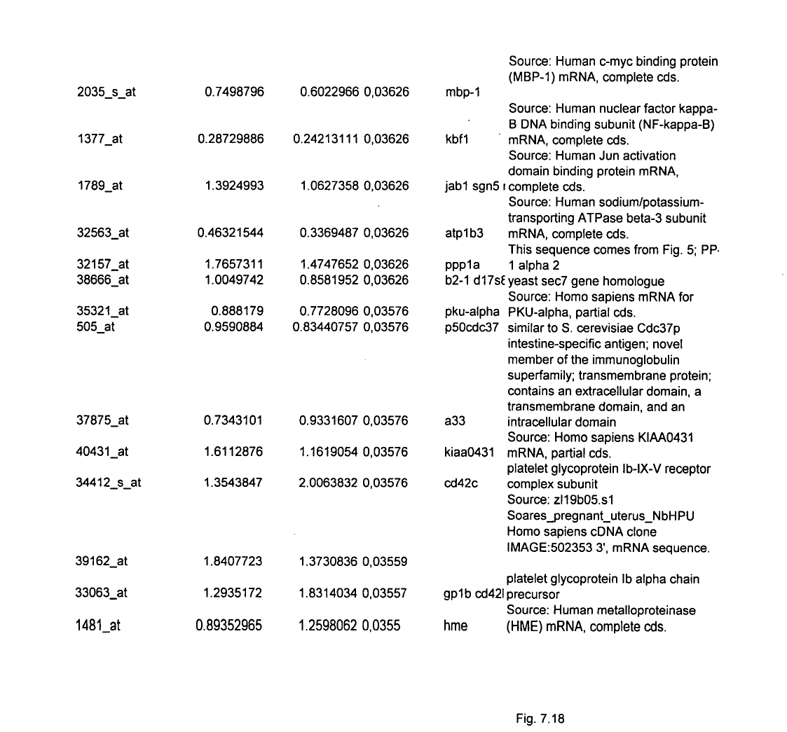

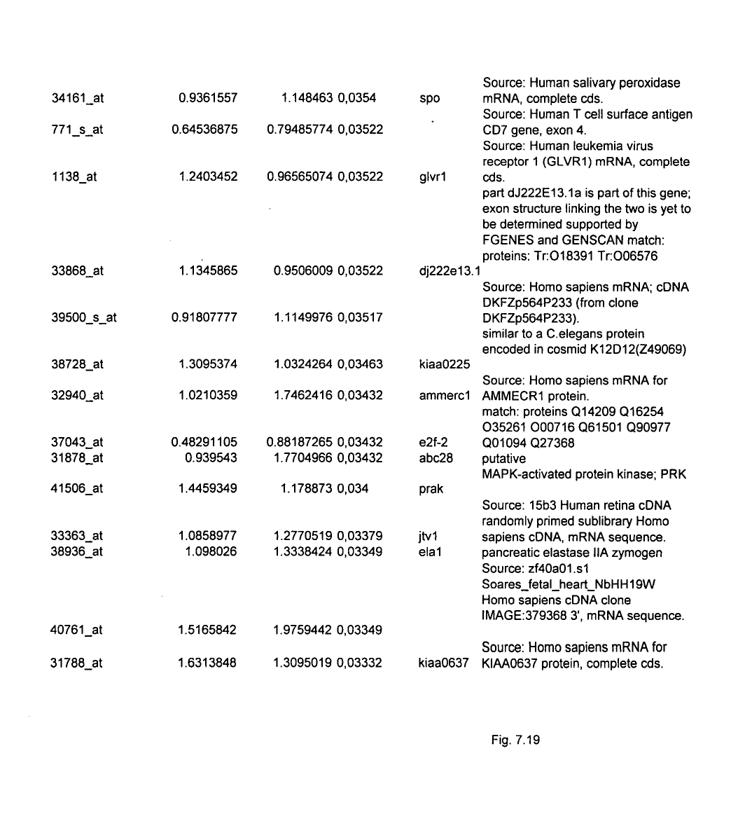

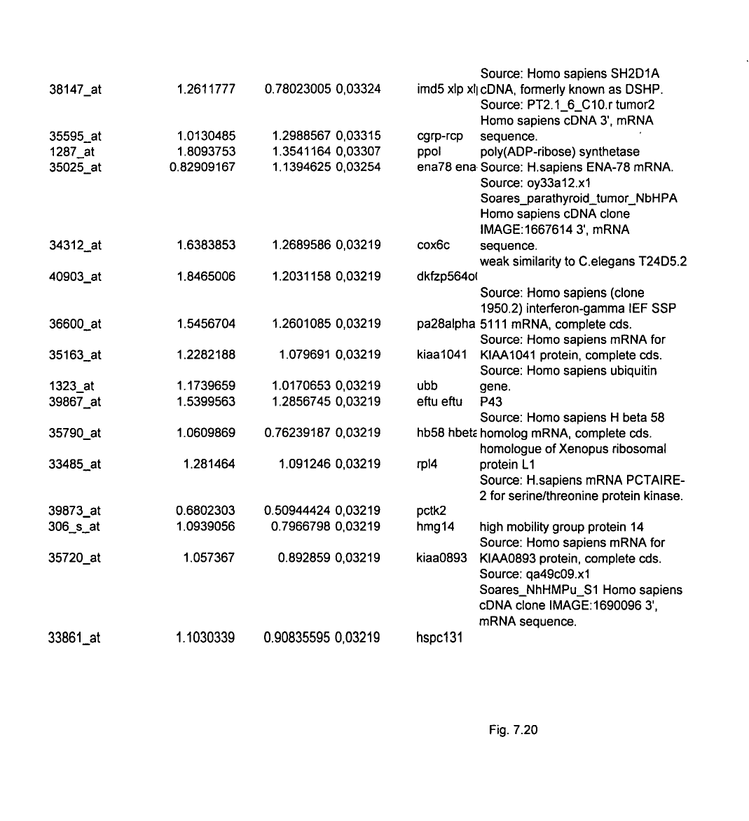

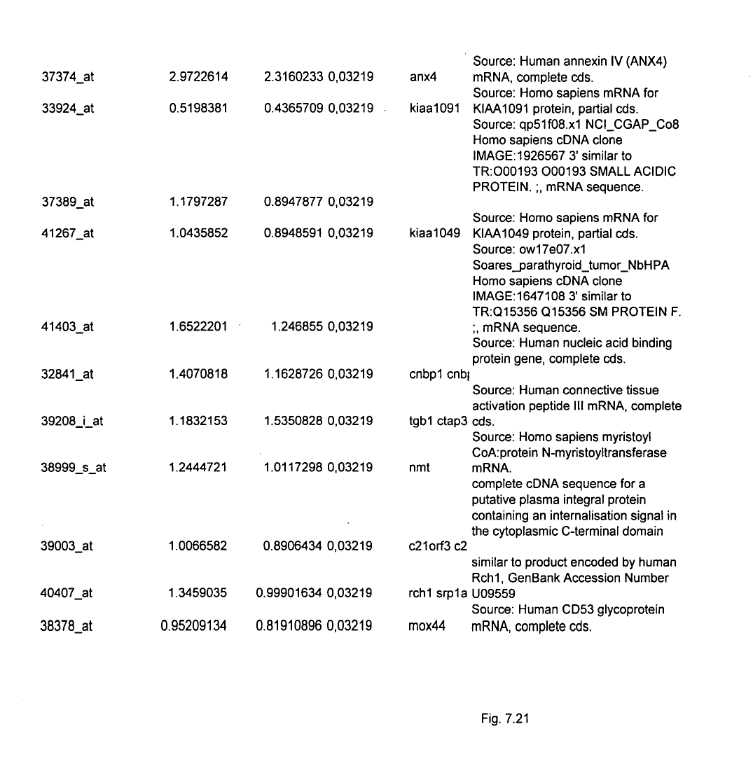

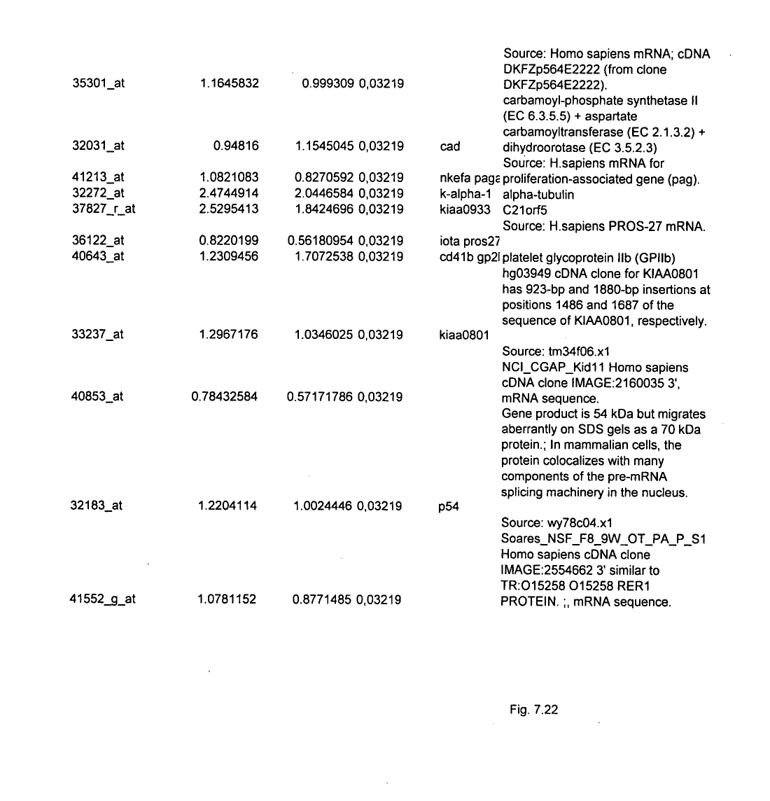

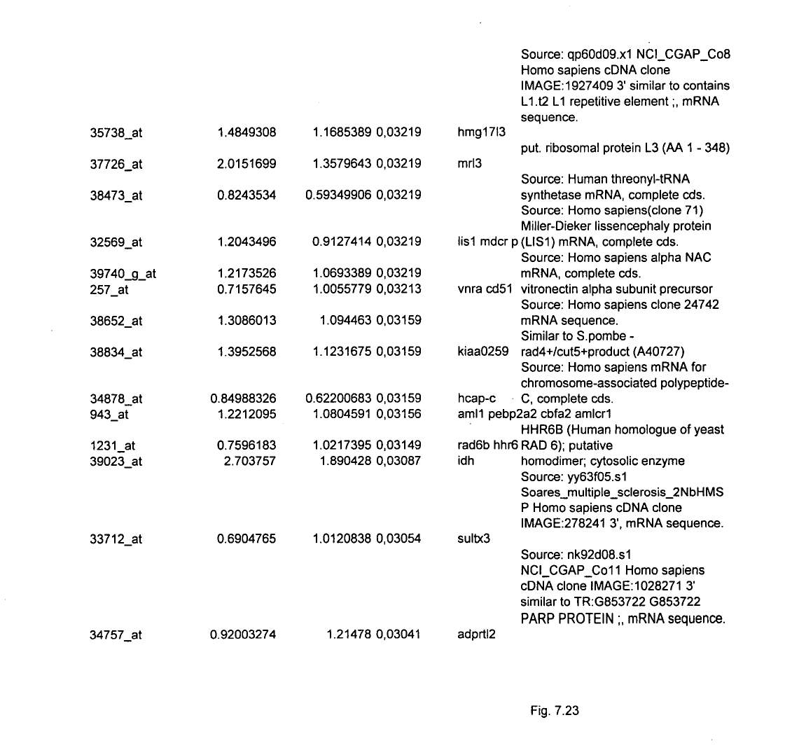

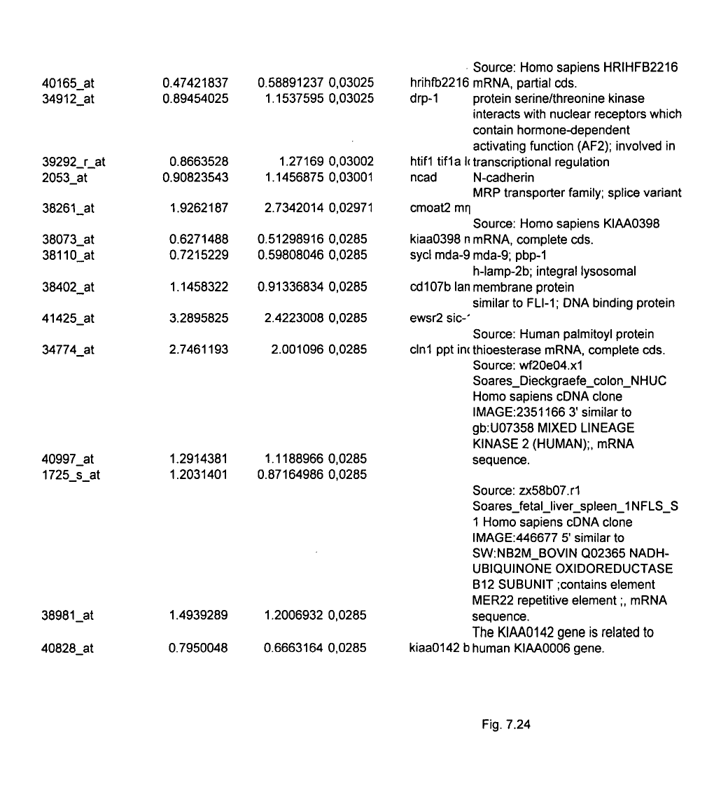

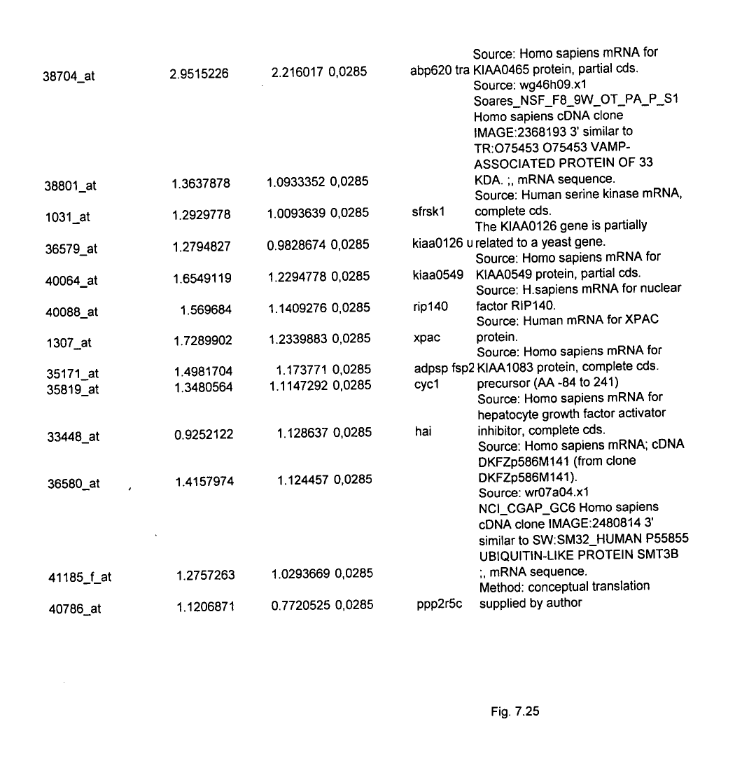

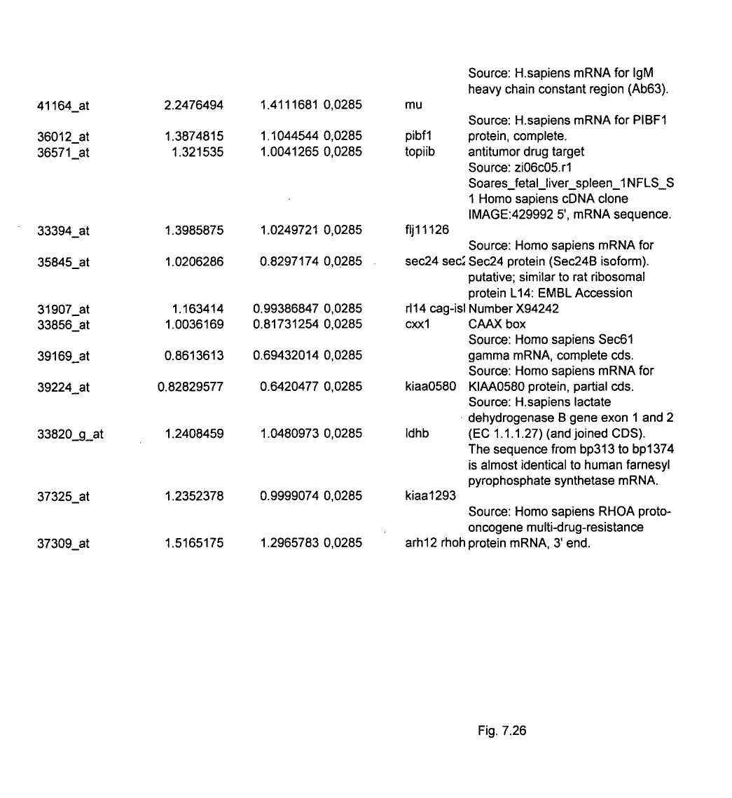

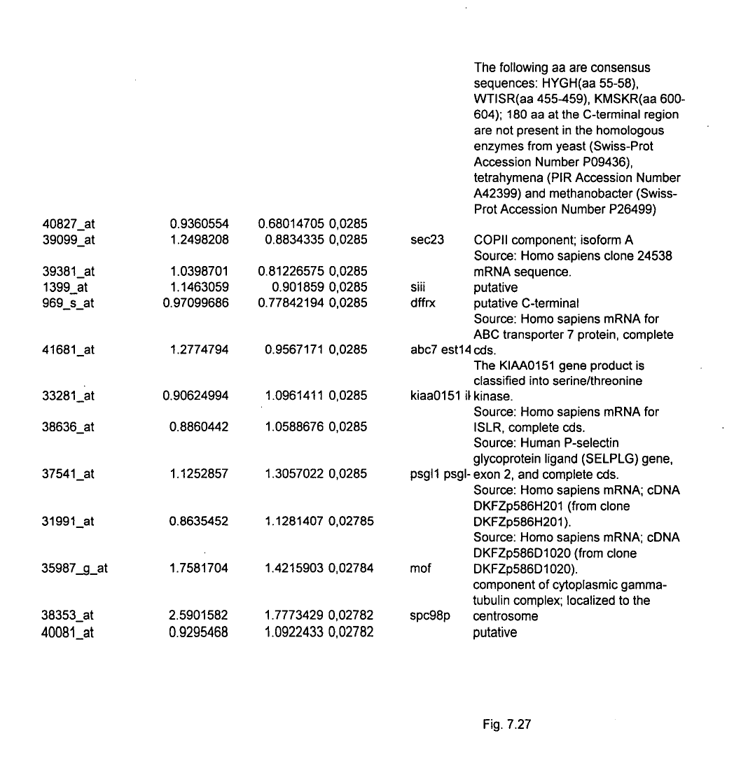

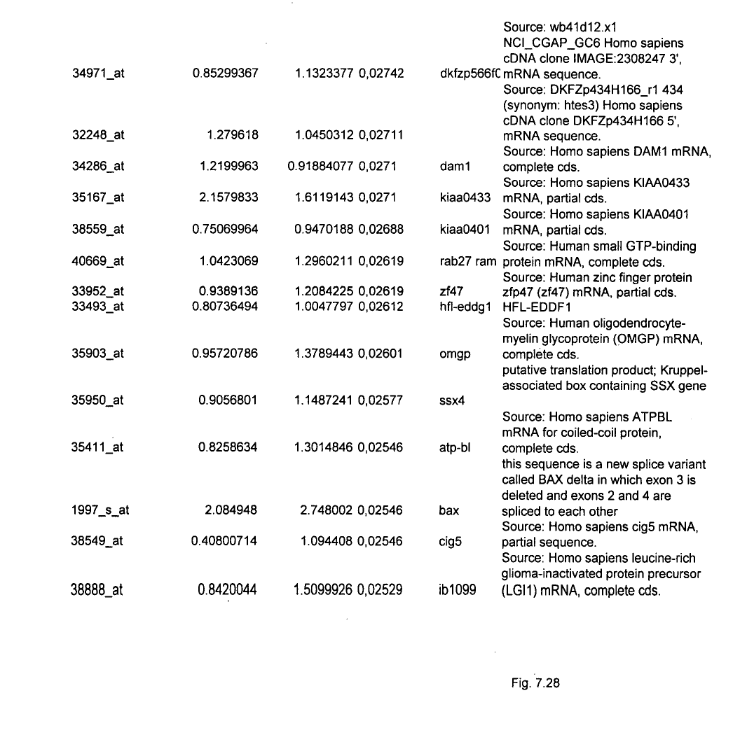

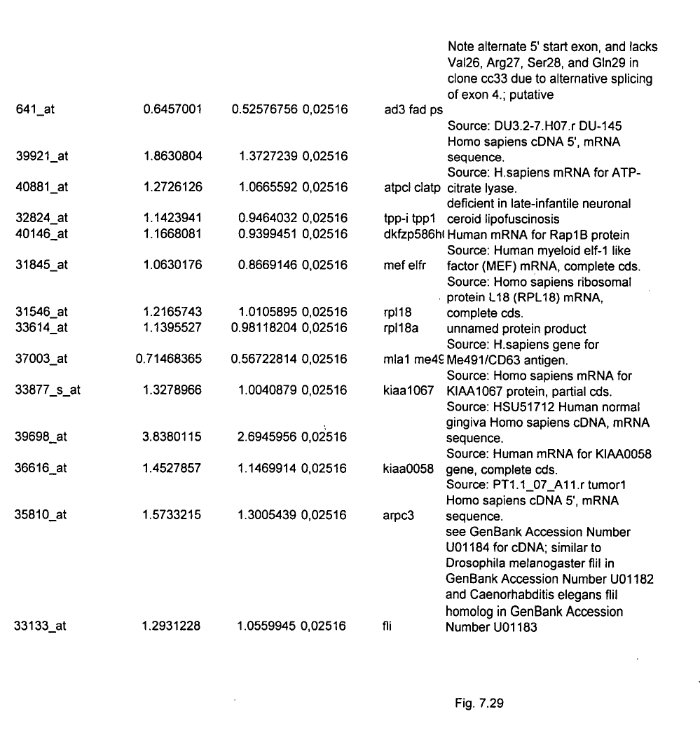

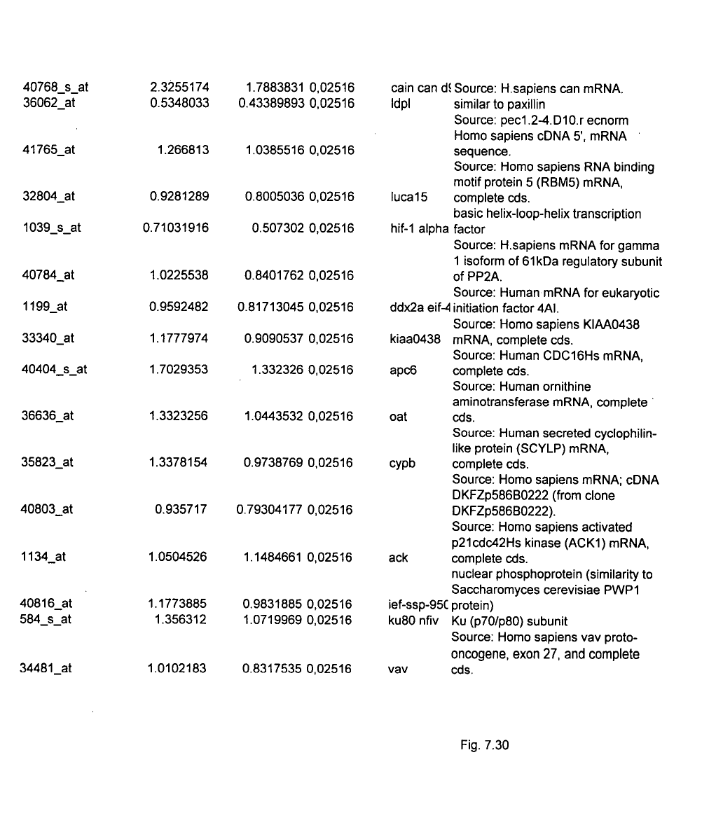

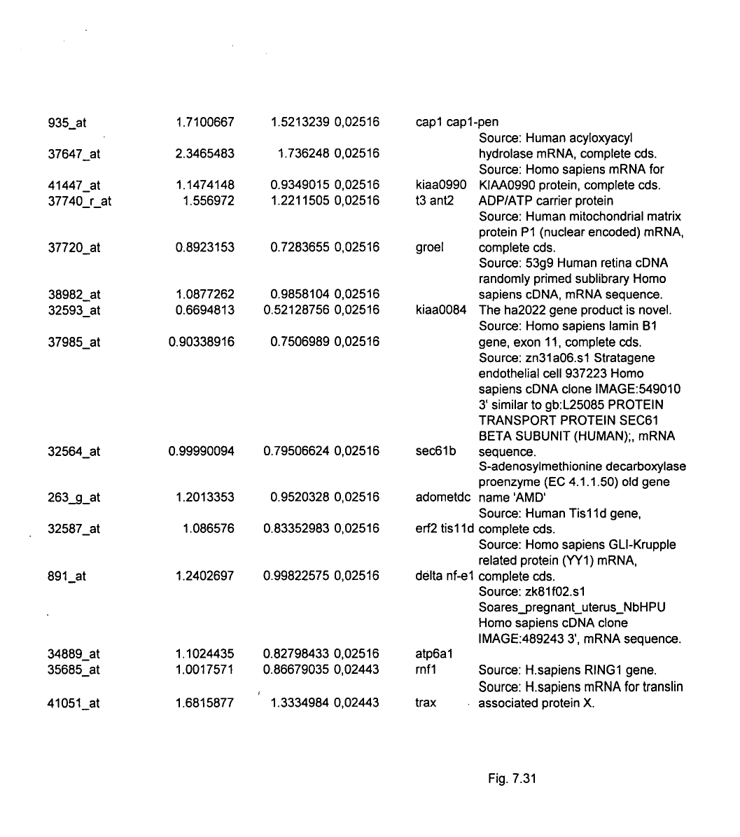

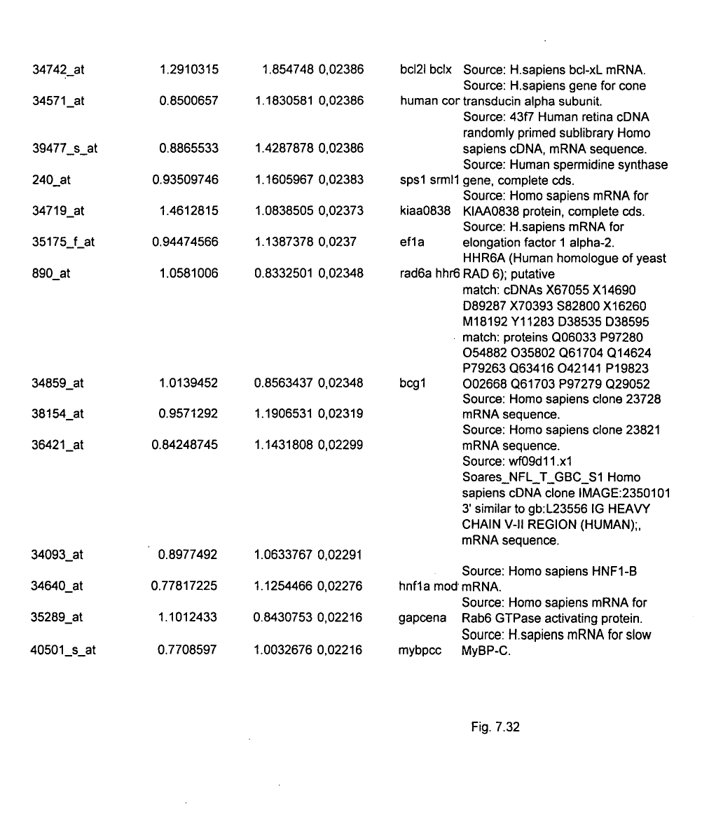

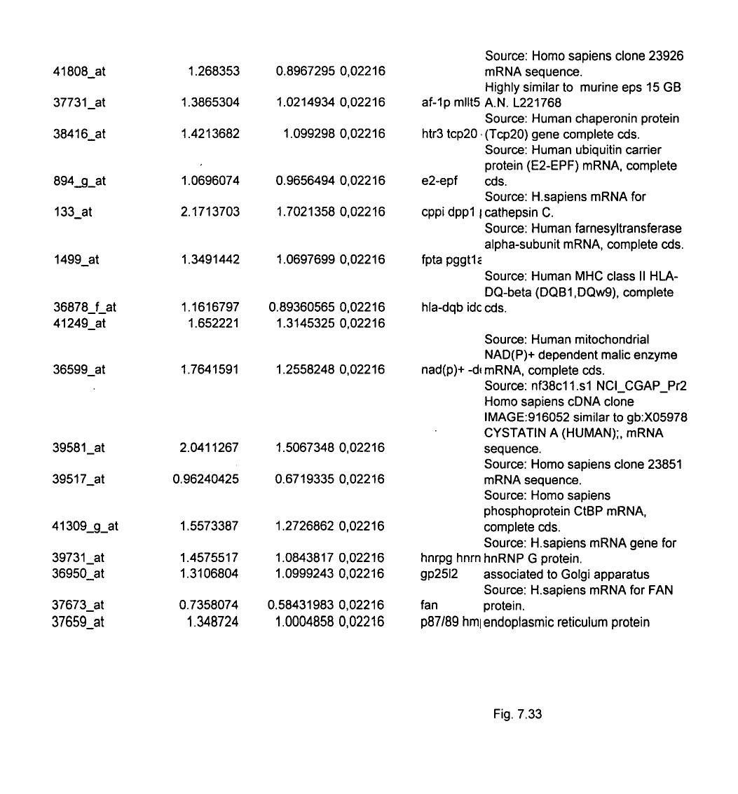

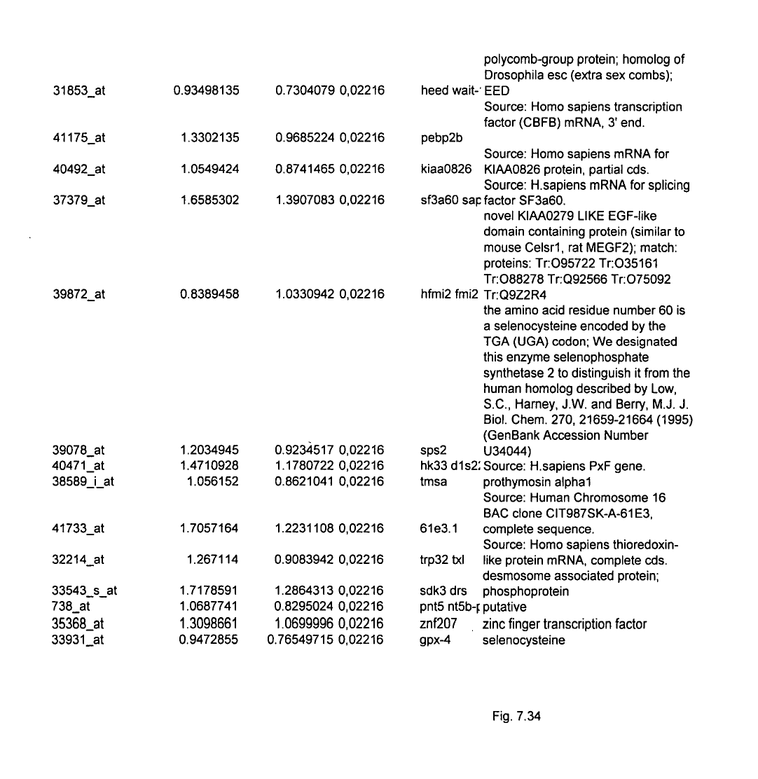

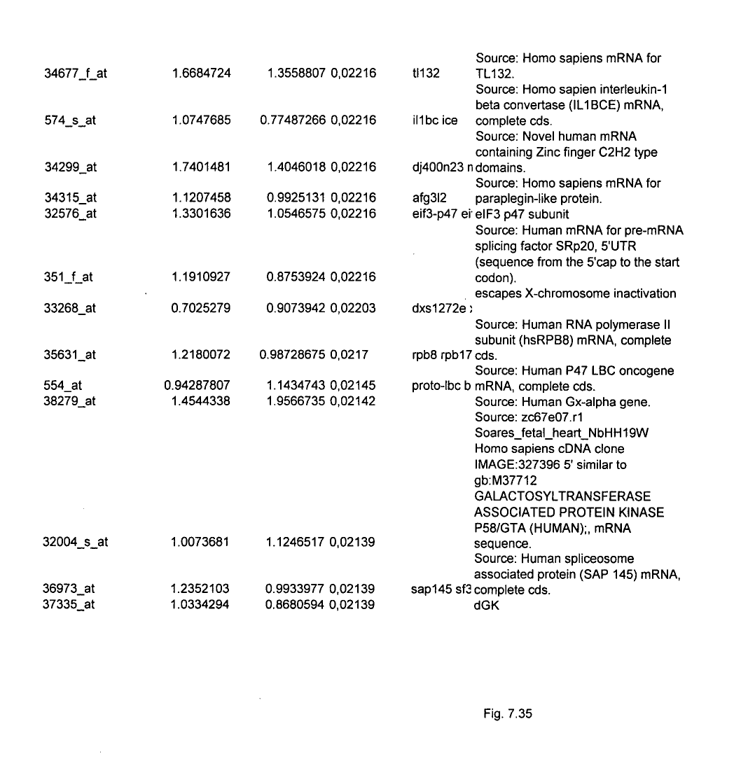

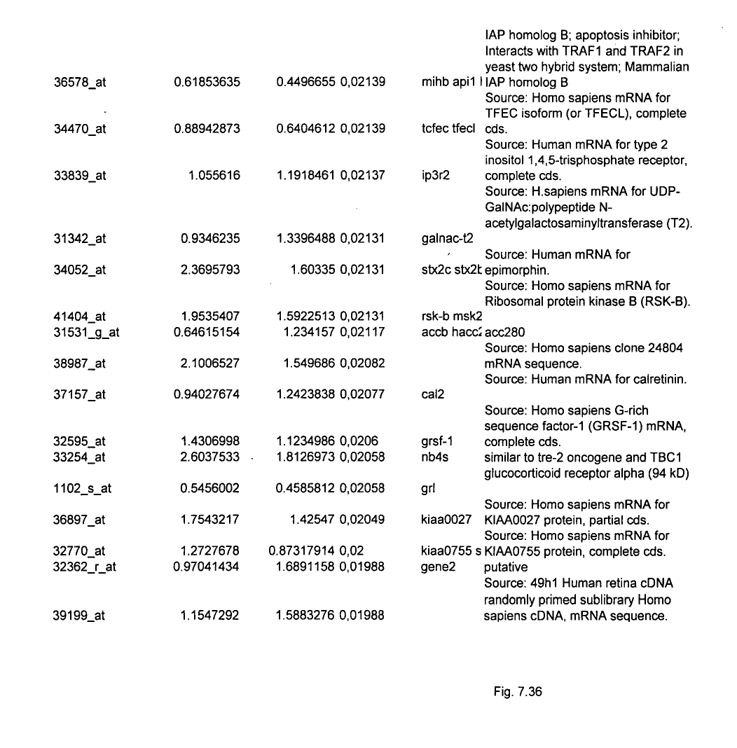

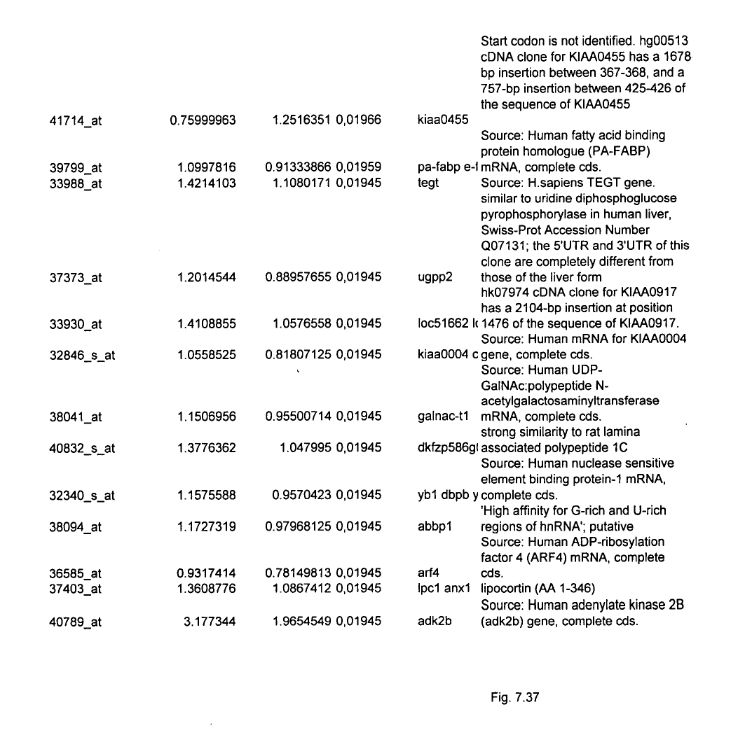

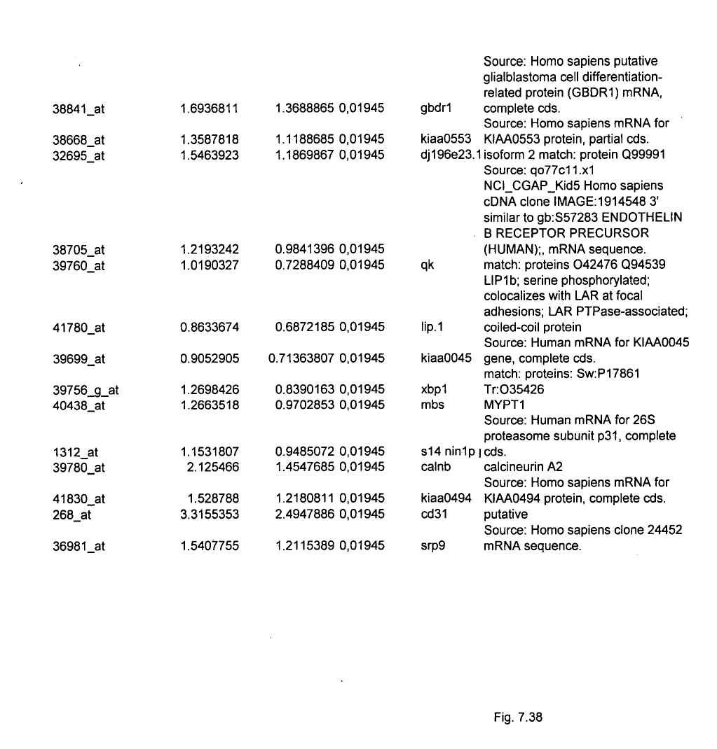

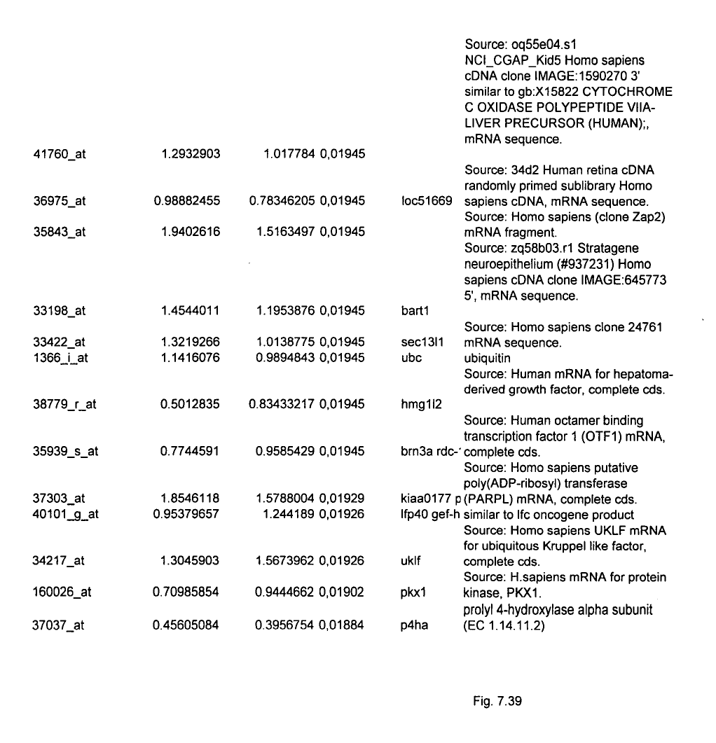

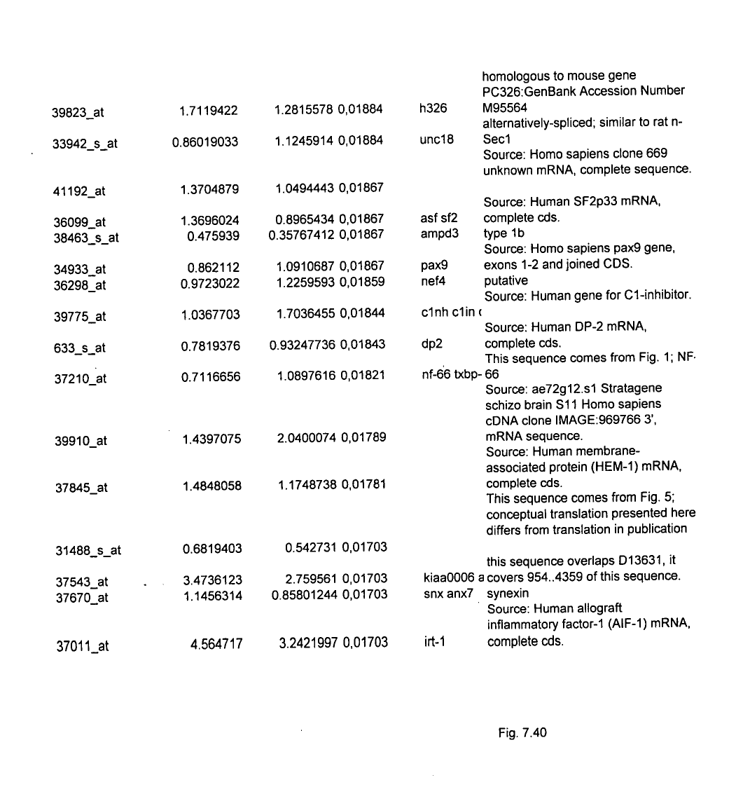

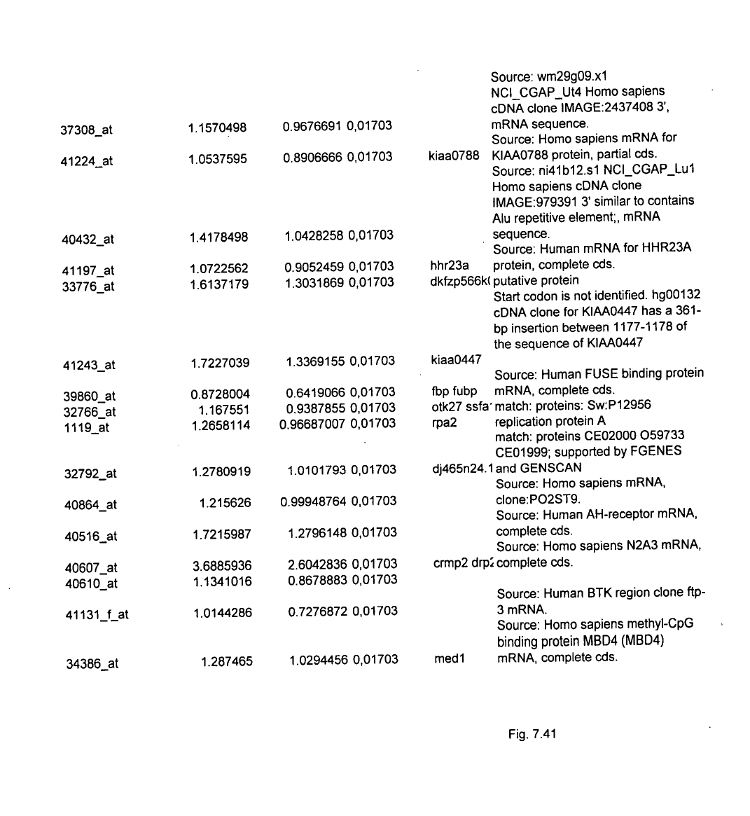

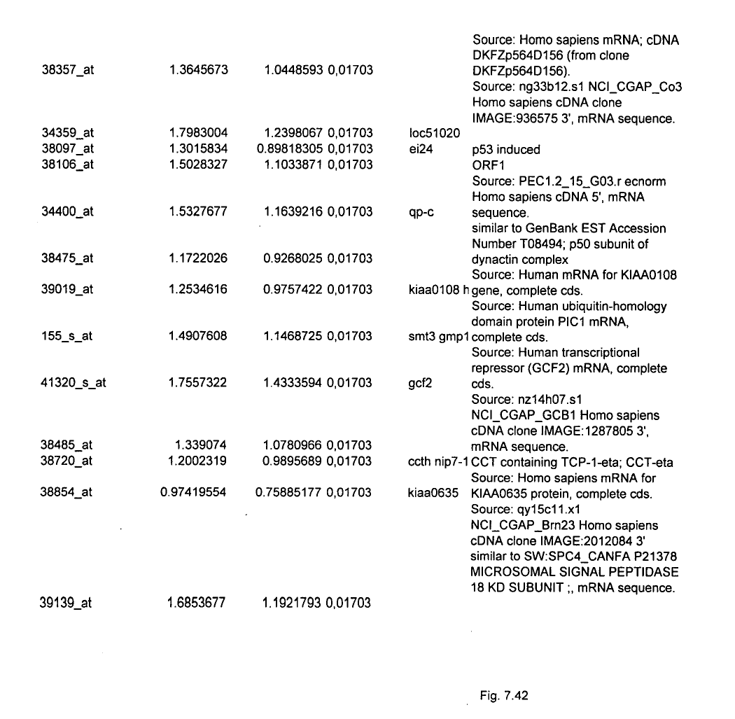

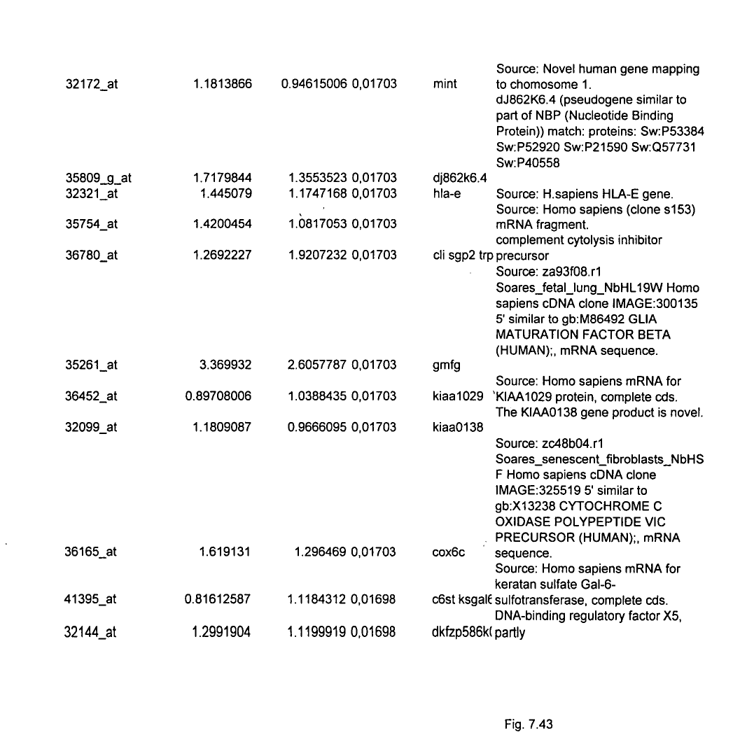

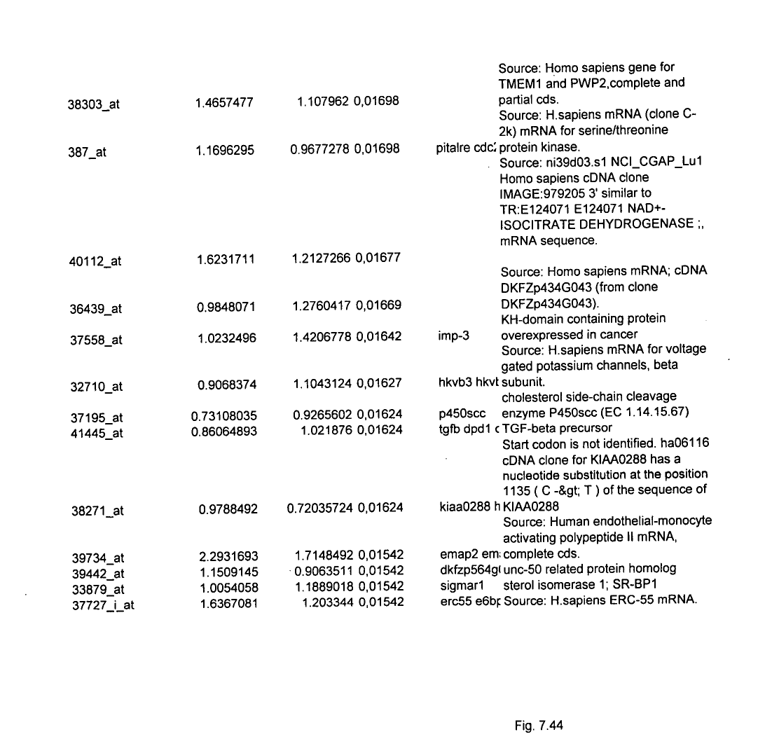

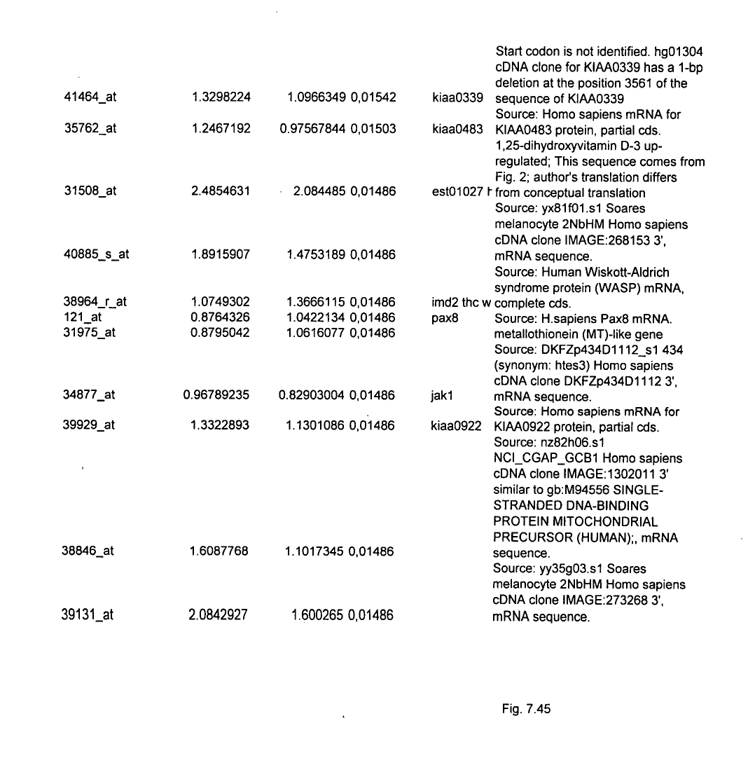

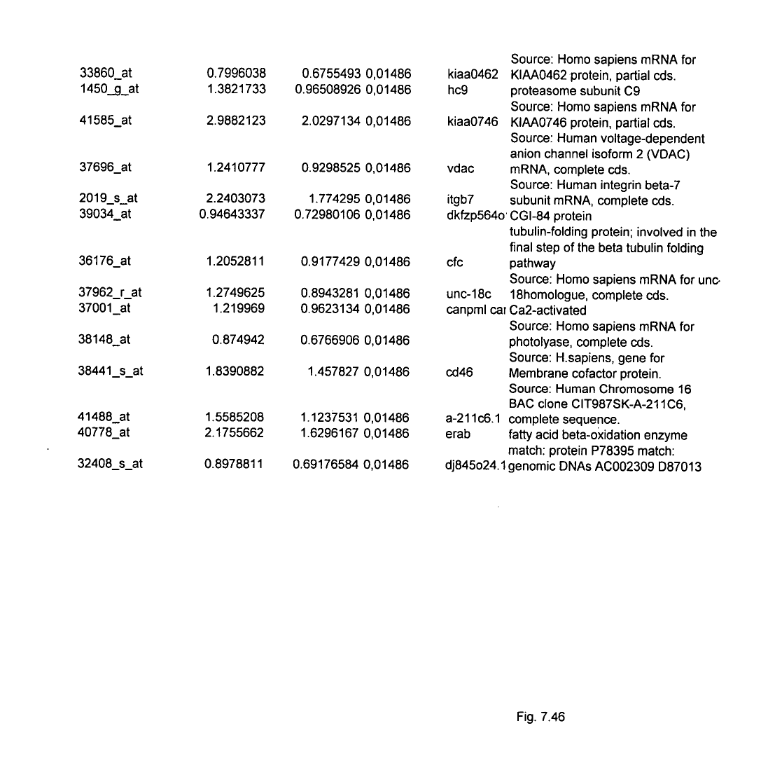

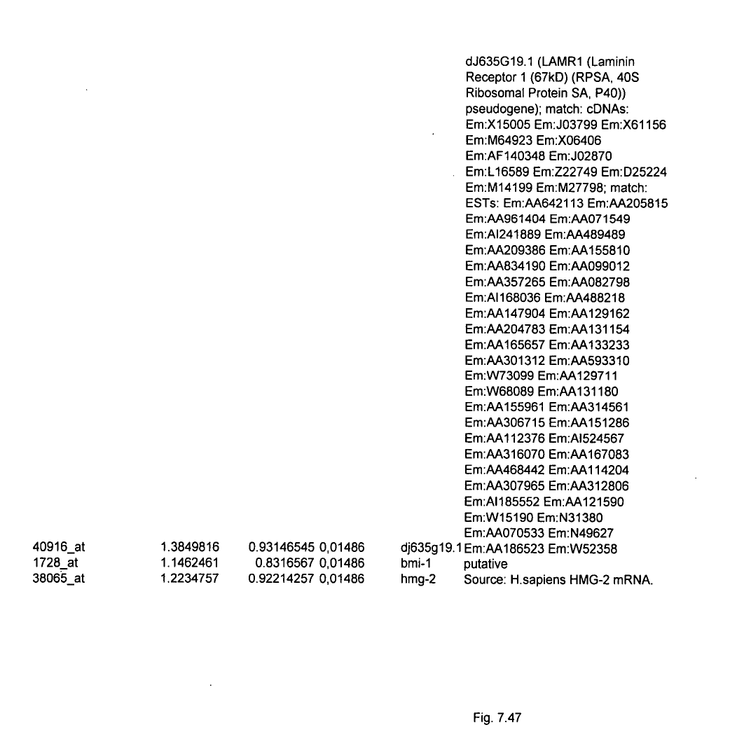

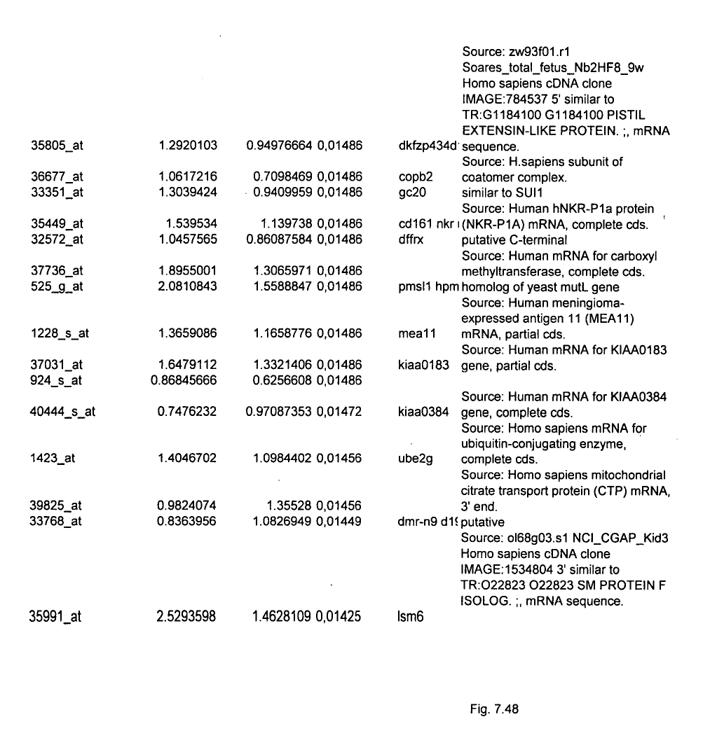

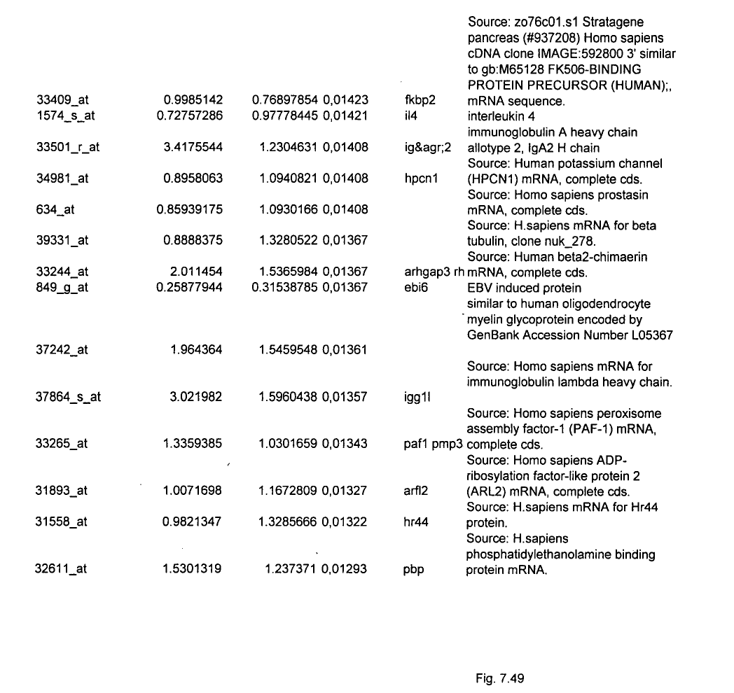

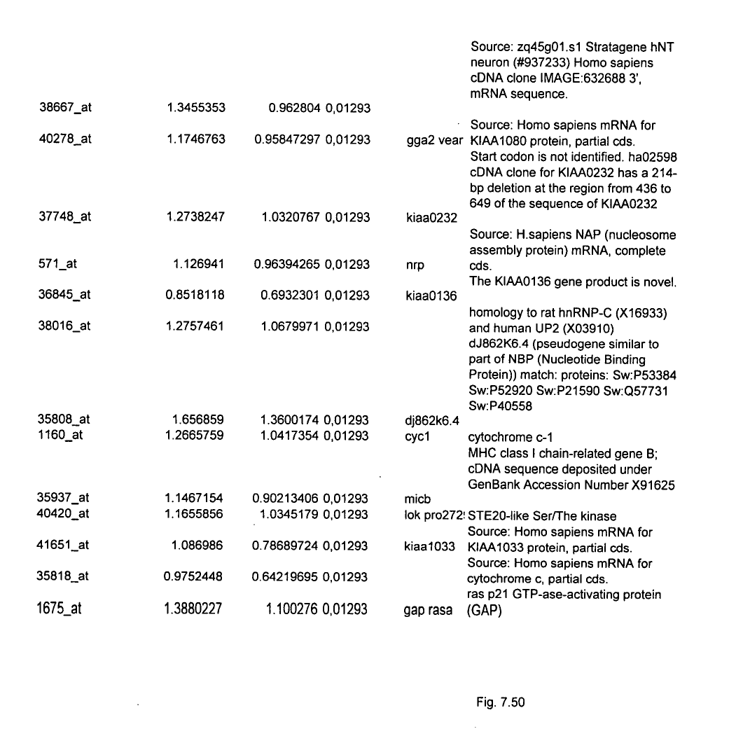

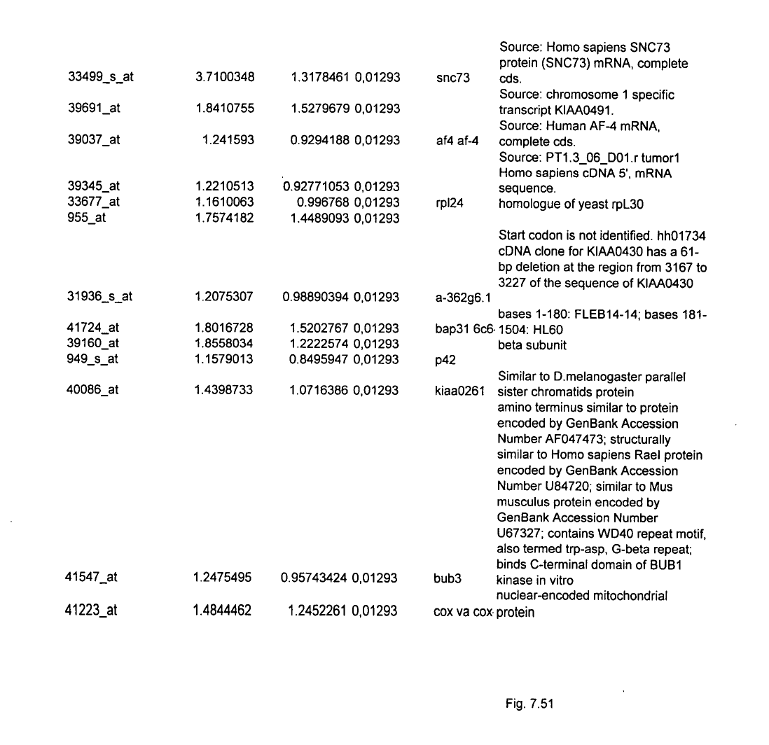

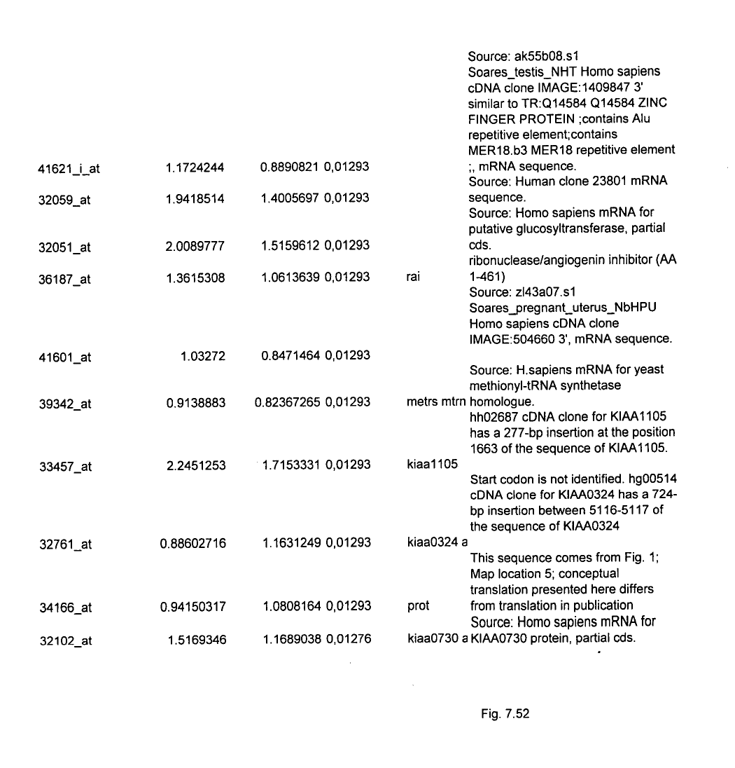

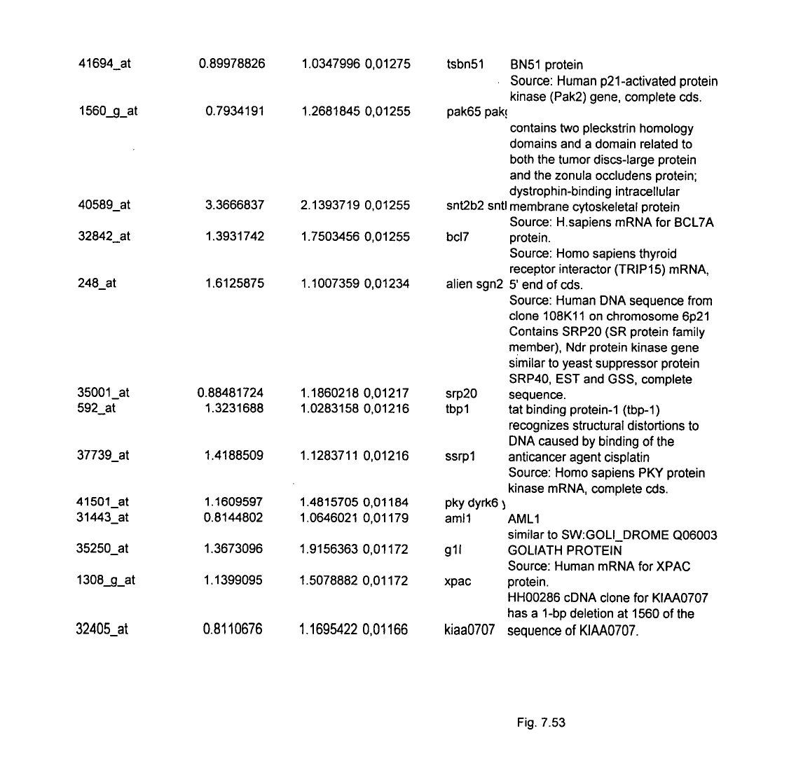

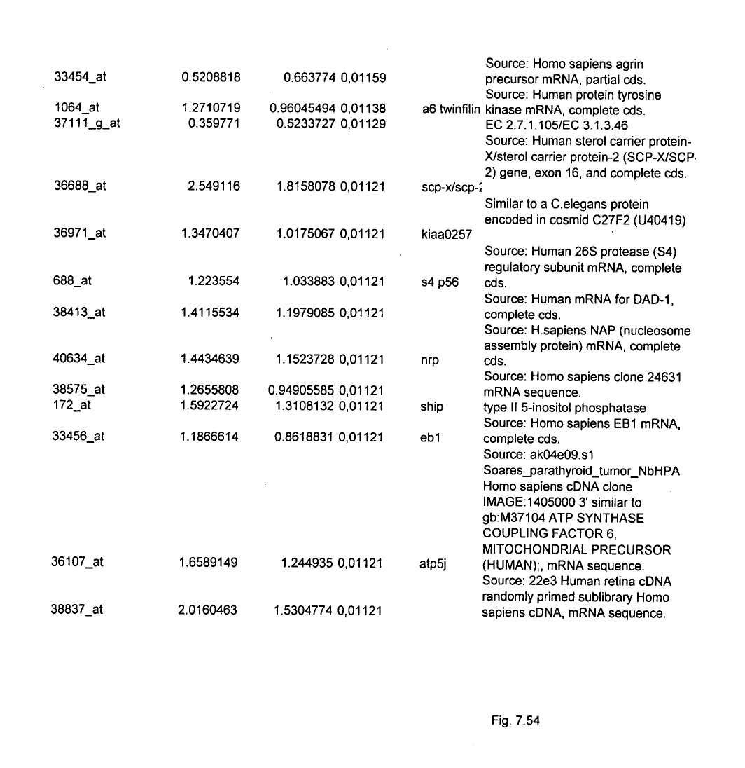

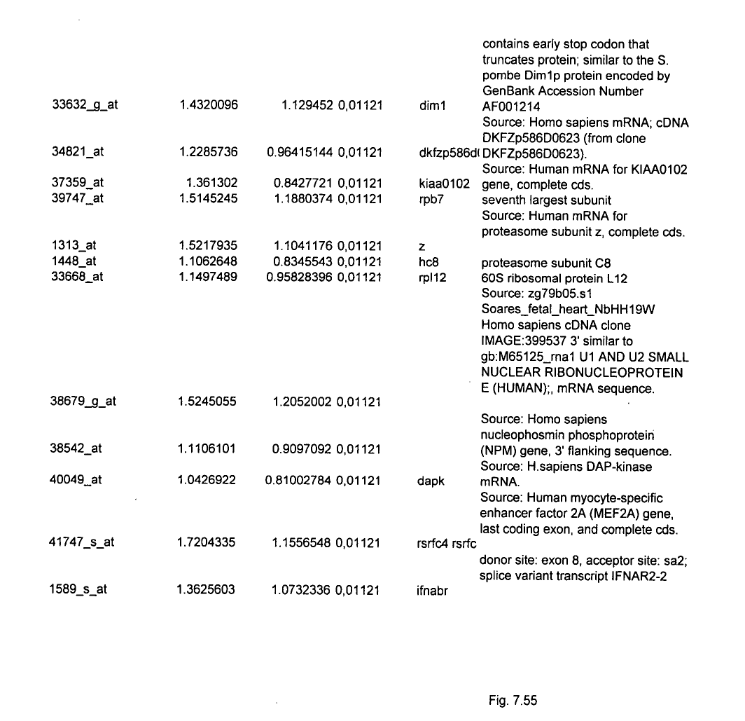

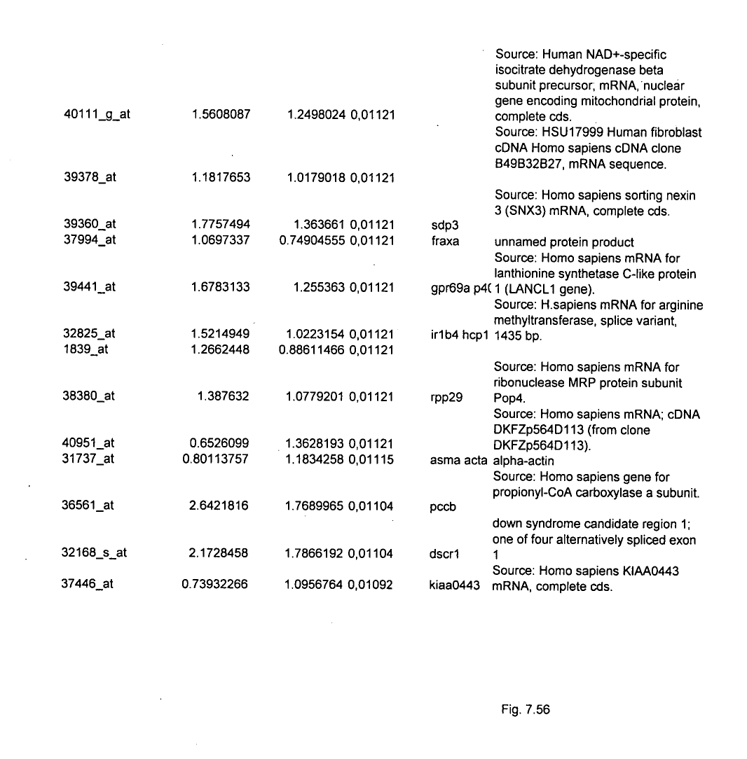

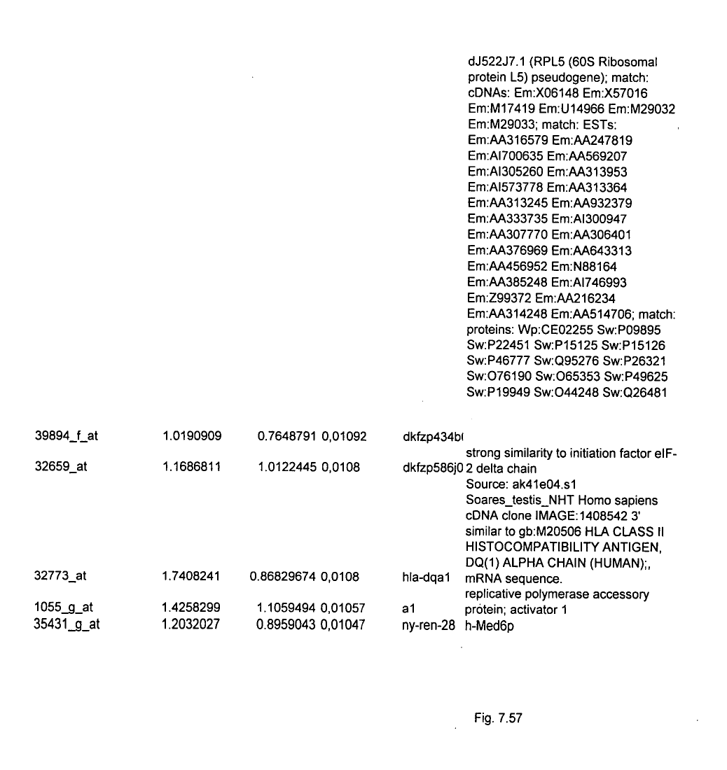

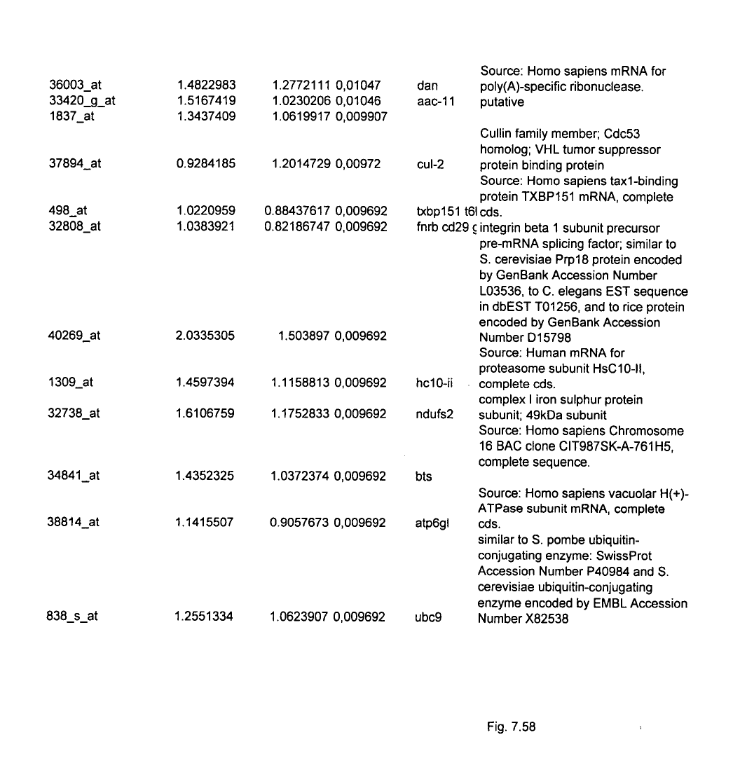

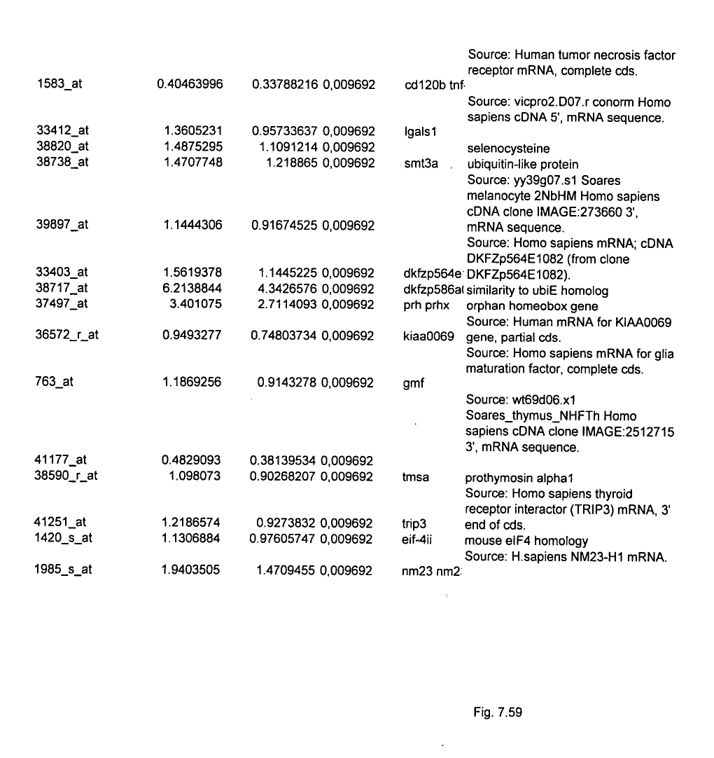

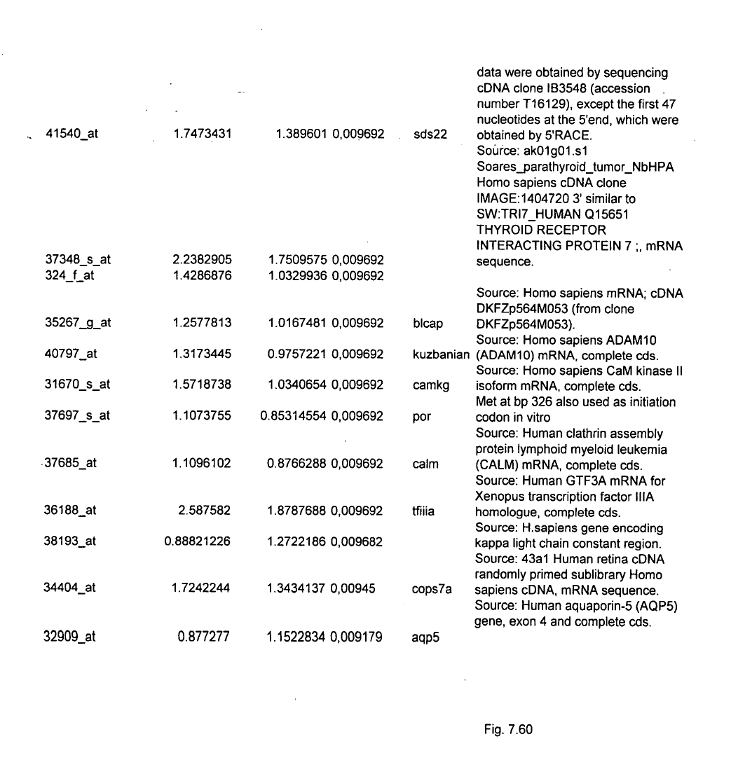

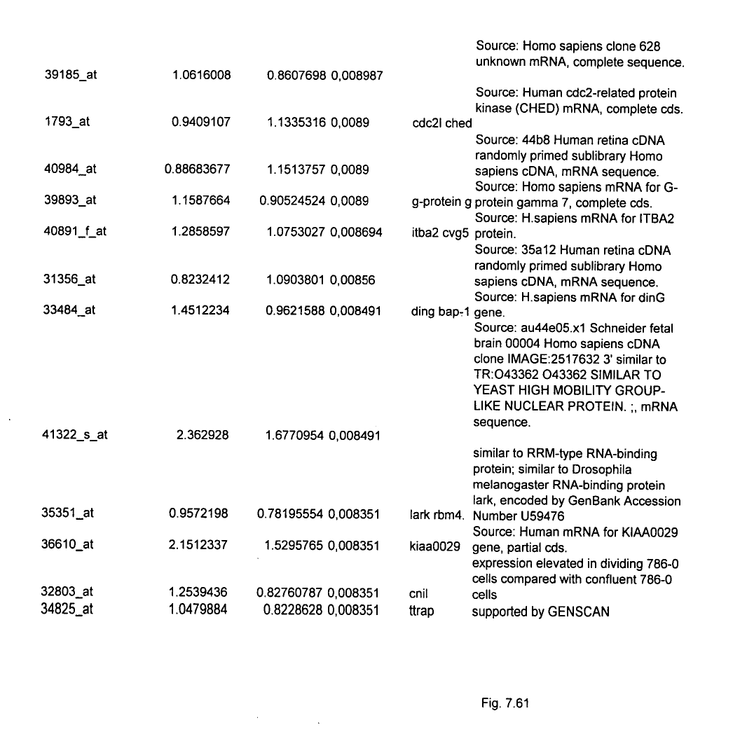

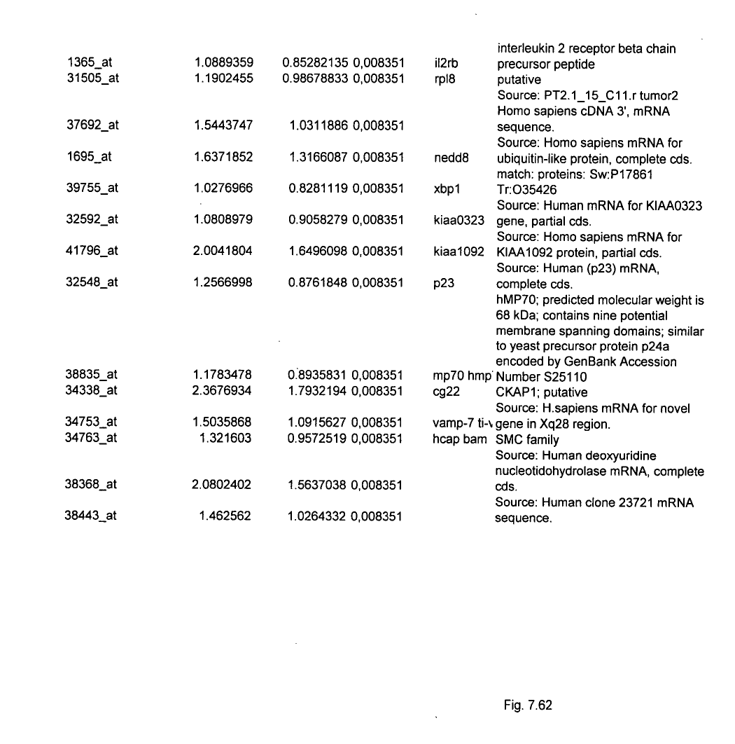

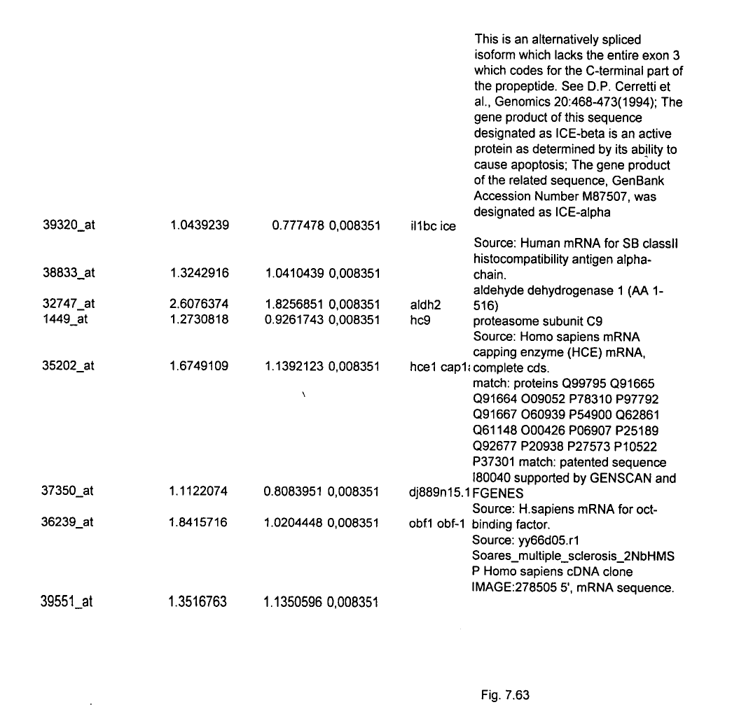

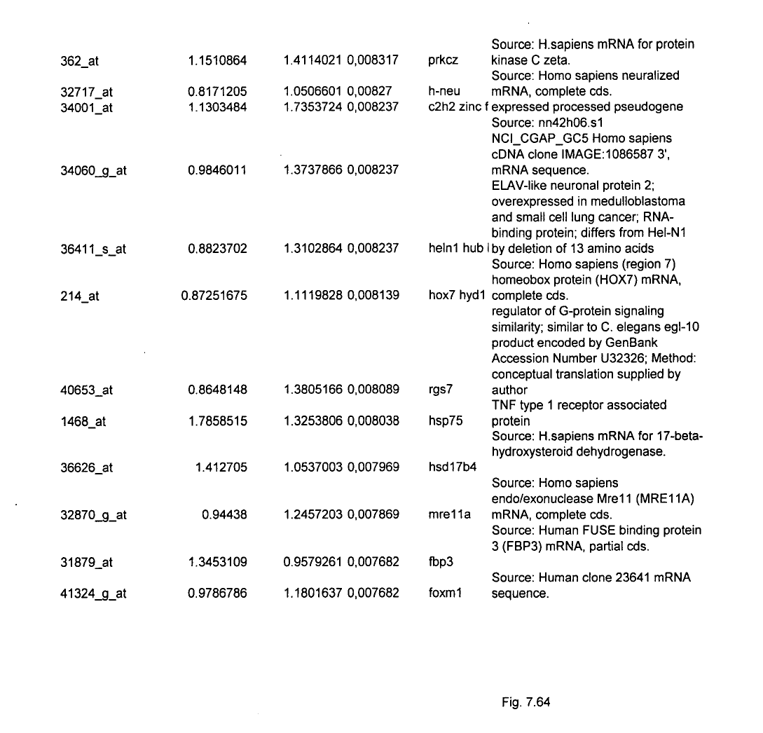

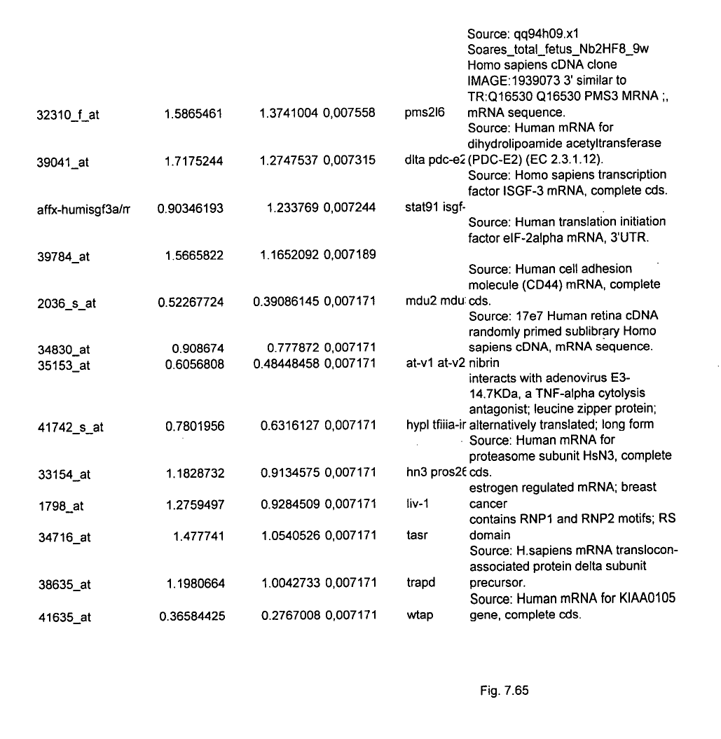

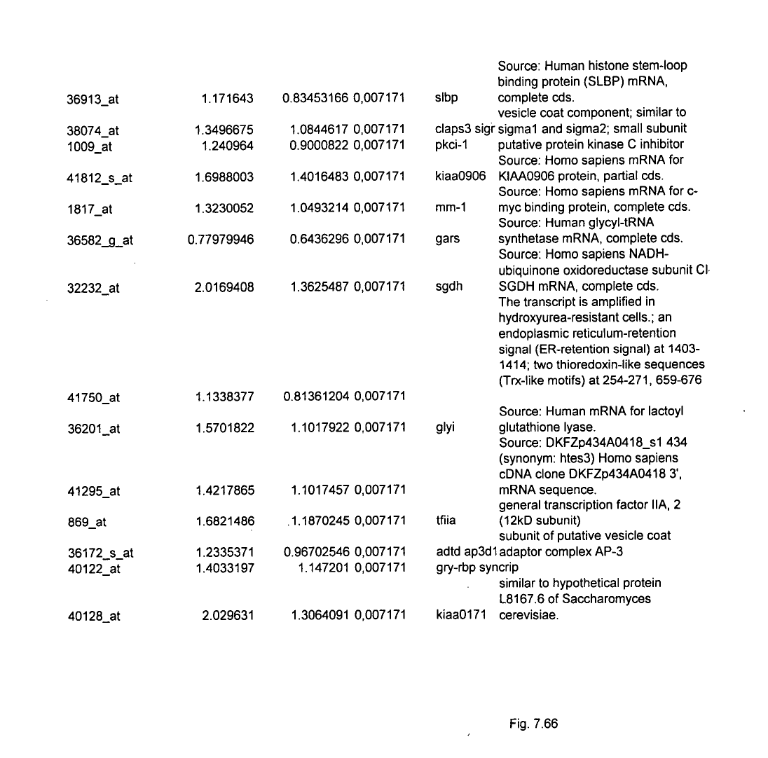

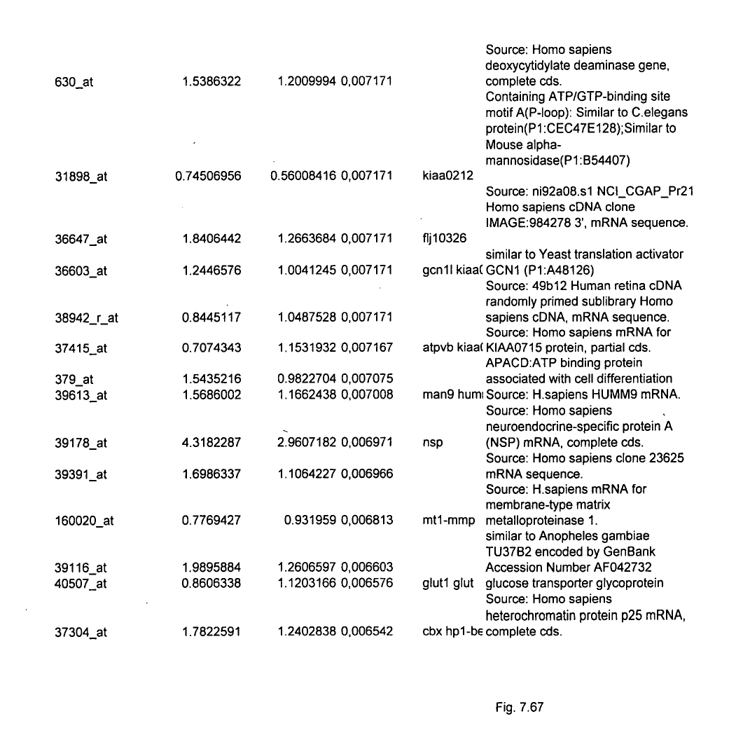

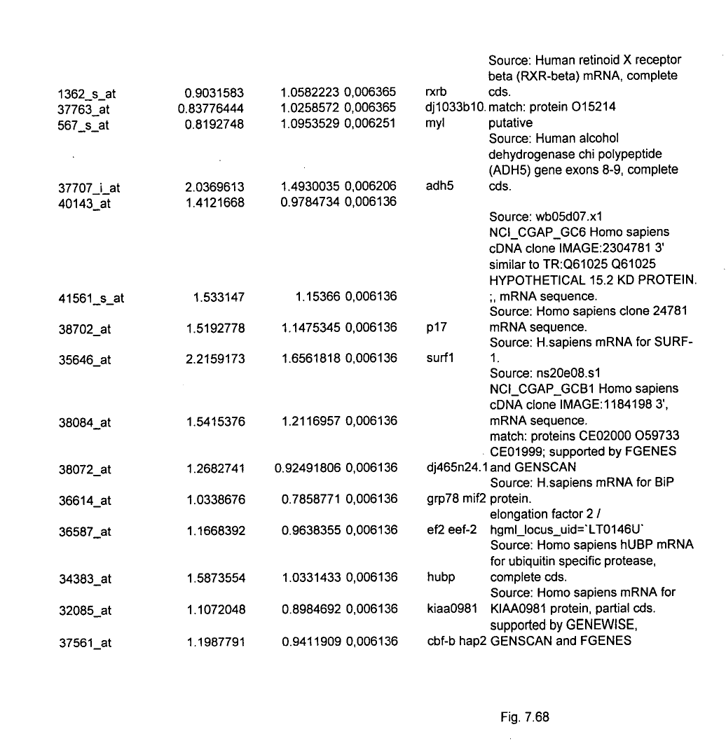

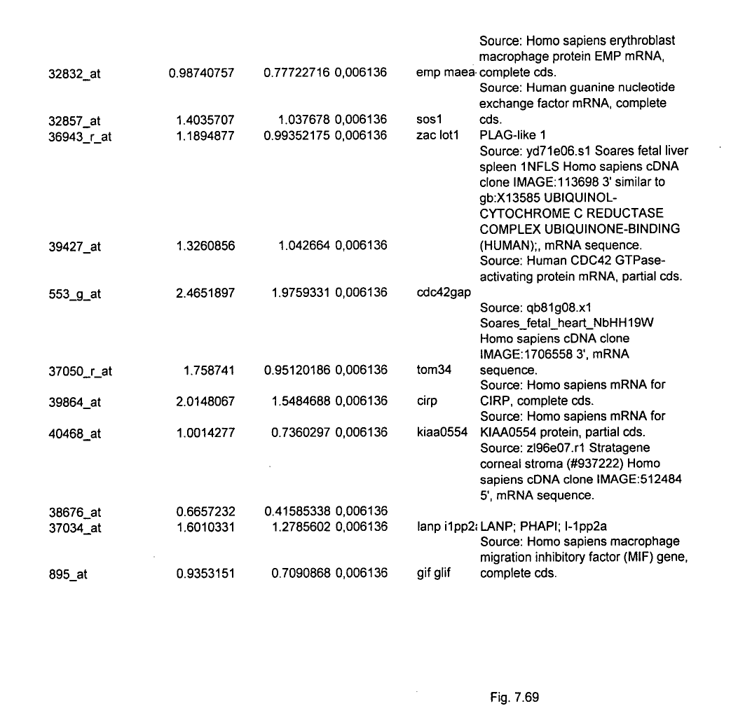

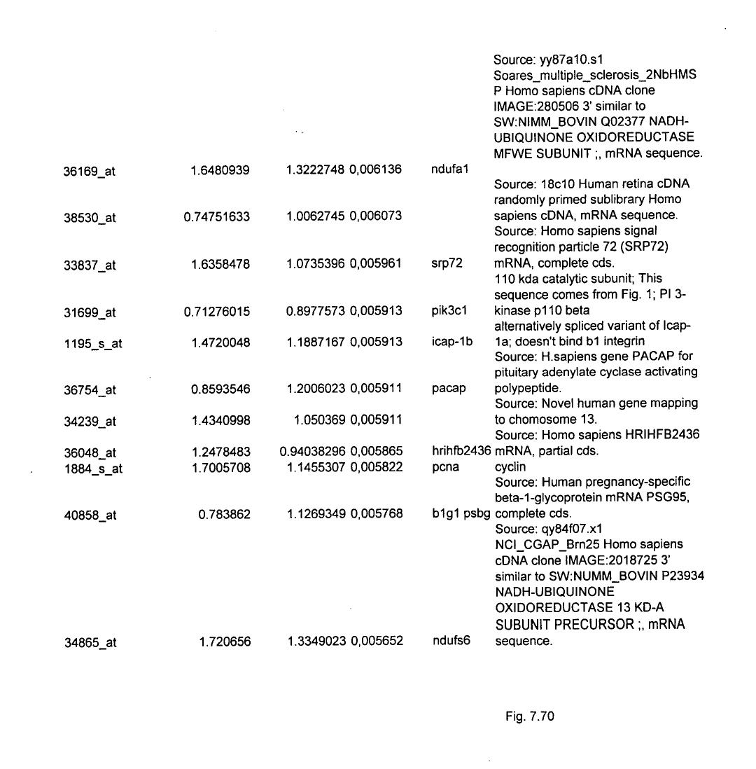

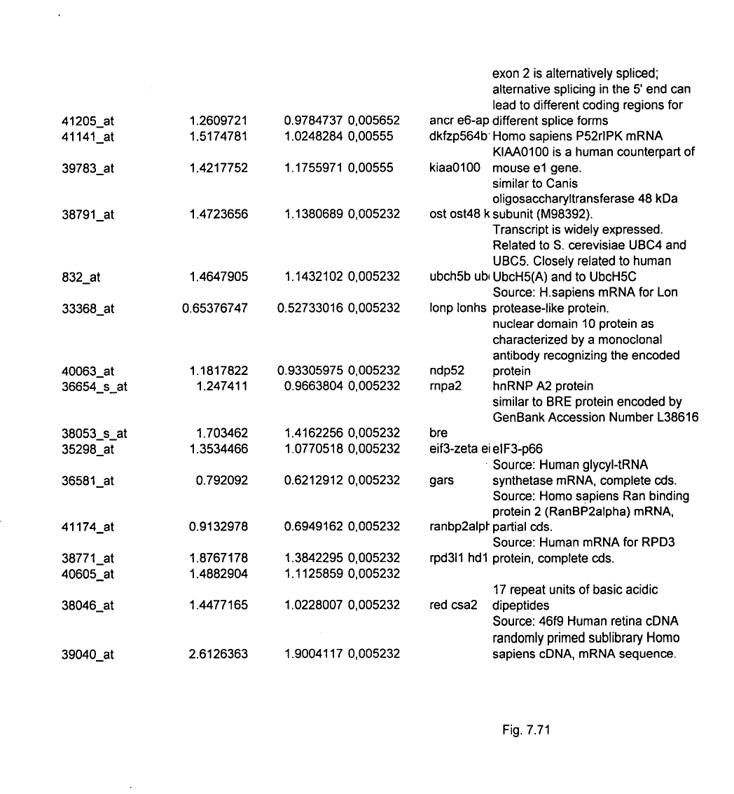

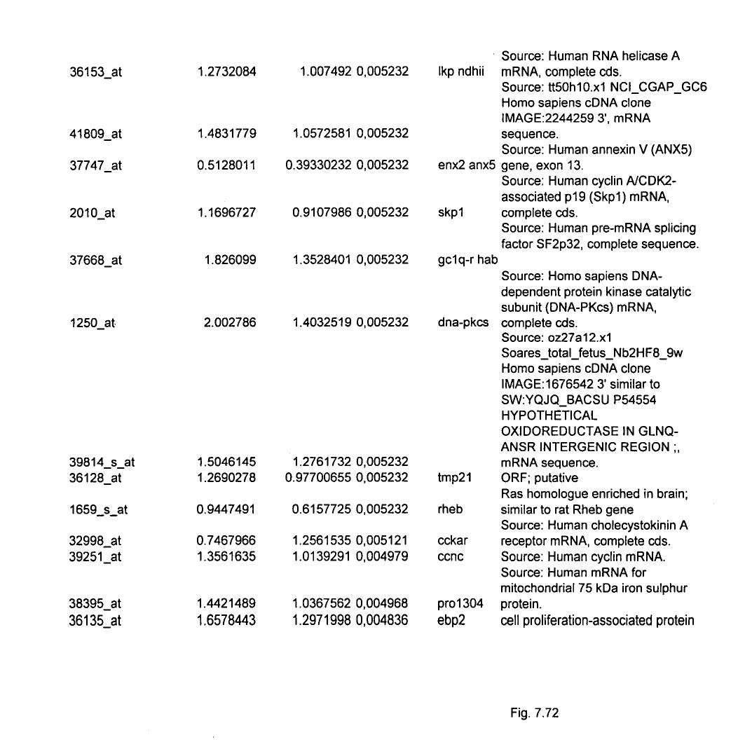

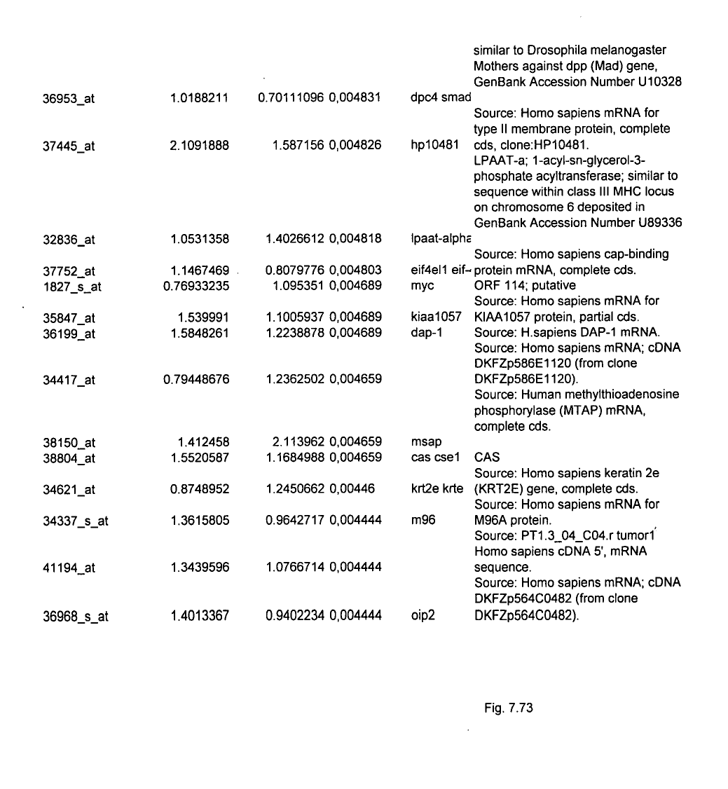

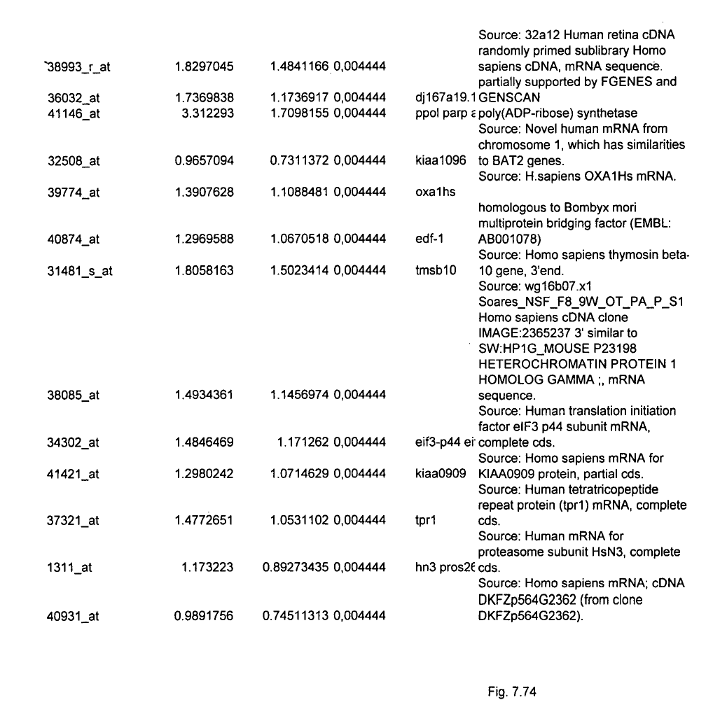

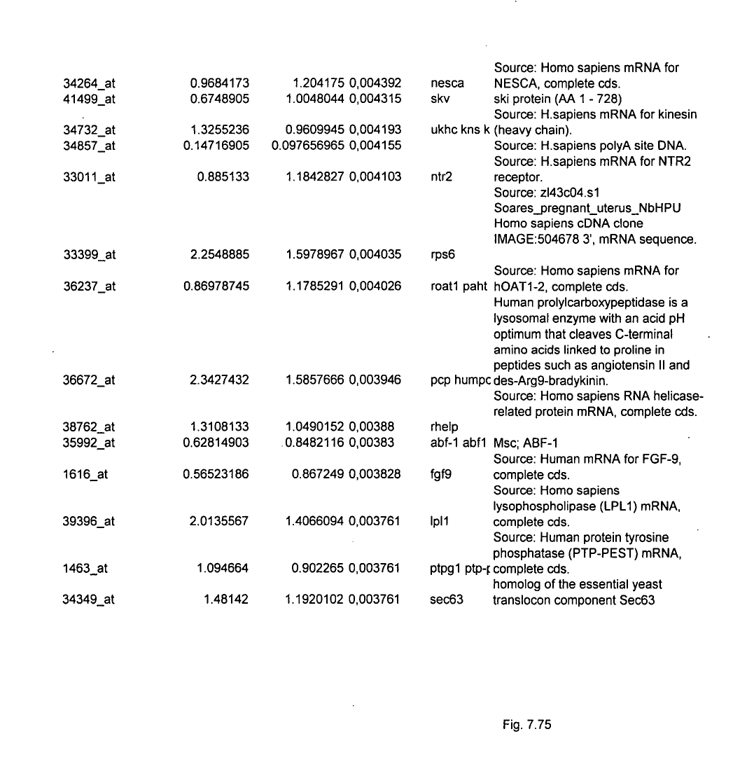

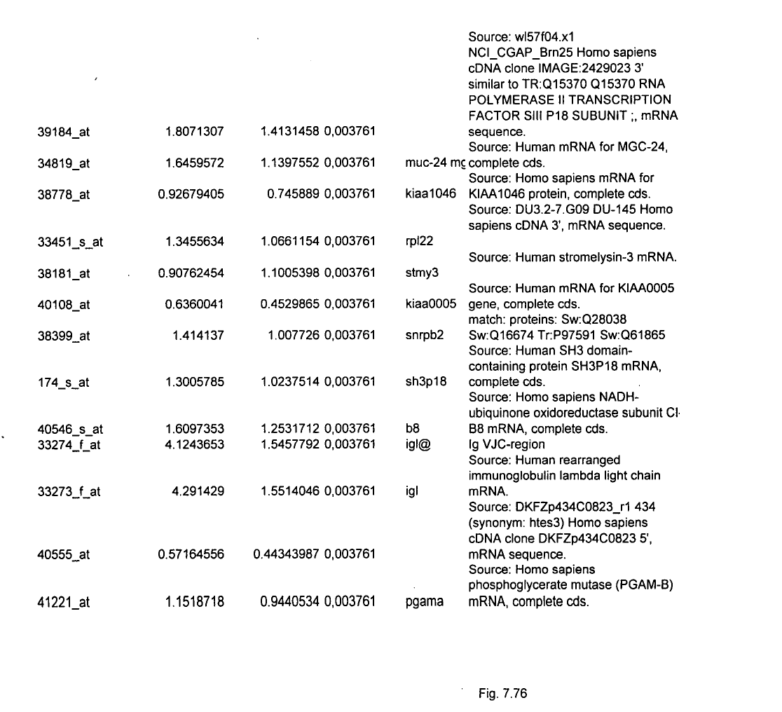

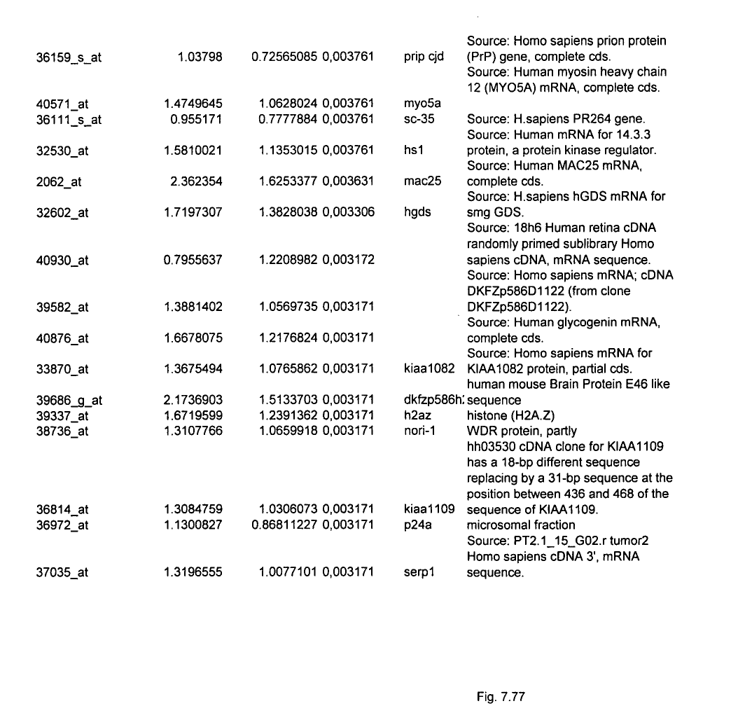

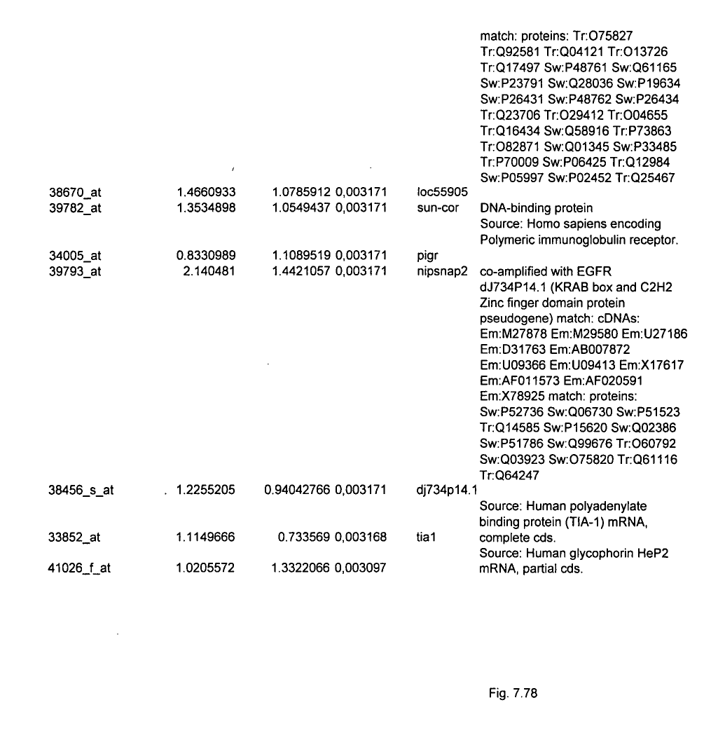

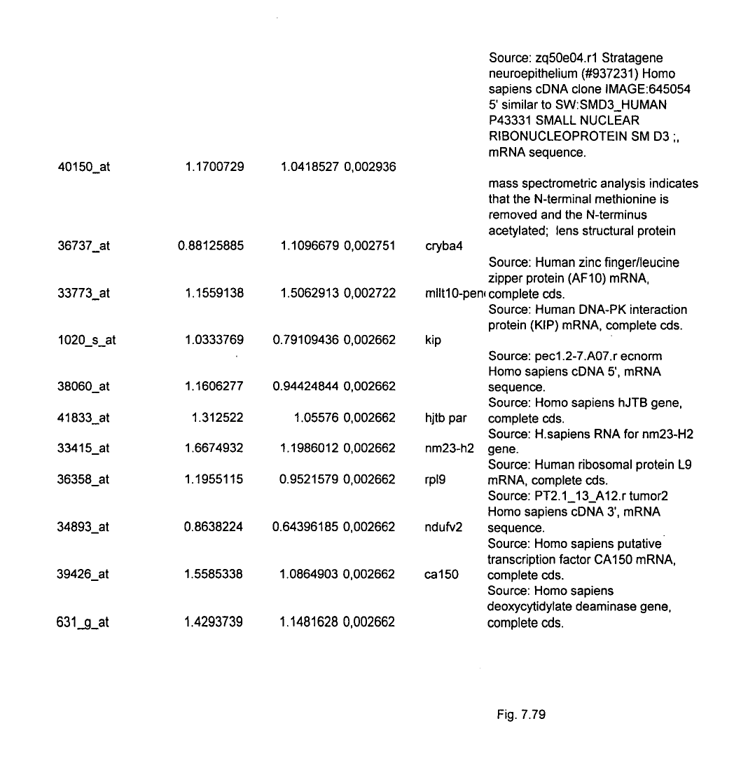

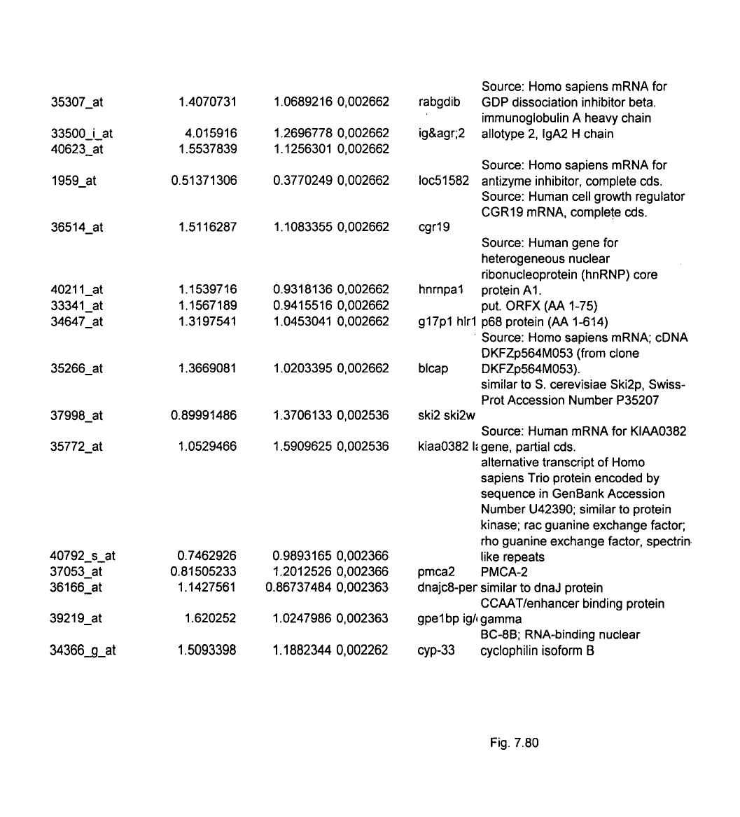

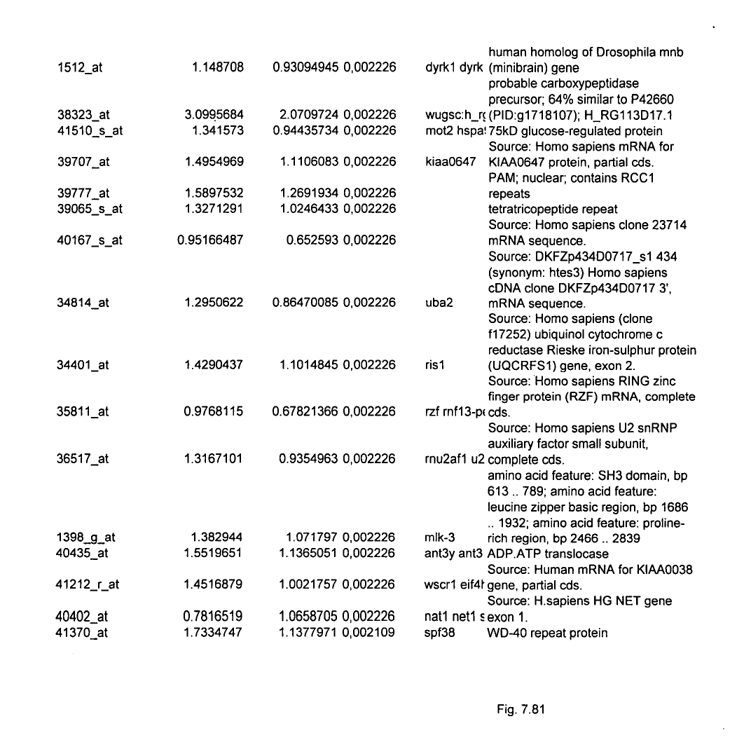

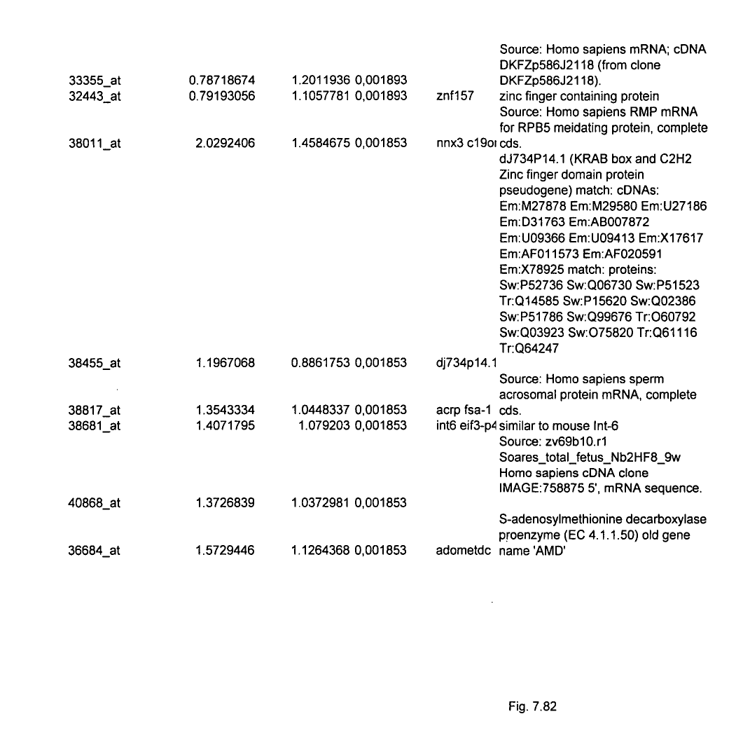

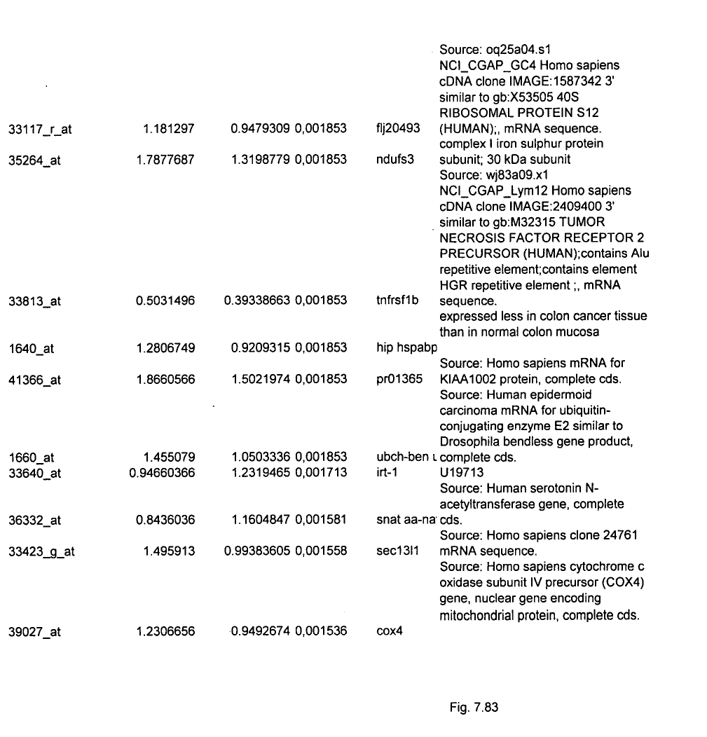

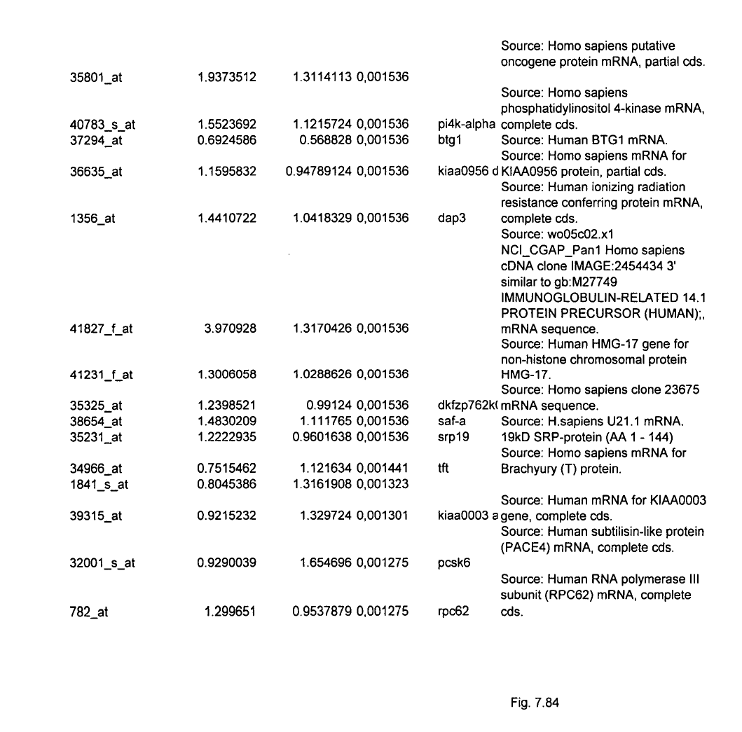

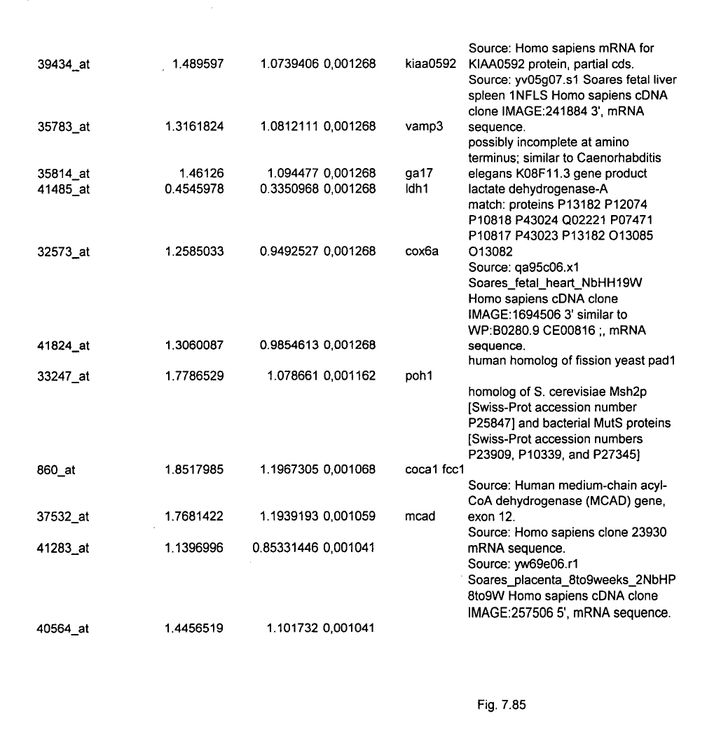

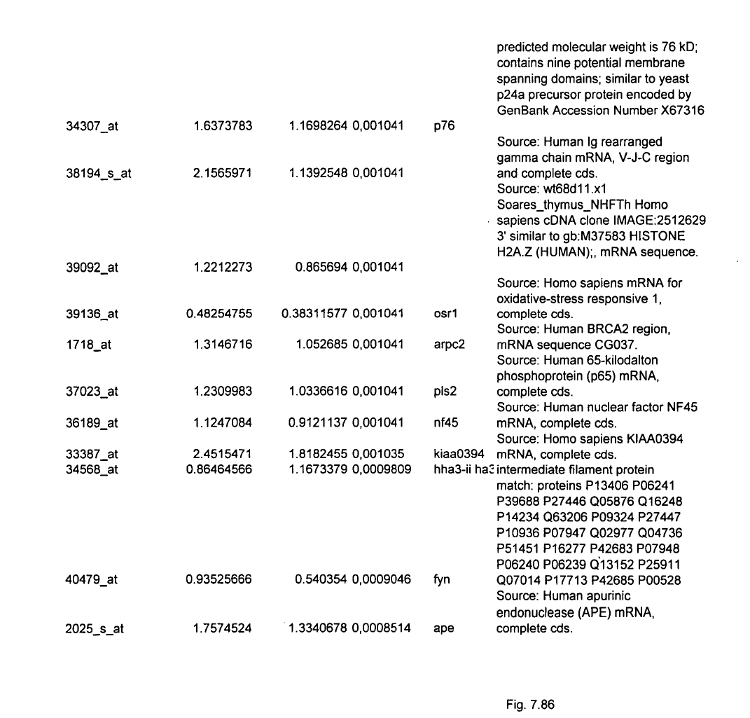

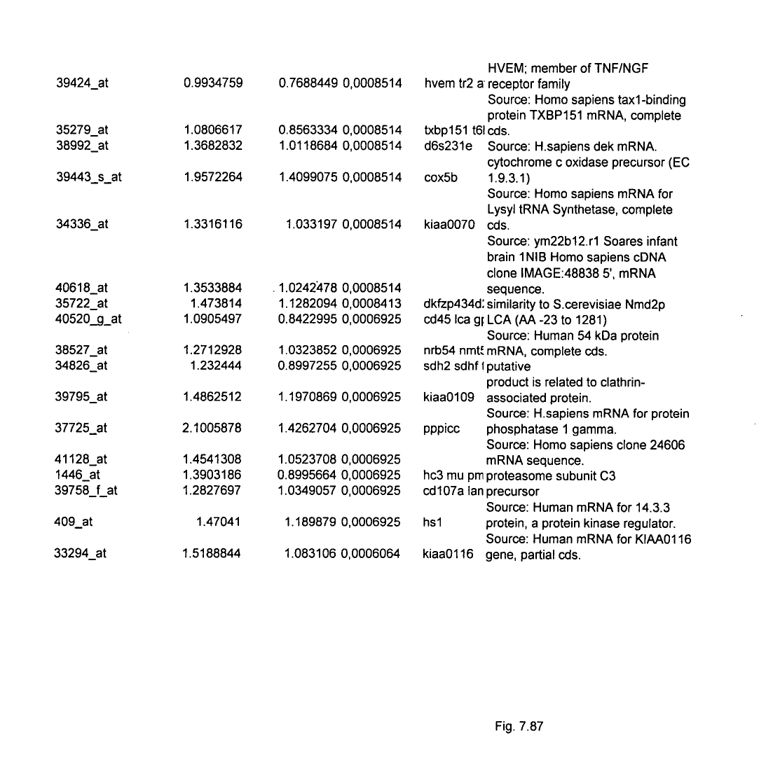

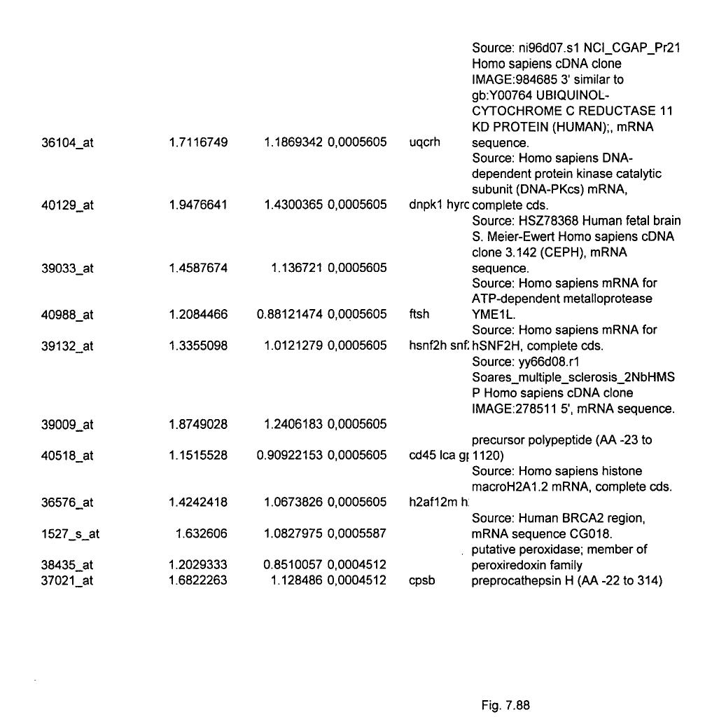

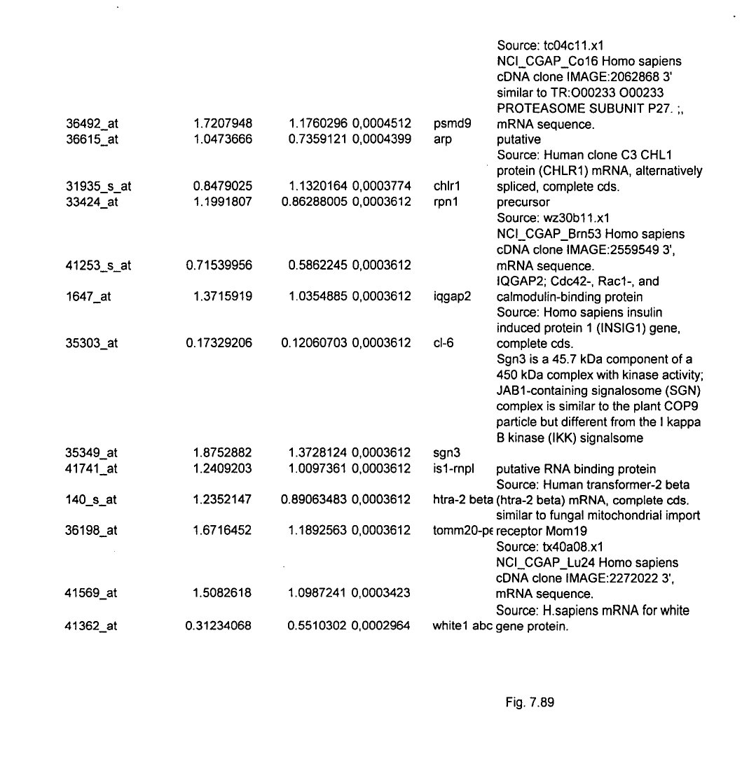

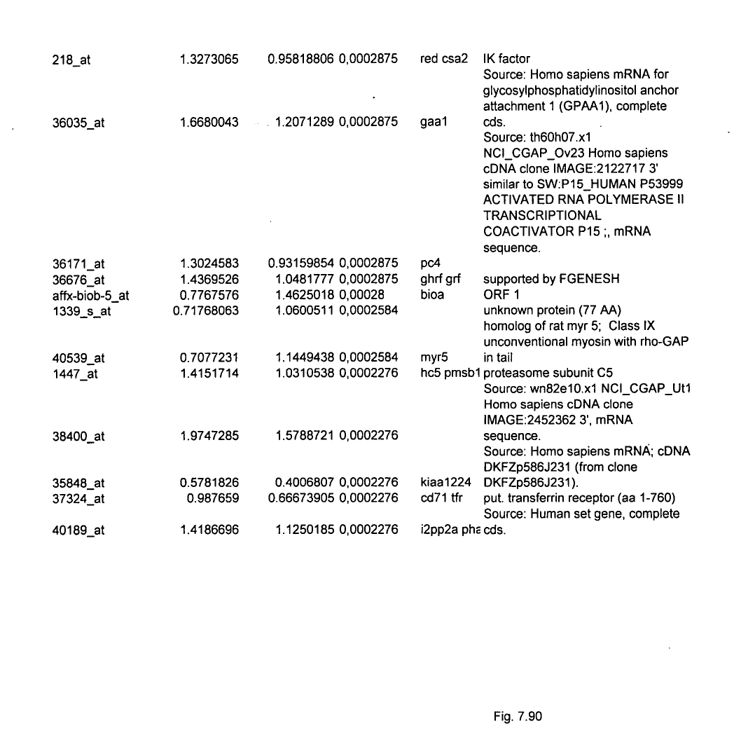

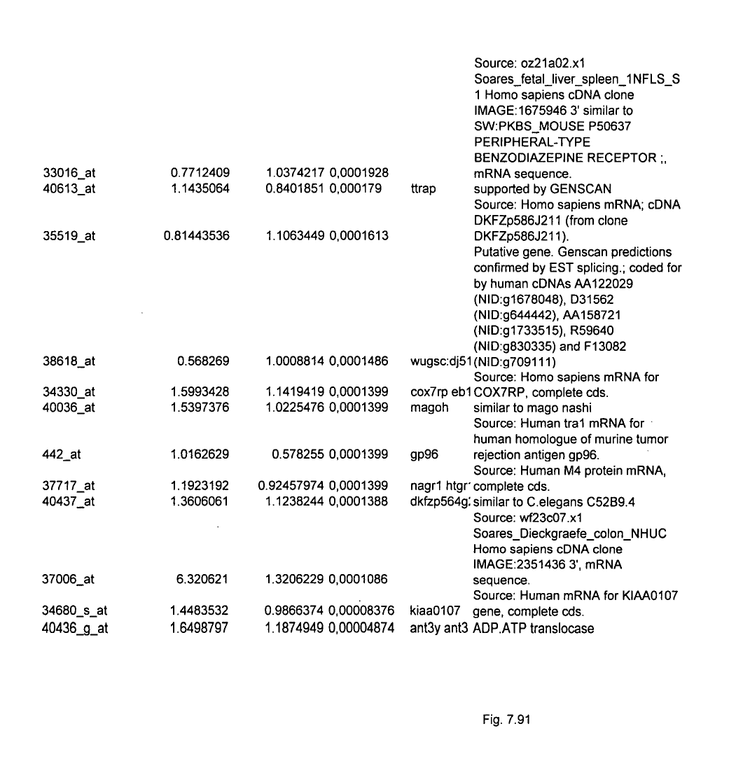

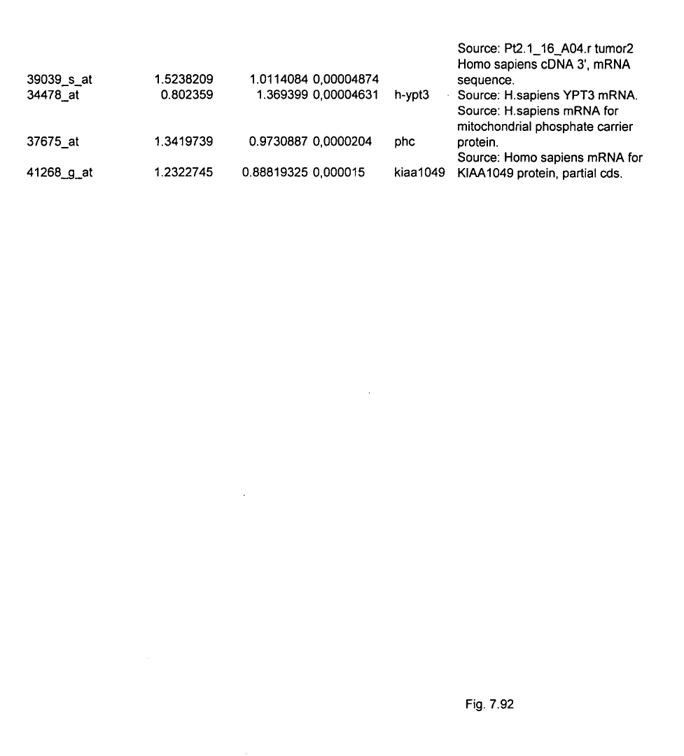

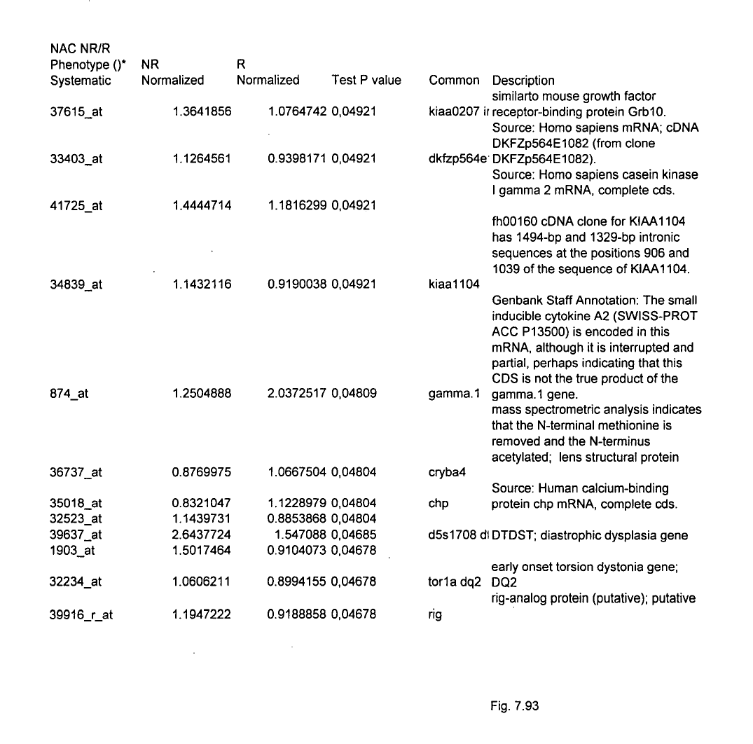

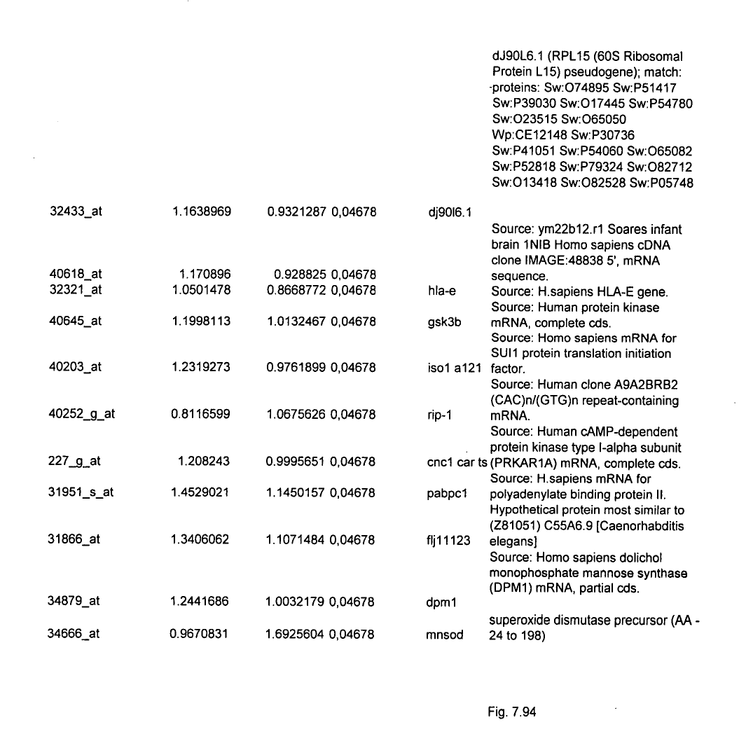

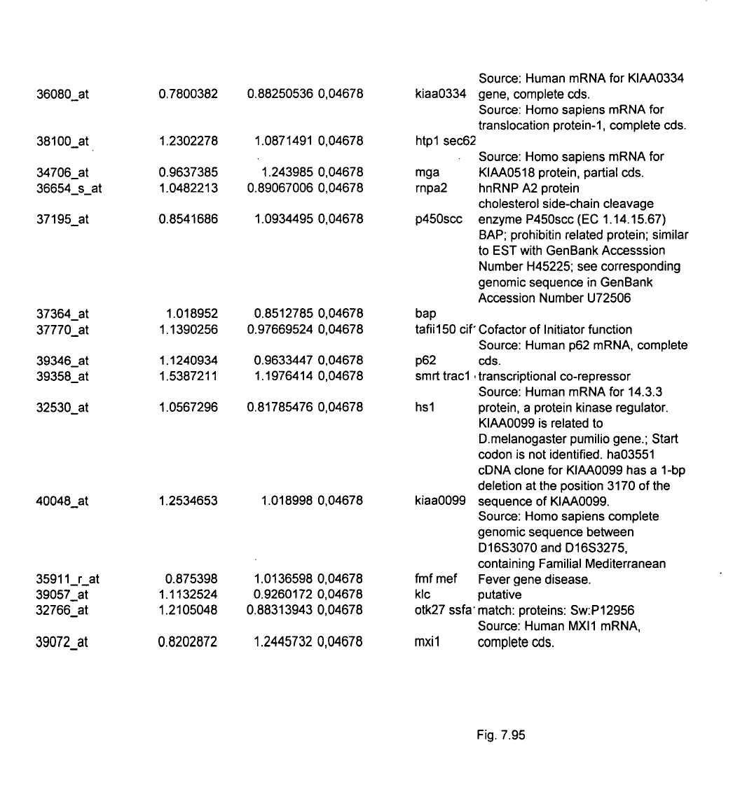

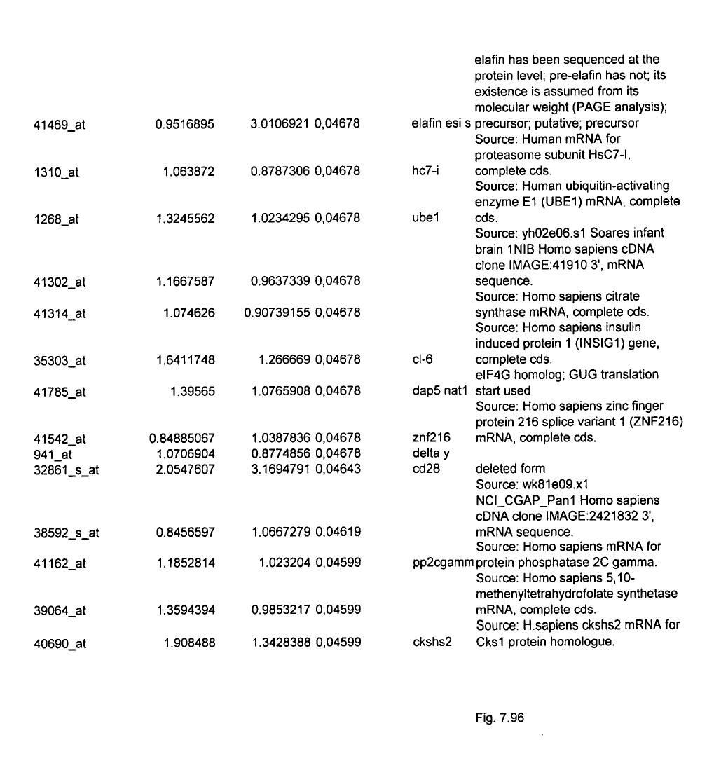

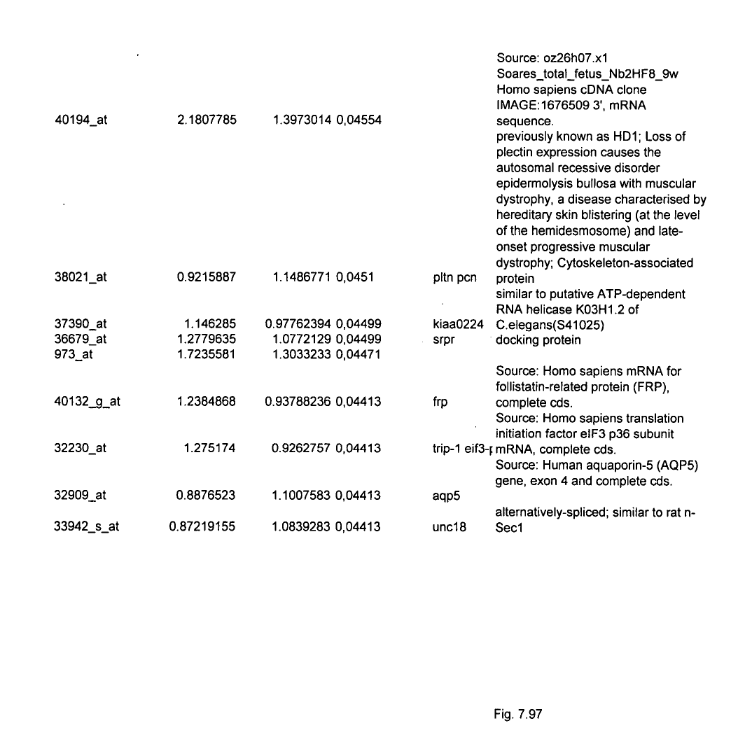

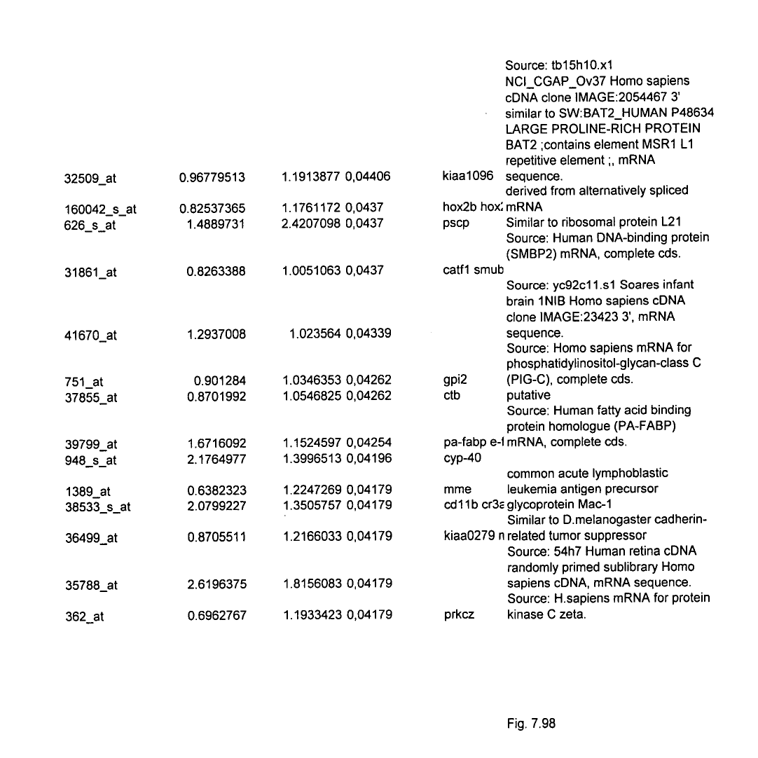

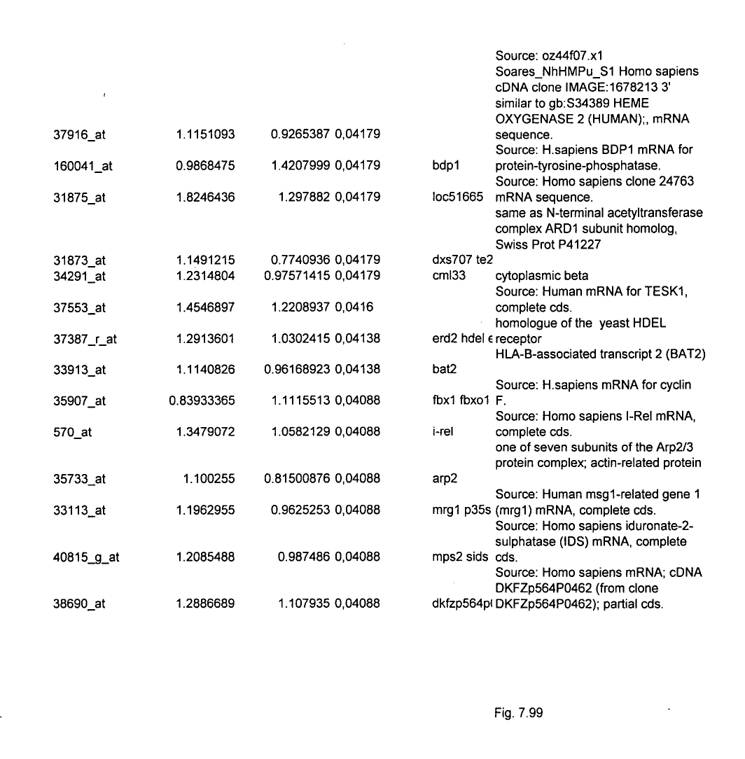

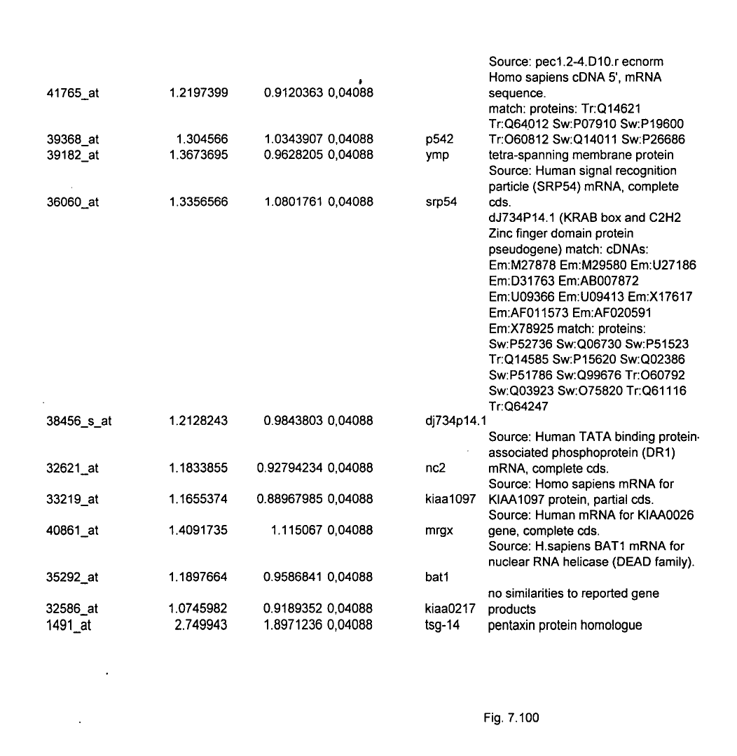

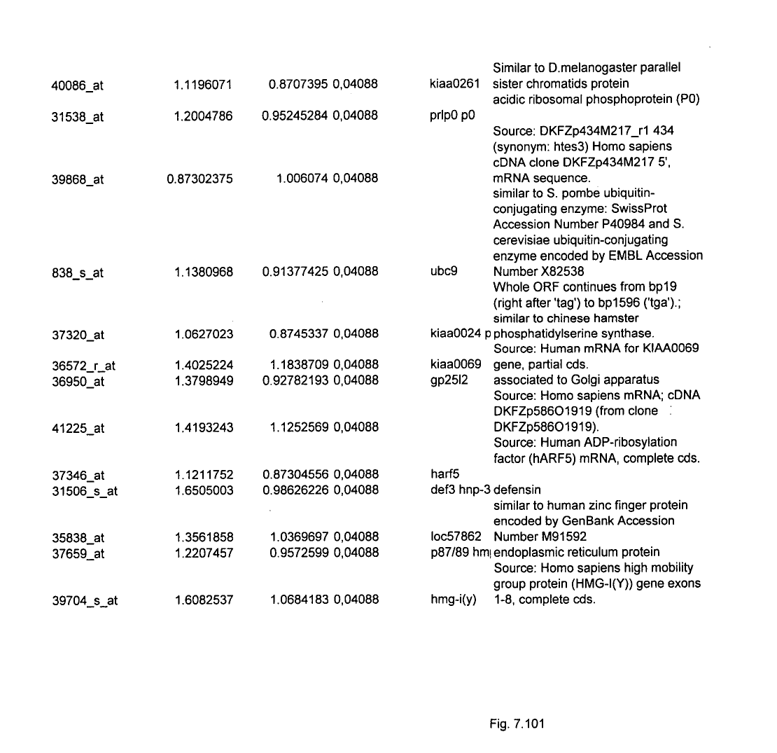

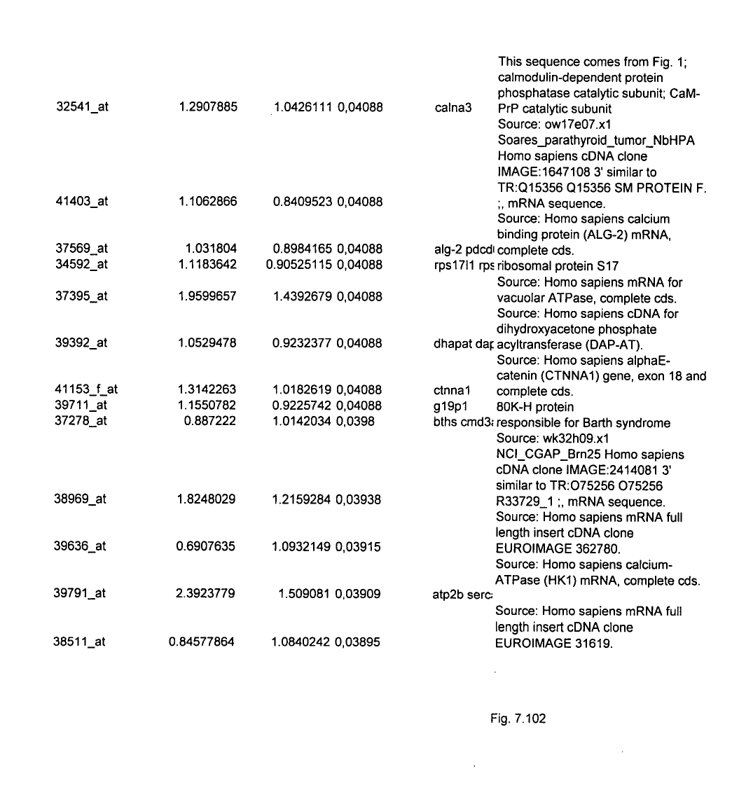

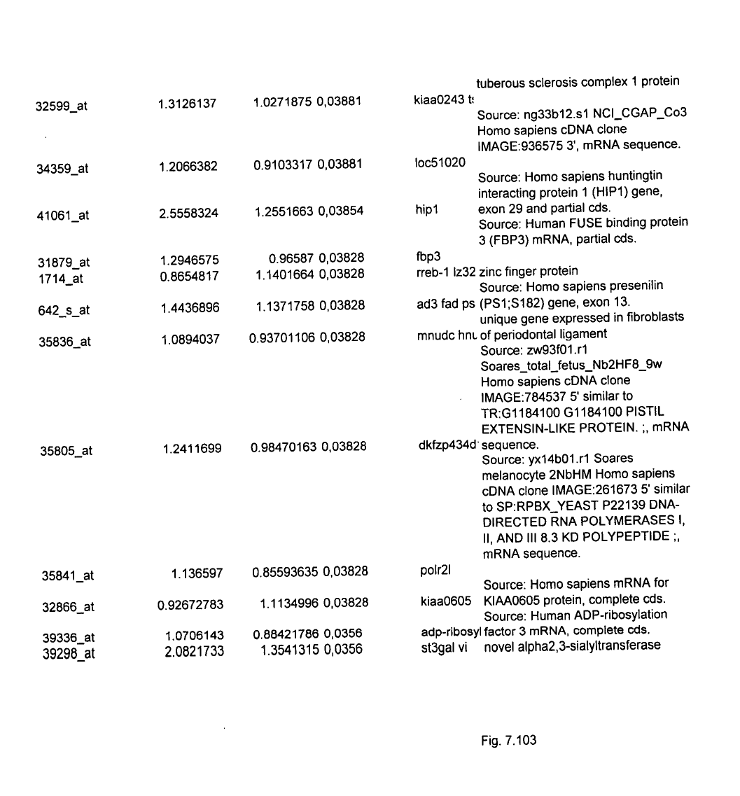

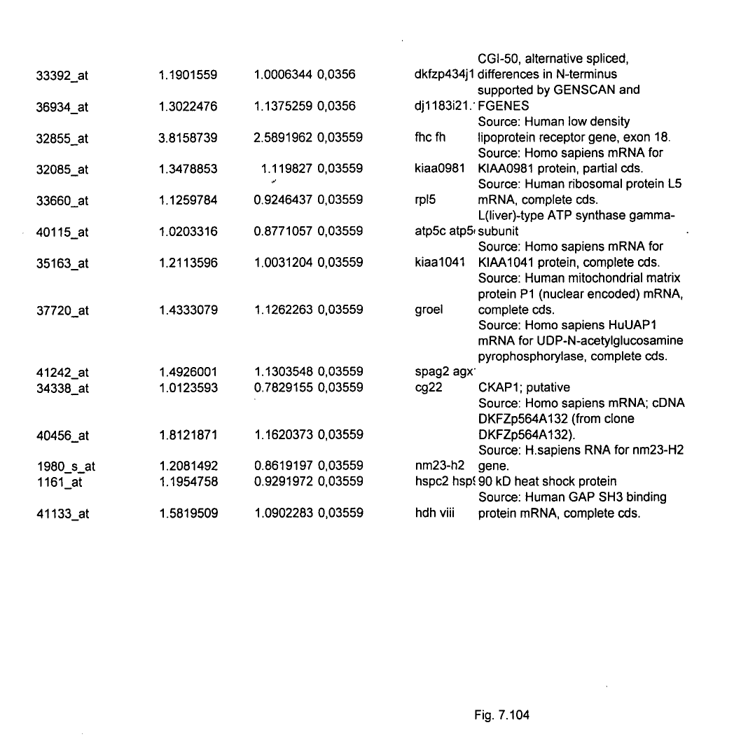

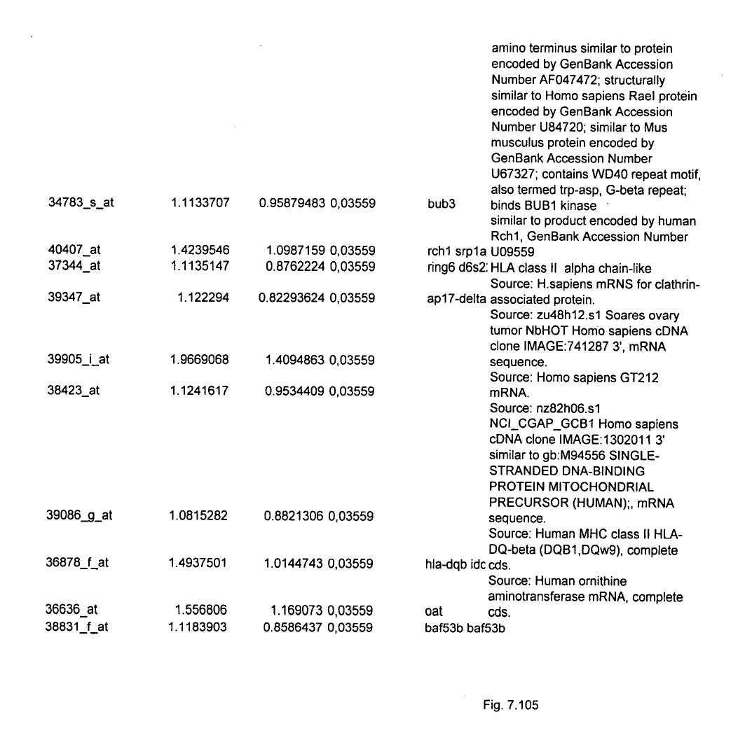

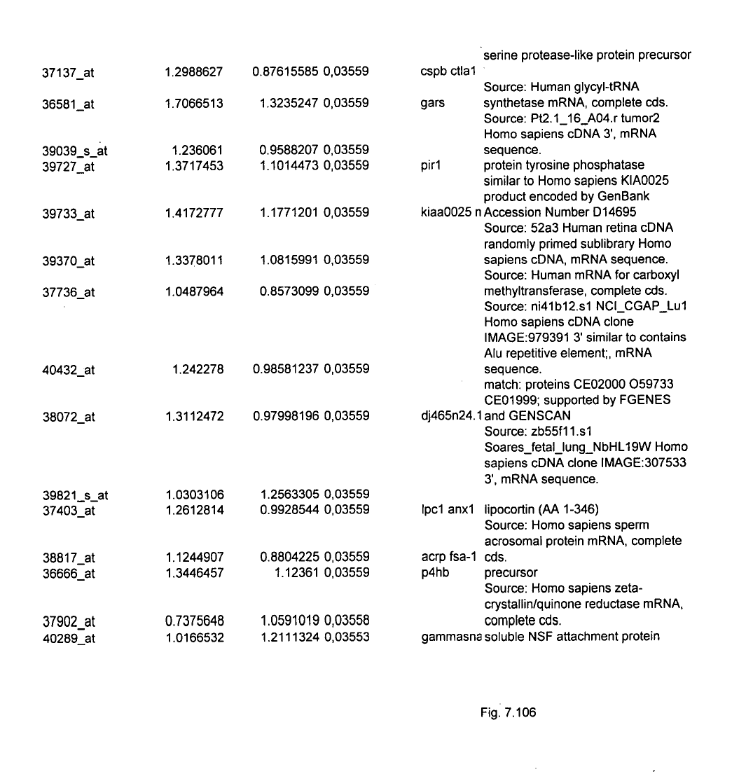

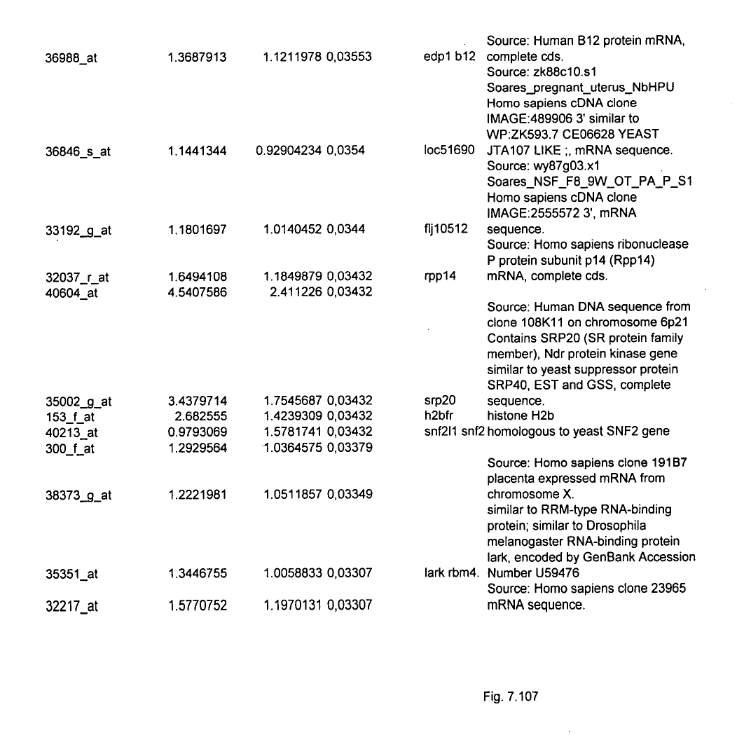

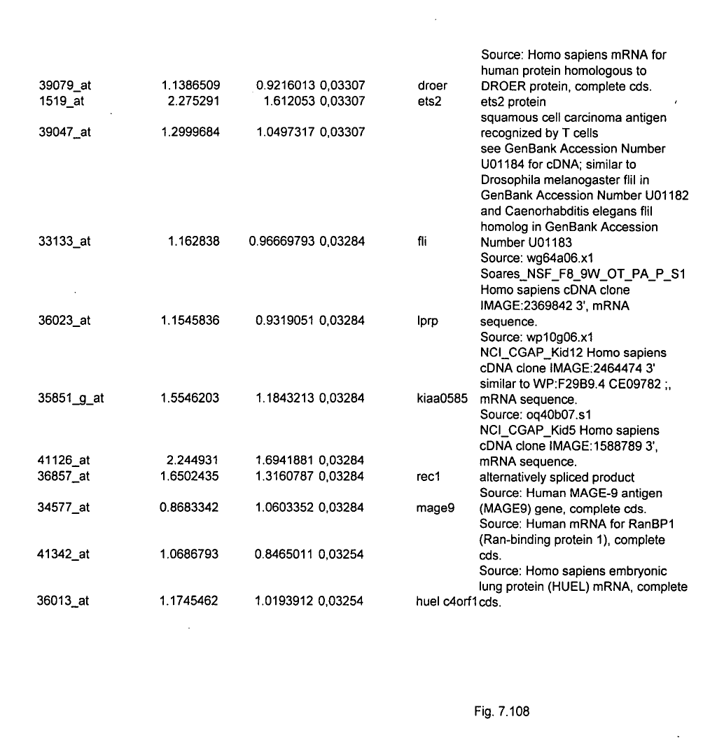

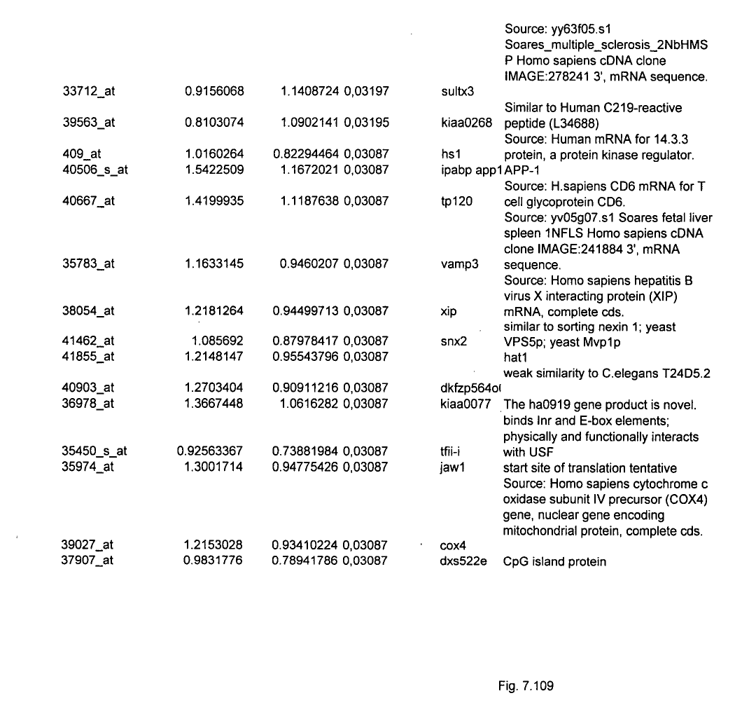

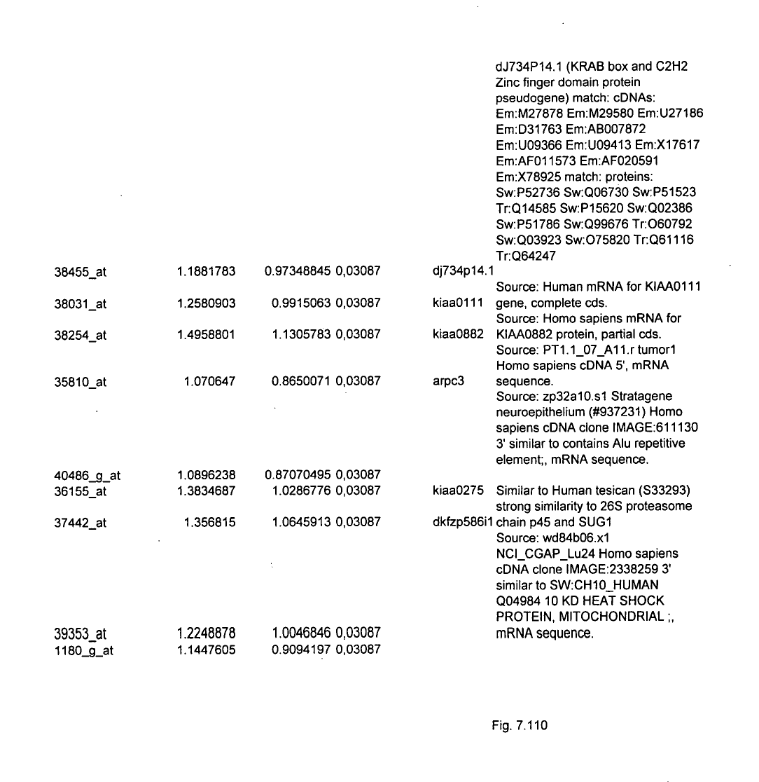

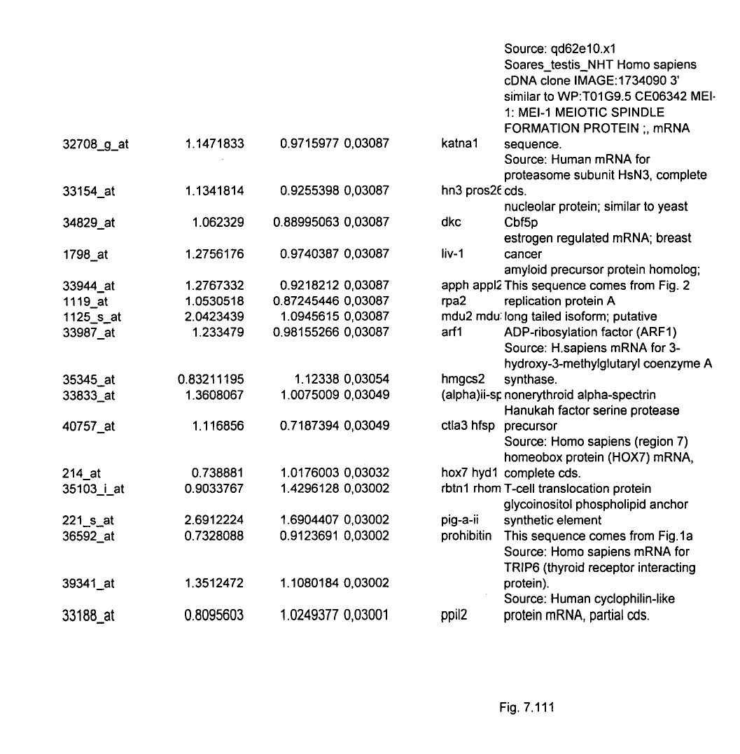

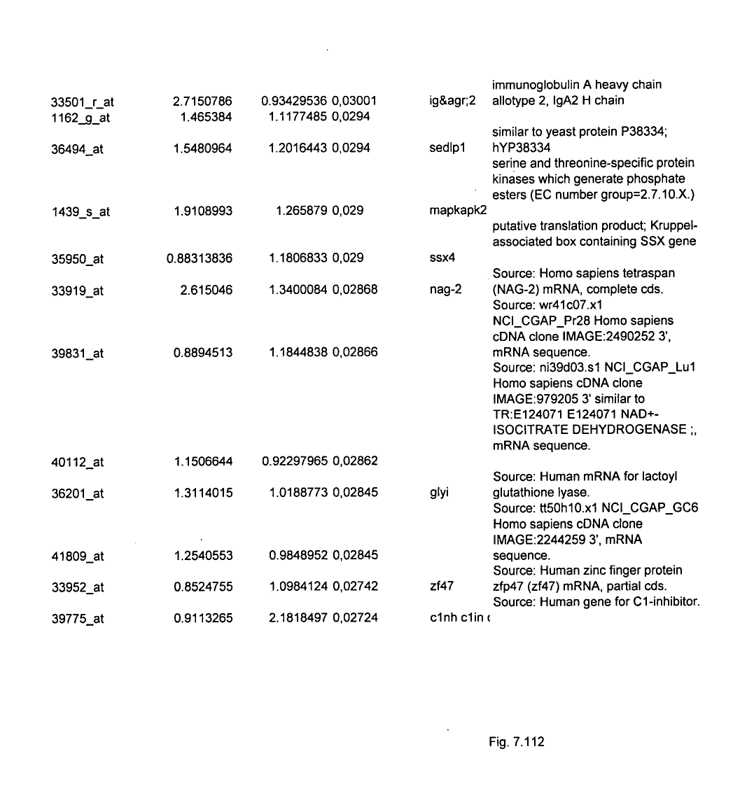

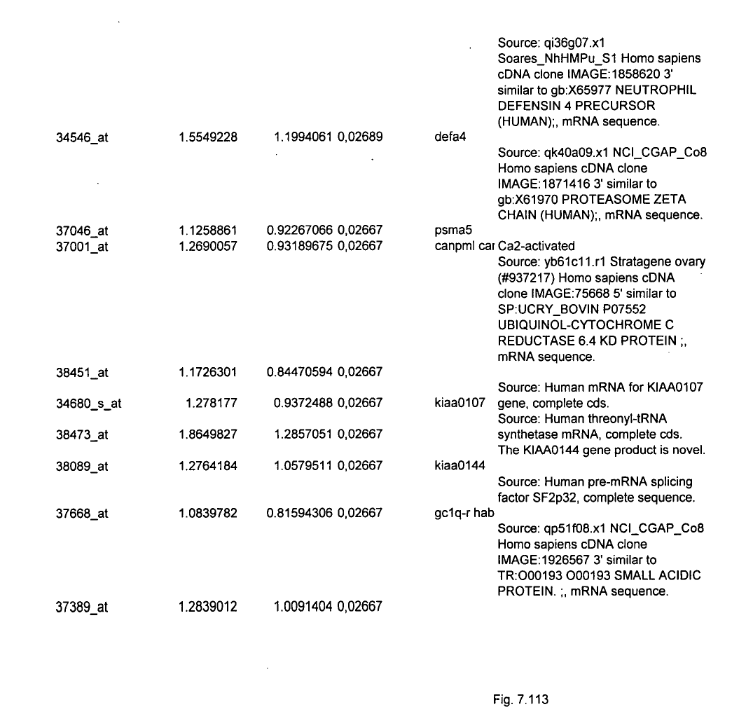

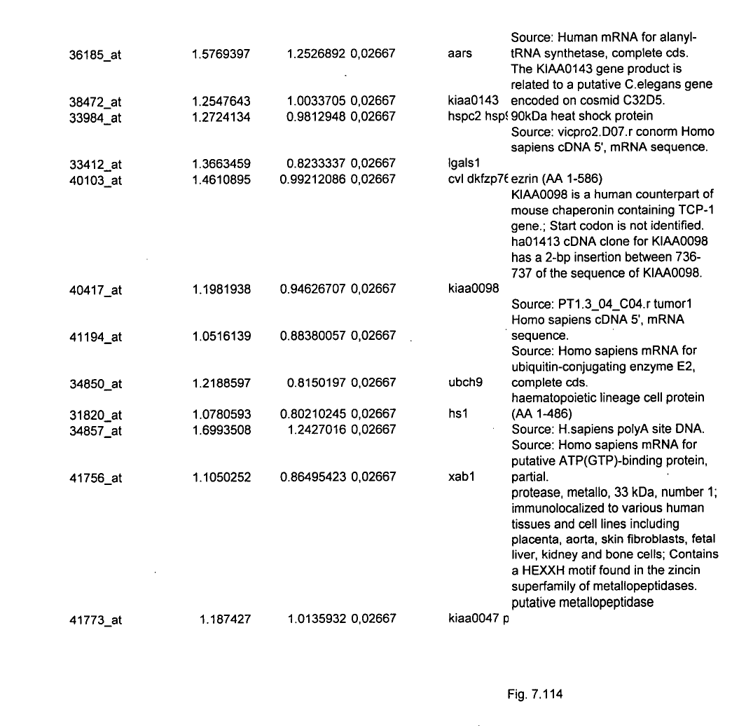

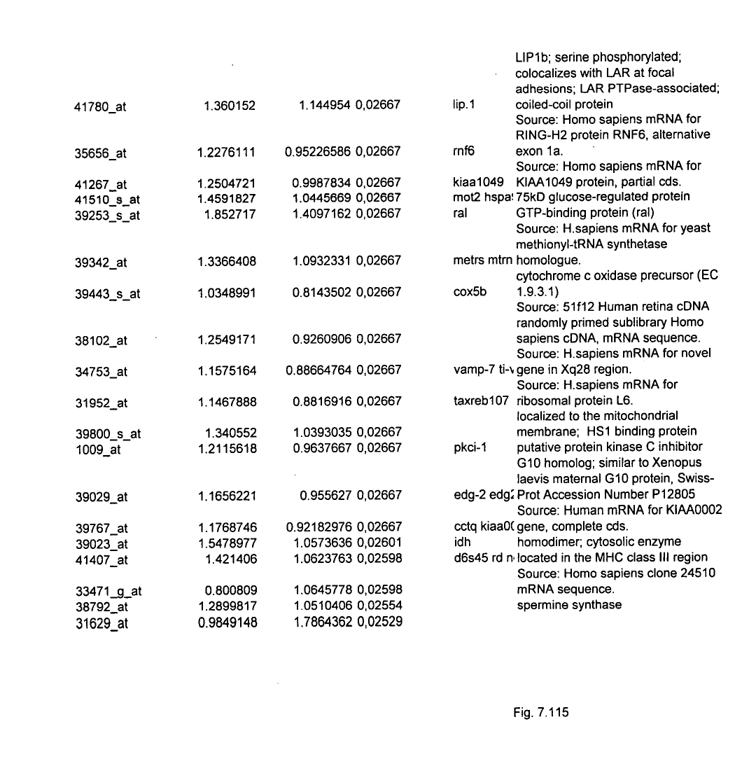

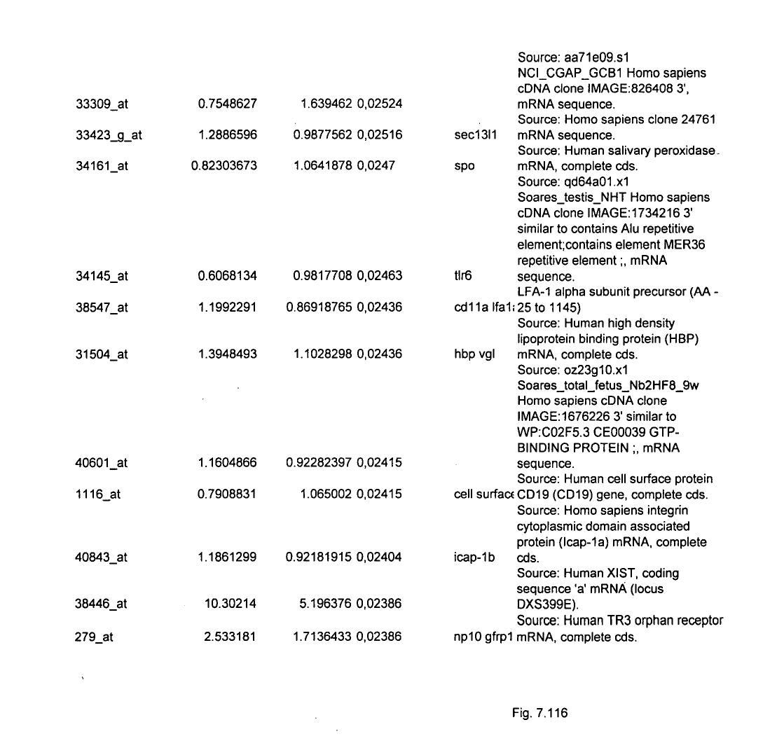

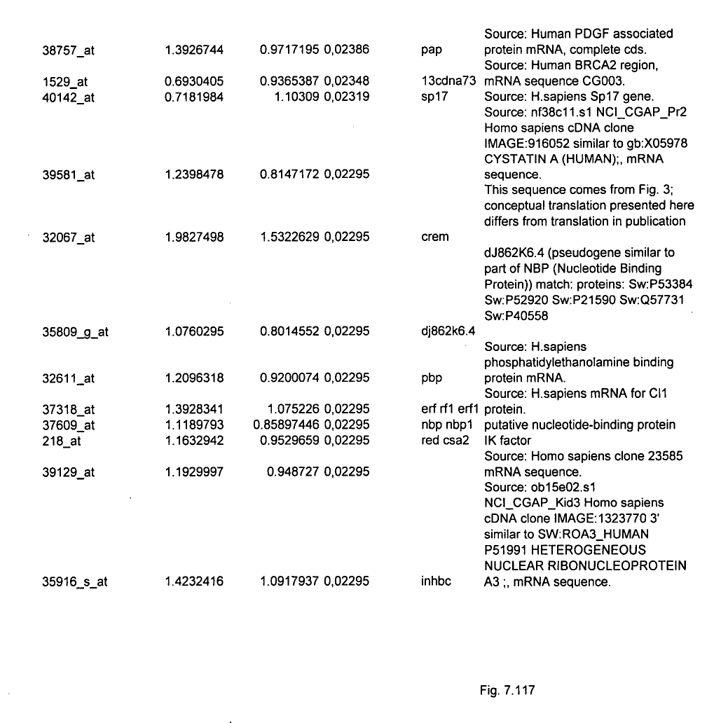

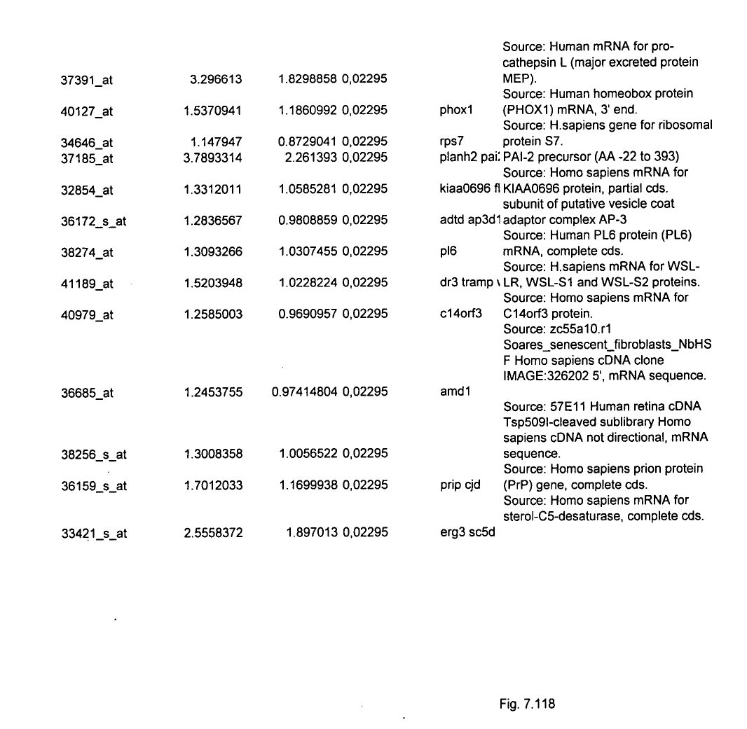

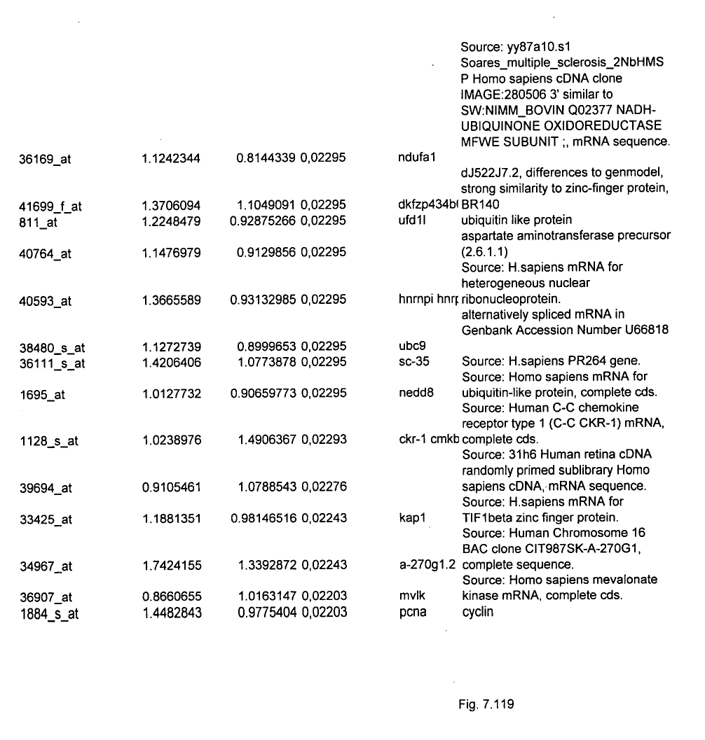

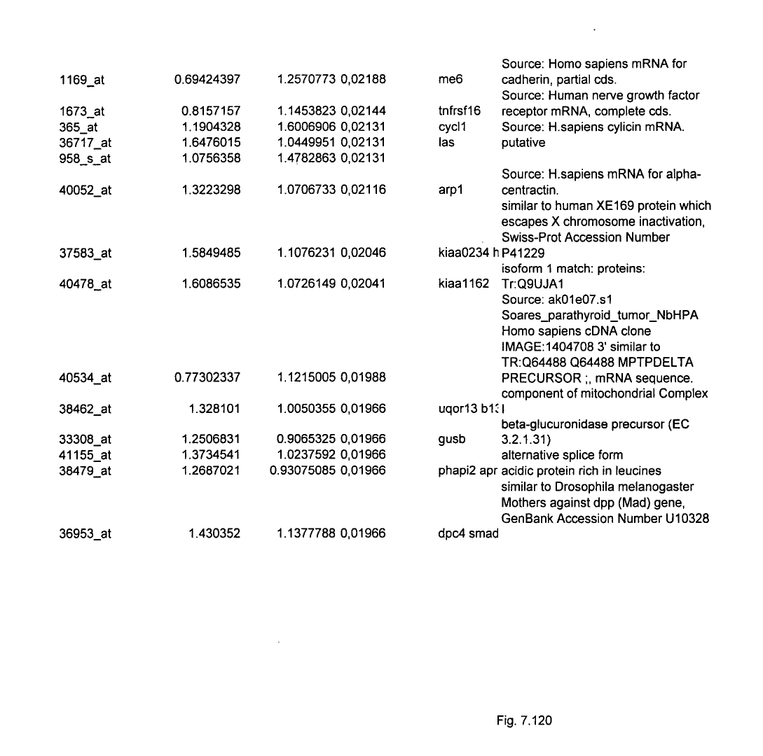

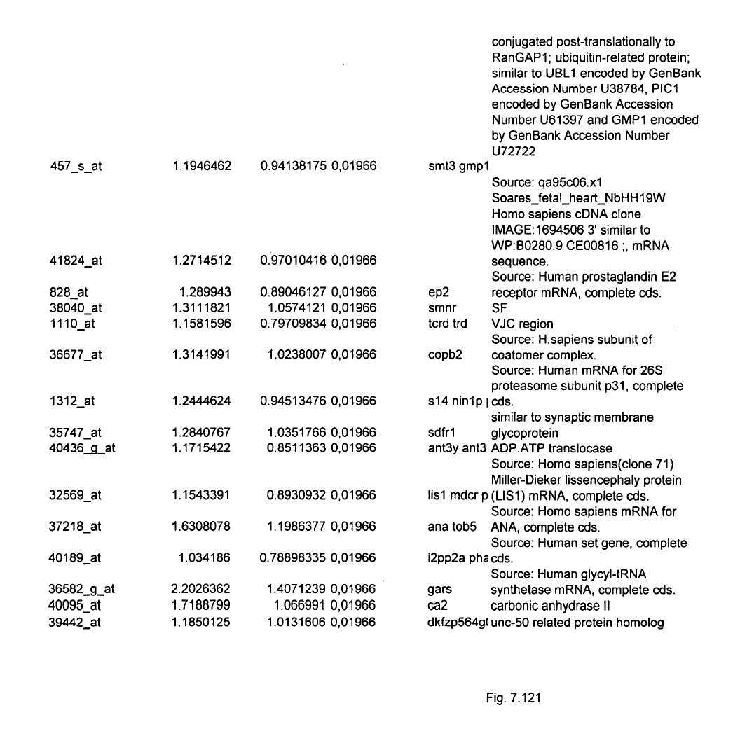

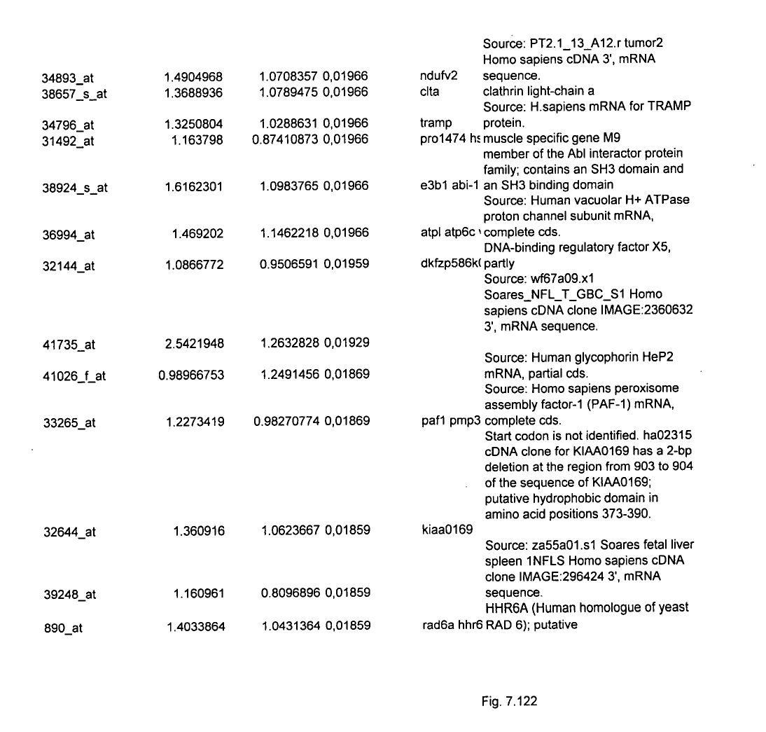

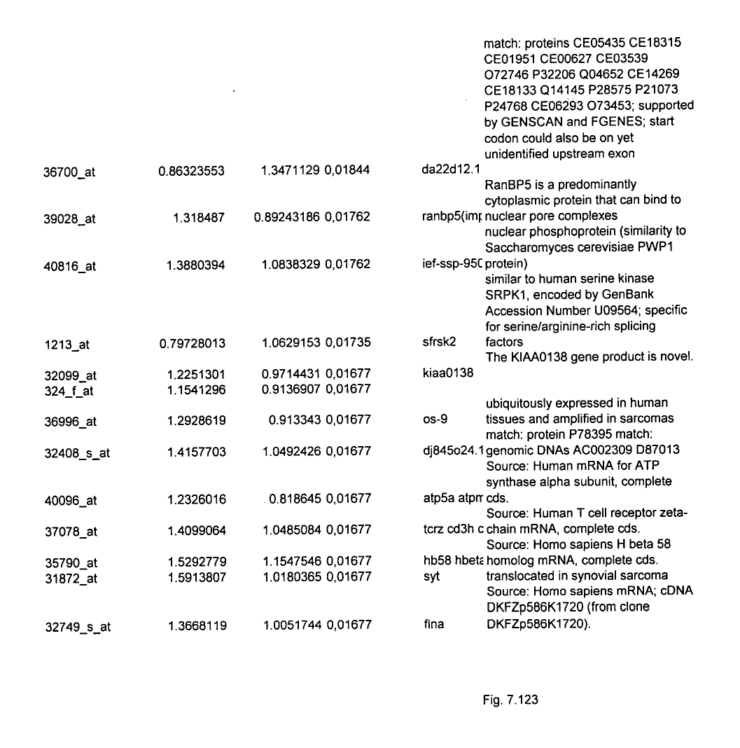

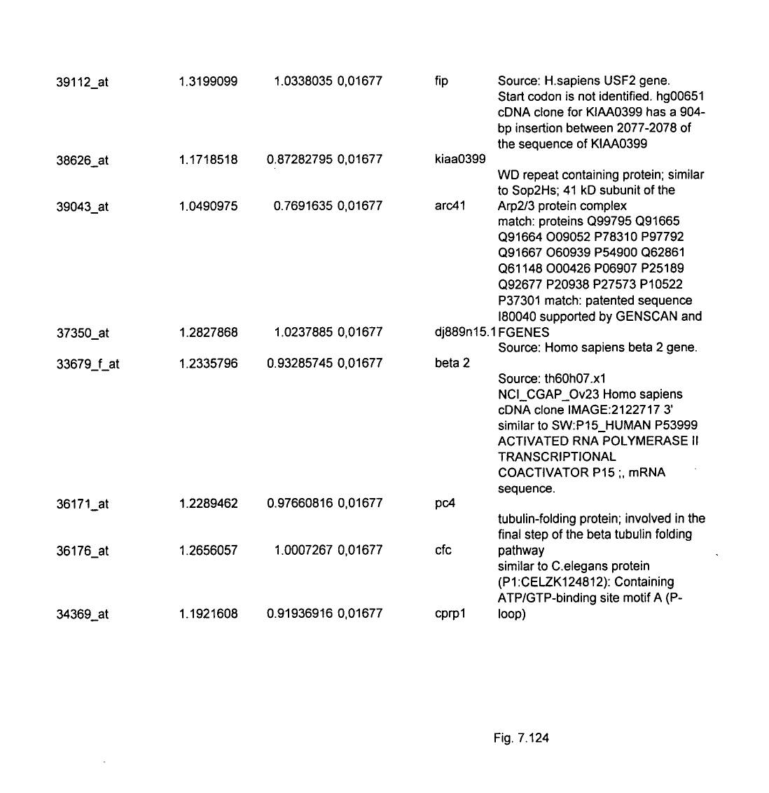

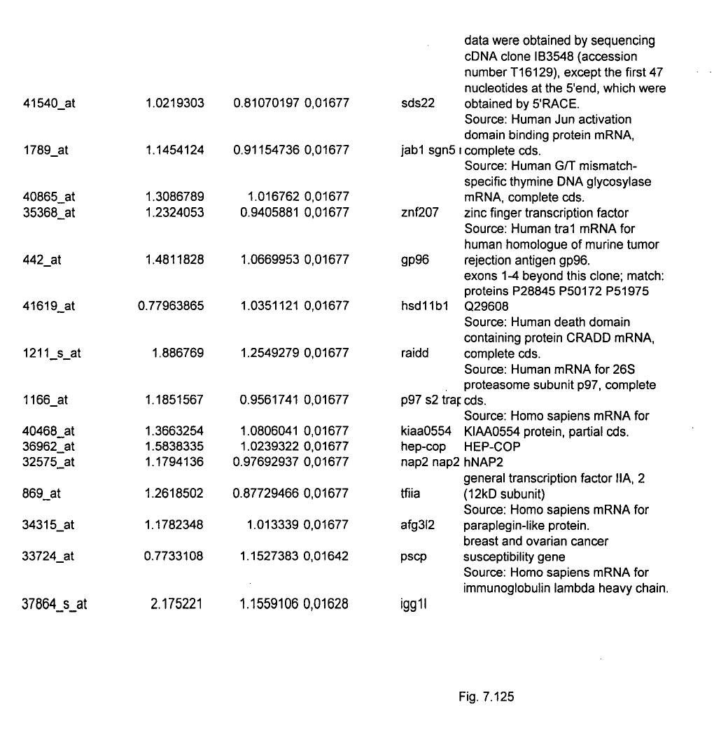

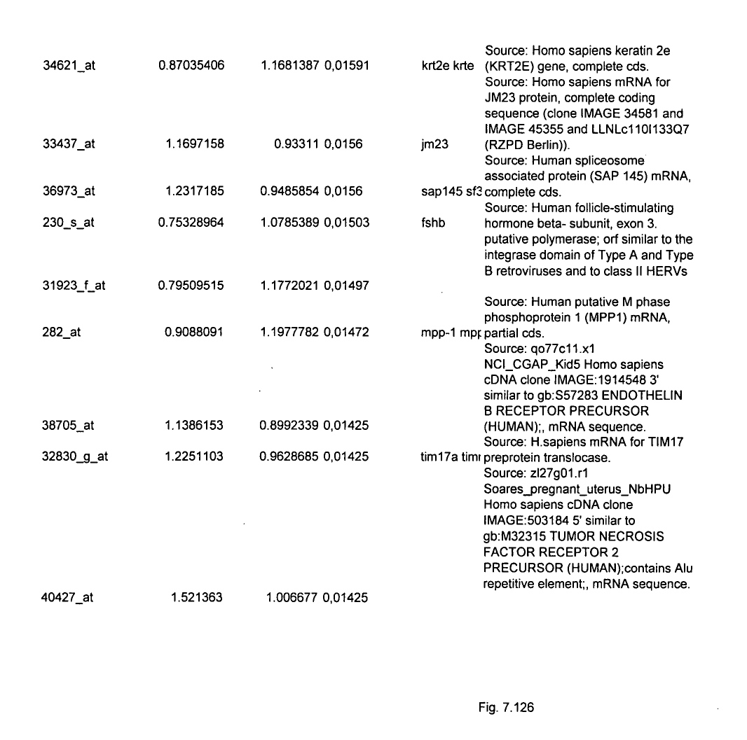

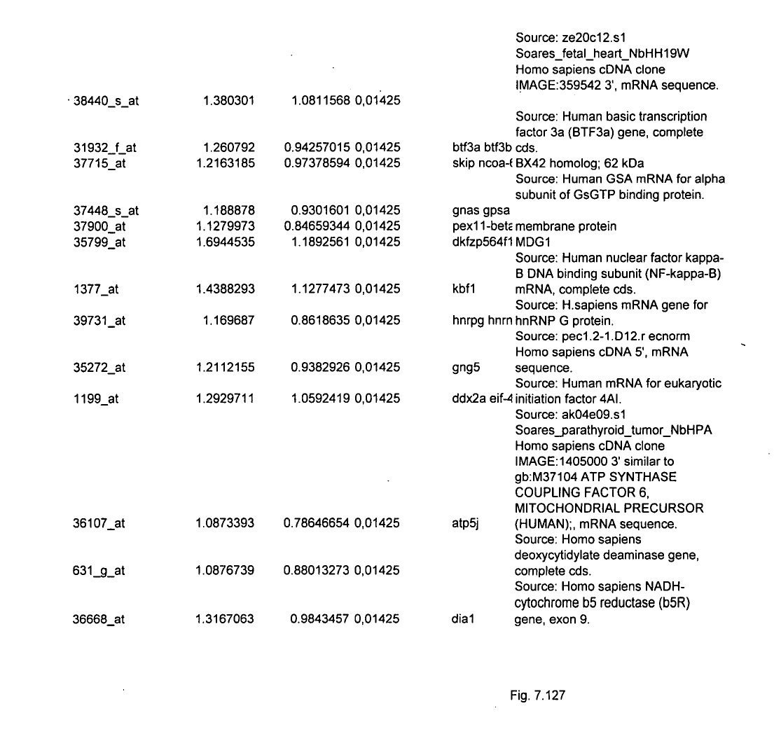

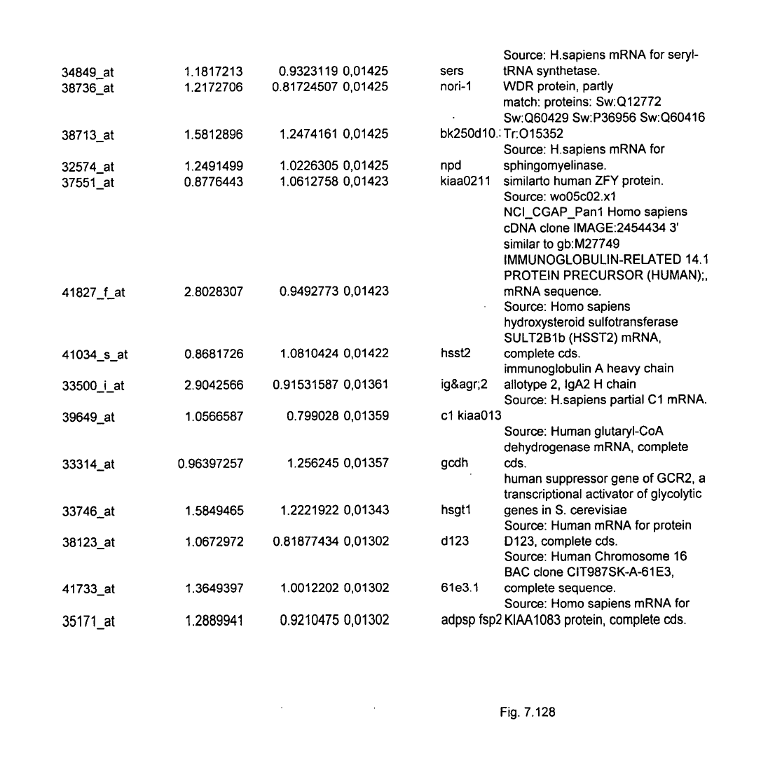

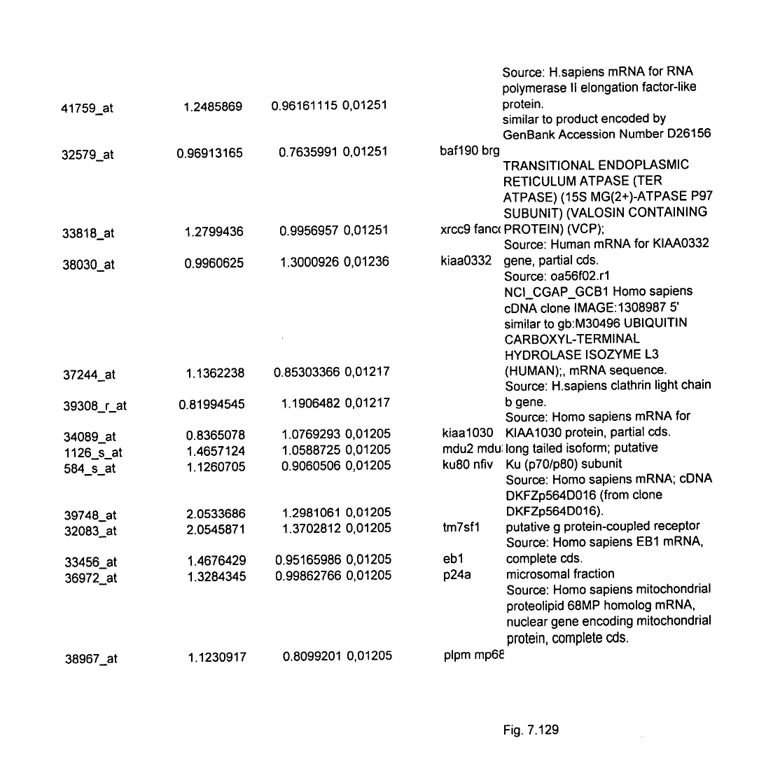

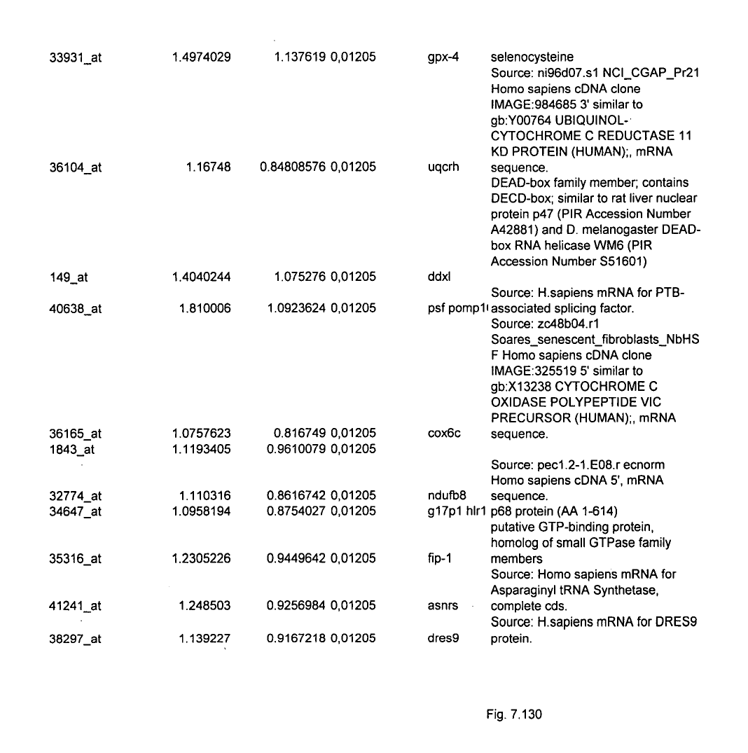

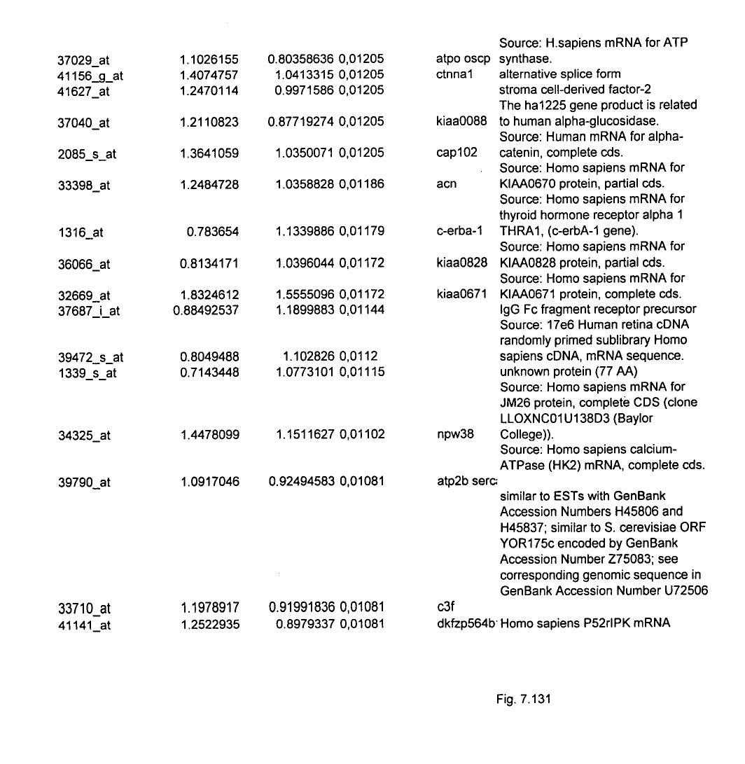

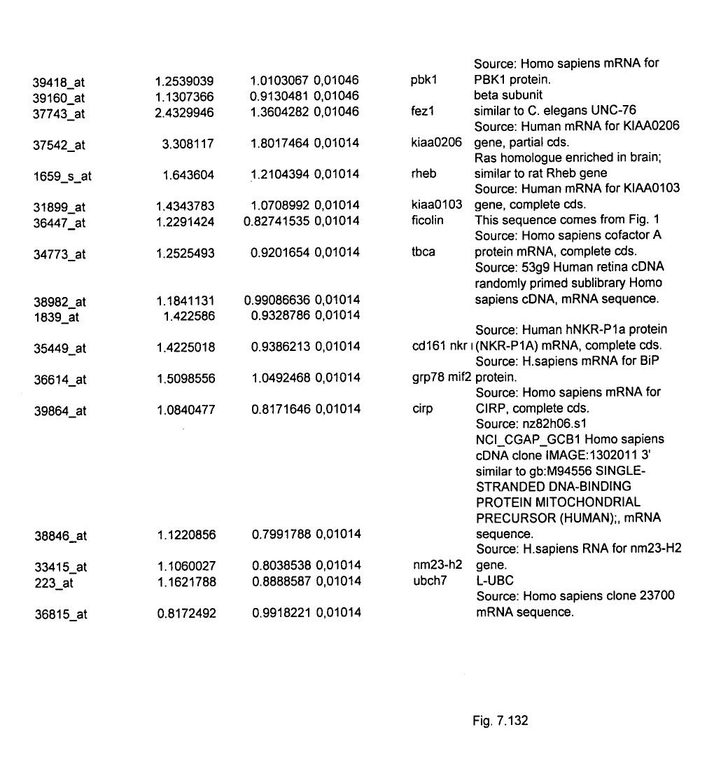

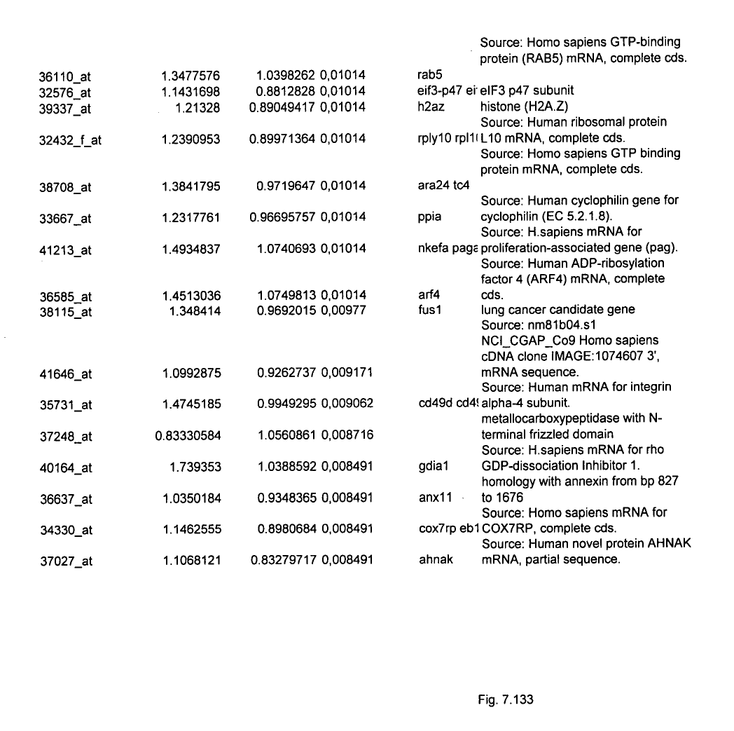

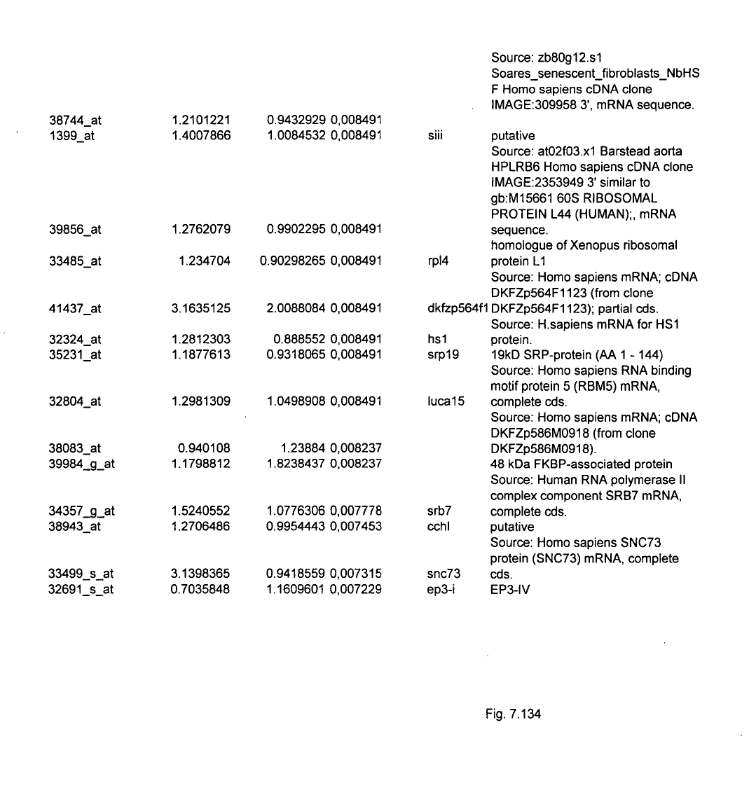

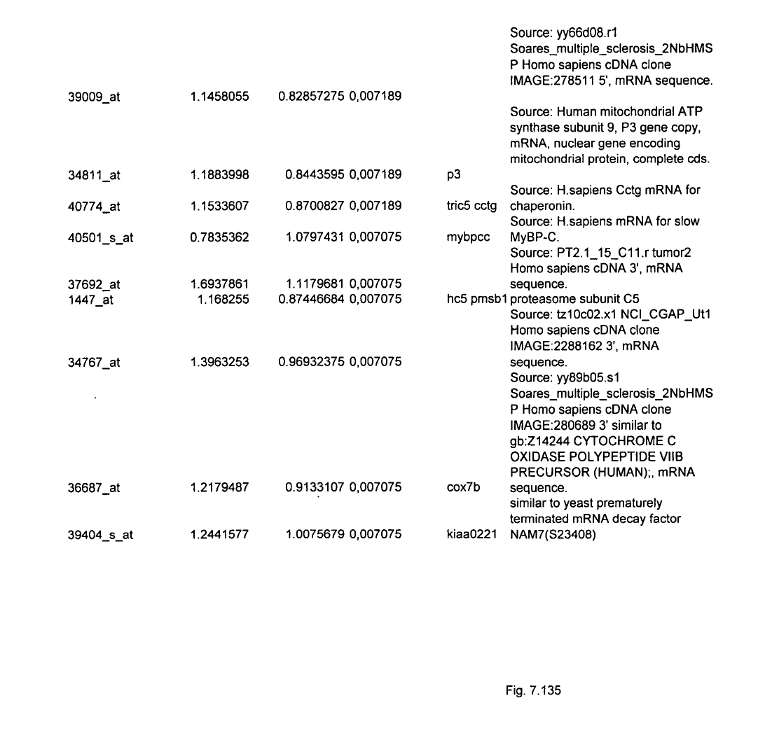

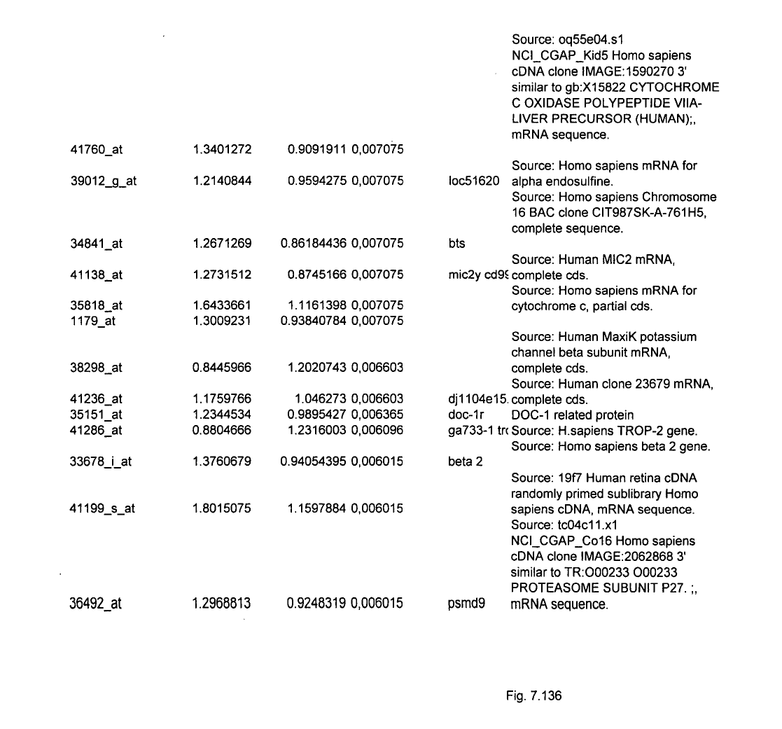

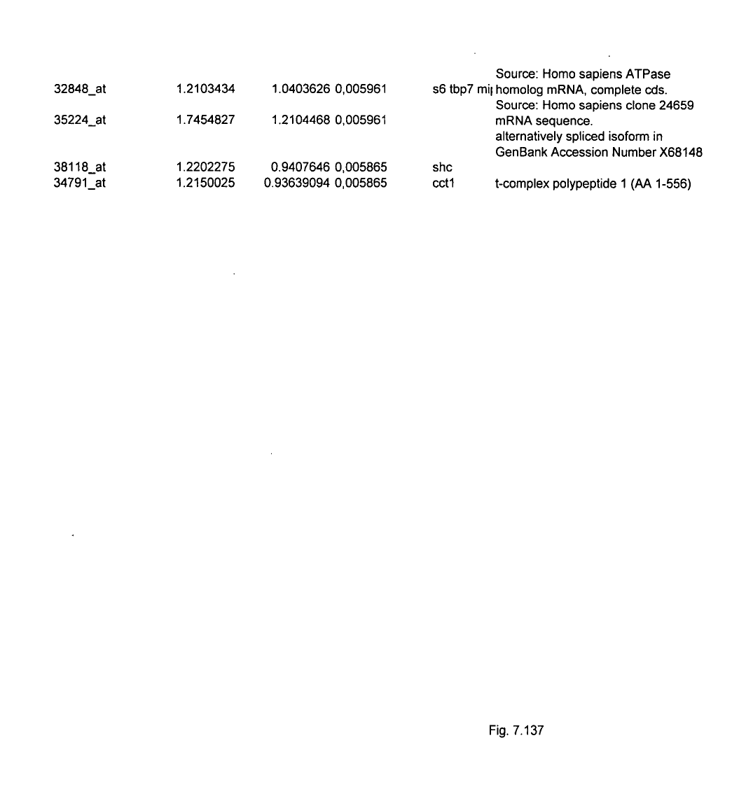

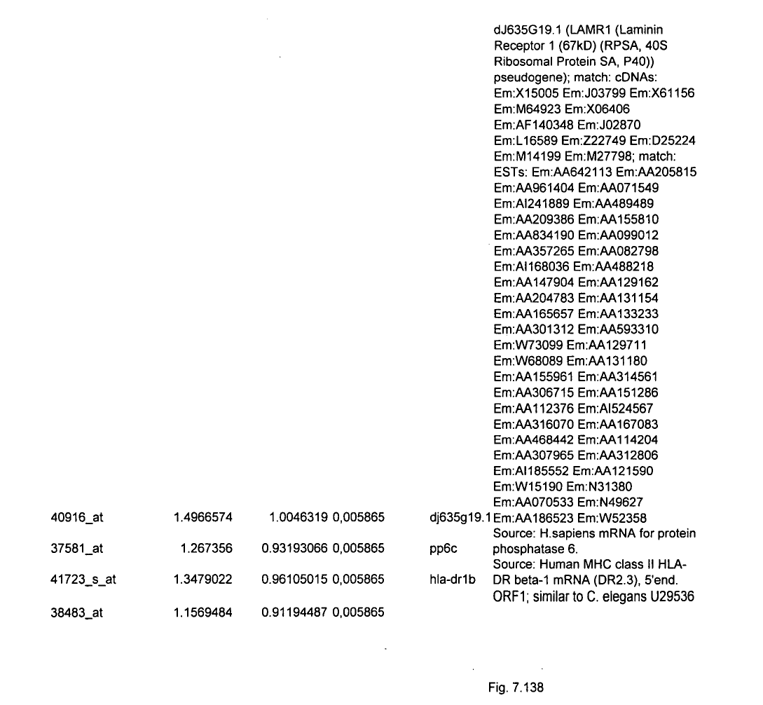

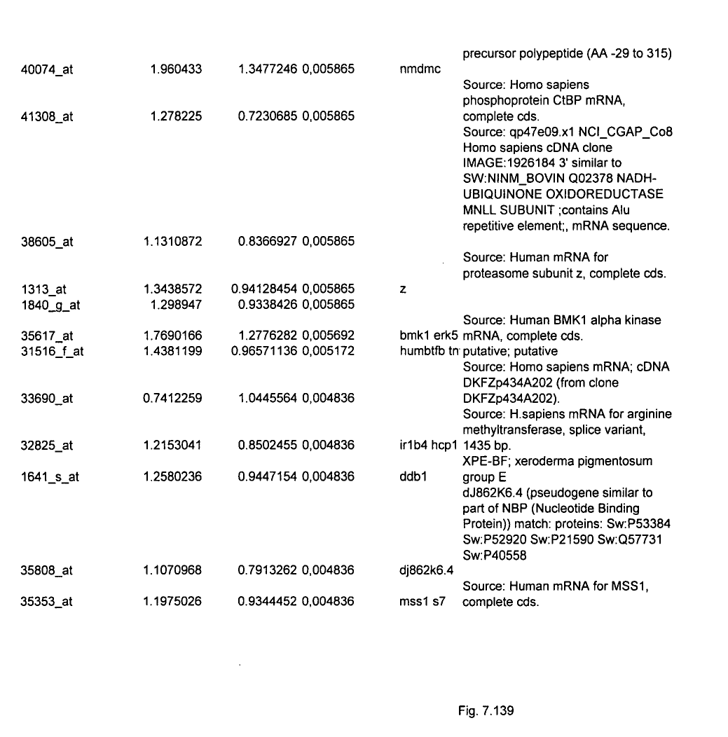

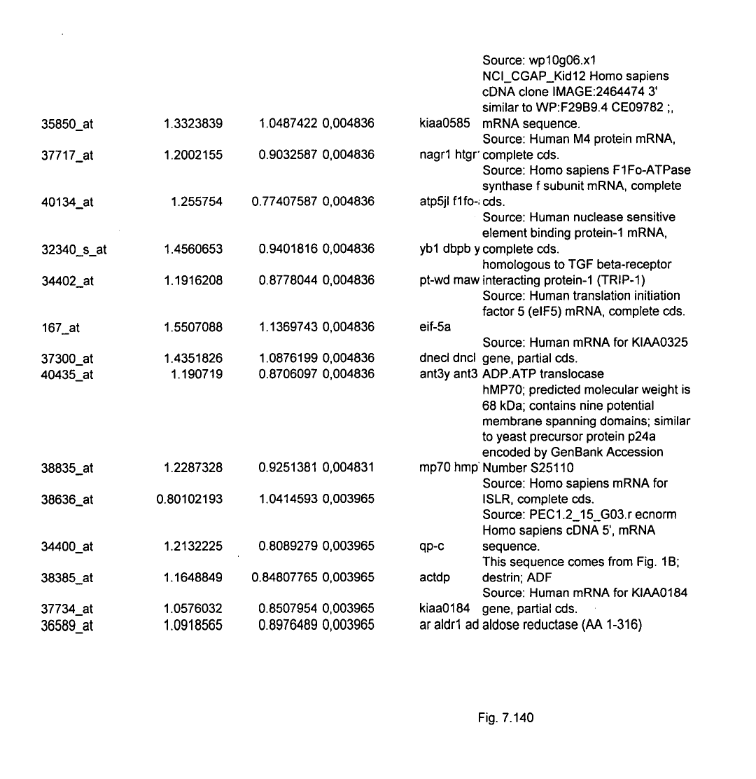

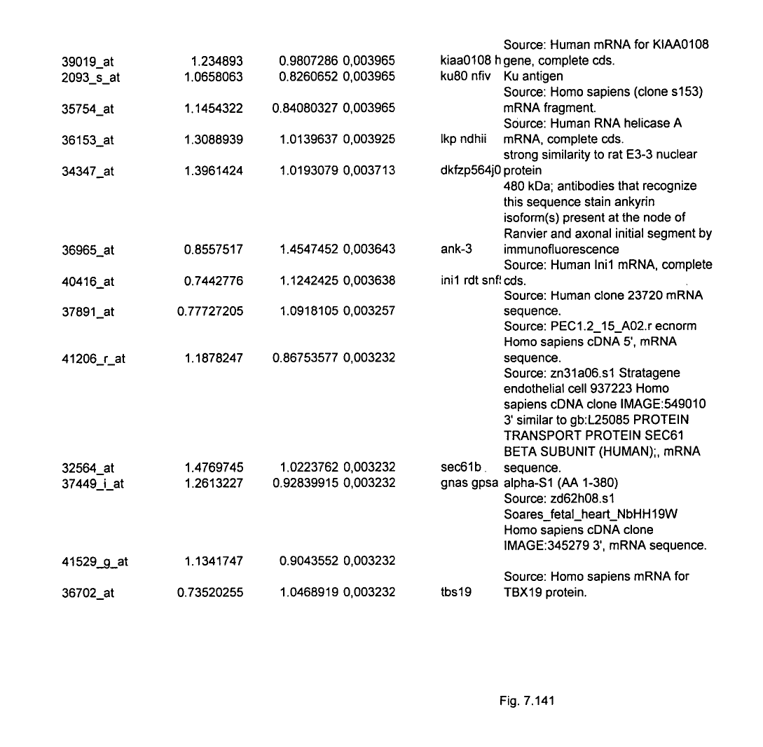

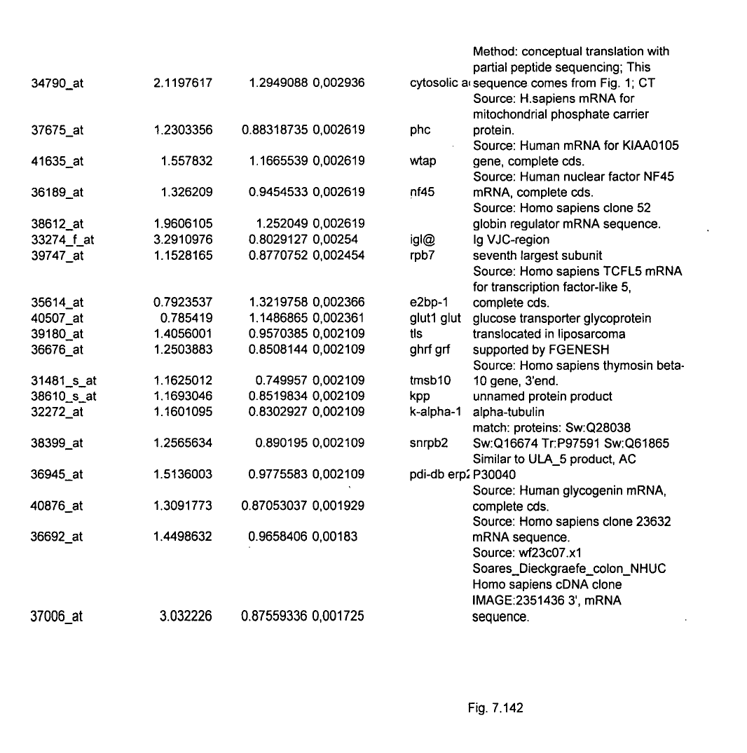

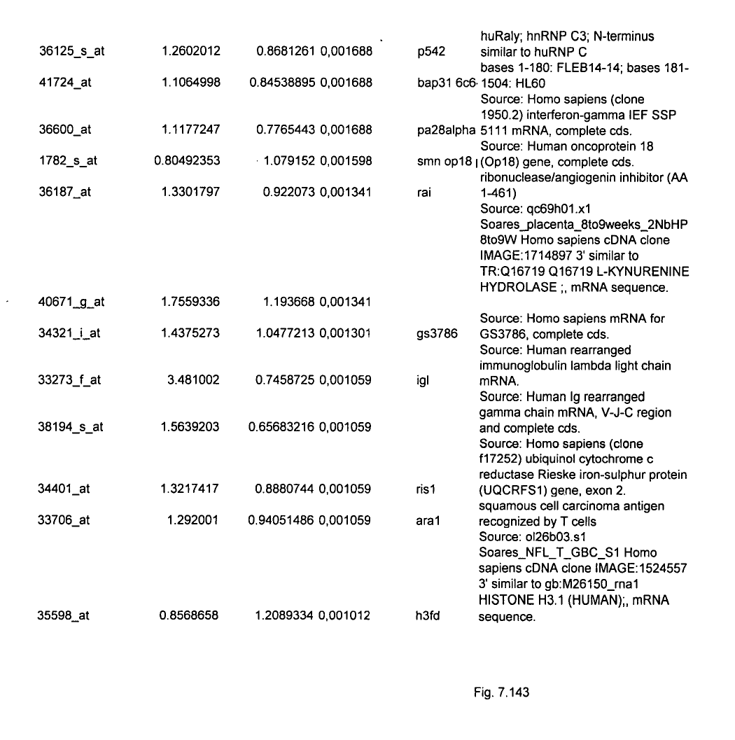

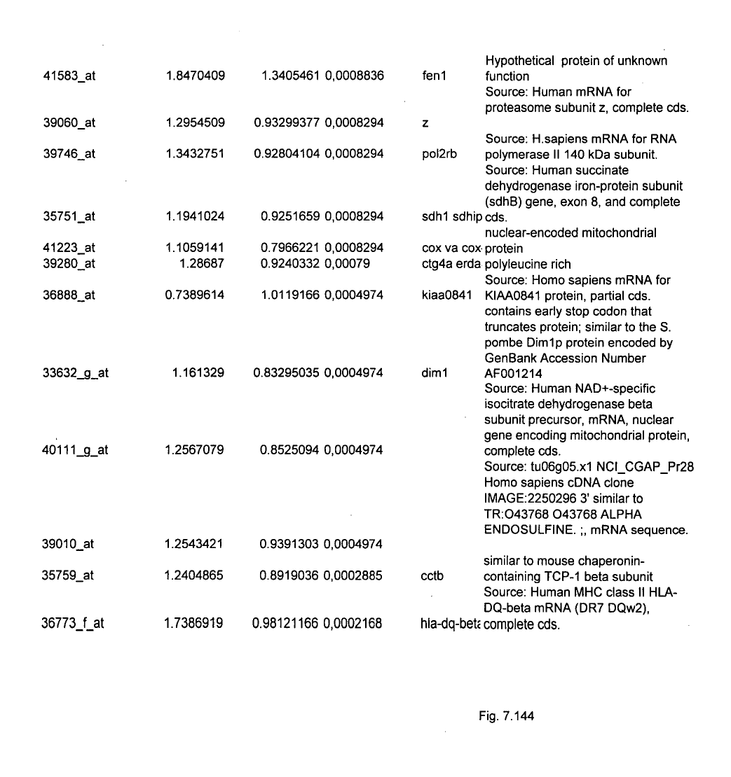

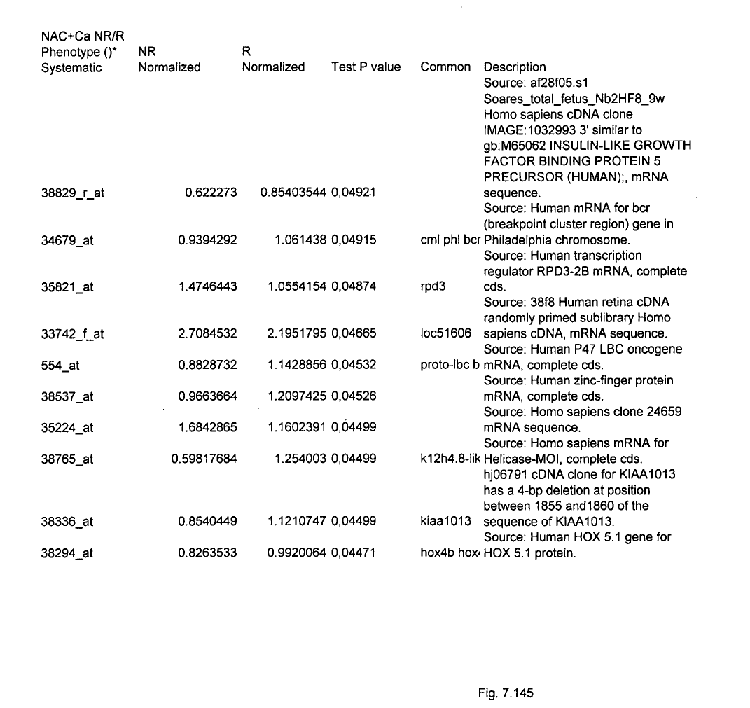

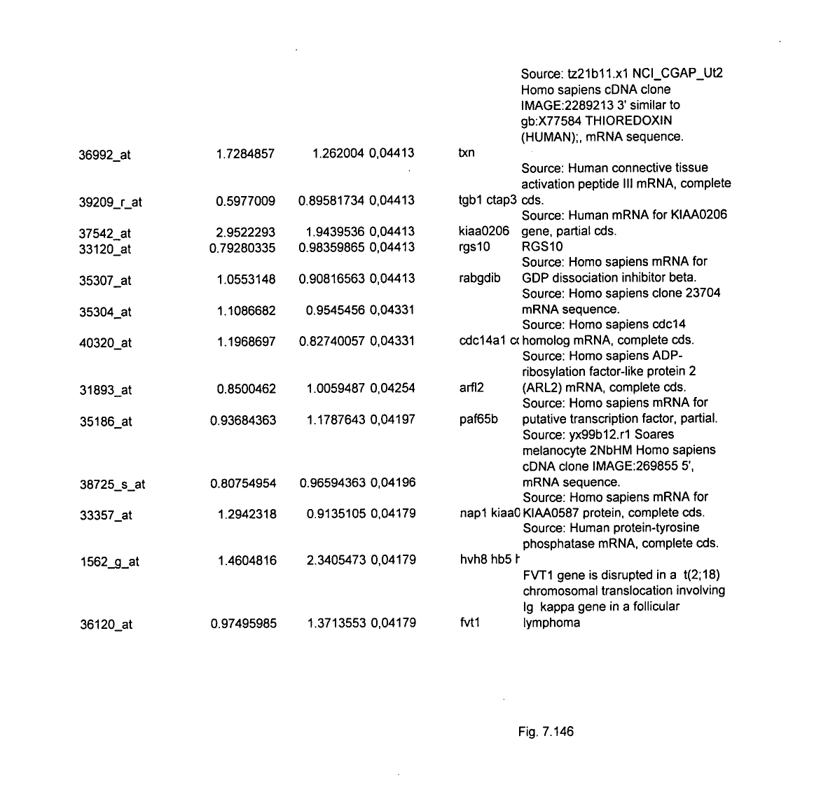

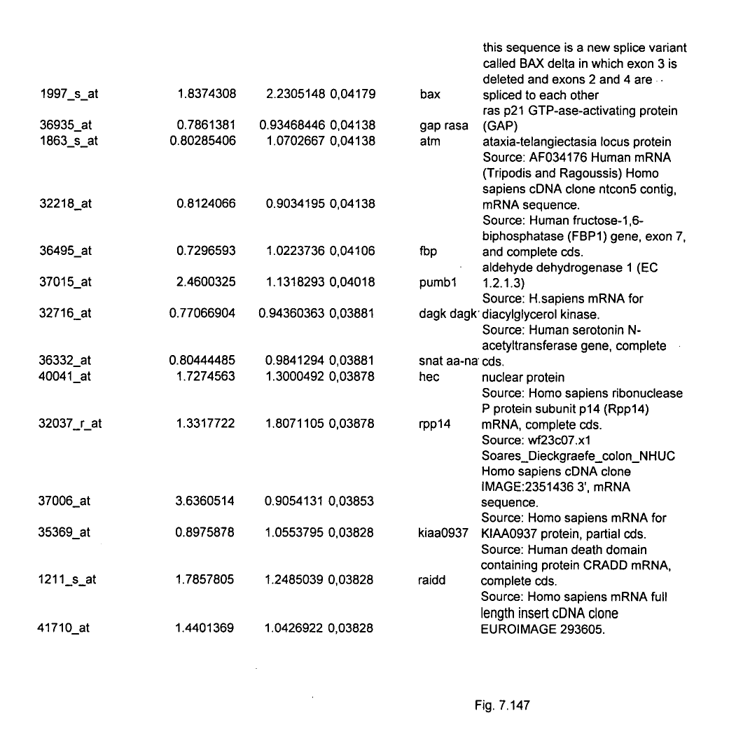

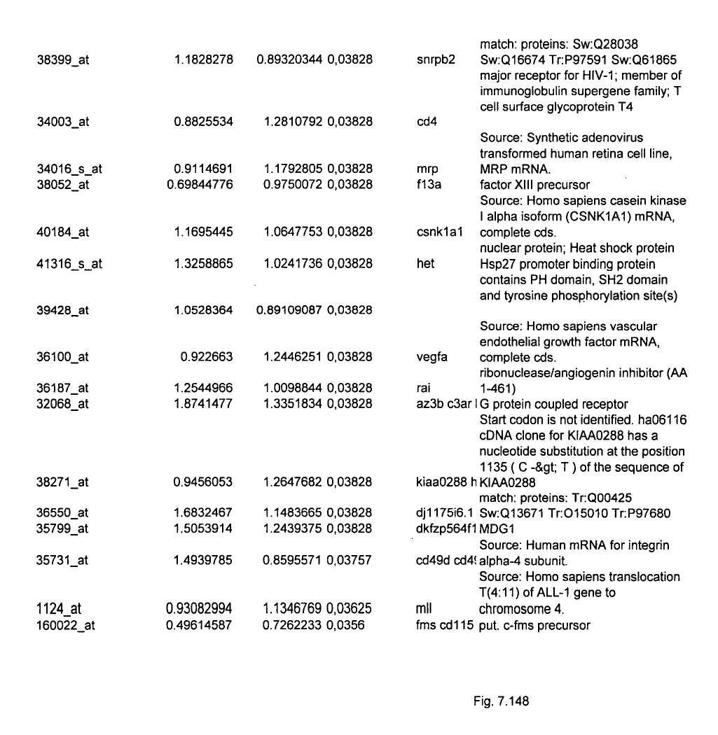

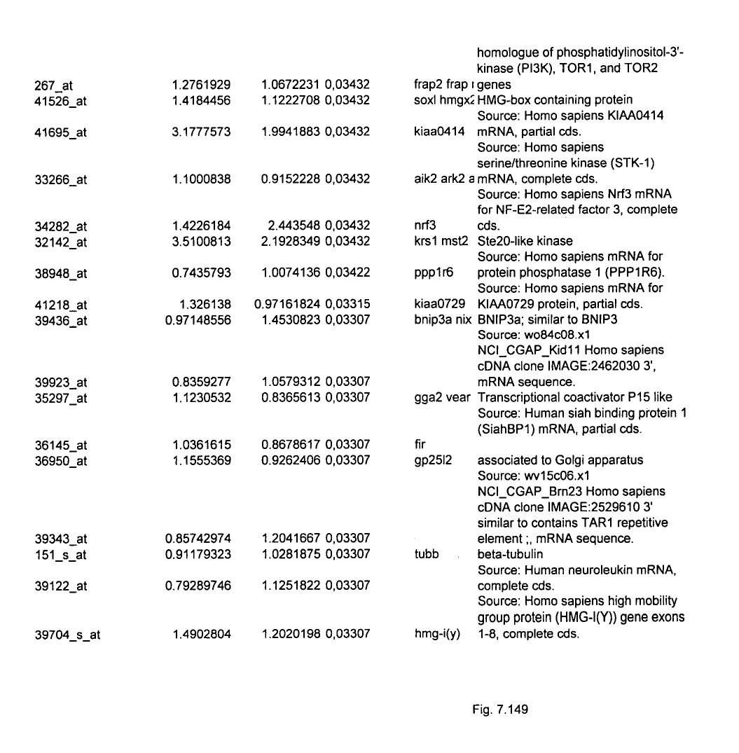

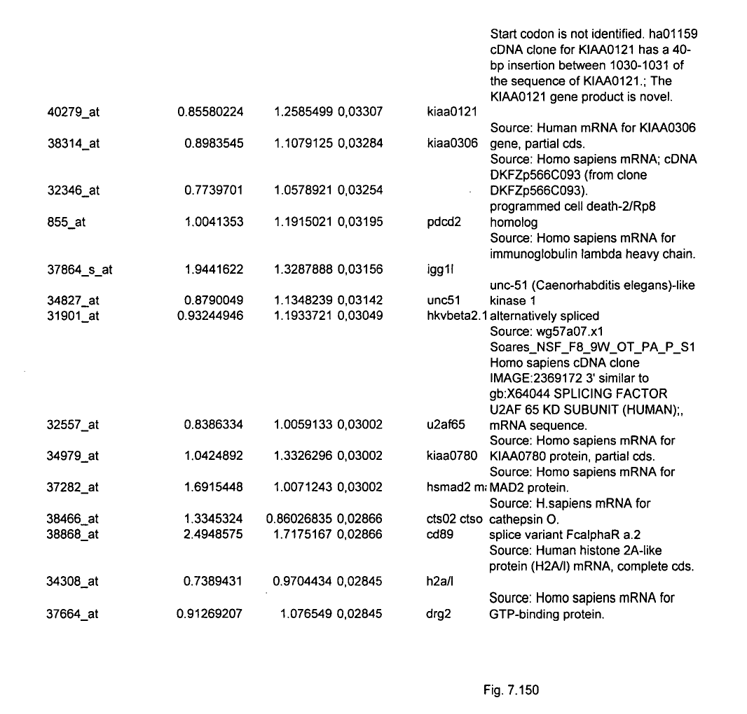

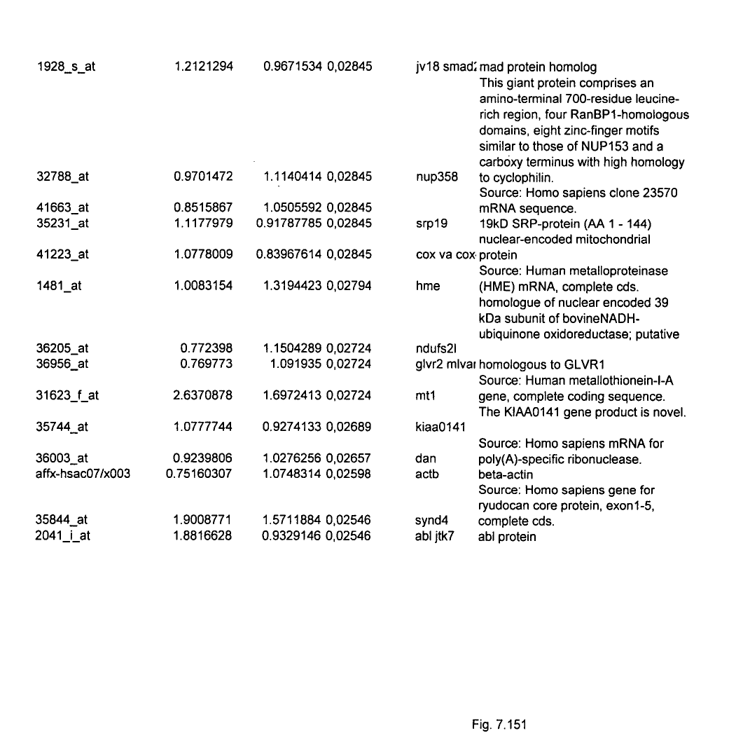

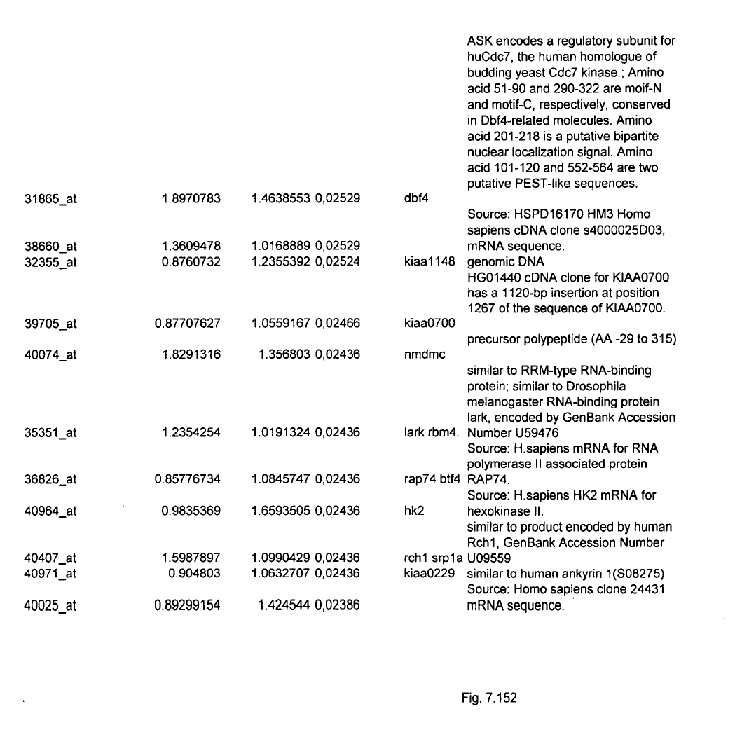

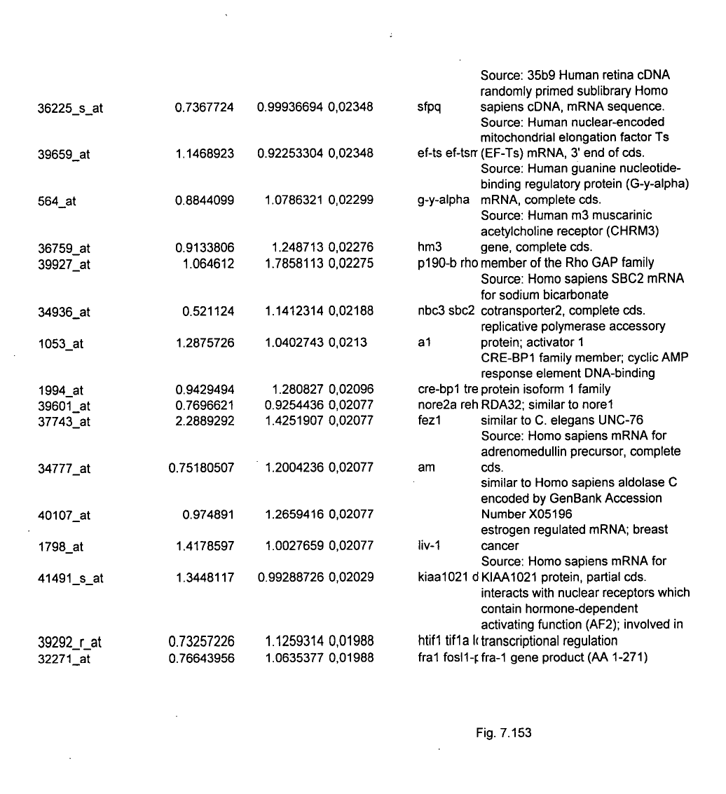

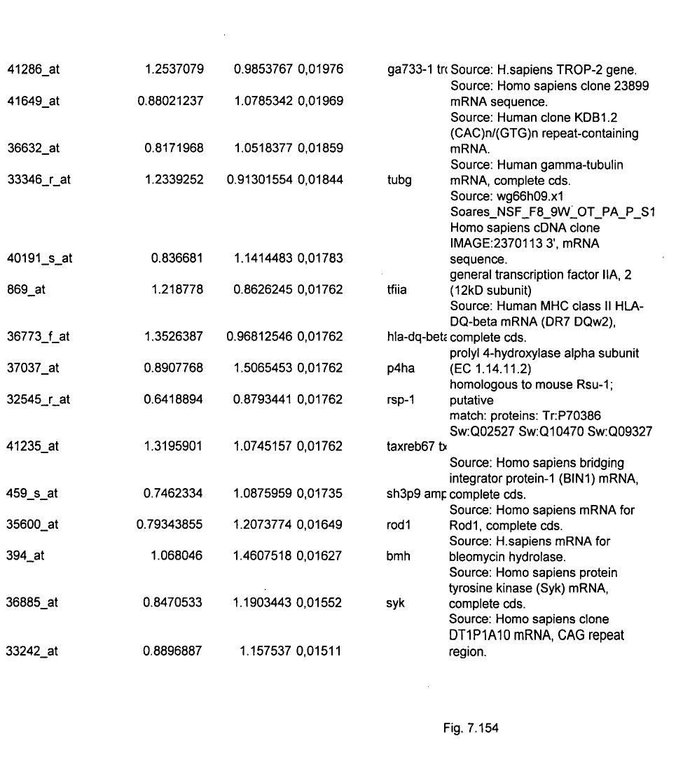

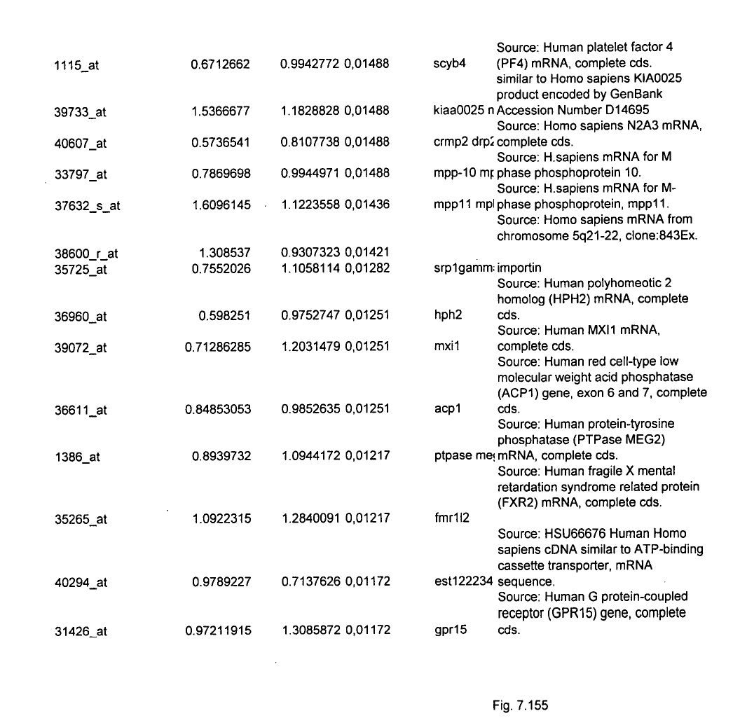

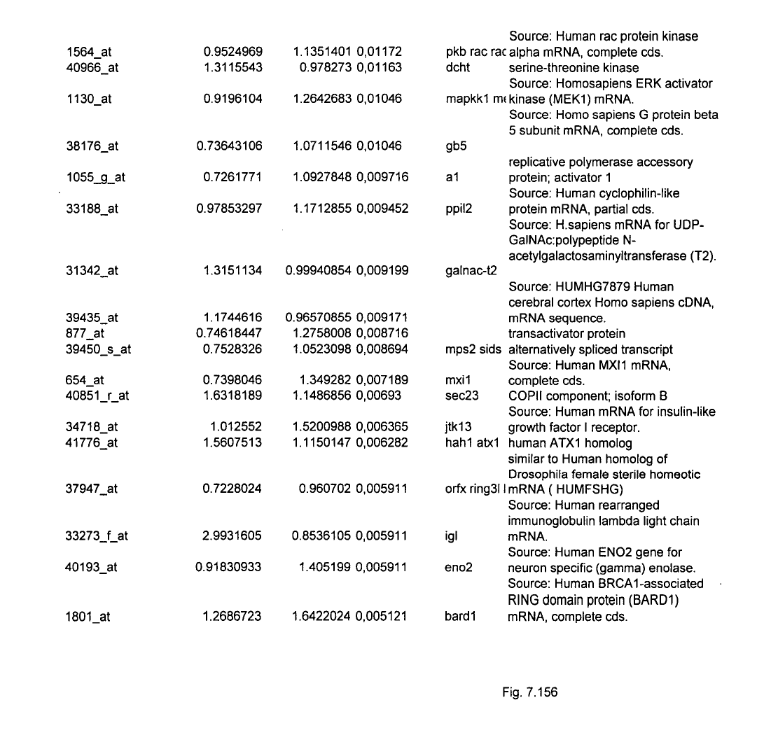

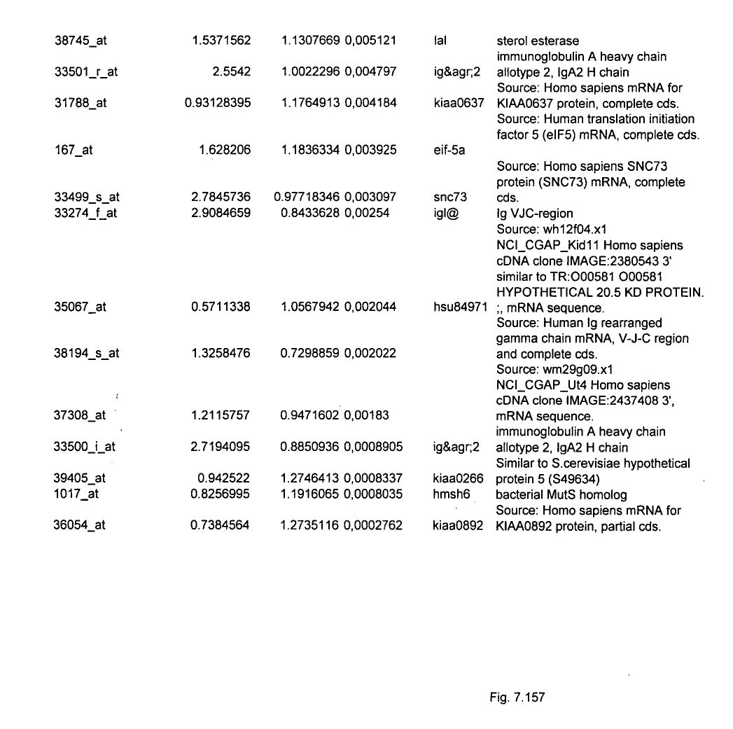

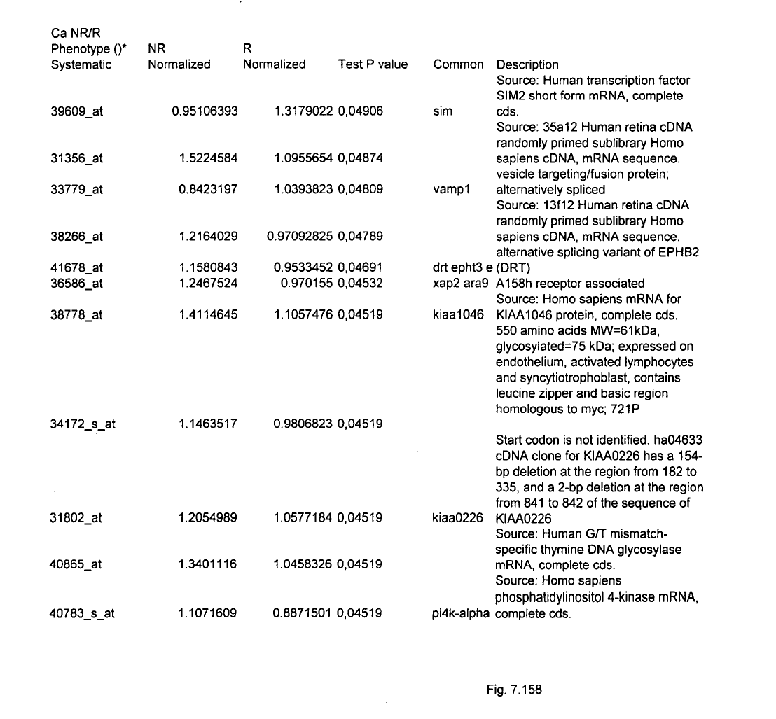

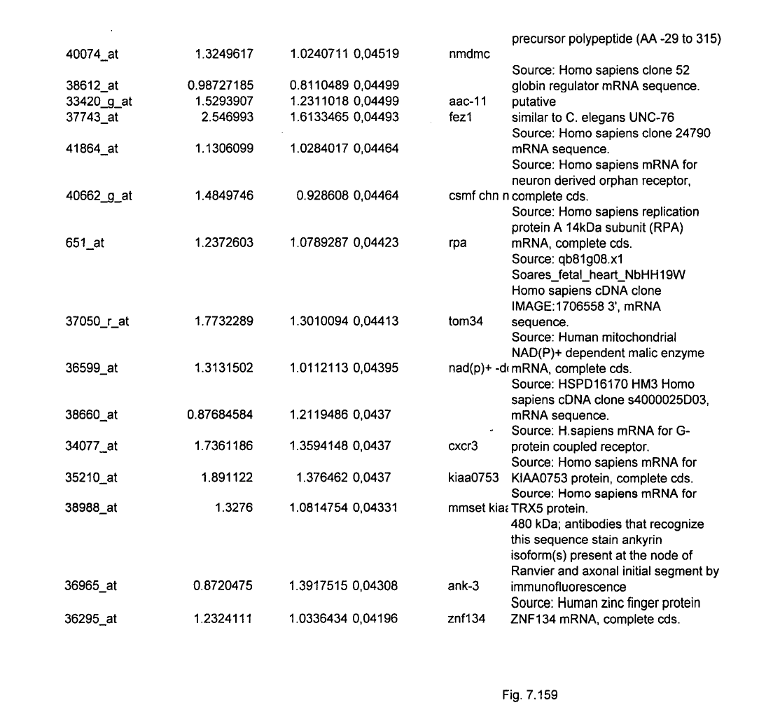

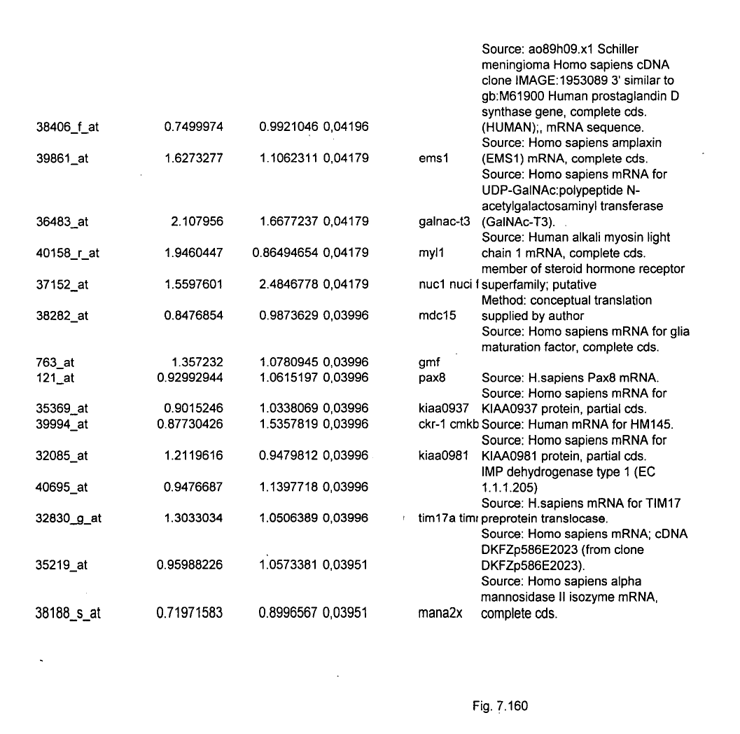

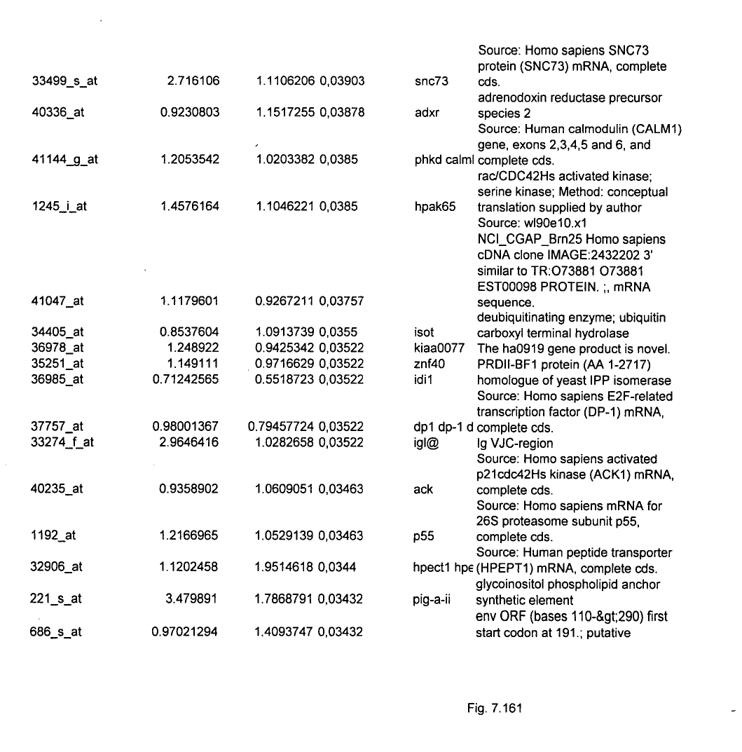

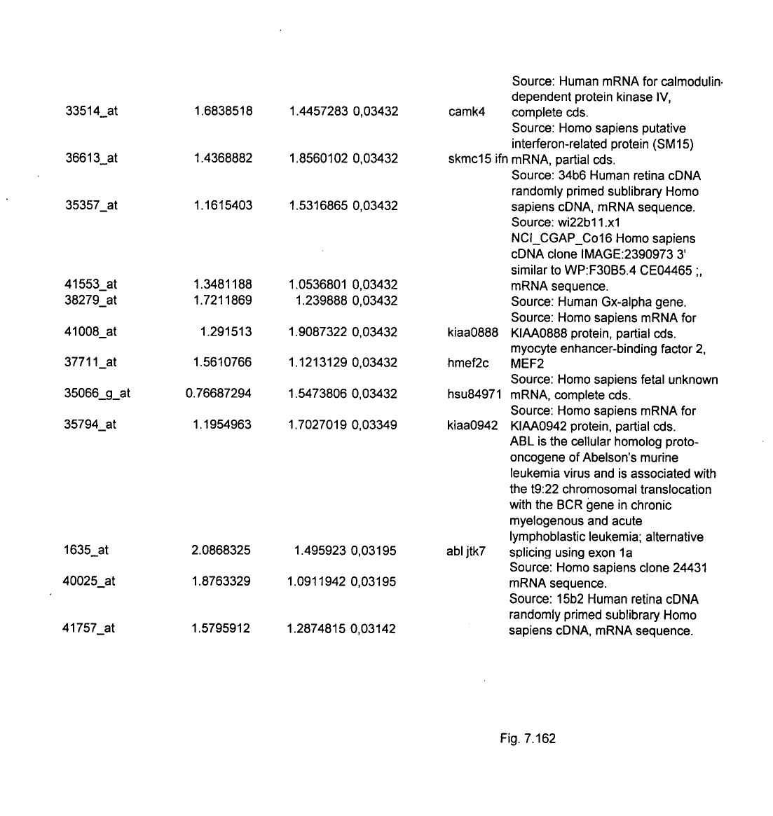

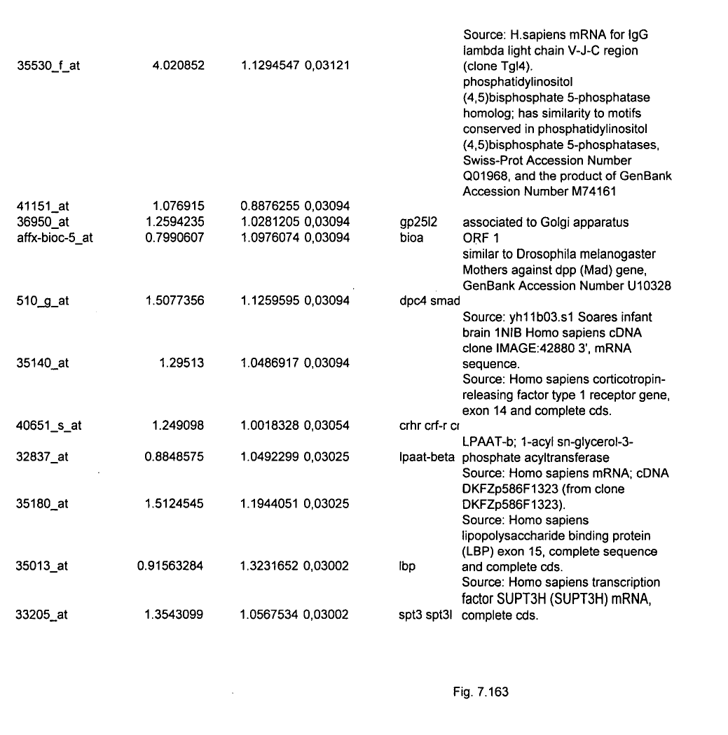

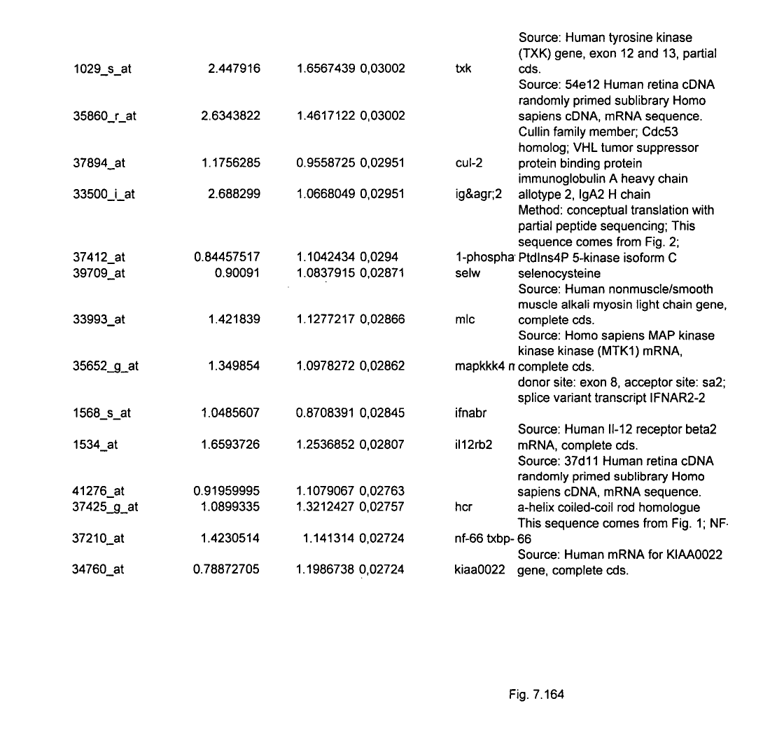

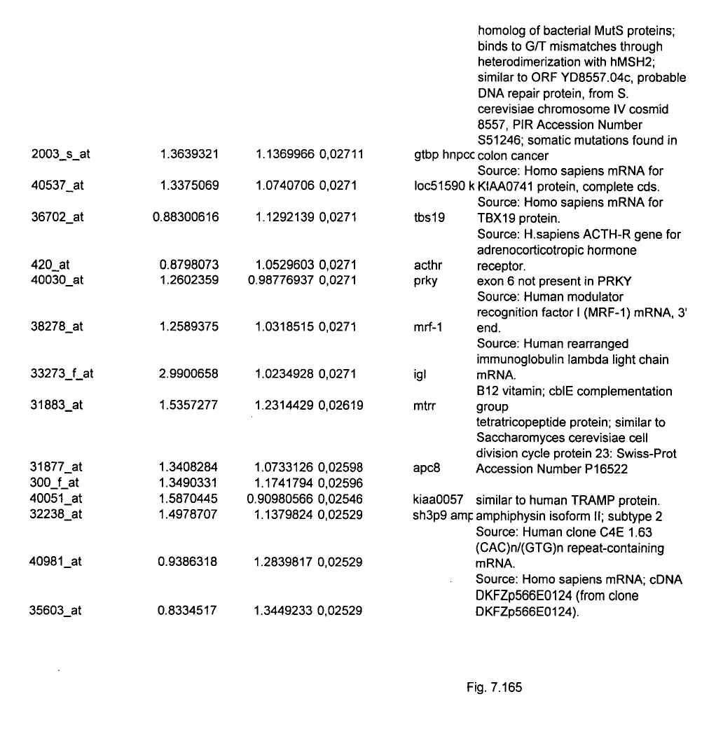

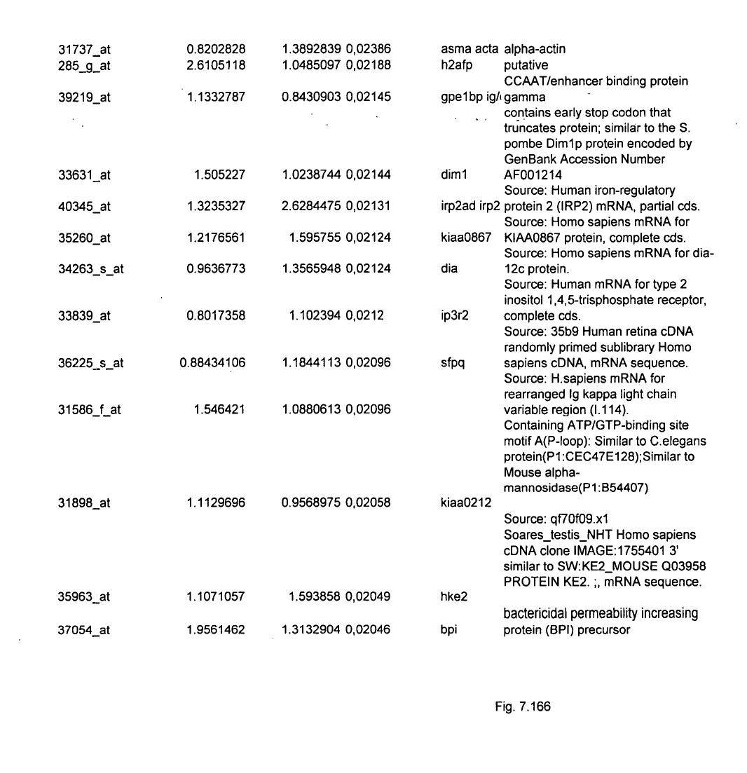

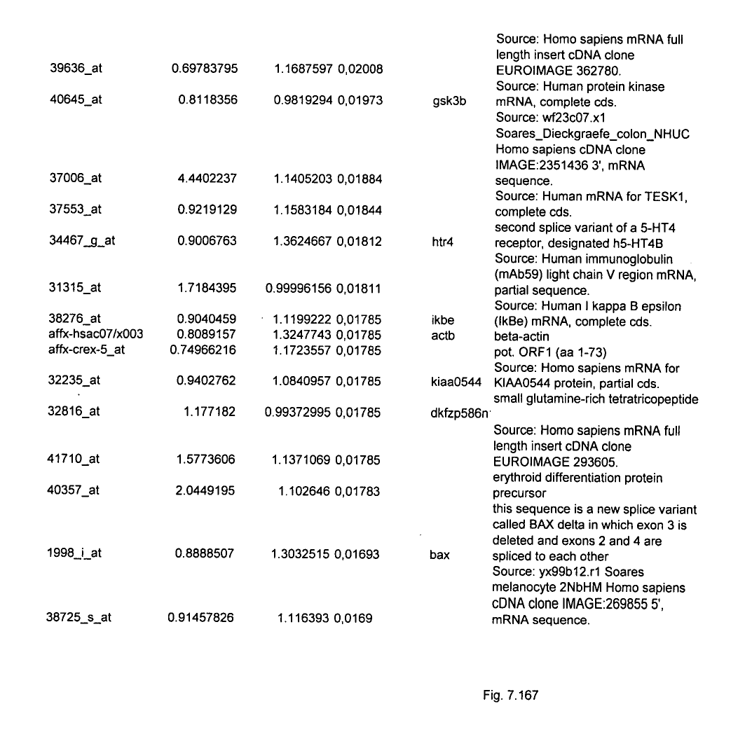

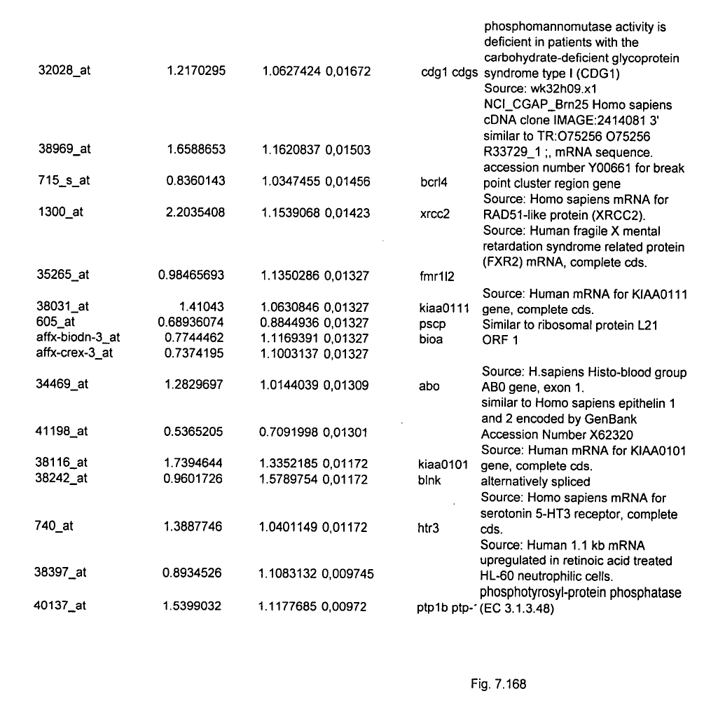

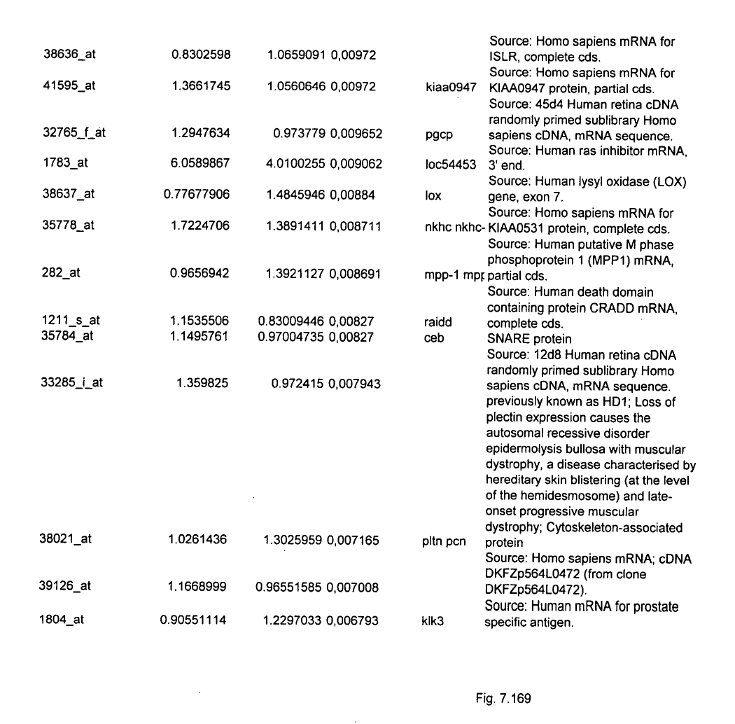

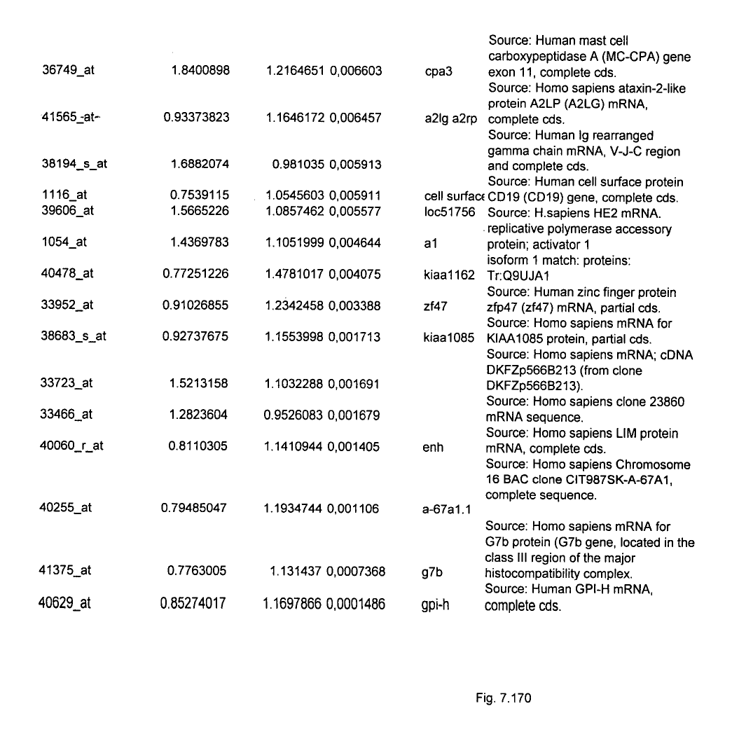

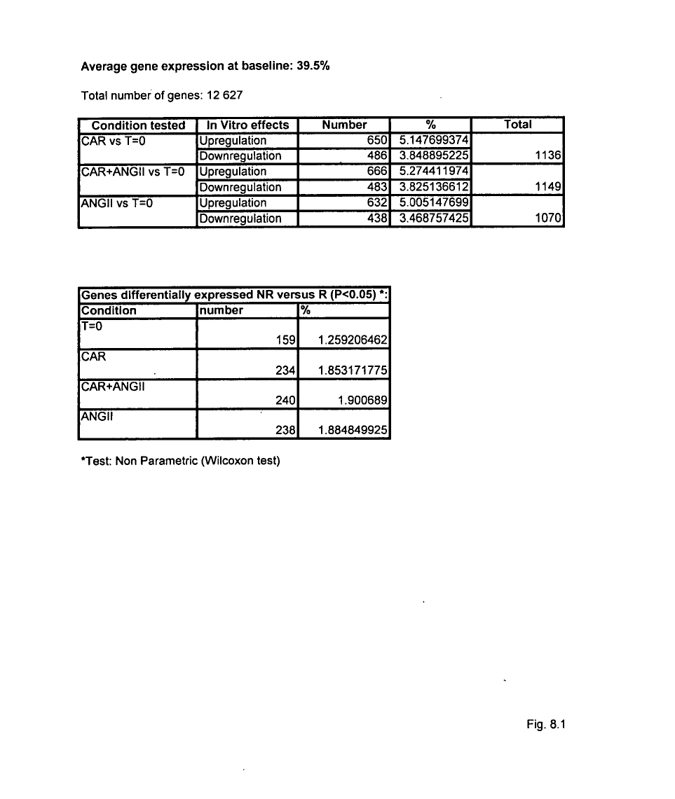

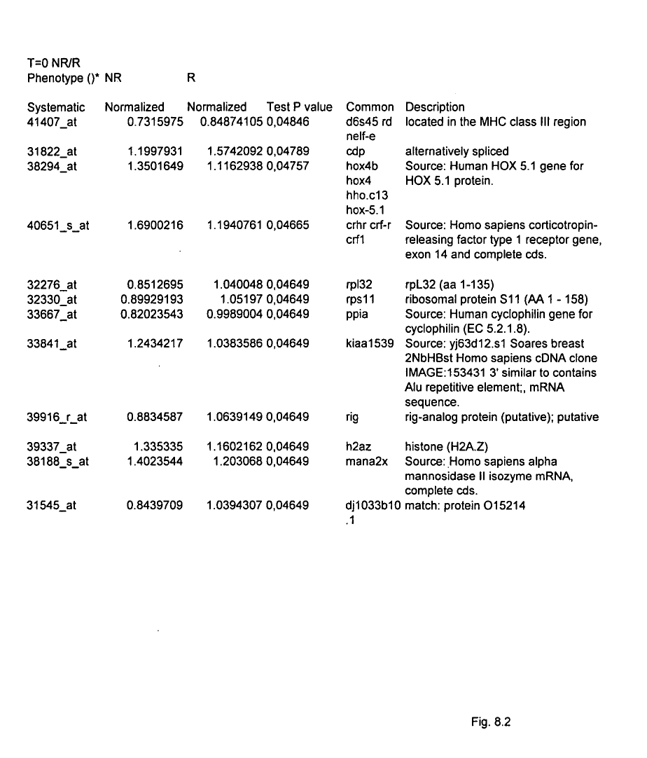

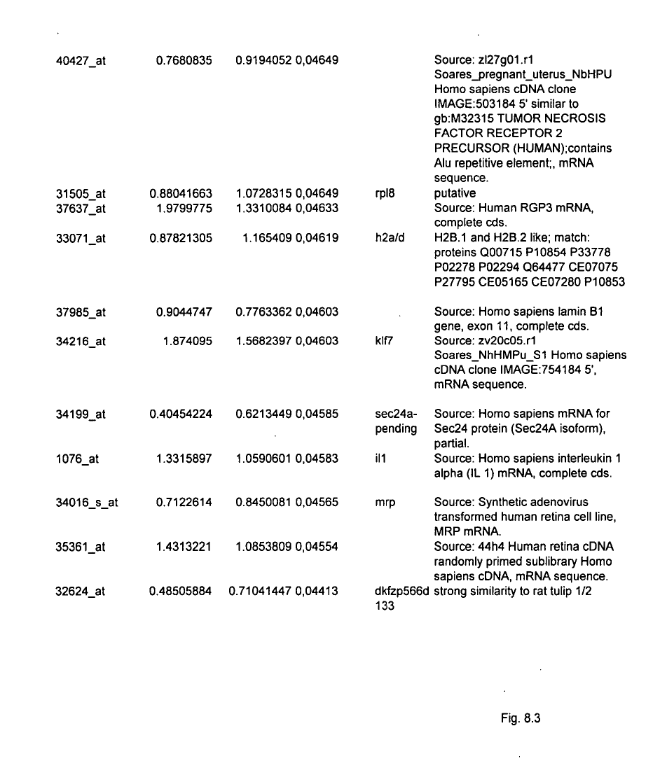

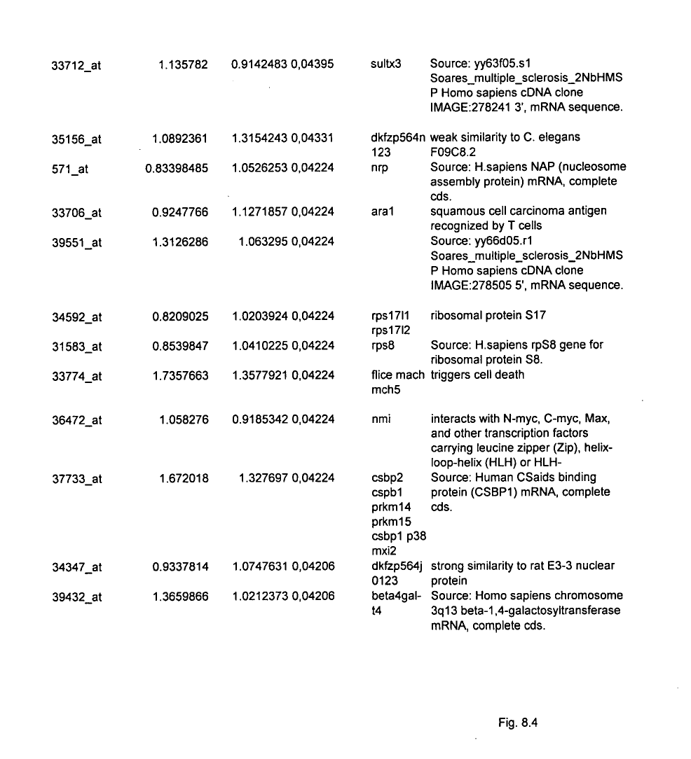

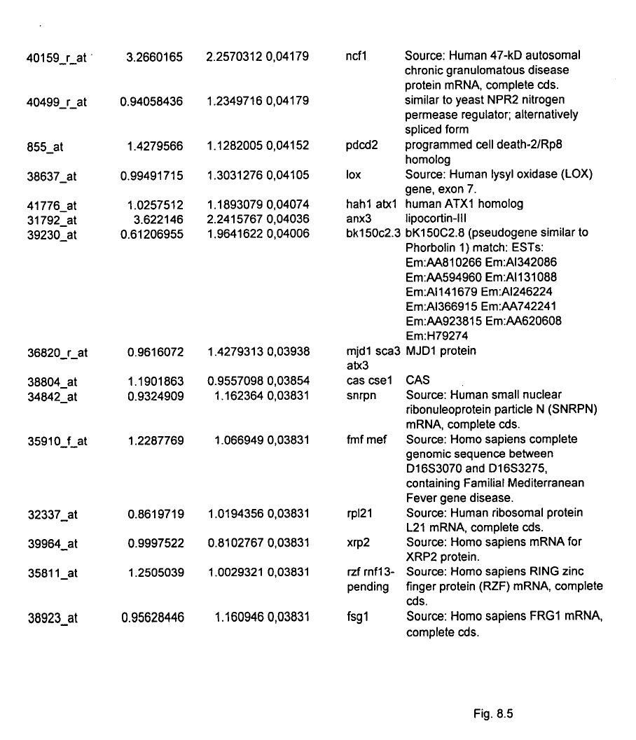

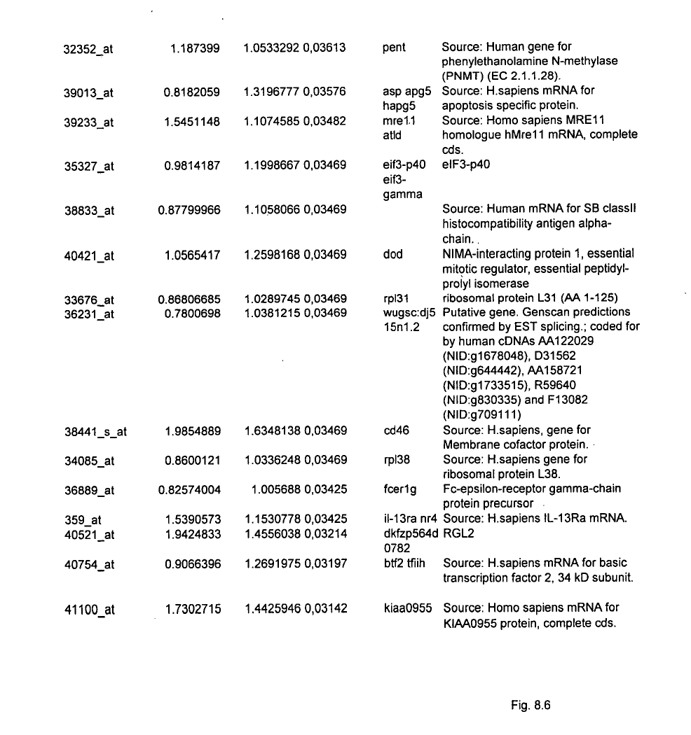

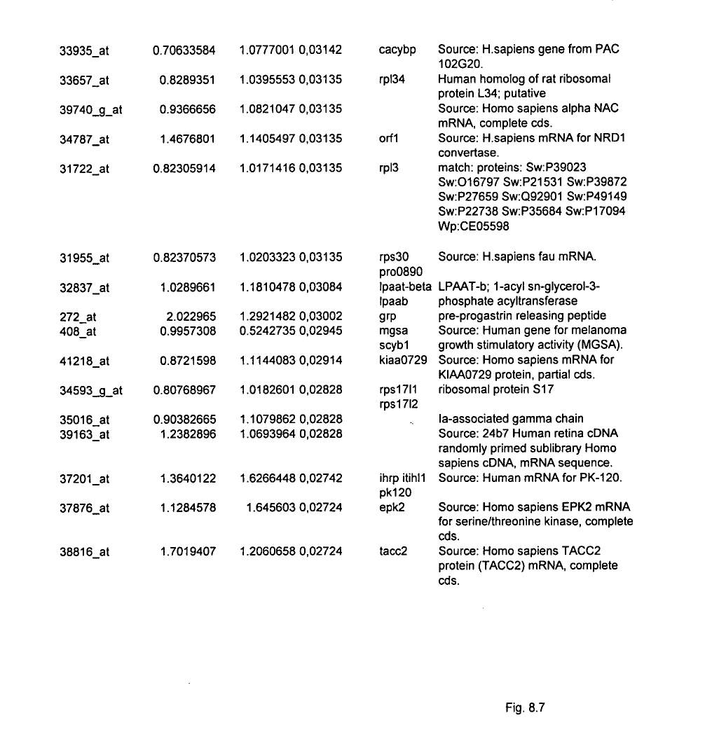

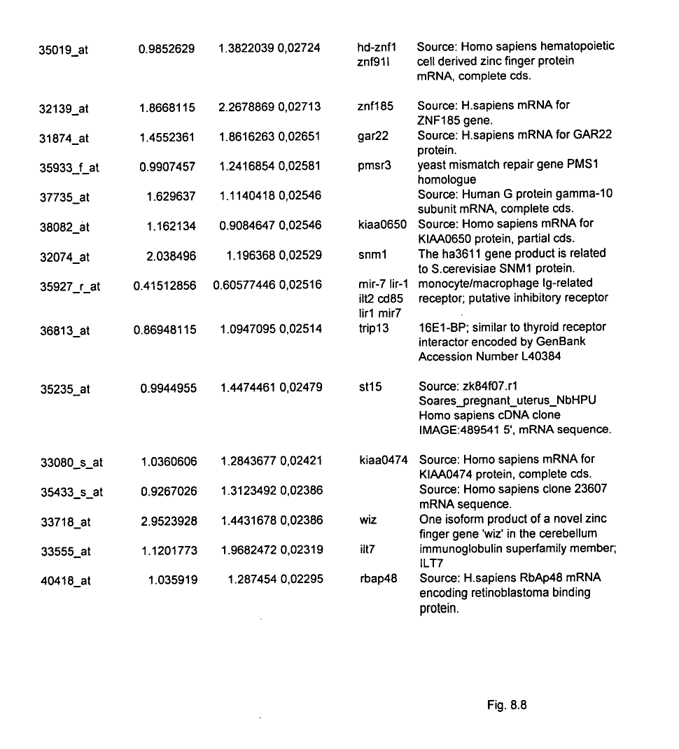

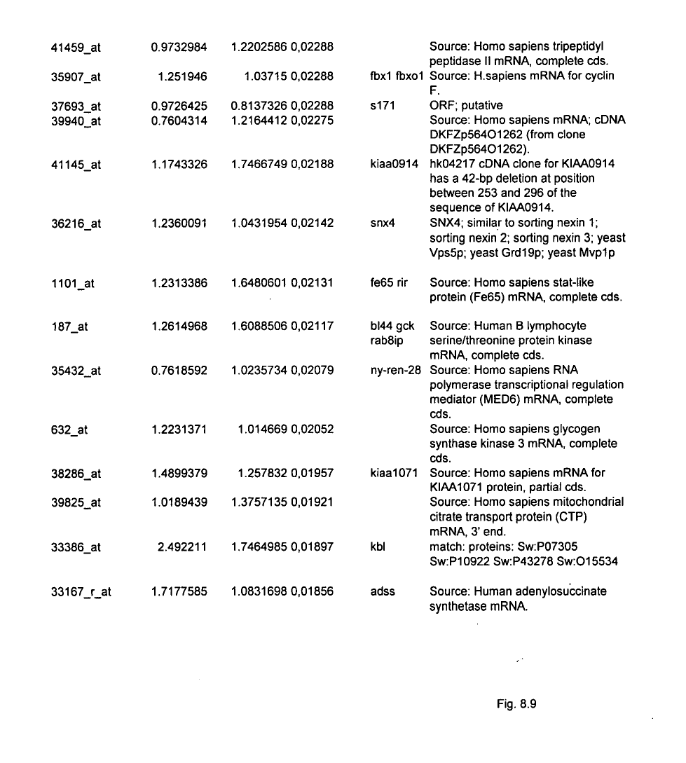

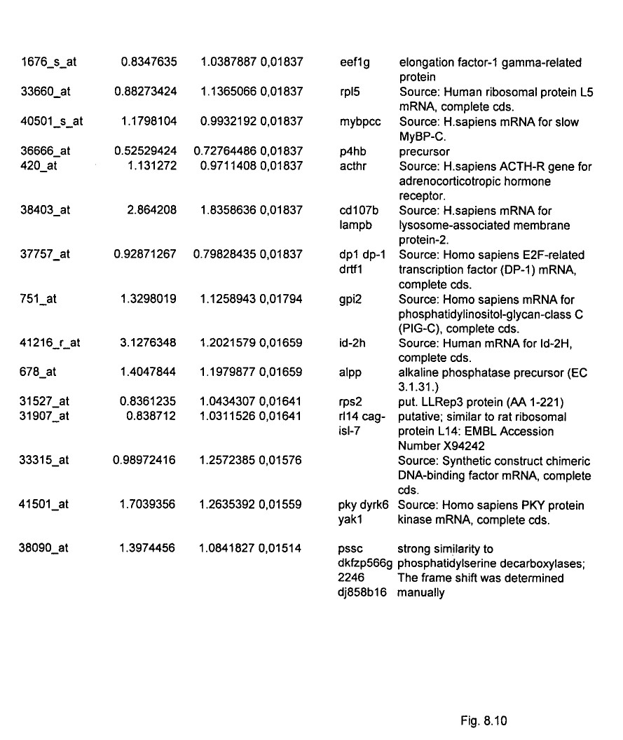

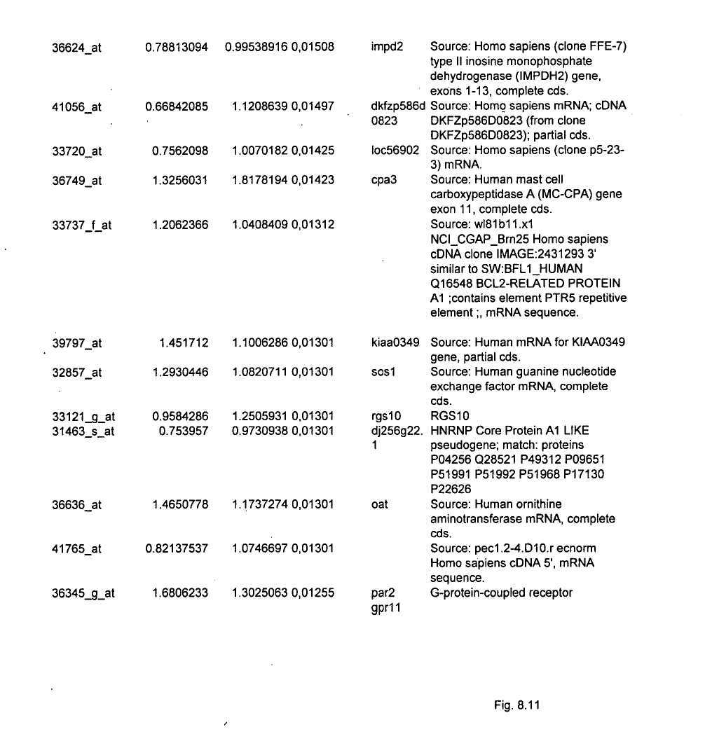

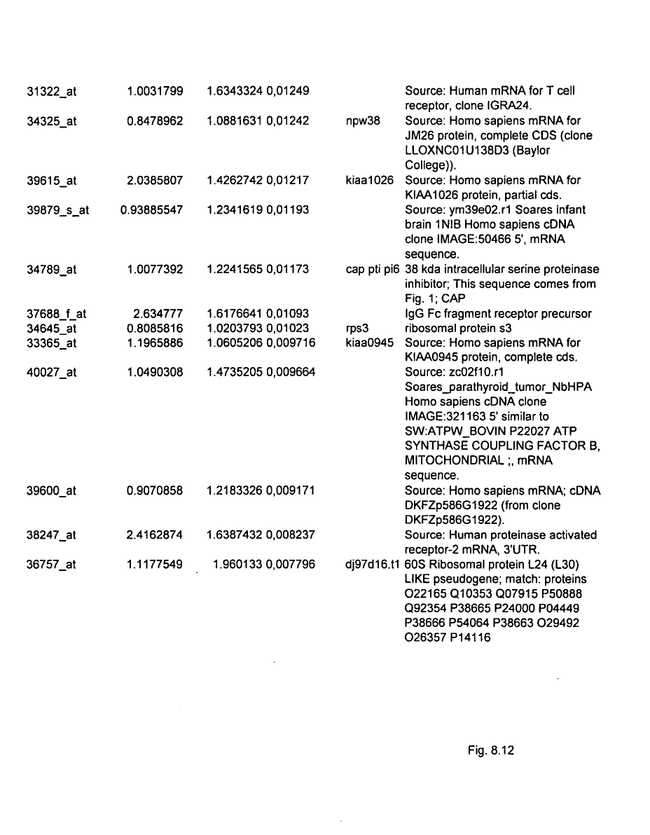

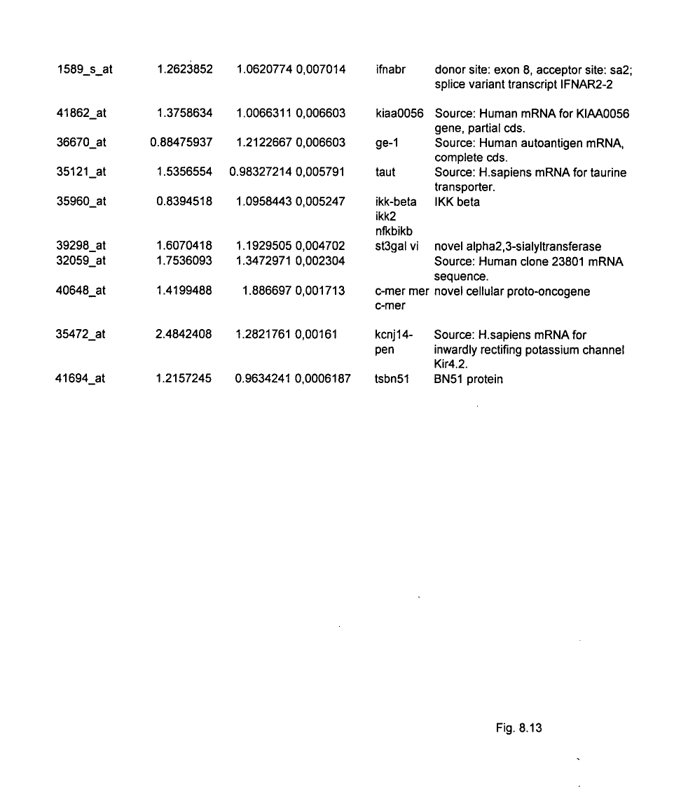

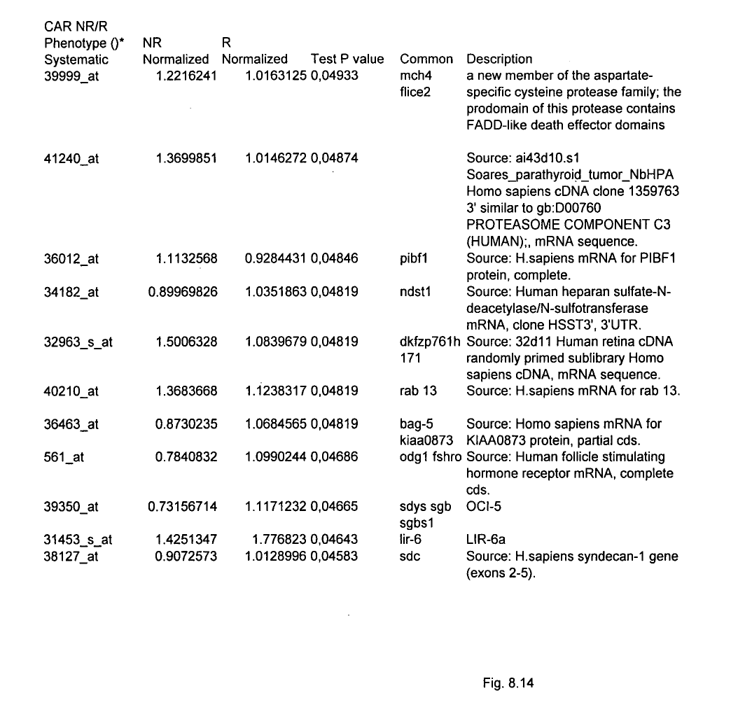

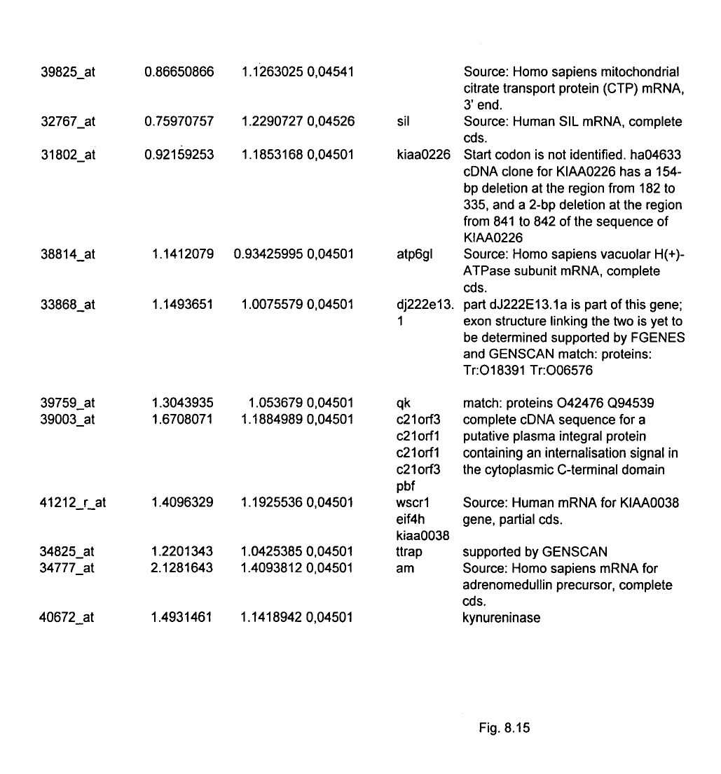

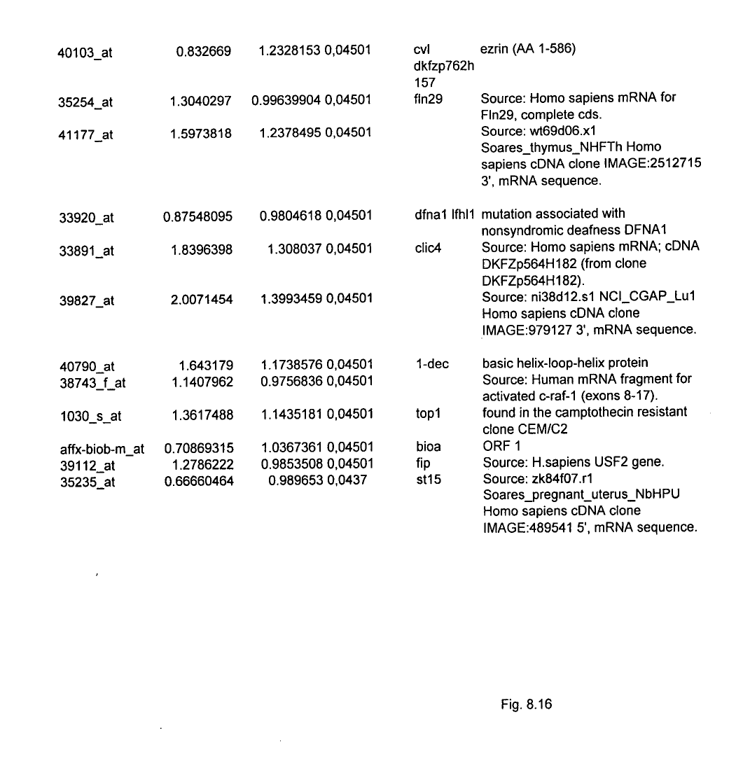

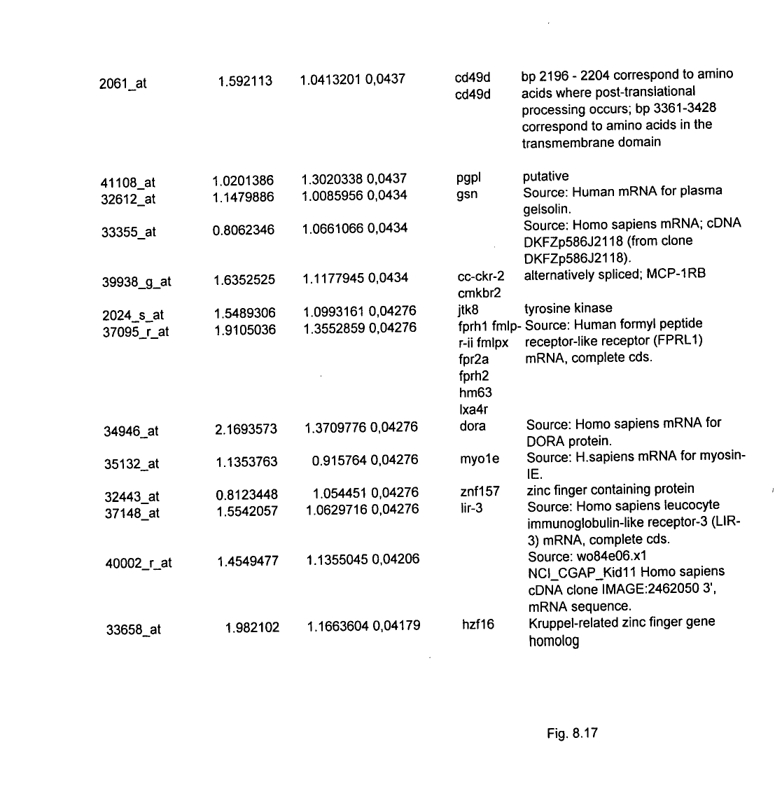

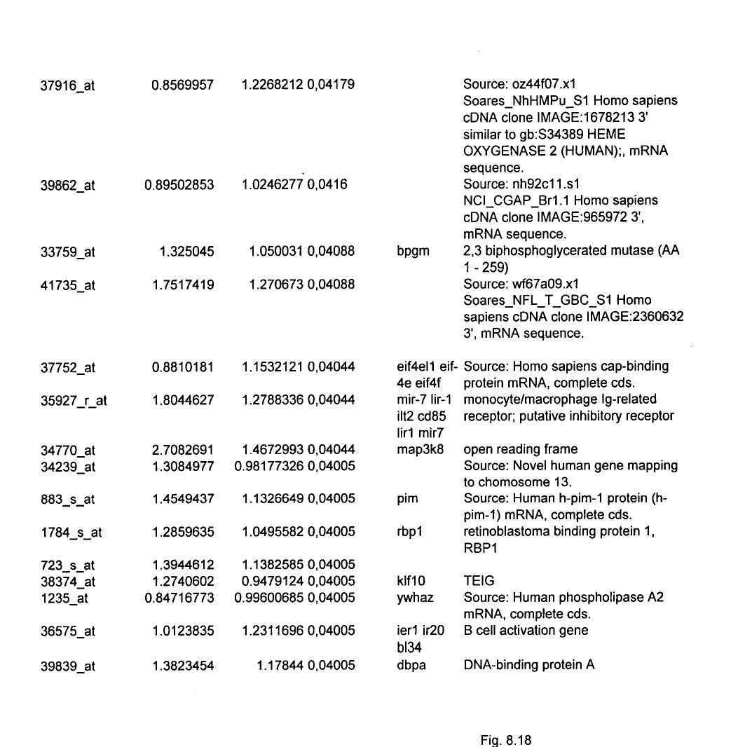

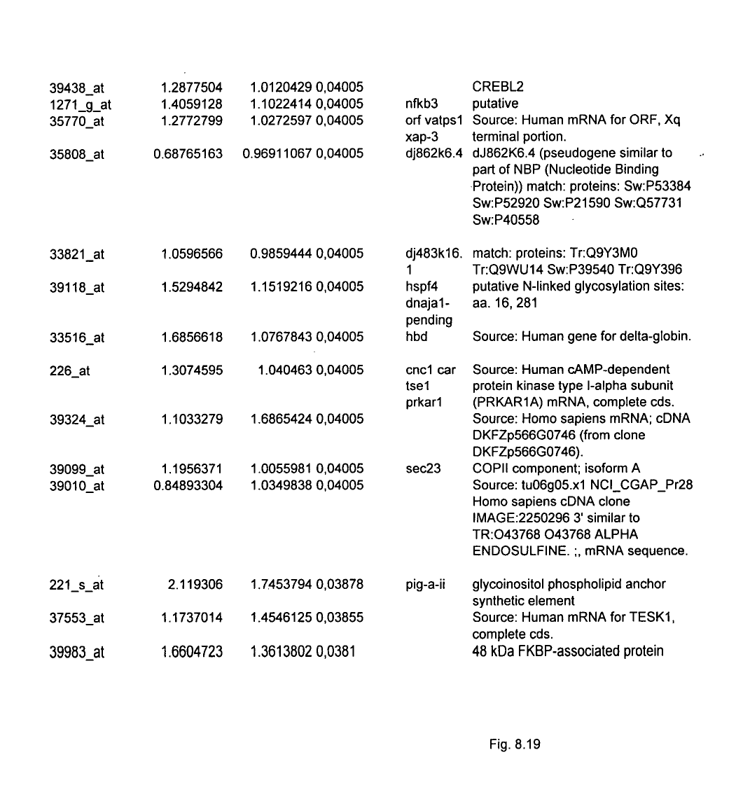

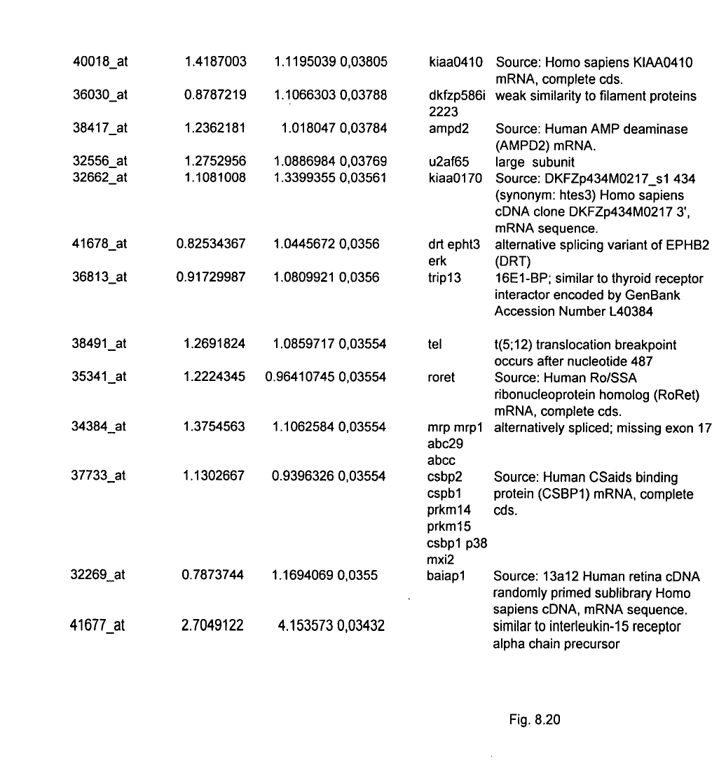

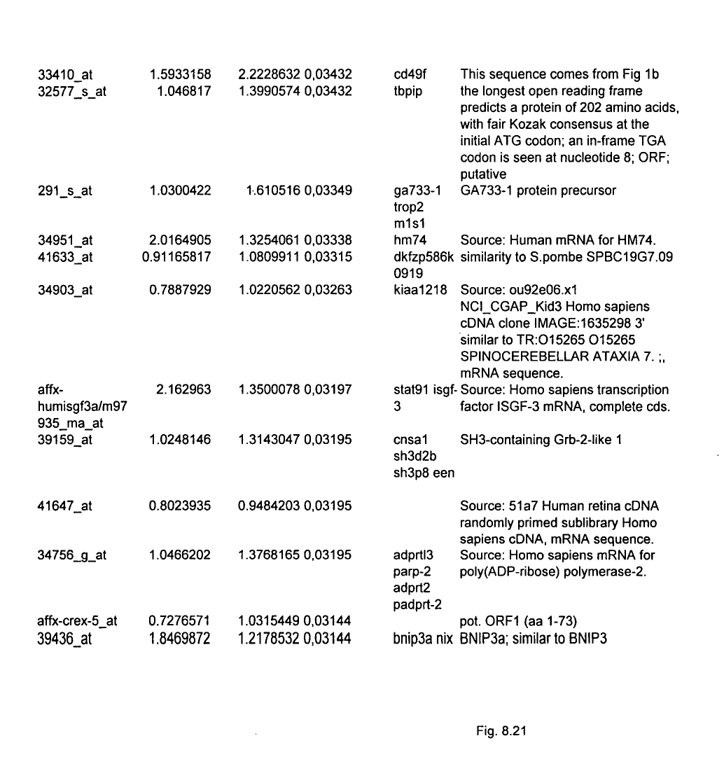

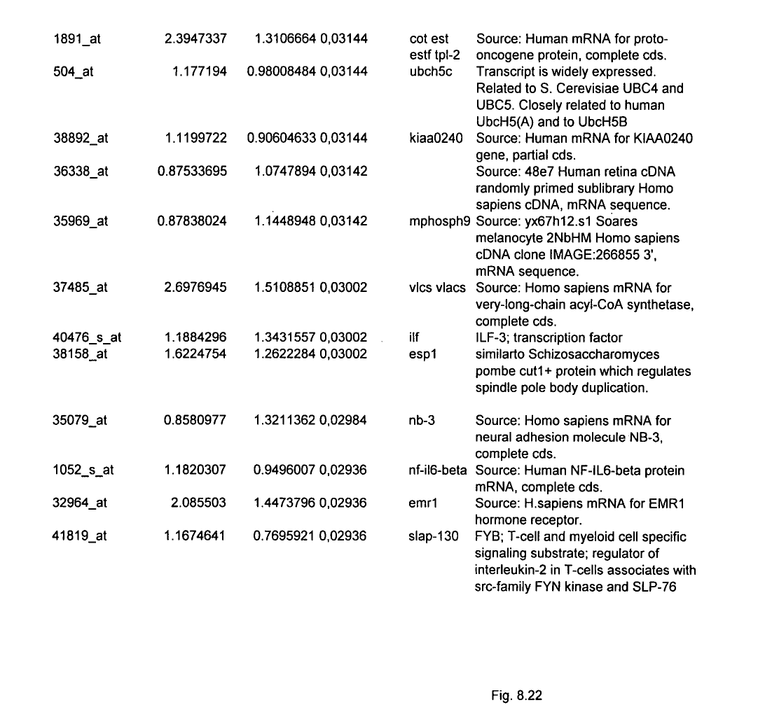

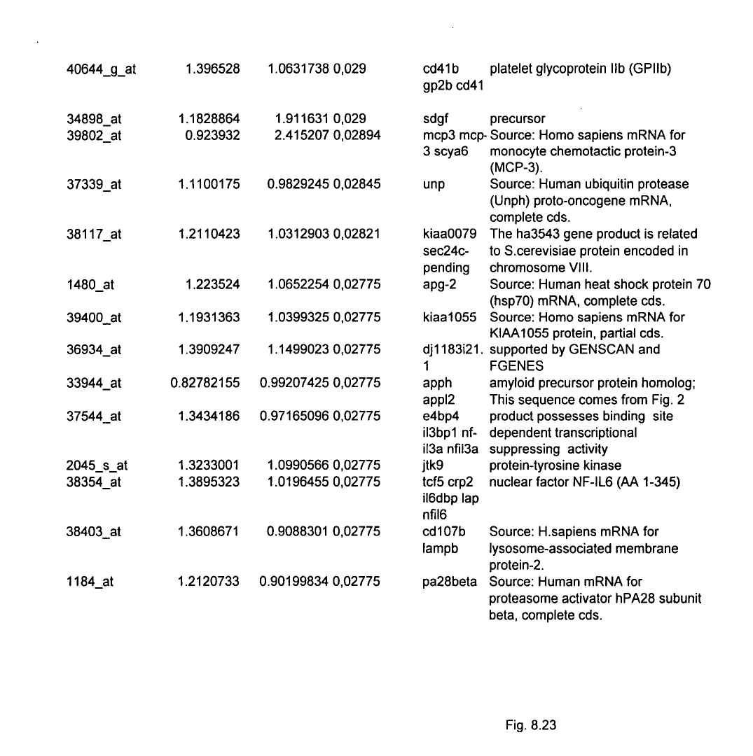

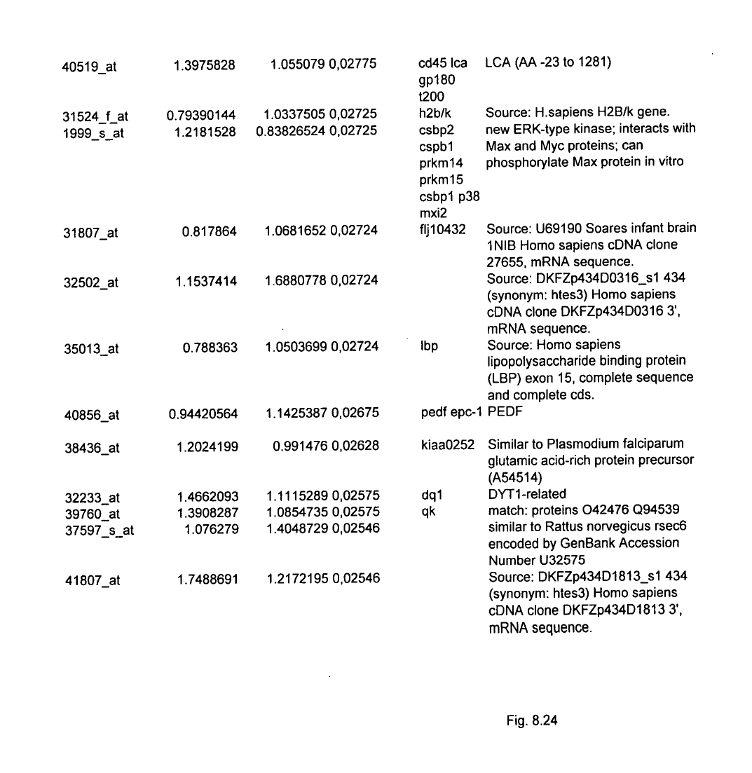

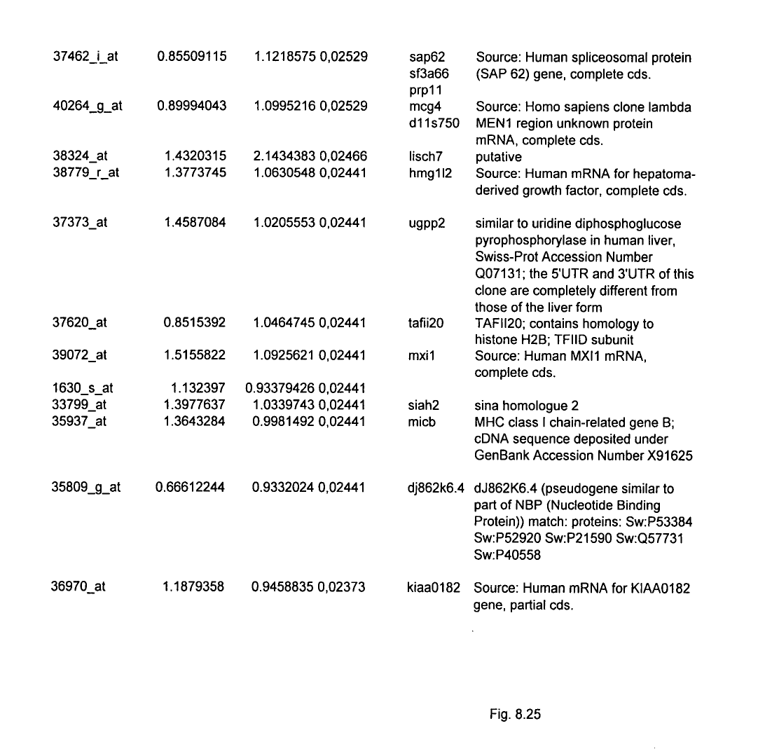

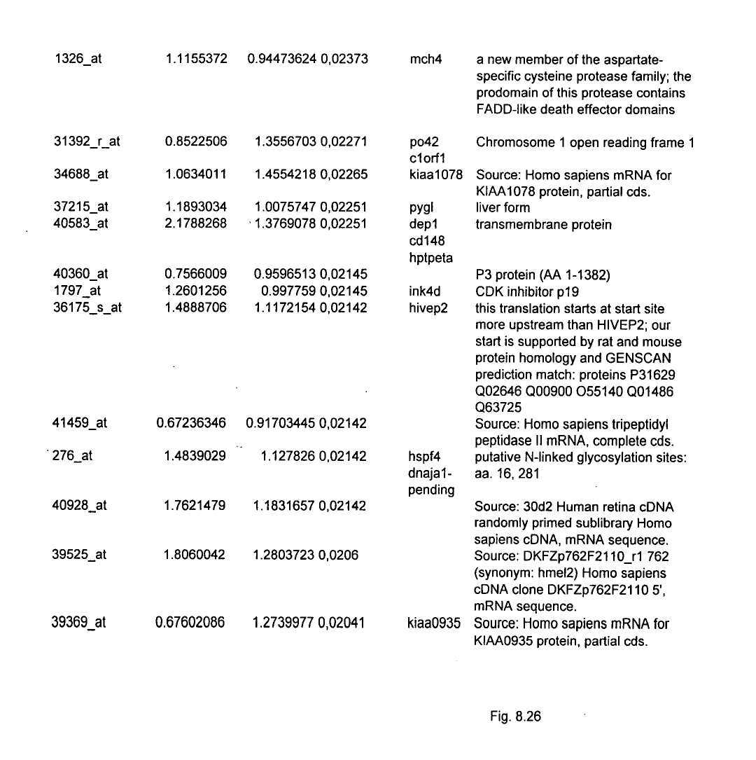

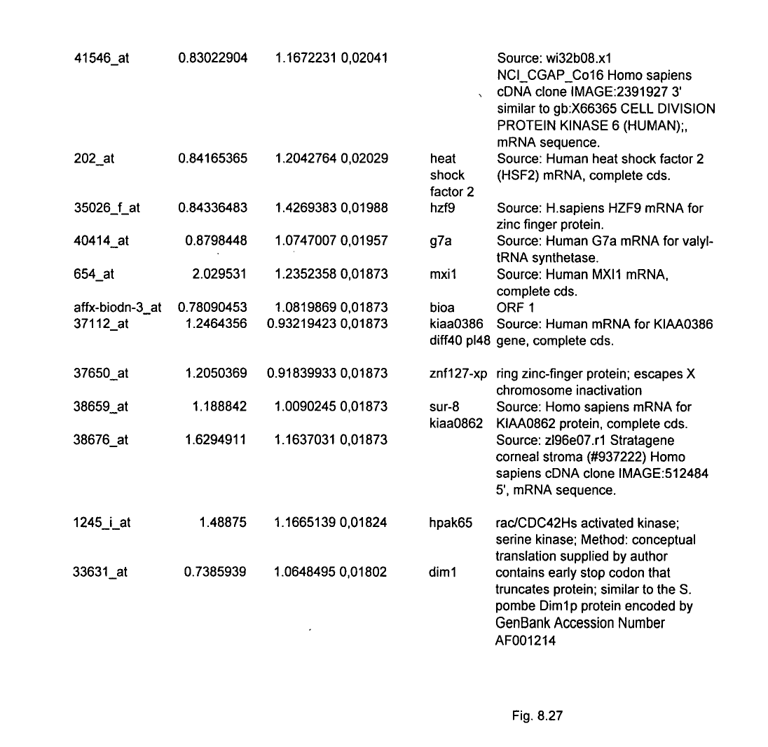

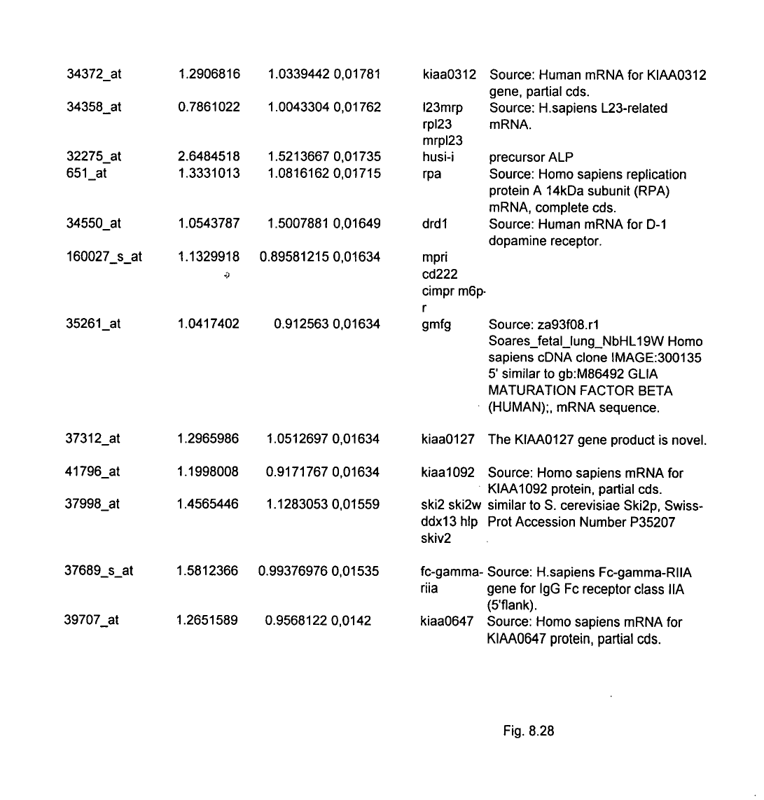

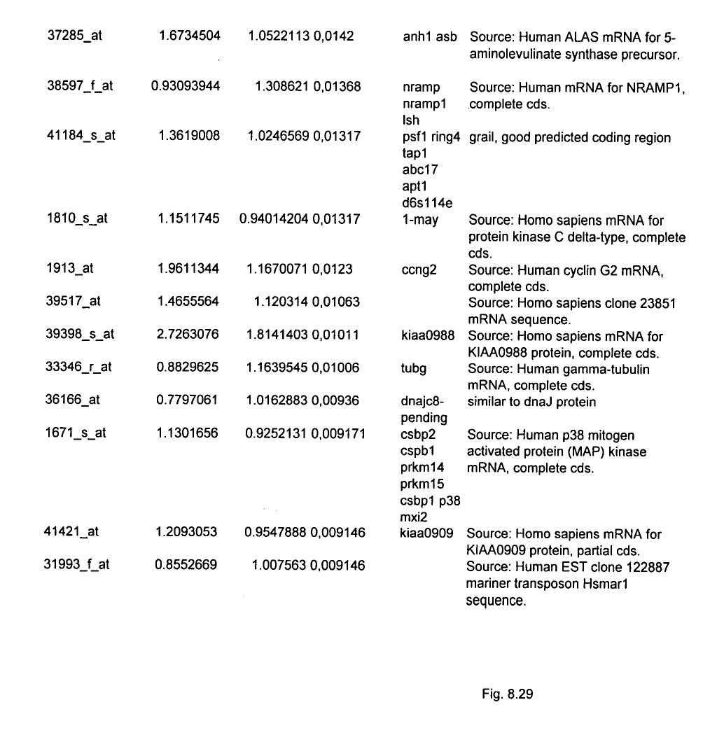

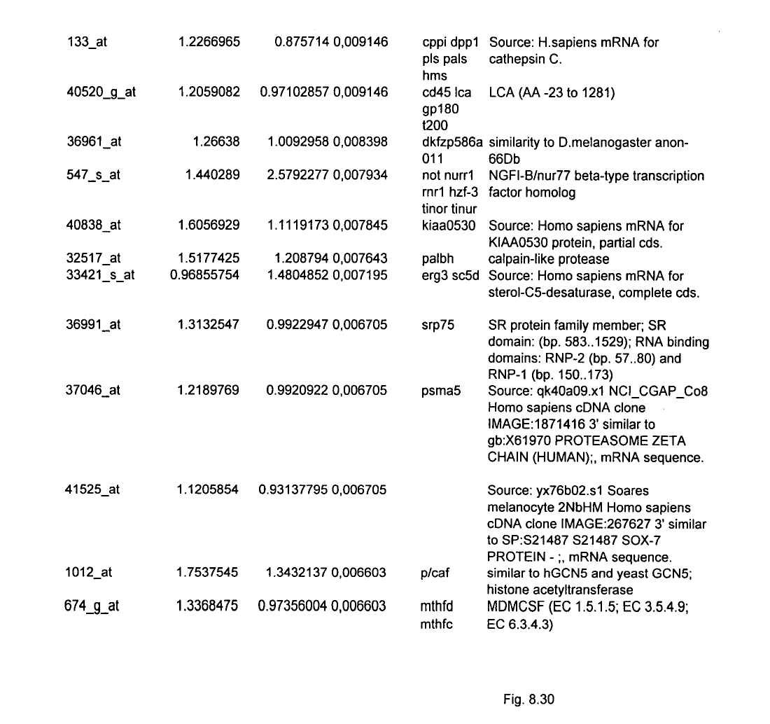

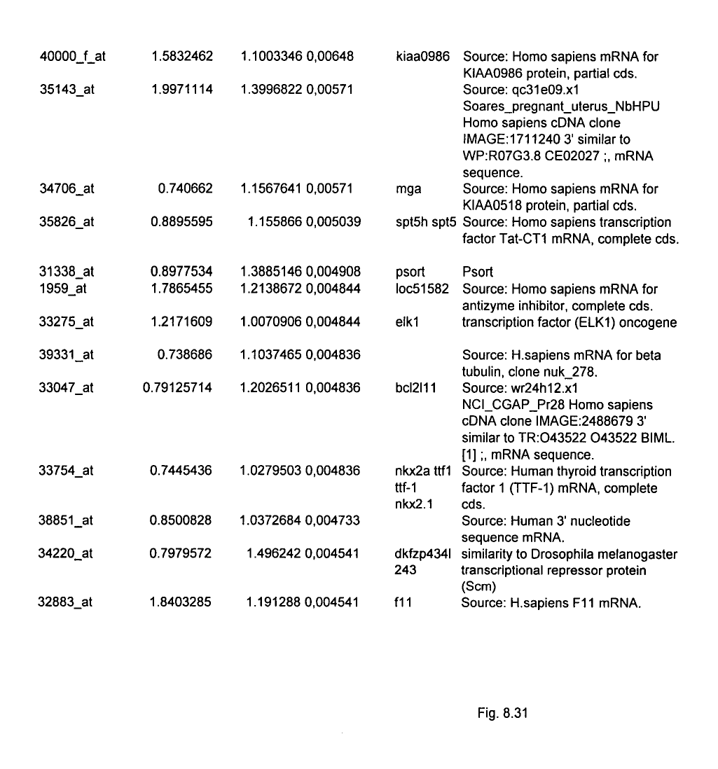

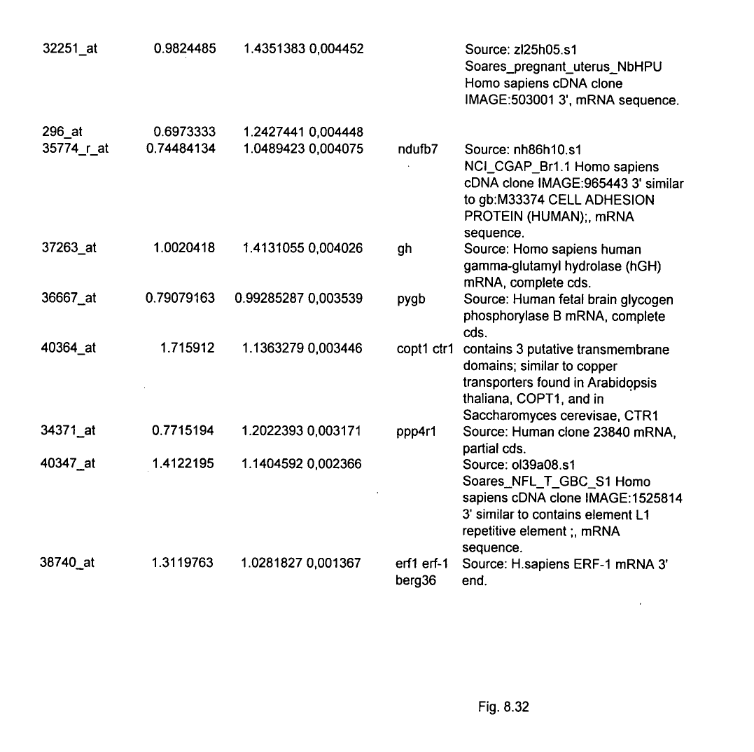

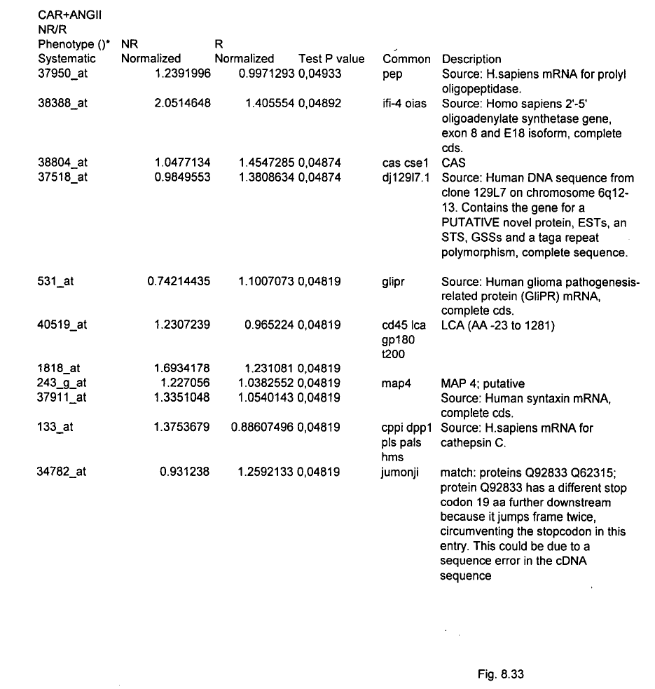

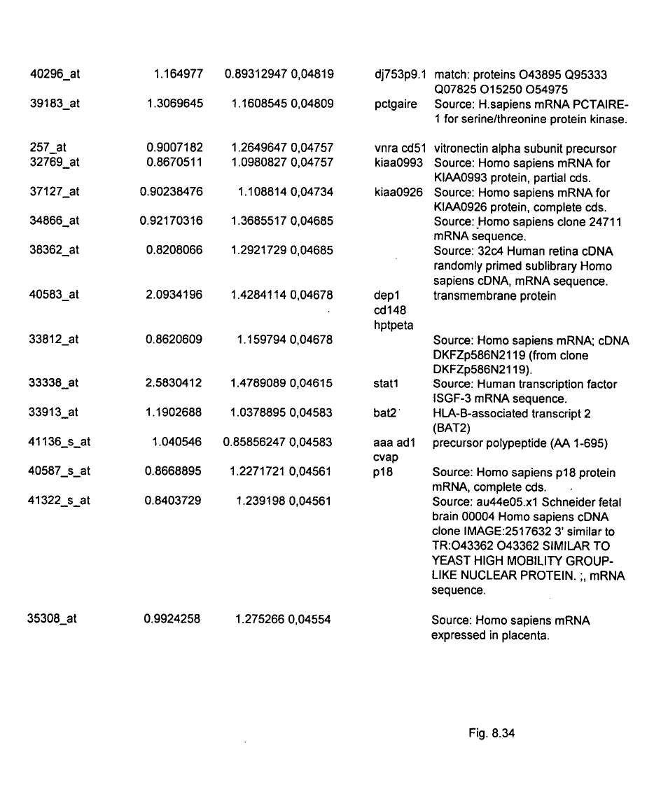

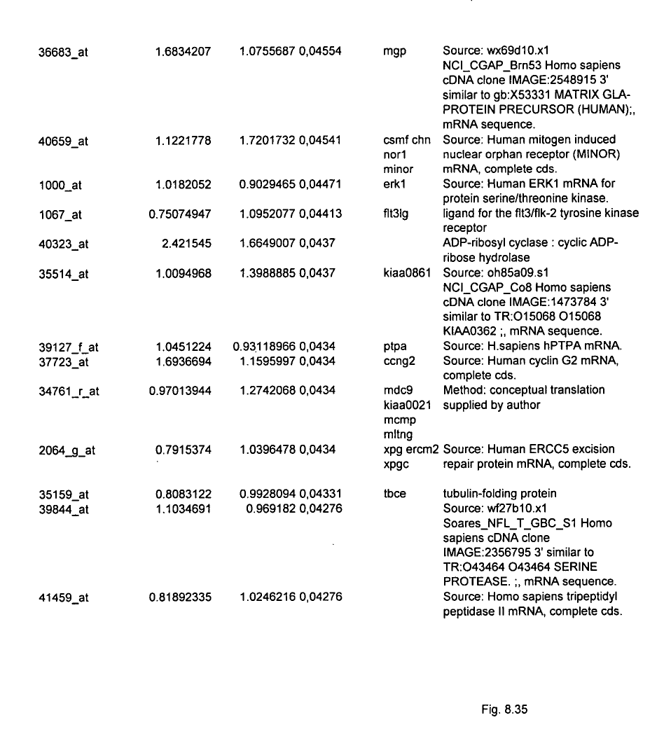

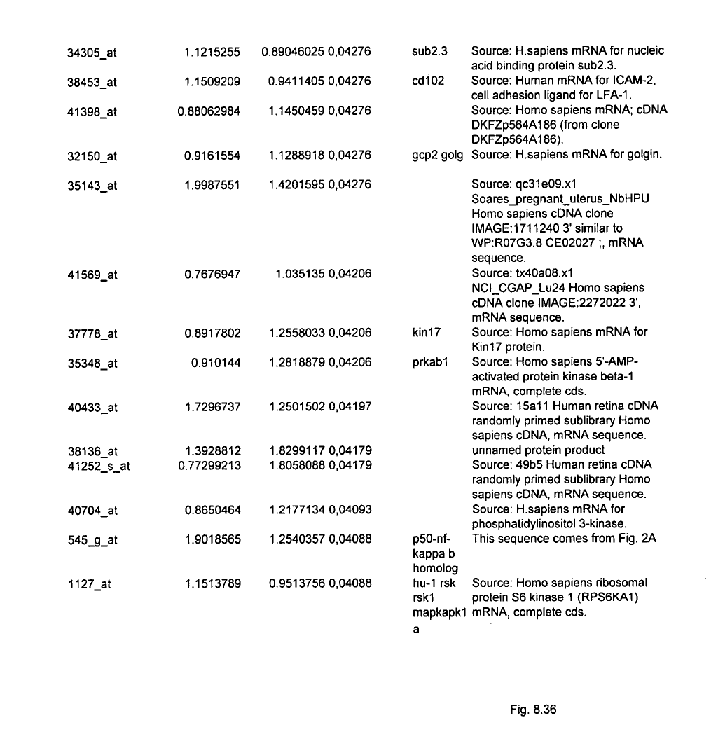

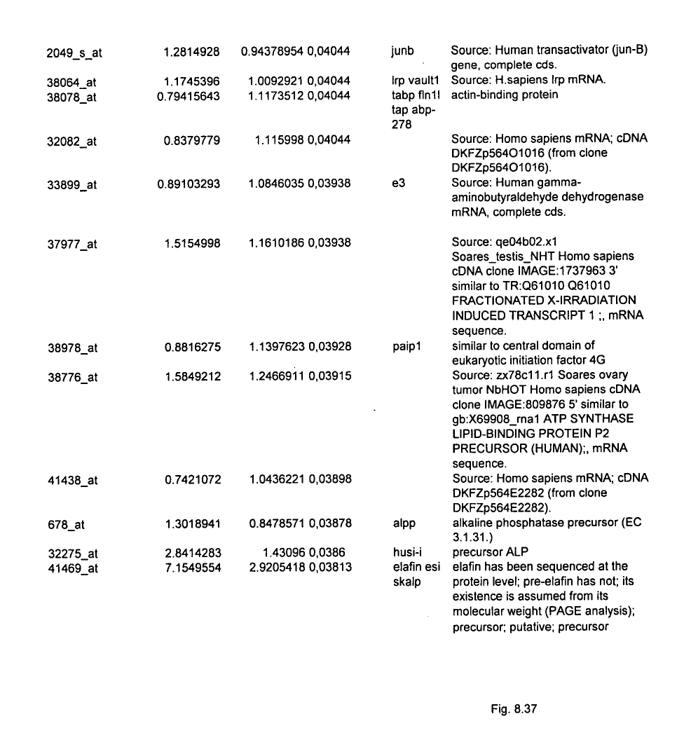

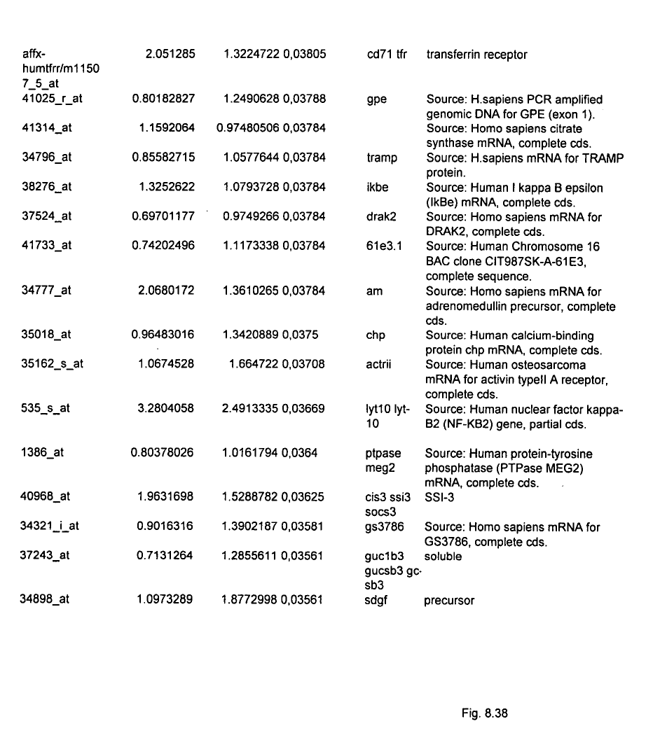

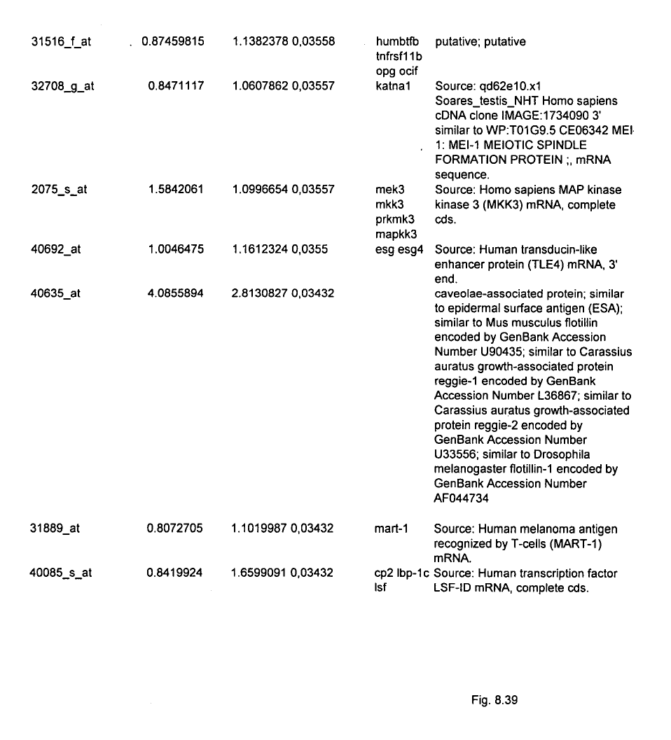

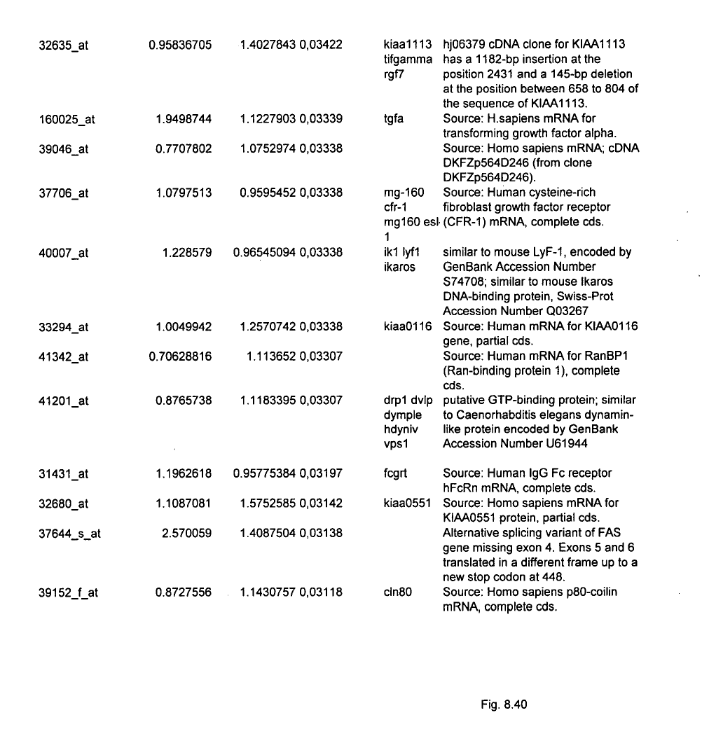

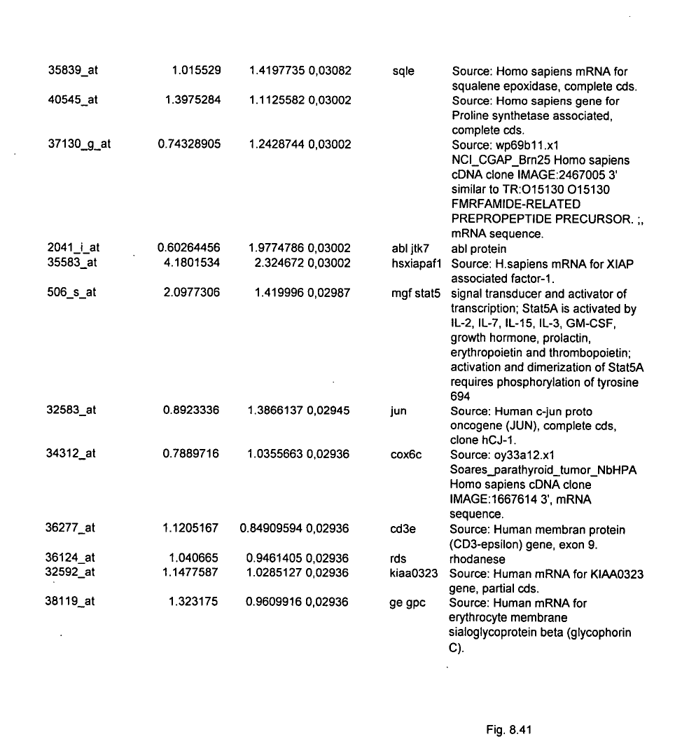

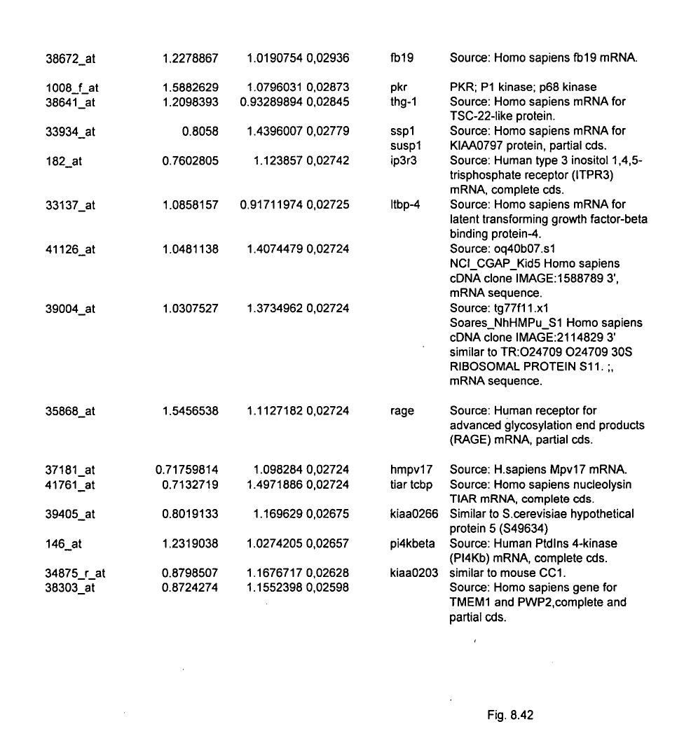

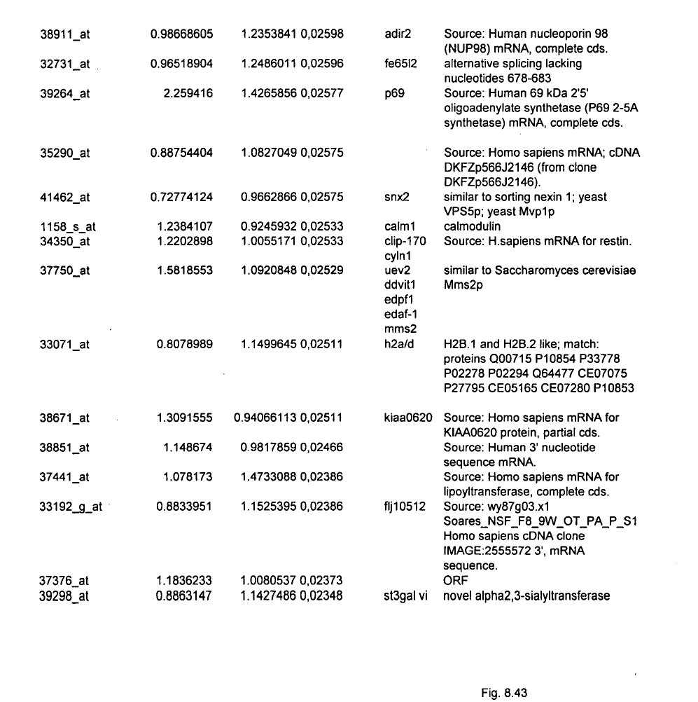

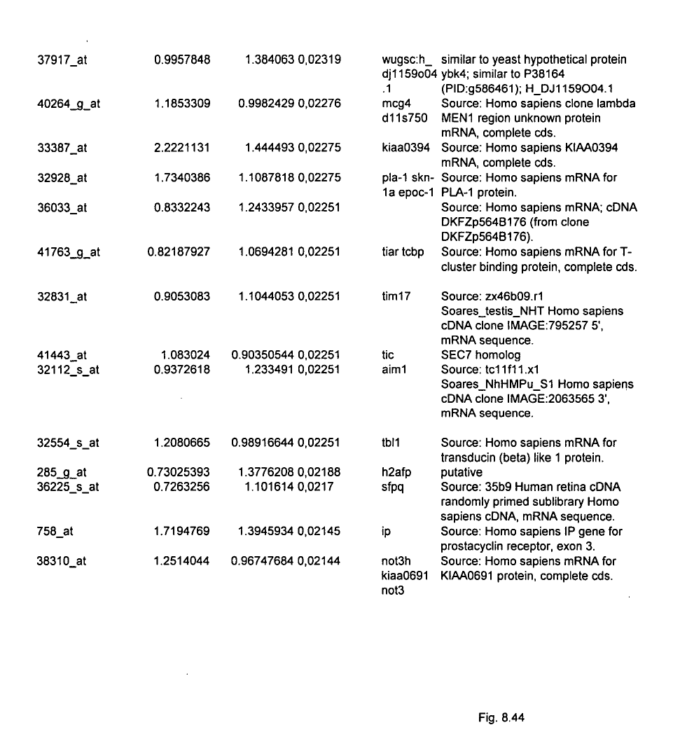

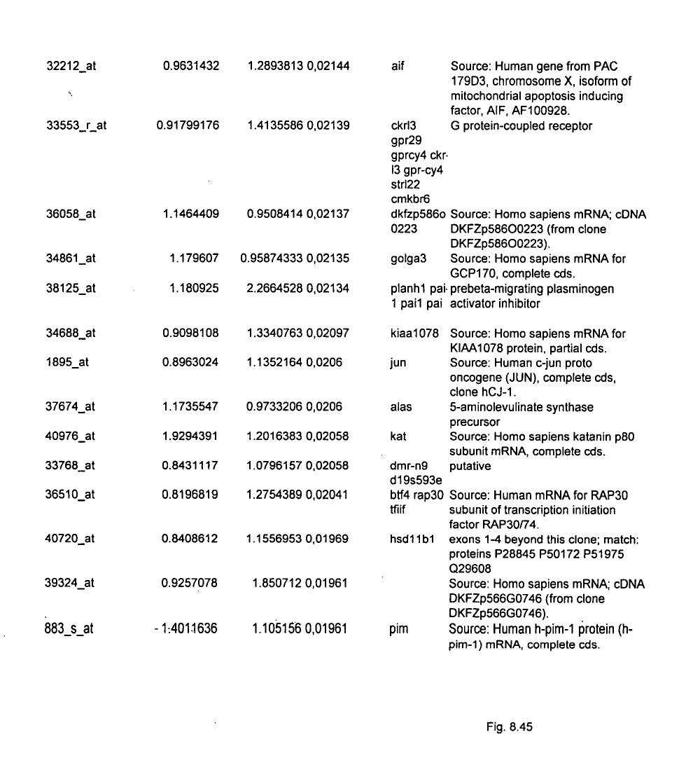

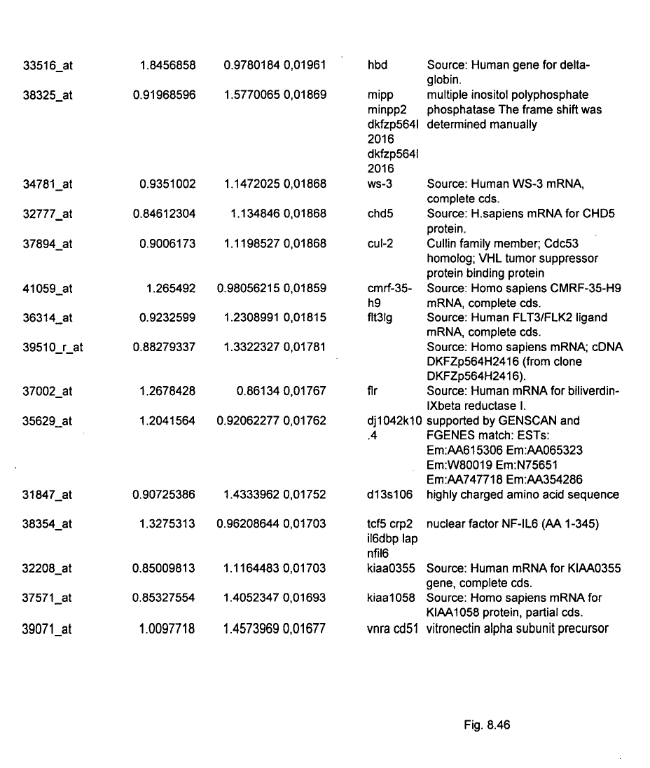

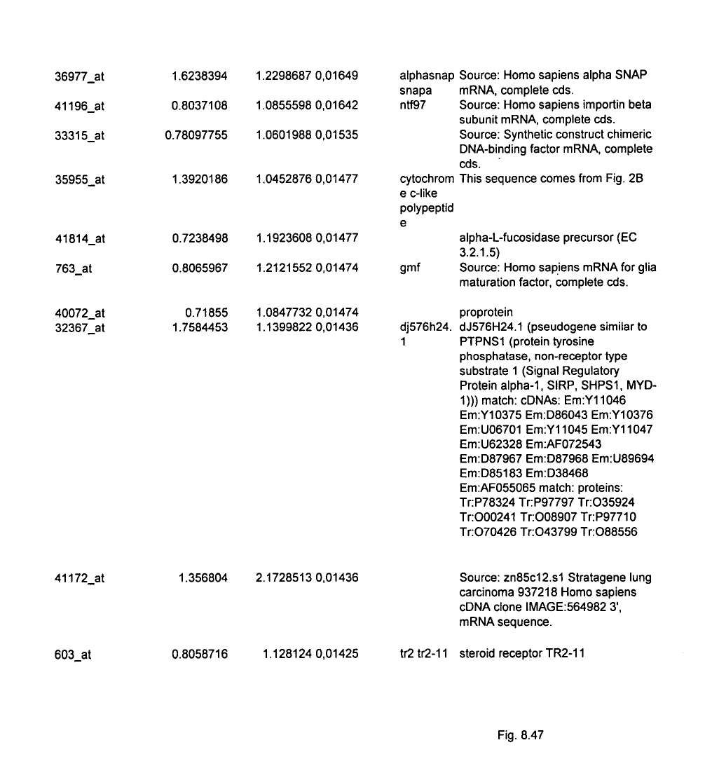

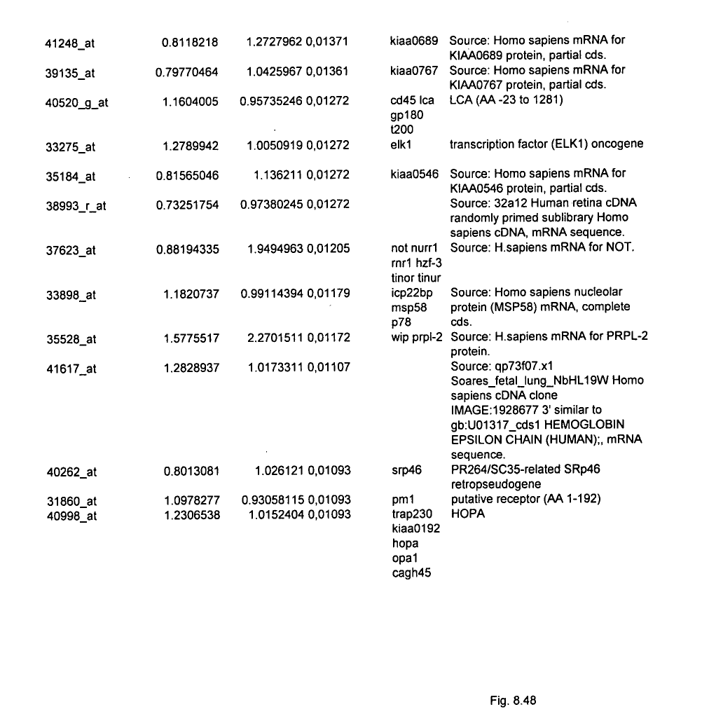

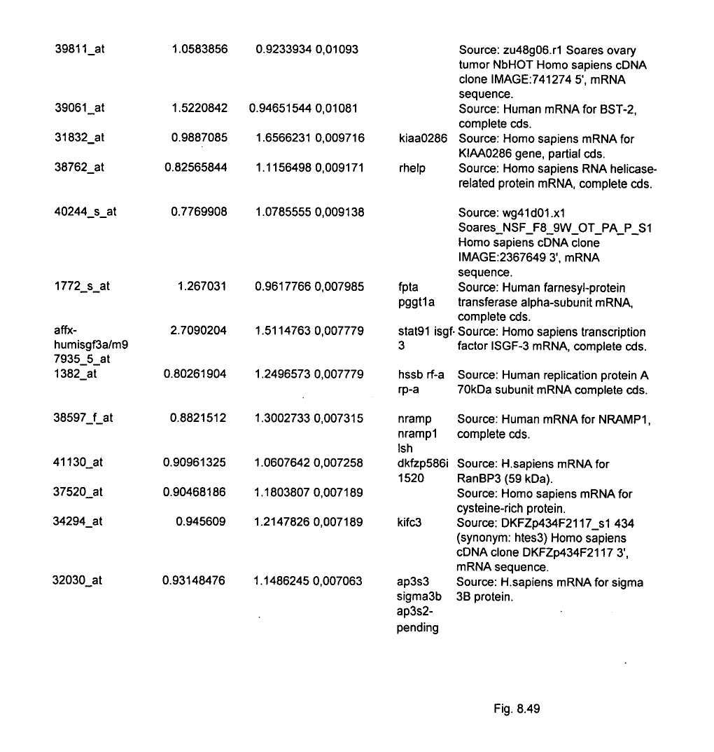

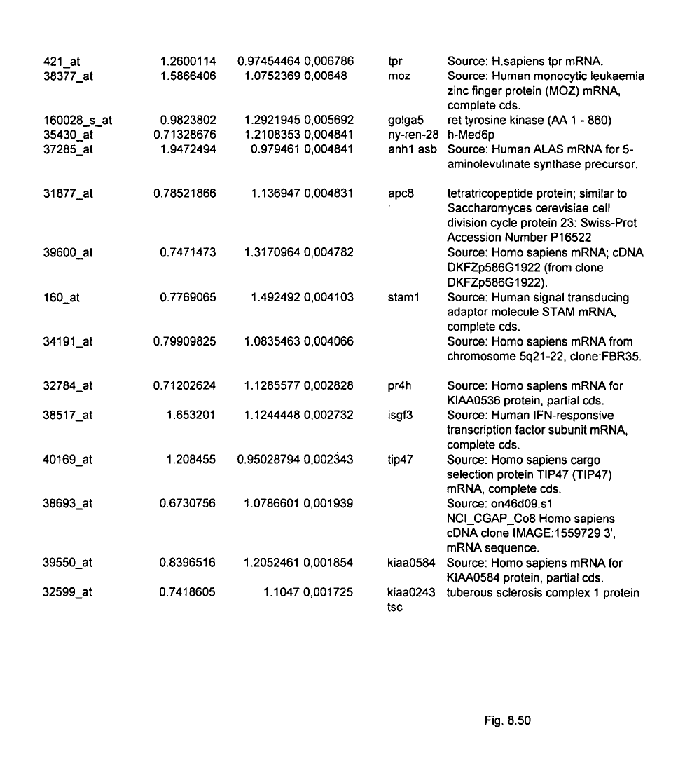

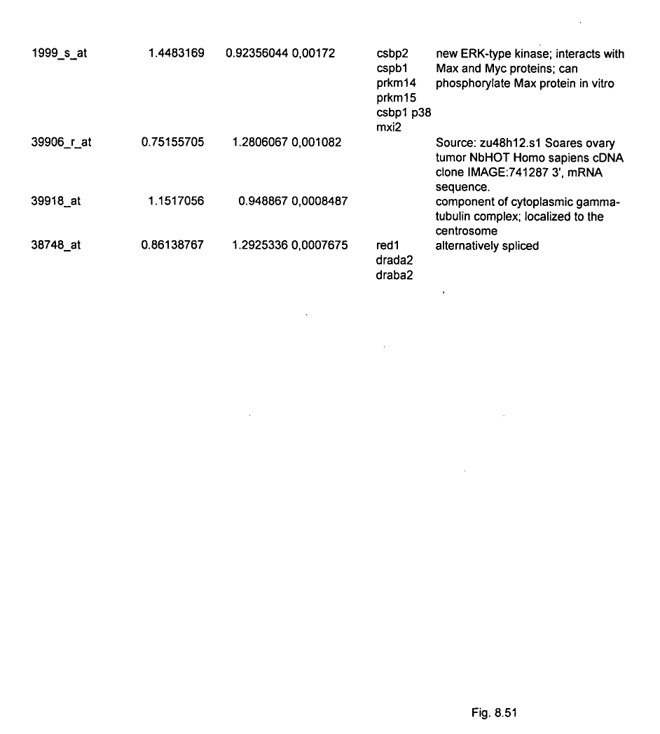

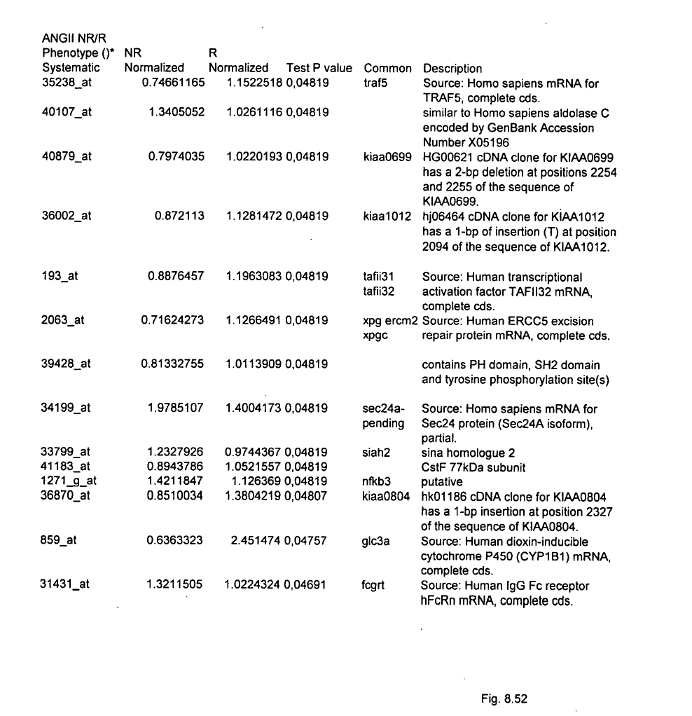

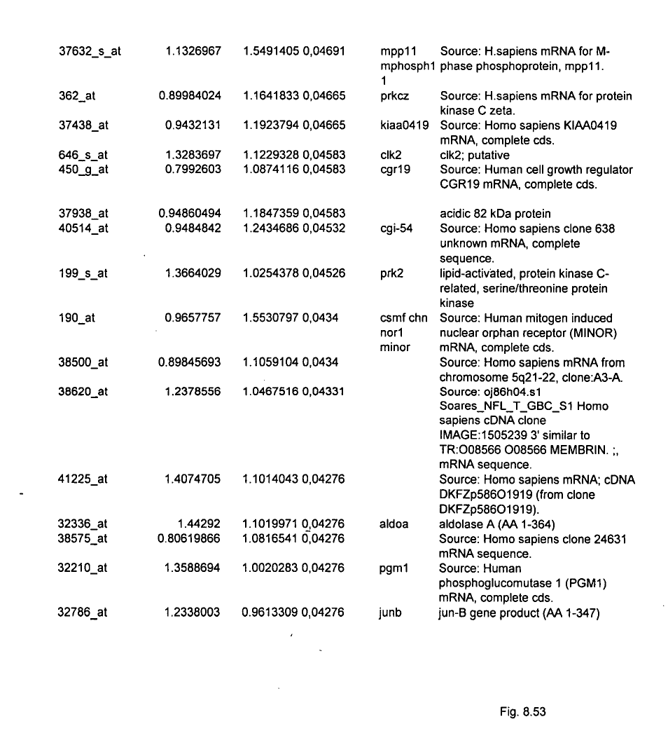

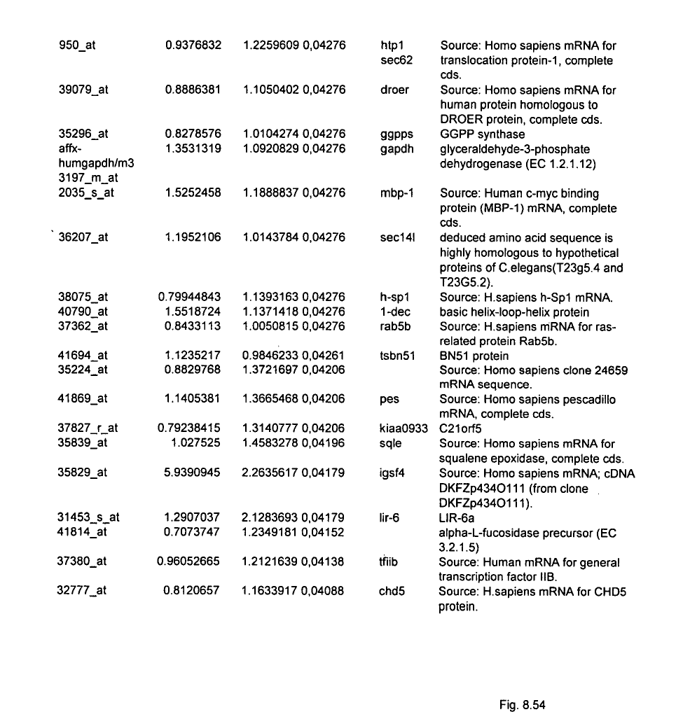

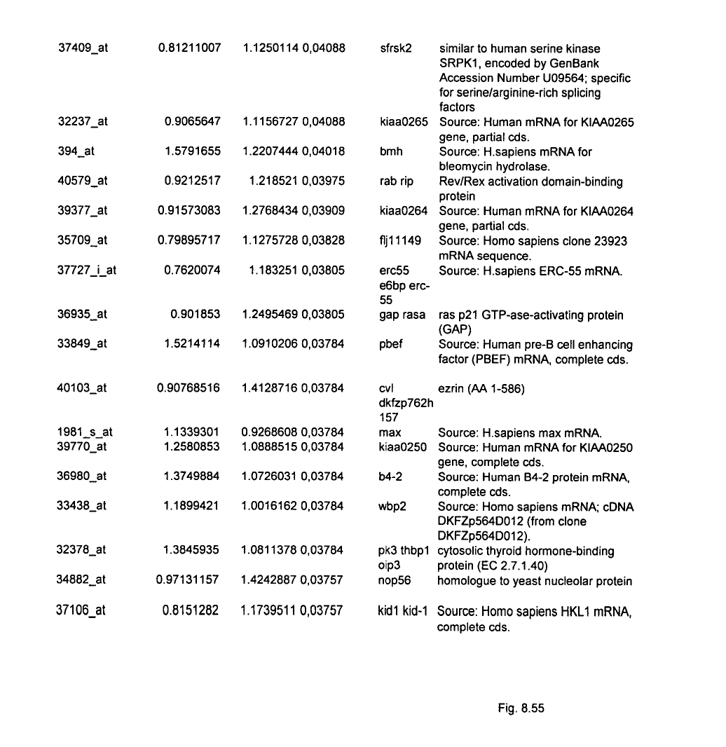

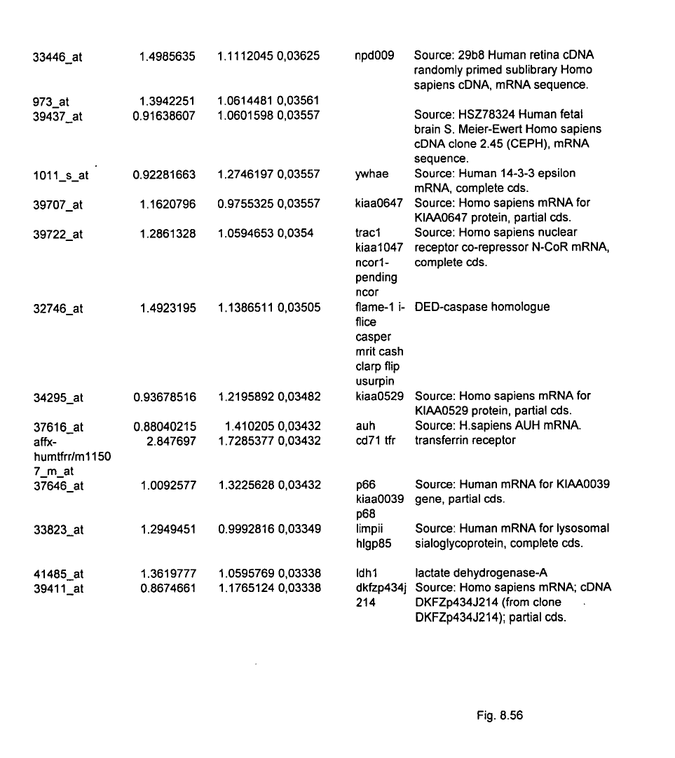

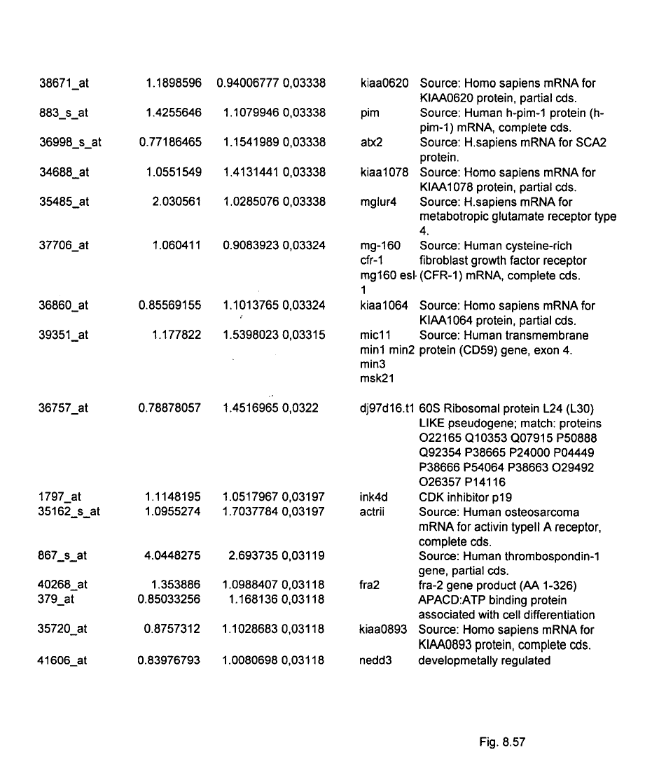

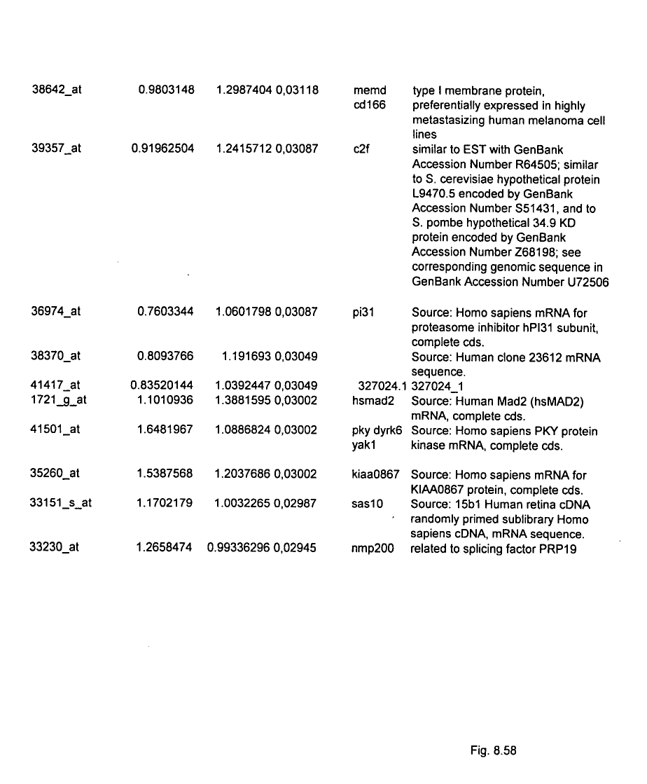

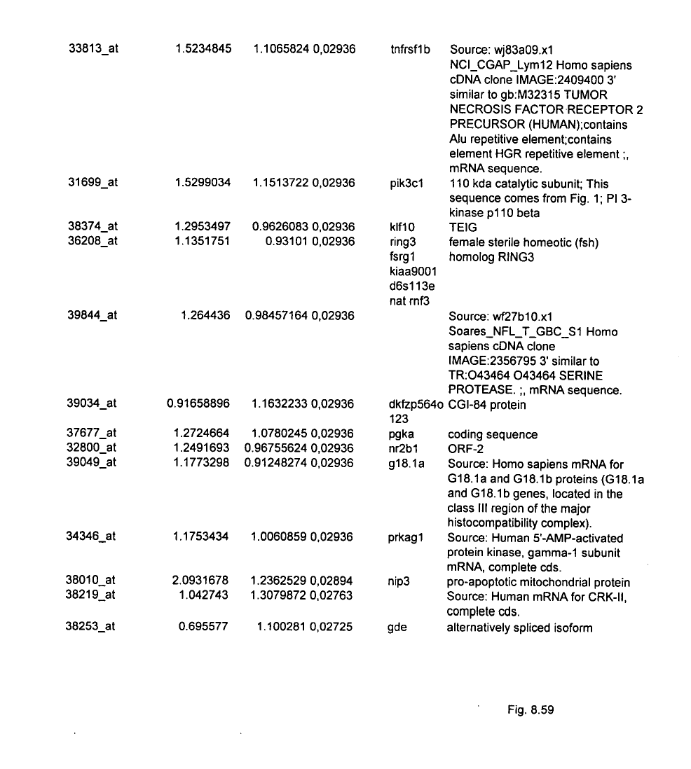

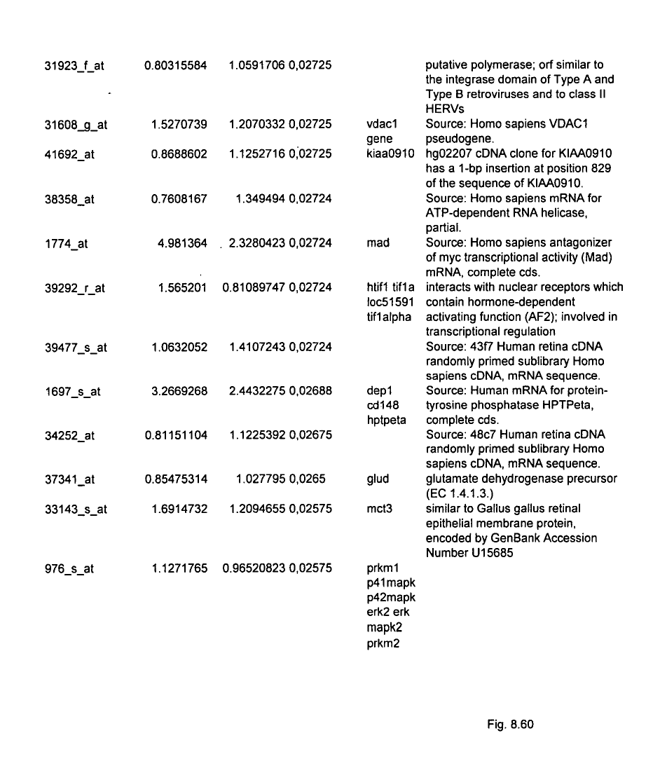

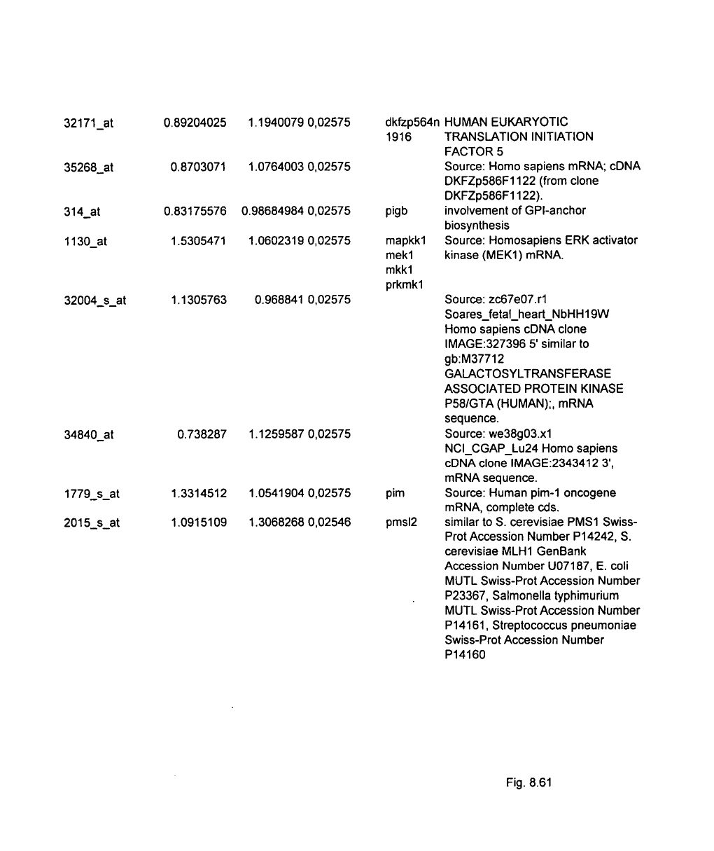

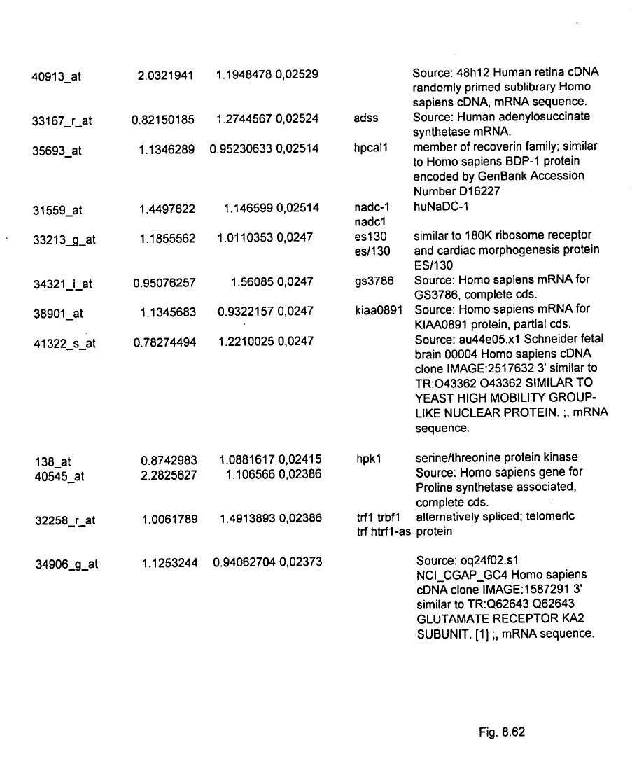

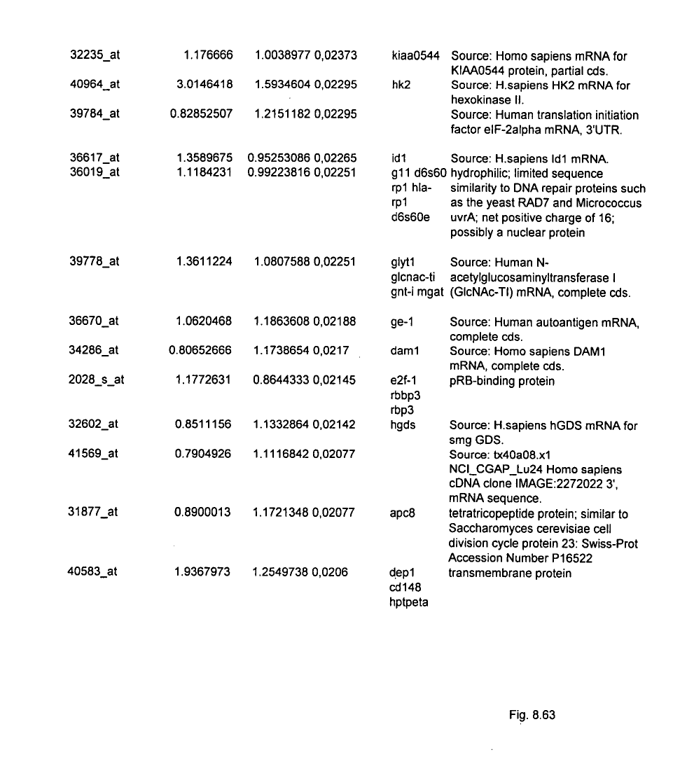

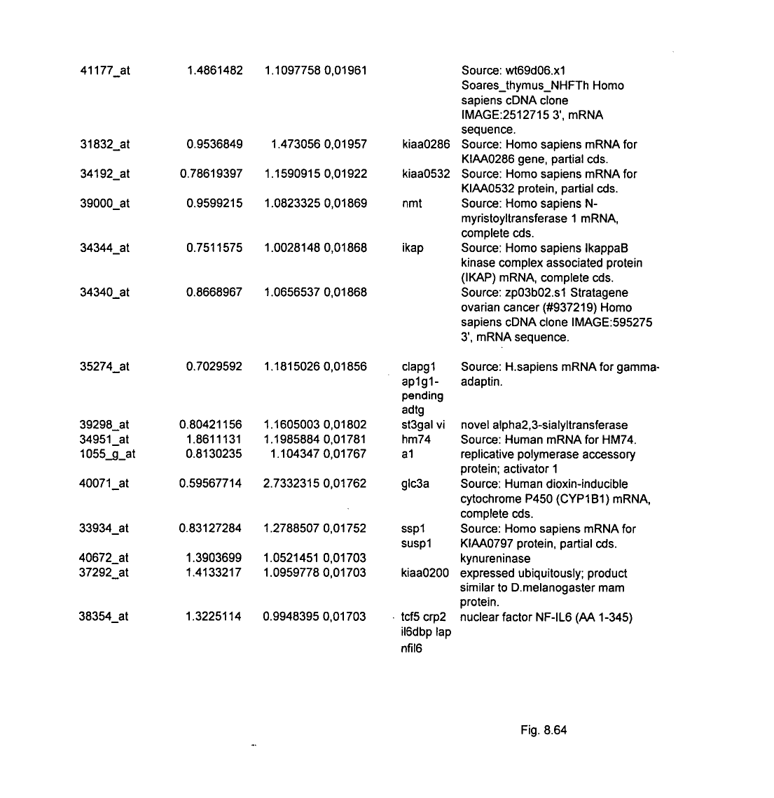

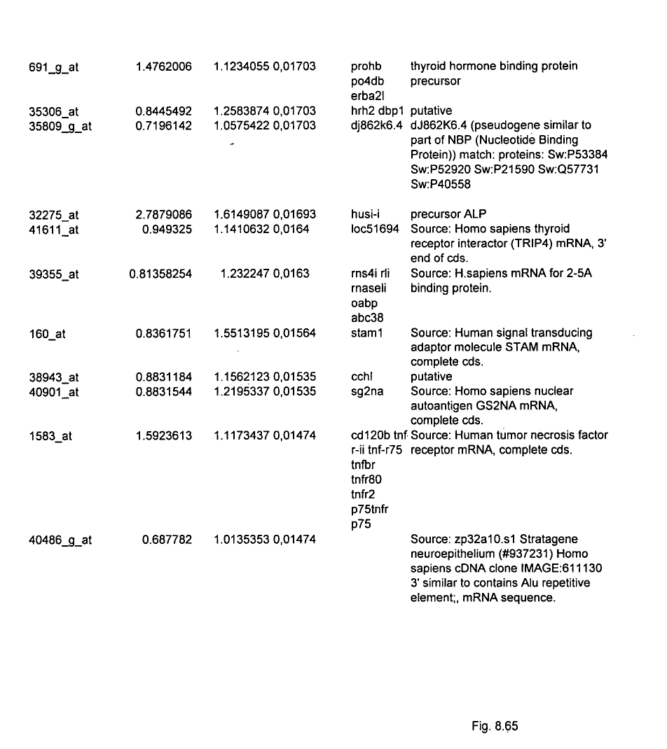

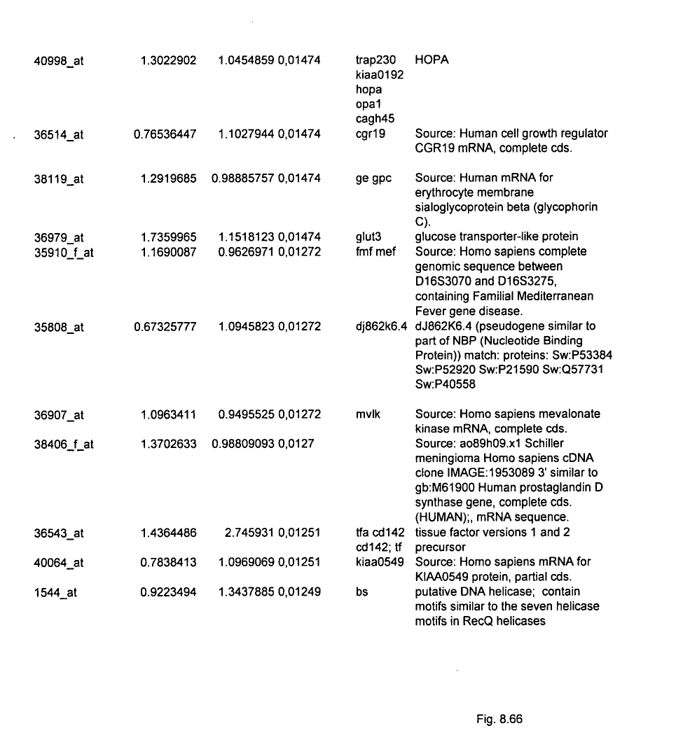

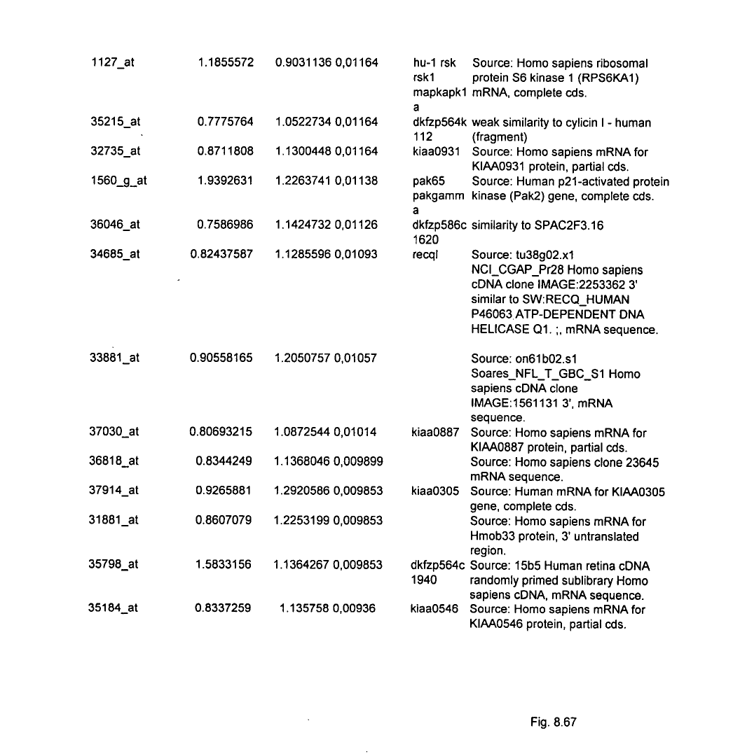

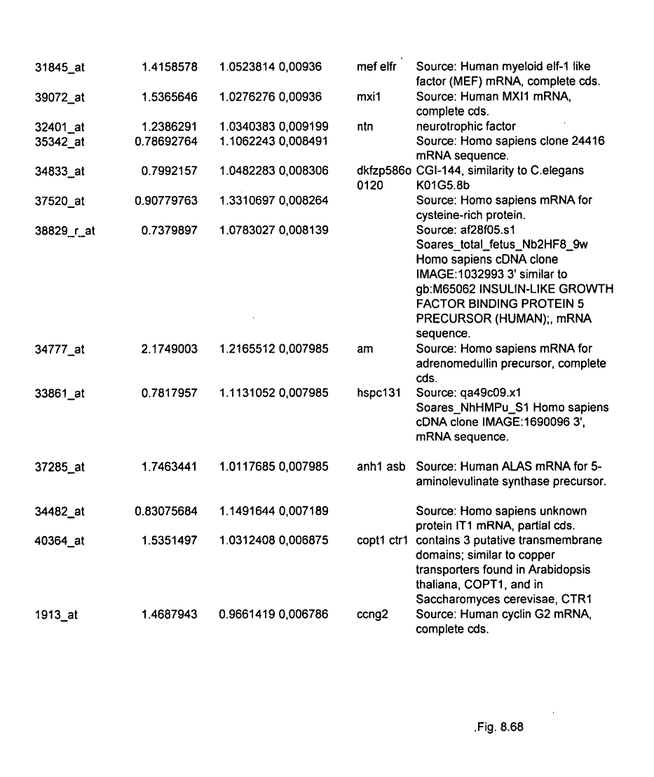

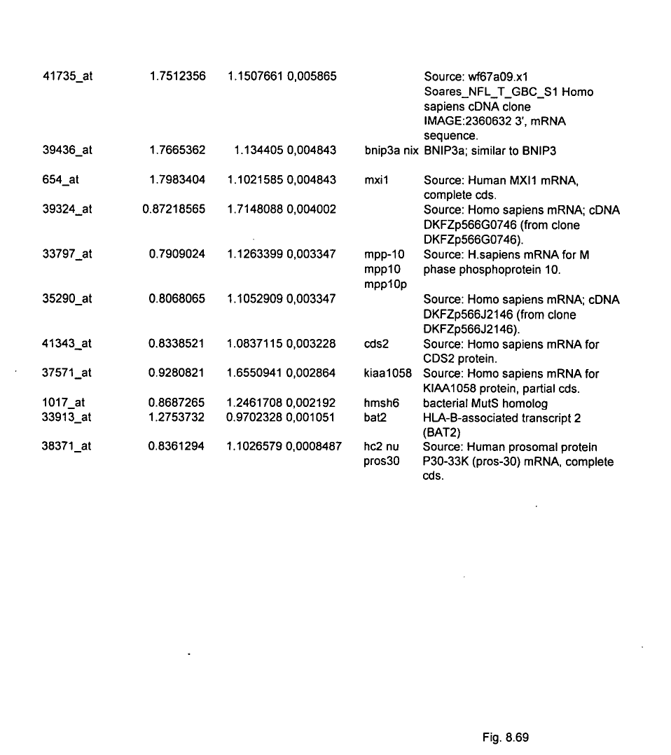

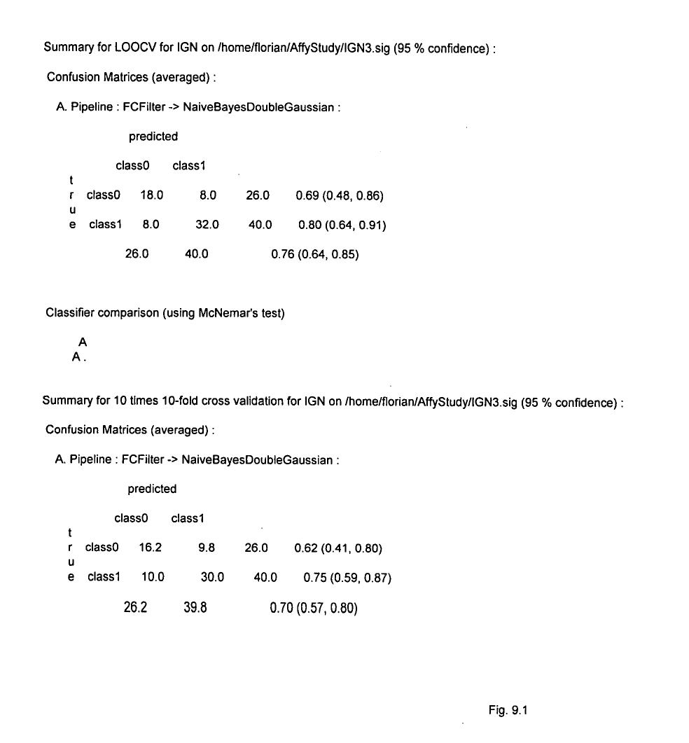

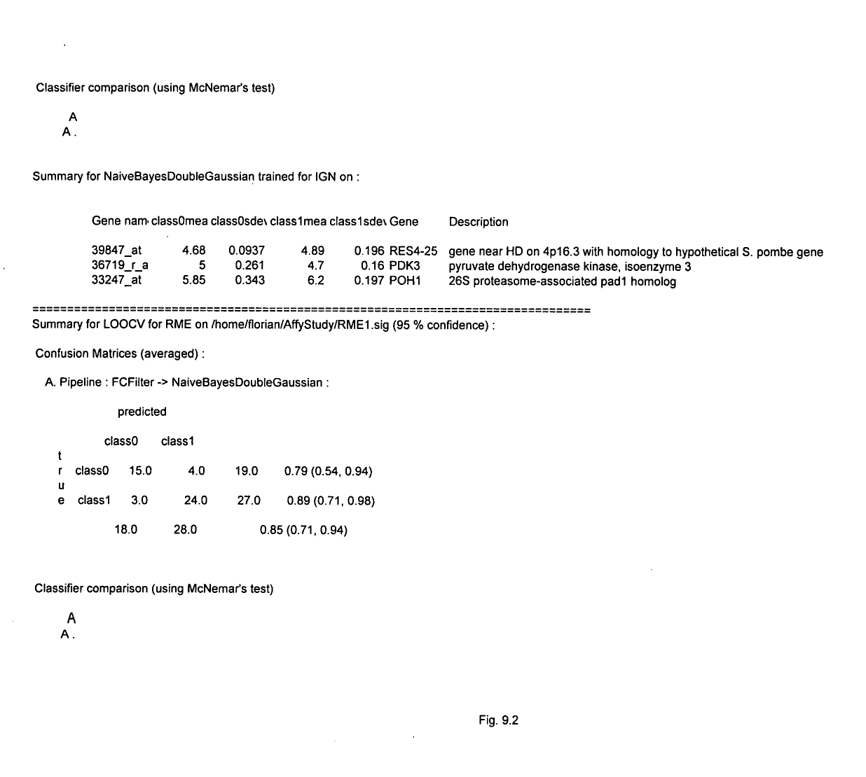

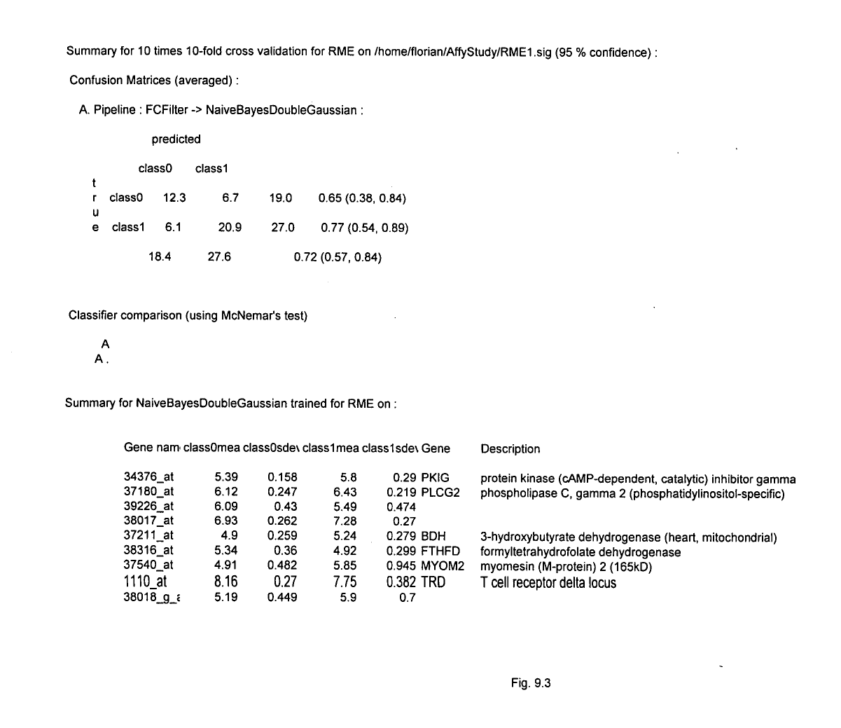

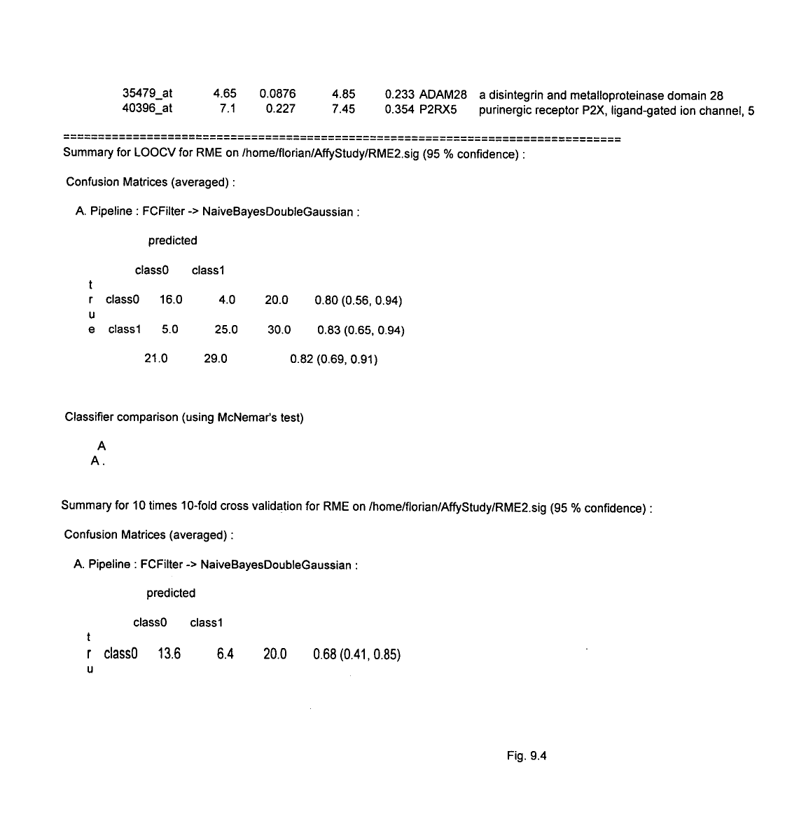

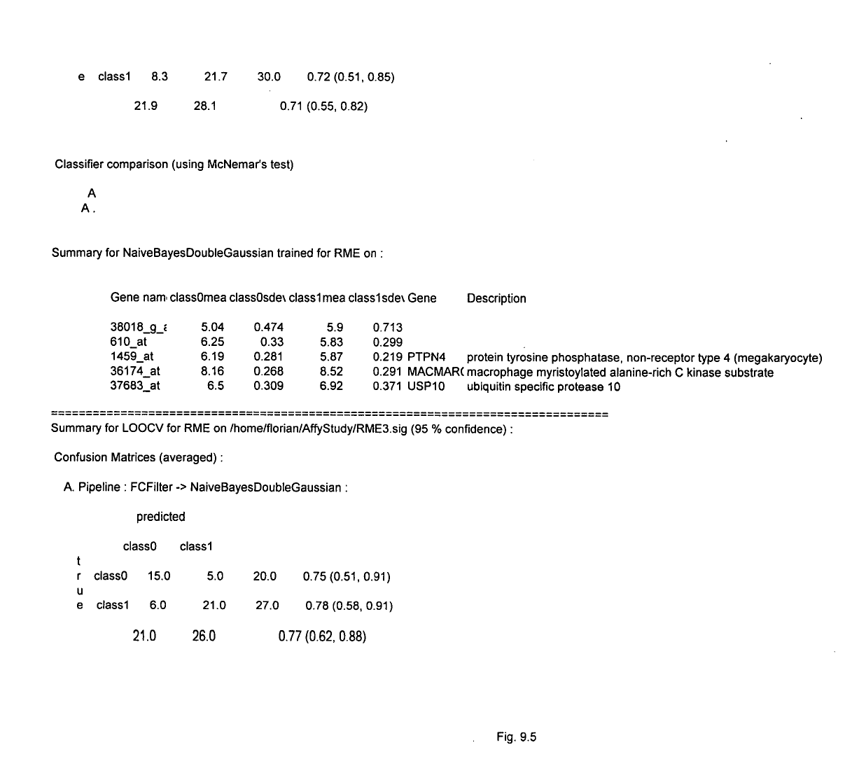

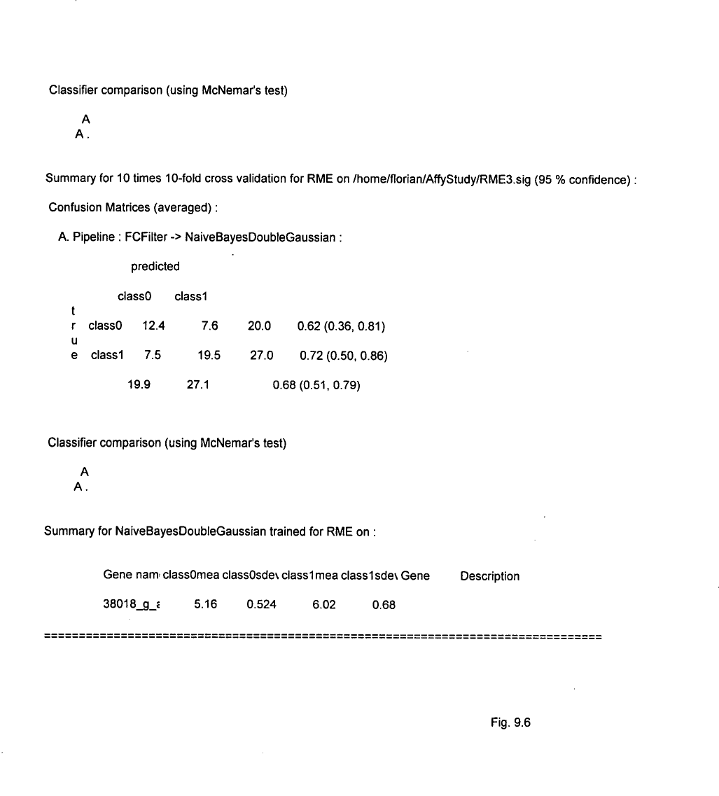

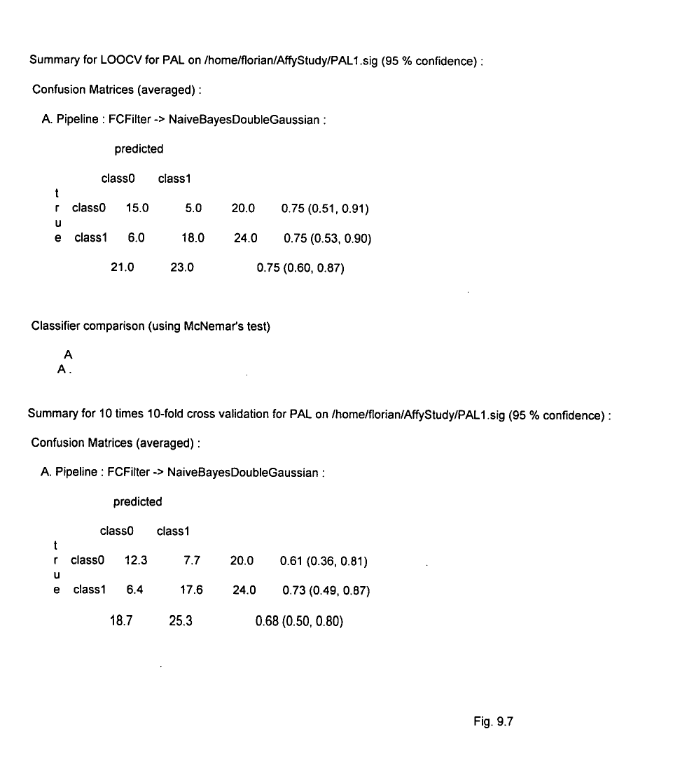

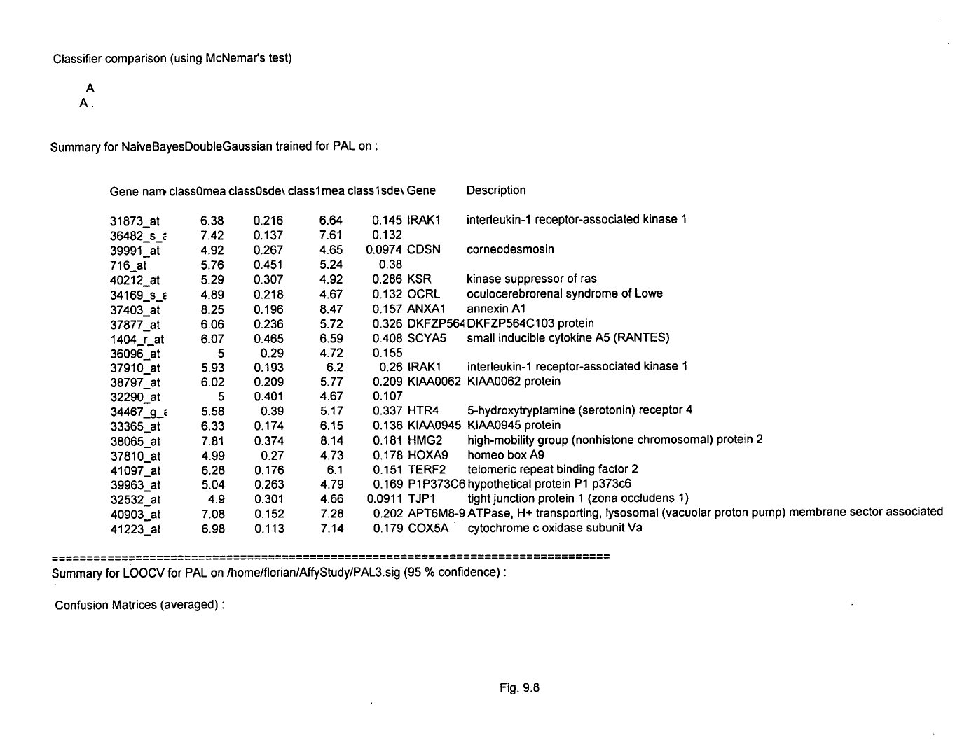

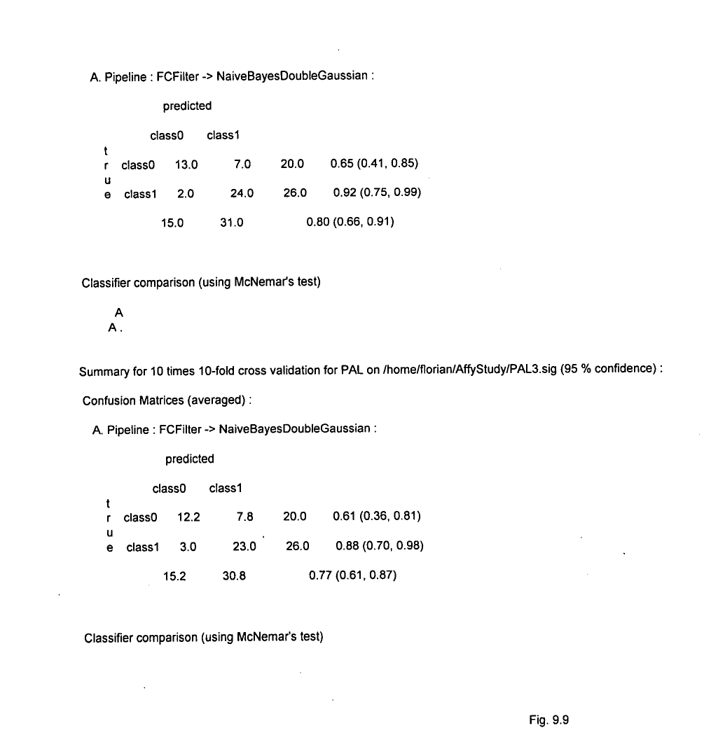

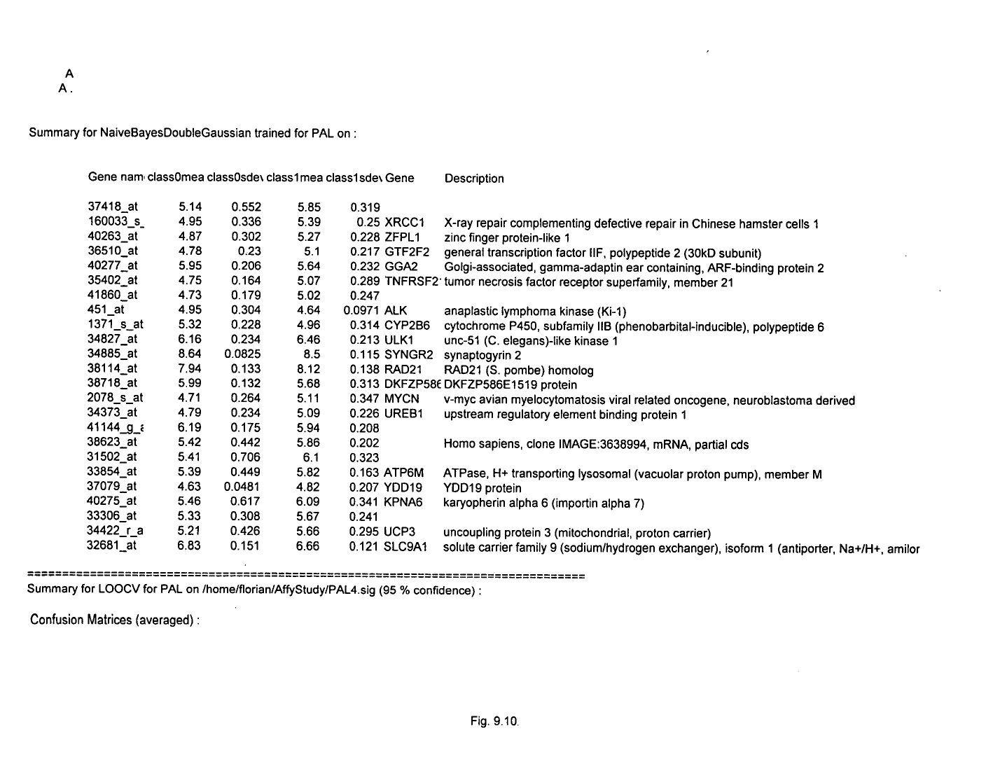

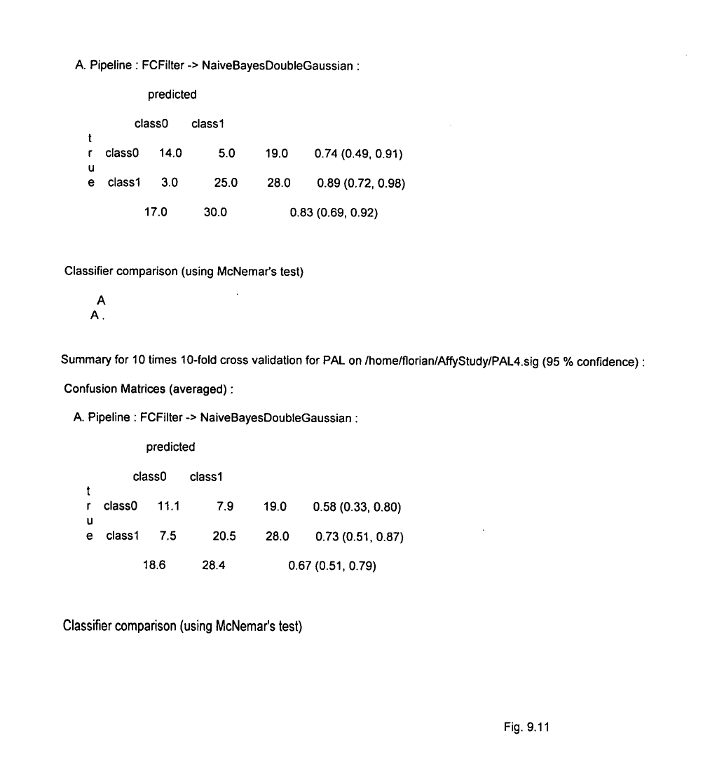

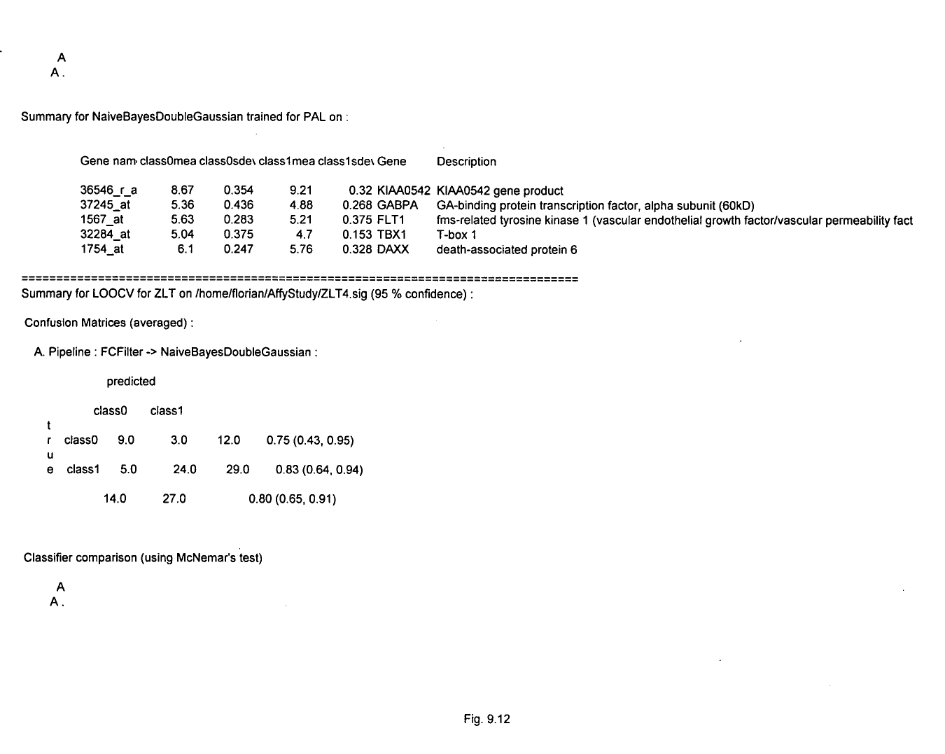

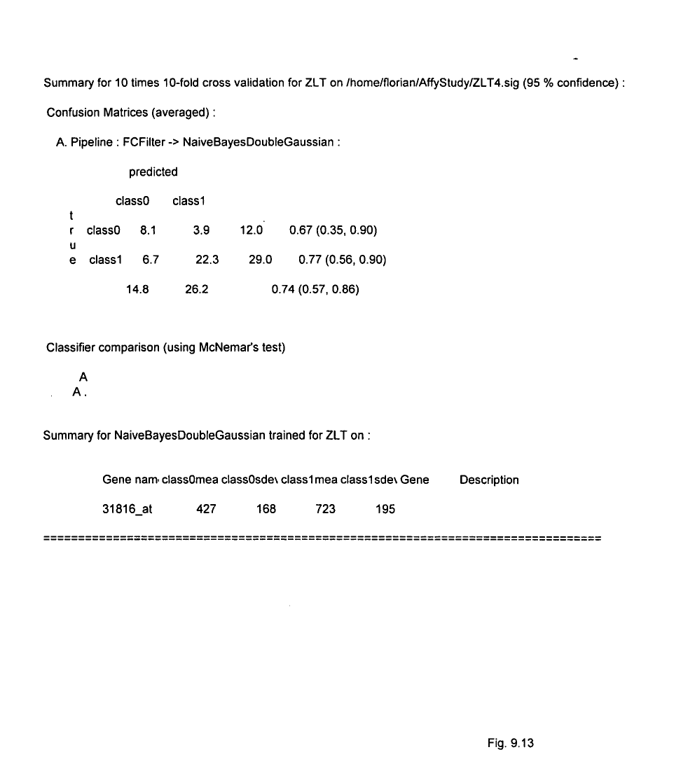

The data mining methods used in the following non-limiting examples are known in the art and include, for example, weighted voting algorithms and k-Nearest Neighbor algorithms (Golub, T. et al., 1999. Science, 286:531-537; Tamayo, P. et al., 1999. Proc. Natl. Acad. Sci. USA, 96:2907-2912; the contents of which are incorporated herein by reference in their entirety). Genes showing differential expression in one or more classes are suitable for use in the methods of the present invention as informative genes (for example, the genes described in FIGS. 6-9).

Prediction of an independent patient set using the weighted voting method described by Golub et al. Responder and non-responder patient cohorts are each randomly split into two cohorts. The random split procedure is performed 10 times with separate analyses each time. A predictor is determined for the first cohort using a weighted voting algorithm similar to that described previously (Golub, T. et al., 1999., Science, 286:531-537) by selecting genes that are deemed most relevant in distinguishing responders from non-responders. The weighted voting algorithm makes a weighted linear combination of relevant “informative” genes obtained in the training set to provide a classification scheme for new samples. A brief description of the algorithm follows. The mean (μ) and standard deviation (ε) for each of the two classes (responders and non-responders) in the training set is first calculated. The Euclidean distance between the two classes is then calculated for each gene (x) such that EDₓ=√(μ_{R}−μₛ)² The variance of each class is equal to the standard deviation within that class squared, V_{R,S}=ε². Next, the metric used to choose the most informative genes and for the calculation of weight, for each gene, is calculated as follows: Mx=(ED_{X}²)/(Vₛ+V_{R}). To predict the class of a test sample Y, each gene X casts a vote for each class: W_{XR}=Mₓ√(Y−μ_{R})² and W_{XS}=M_{X}√(Y−μ_{S})². The final class of test Y is found by the lesser of (ΣW_{XR}) and (ΣW_{XS}). Thus, each gene has a vote based on its metric and the class to which its signal is closest. The class with the smallest vote at the end is the predicted class, e.g., the class the test sample is closest to using the Euclidean distance as the measure, is the predicted class. It is then determined whether an accurate prediction of drug response can be achieved by expression values of this limited set of genes for the independent cohort with no prior information using commercially available software as described previously (Shipp, M. et al., 2002. Nat. Med., 8:68-74; Golub, T. et al., 1999., Science, 286:531-537).

Prediction of an independent patient set using the k-Nearest Neighbor algorithm. The k-Nearest Neighbor (k-NN) algorithm (Cover, T. et al., 1967. IT, 13:21-27) is also implemented to predict the class of a sample by calculating the Euclidean distance of the sample to the k “nearest neighbor” standardized samples in “expression” space in the training set. The class memberships of the neighbors are examined, and the new sample is assigned to the class showing the largest relative proportion among the neighbors after adjusting for the proportion of each class in the training set. The marker gene selection process is performed by feeding the k-NN algorithm only the features with higher correlation to the target class. To select genes for use in the predictor, all genes are examined individually and ranked based on their ability to discriminate one class from the other using the information on that gene alone. For each gene and each class, all possible cutoff points on gene expression levels for that gene are considered to predict class membership either above or below that cutoff. Genes are scored on the basis of the best prediction point for that class. The score function is the negative logarithm of the p-value for a hypergeometric test (Fisher's exact test) of predicted versus actual class membership for this class versus all others. Prediction using Leave One Out Cross Validation (LOOCV). In addition to confirmation in an independent set of patients, the genes that separate, for example, the class of responders from non-responders can be selected and tested by cross-validation, wherein one patient is excluded from the data set and then a new predictor is generated from all the genes based on the remaining patients. The new predictor is then used to predict the excluded sample.

### EXEMPLIFICATION

### Example 1

### Characterization of gene expression profiles in Ramace responsive vs. non-responsive hypertensive patients using Affymetrix gene array.

ACE is a peptidyl dipeptidase that catalyzes the conversion of angiotensin I to the vasoconstrictor substance, angiotensin II. Angiotensin II also stimulates aldosterone secretion by the adrenal cortex. The beneficial effects of Ramace™ in hypertension and heart failure appear to result primarily from suppression of the renin-angiotensin-aldosterone system. Inhibition of ACE results in decreased plasma angiotensin II, which leads to decreased vasopressor activity and to decreased aldosterone secretion. The latter decrease may result in a small increase of serum potassium. ACE is identical to kininase, an enzyme that degrades bradykinin. Whether increased levels of bradykinin, a potent vasodepressor peptide, play a role in the therapeutic effects of Ramace™ remains to be elucidated.

In hypertensive patients with normal renal function treated with ACE inhibitors alone for up to 24 weeks, the mean increase in serum potassium was observed to be approximately 0.1 mEq/L; however, approximately 15% of patients exhibited increases greater than 0.5 mEq/L and approximately 6% exhibited a decrease greater than 0.5 mEq/L. In the same study, patients treated with an ACE inhibitor and hydrochlorothiazide for up to 24 weeks exhibited a mean decrease in serum potassium of 0.1 mEq/L; approximately 4% of patients exhibited increases greater than 0.5 mEq/L, and approximately 12% exhibited a decrease greater than 0.5 mEq/L. Removal of angiotensin II negative feedback on renin secretion leads to increased plasma renin activity.

While the mechanism through which ACE inhibitors, such as Ramace™, lowers blood pressure is believed to be primarily suppression of the renin-angiotensin-aldosterone system, Ramace™ is anti-hypertensive even in patients with low renin hypertension. Although ACE inhibitors confer anti-hypertensive properties to all races studied, African American hypertensive patients (usually a low-renin hypertensive population) had a smaller average response to monotherapy than patients in other populations.

In view of the above considerations, the following hypotheses were addressed:
A: that hypertensive patients who demonstrate clinical improvement when taking ACE inhibitors (e.g., Ramace™) can be identified by specific gene expression profiles in white blood cells, which are characteristic for patients who do respond to the drug and differ from the expression profiles in non-responders.
B: that these expression profiles can be used to develop an expression or DNA-based test that accurately predicts the responsiveness to ACE inhibitors (e.g., Ramace™).

To address these hypotheses, 57 patients were identified and recruited, 35 of whom demonstrate a clinical response (“responders”) to the ACE inhibitor, Ramace™, and 22 of whom are “non-responders”. Ramace™ response was be measured by a scoring function taking into account both clinical and laboratory parameters as described below. Gene expression profiles that accurately predict the responsiveness of these hypertensive patients to Ramace™, using a gene expression array chip system and data mining algorithm(s), were obtained. The goal, confirmed below, was that the results from the expression studies would demonstrate distinctive gene expression profiles that allow for characterization between Ramace™ responders and non-responders with predictive accuracy 85%, and, based on these gene expression profiles, a diagnostic test to identify patients who are best suited for Ramace™ therapy can be developed (FIG. 2).

The diagnostic test can be developed from such gene expression profile data to include one or more of the following aspects:
a) the genes whose expression patterns are highly predictive can be re-arrayed on a more cost-effective expression platform and can be used as a predictor of response in the clinic;
b) finding associated variants within the genes whose expression pattern is predictive and using the latter as a DNA-based predictor of response in the clinic.
   One of skill in the art would appreciate that such data can be used in a variety of ways to design a test that accurately separates responders from non-responders.
   Subject/Patient Criteria

Inclusion Criteria

The criteria the subject/patient had to satisfy to enter the study, includes the following:

a. Hypertension diagnosed by a Cardiologist. The method used to diagnose hypertension is based on the ICE-9 and ICD-10 classification system, and concurs with the diagnostic hypertension criteria outlined by the American Heart Association and includes the following measures: Blood pressure greater than 140 systolic and 90 diastolic (adult person). For this study, individuals will be recruited primarily with moderate to severe hypertension.
b. Patients with hypertension with a severity level that is judged to be appropriate to treatment with Ramace™ were recruited to participate in the study. Study patients signed a consent and donate a blood sample for the study.
c. Phenotype was confirmed by measurement of blood pressure levels at different time points.
d. Age 12-80 years.
e. Both males and females were recruited.
f. White Caucasian (Icelandic hypertensive patient population).
g. Ramace™ therapy for 8 weeks. Examination by a study cardiologist every 3 weeks who confirmed clinical response to Ramace™ therapy.
h. Response to Ramace™: Patients were categorized as either Ramace™ responders or Ramace™ non-responders. Ramace™ response is defined by any of the following:
   i) improved control of hypertension symptoms (blood pressure reduced by 10 mm/Hg in systole or more and/or 6 mm/Hg in diastole or more) when taking Ramace™,
   ii) improved quality of life/well being on Ramace™ therapy as judged by the patient and his/her cardiologist.
   iii) Ramace™ non-responders do not experience improvement in the above measures. The same cardiologists examined all 57 patients before and after Ramace™ trial.

Exclusion Criteria

A precise list of criteria that would exclude the subject/patient from entering the study, included:
a. Therapies that could interfere with evaluation of efficacy or the incidence of adverse effects, including:
   i) Other investigational drugs (simultaneously)
   ii) Concurrent anti-hypertensive medication that is not taken regularly
b. Diseases or conditions that could interfere with the evaluation of efficacy or the incidence of adverse effects, including:
   i) Pregnancy or lactation
   ii) Hypersensitivity or serious adverse experiences to hypertension drugs in the past
c. Sensitivity to the study drug or its components
e. If compliance to medication is of question
   Summary of Study Design

Patients with hypertension diagnosed by the above criteria were invited to participate in the study and recruited to take the ACE inhibitor, Ramace™. Sixty patients were recruited to participate. Beforehand, it was expected that 40 patients would be ACE inhibitor responders and 20 non-responders, as judged by the clinical criteria described above. A cardiologist, who was blinded to the expression array studies, confirmed the phenotype of all patients. All patients had a physical examination where their phenotype was confirmed. The patients donated a blood sample for the study. Fifty to sixty mL of EDTA blood was collected for the study in 7×8 mL tubes. PBMCs were isolated from the rest of the blood, their RNA extracted and reverse transcribed to cDNA. Upon transcription to cRNA, the transcripts were hybridized to Affymetrix GeneChip™ microarrays as described below.

Treatment Plan

All patients recruited were treated with therapeutic doses of Ramace™ for minimum of 8 weeks and examined by the study cardiologist before and after starting the drug. Blood samples were drawn at a similar time (e.g., around 9 AM) in the morning from all patients. The dose of the ACE inhibitor was recorded for all patients, before and 4 weeks after the patient was started on the study drug. No diet restrictions are implemented.

Study Procedures

PBMCs were isolated by the standard Ficoll method. The cells were then divided into 4 conditions each:
i) Immediate RNA isolation at baseline (0 hr or TO);
ii) Exposure to a ligand activating the ACE enzyme (e.g., angiotensin I) for 4 hrs, in the absence and presence of 1 hr pre-treatment with Ramace™ (10-6M), followed by immediate RNA isolation;
iii) Exposure to Ramace™ alone; and
iv) Exposure to the stimulant alone.

Upon RNA isolation, the samples were column cleaned, quantified by spectrophotometry, and equal amounts (5 μg) of total RNA will be used for cDNA synthesis for each condition. The cDNAs from the responders and non-responders are subsequently processed on to 12,600 Affymetrix GeneChip™ microarrays (Hu95) and the expression profiles compared between the different treatment conditions.

Data Analysis

Application of unique data mining techniques and preparing algorithm with respect to the in vitro modulation allowed for characterization of specific gene expression profiles in the context of patients phenotypes (see above). The pool of genes identified was then examined to determine which genes are able to separate responders from non-responders with high accuracy (>80%). Using these algorithms, the predictive power of each gene is provided numerically and a p value is given for each gene (p<0.05 is considered significant). Based on data from a comparable project, which accurately predicts glucocorticoid responders from non-responders (p values for the 20 predictive genes range from 0.05-0.001), the following analysis procedures are anticipated:
1. 12,600 genes measured (Affymetrix Genechip)
2. ˜8,000 expressed in PBM cells
3. ˜400 genes are differentially expressed
4. ˜20-30 genes correlate with drug response
5. >80% predictive accuracy between groups (p<0.05-0.001)

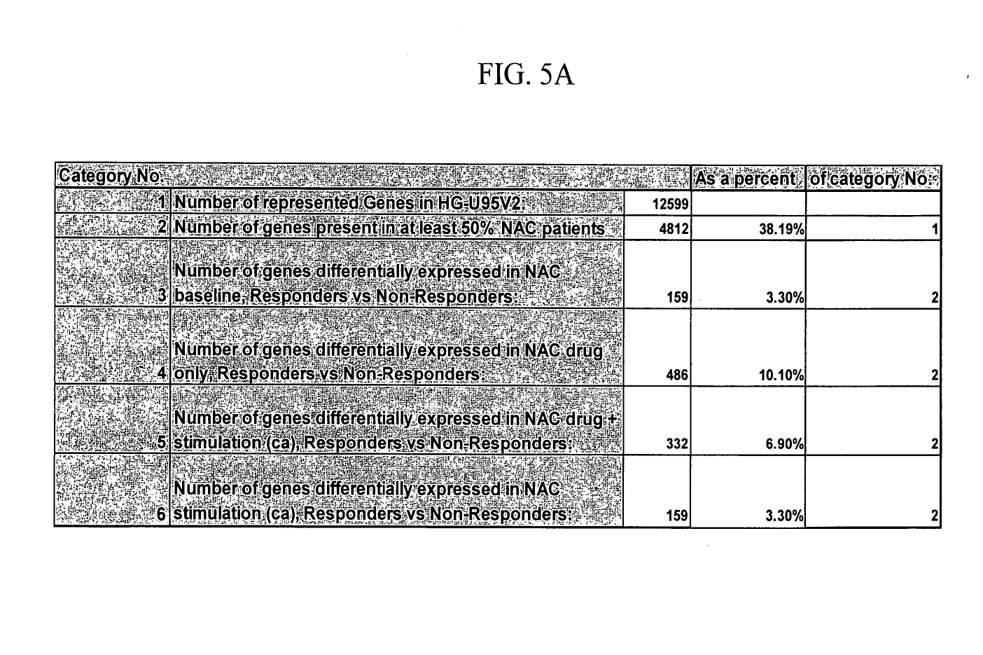

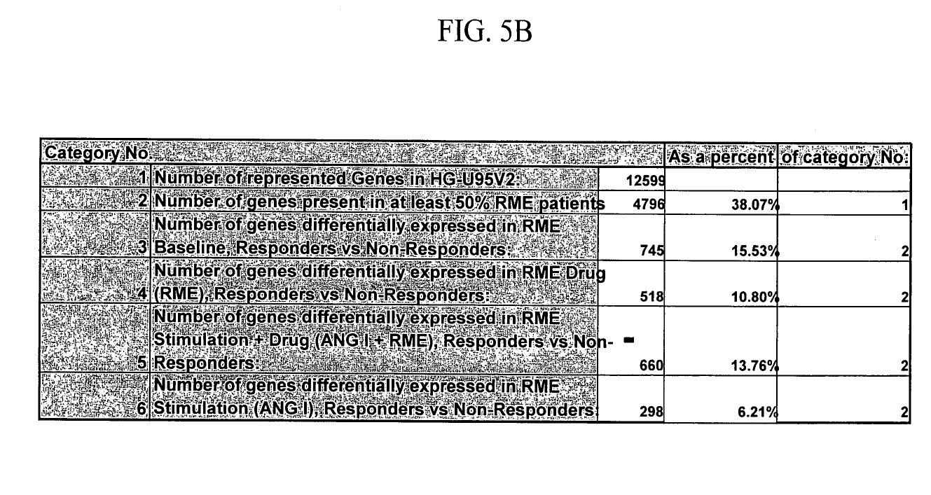

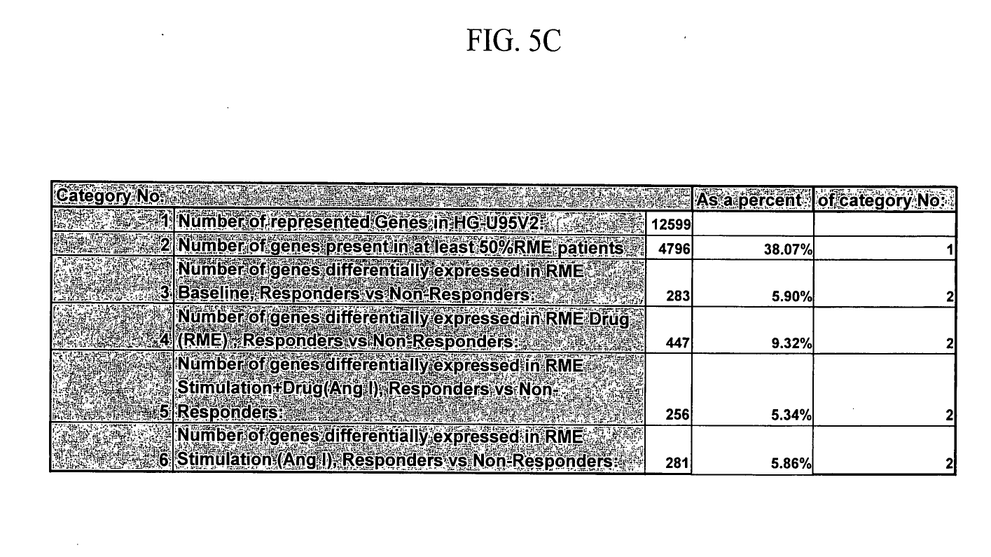

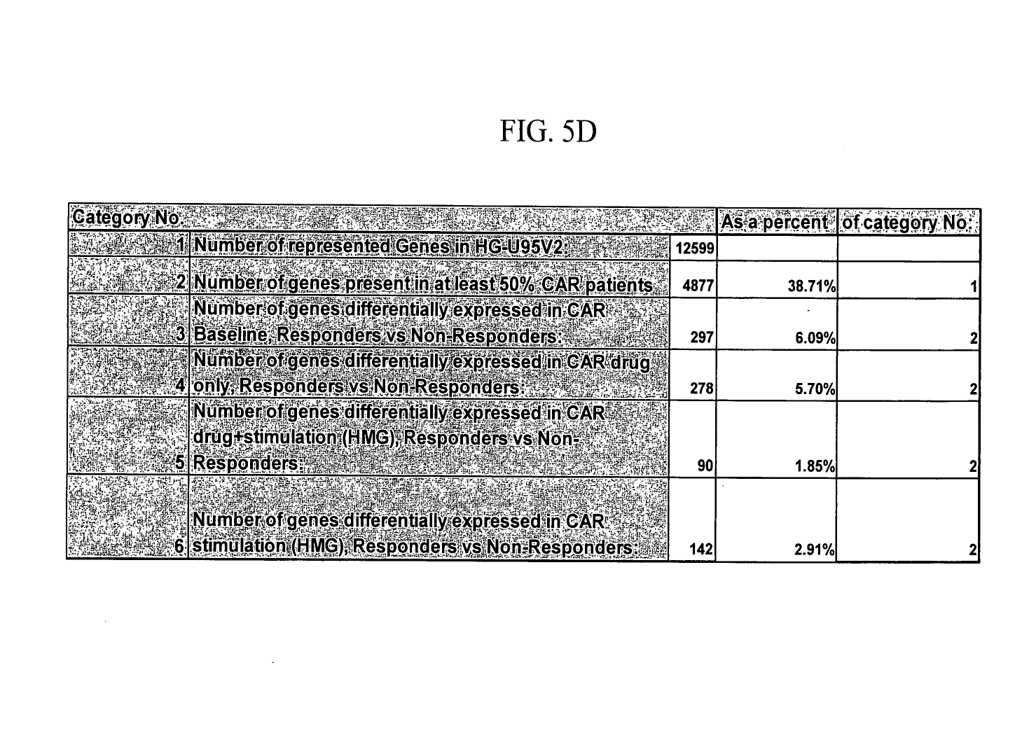

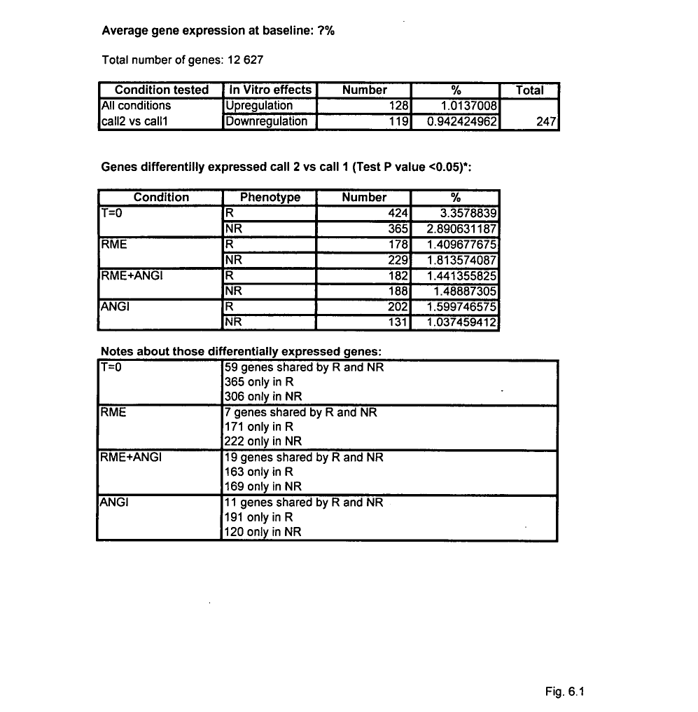

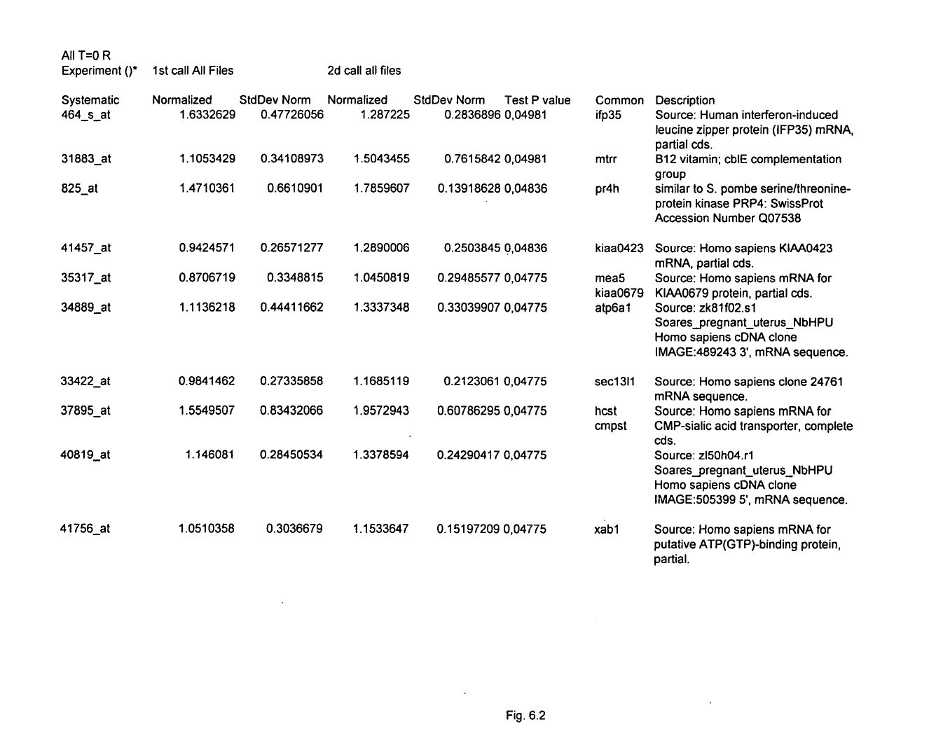

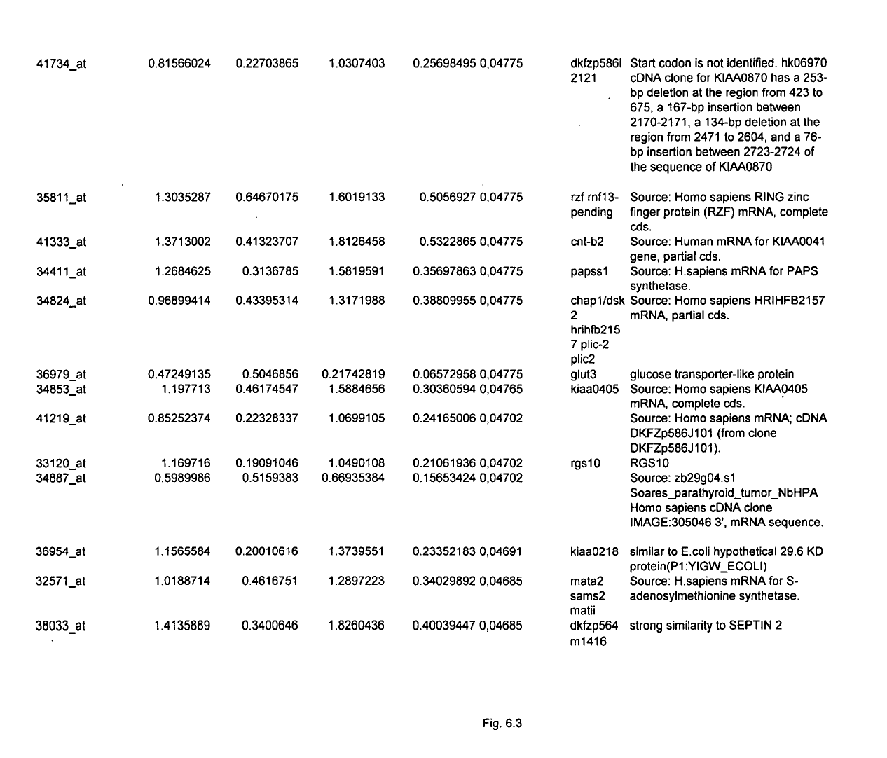

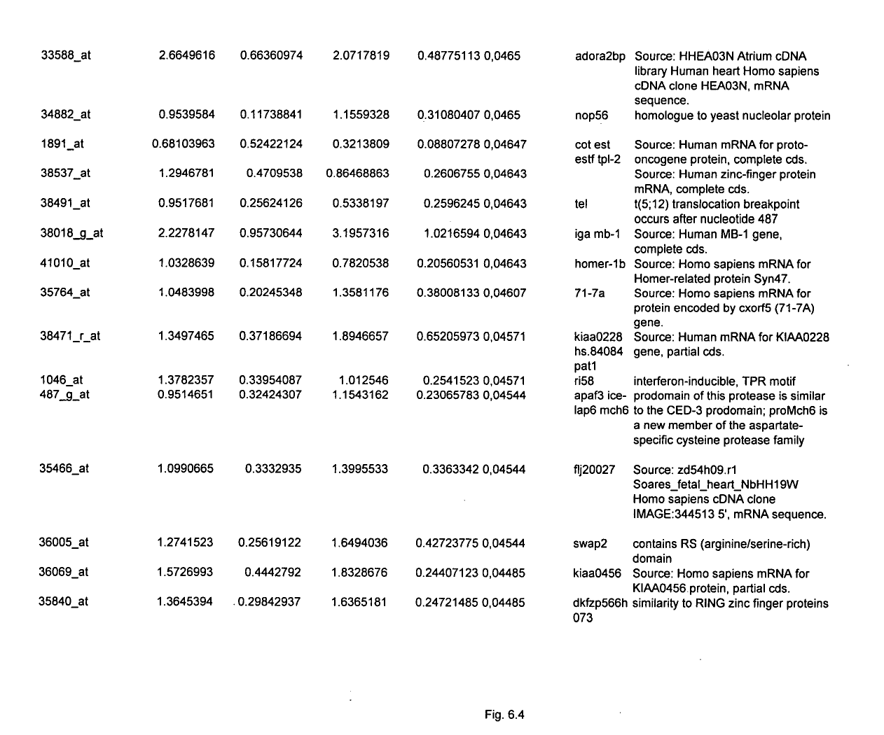

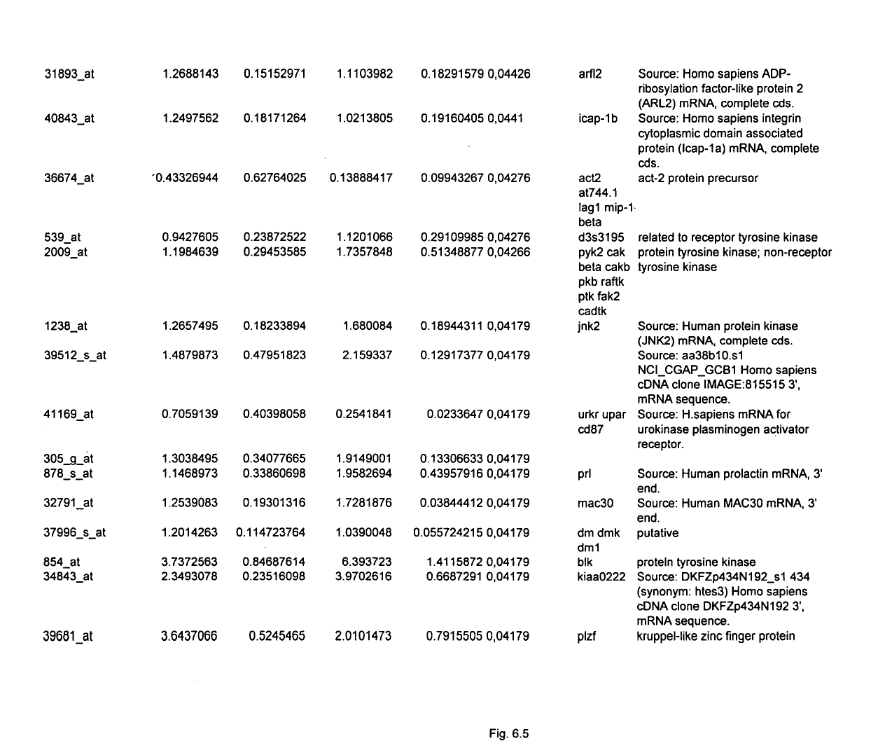

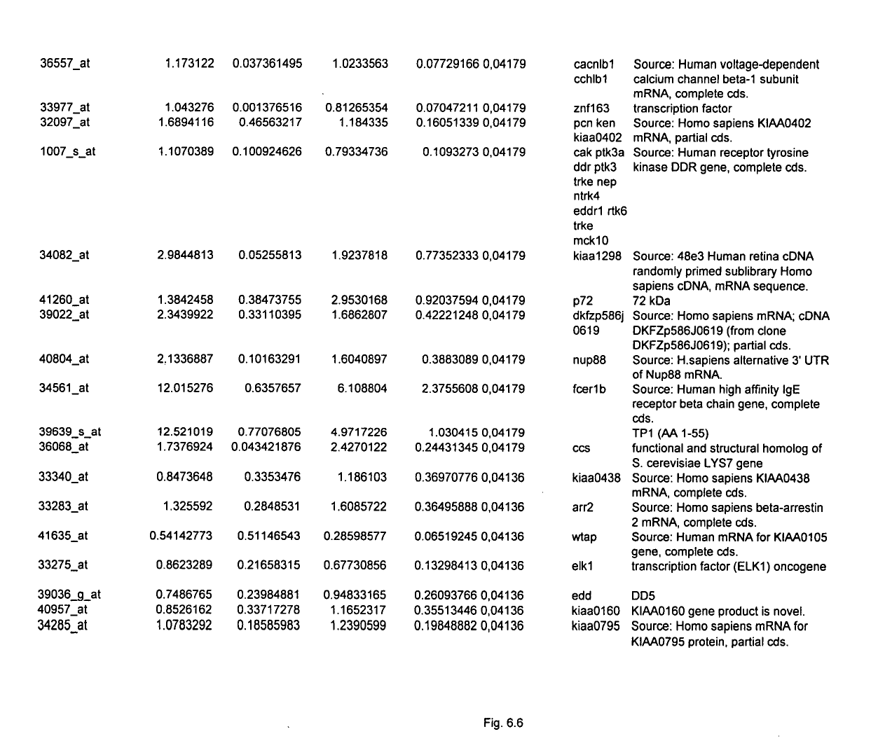

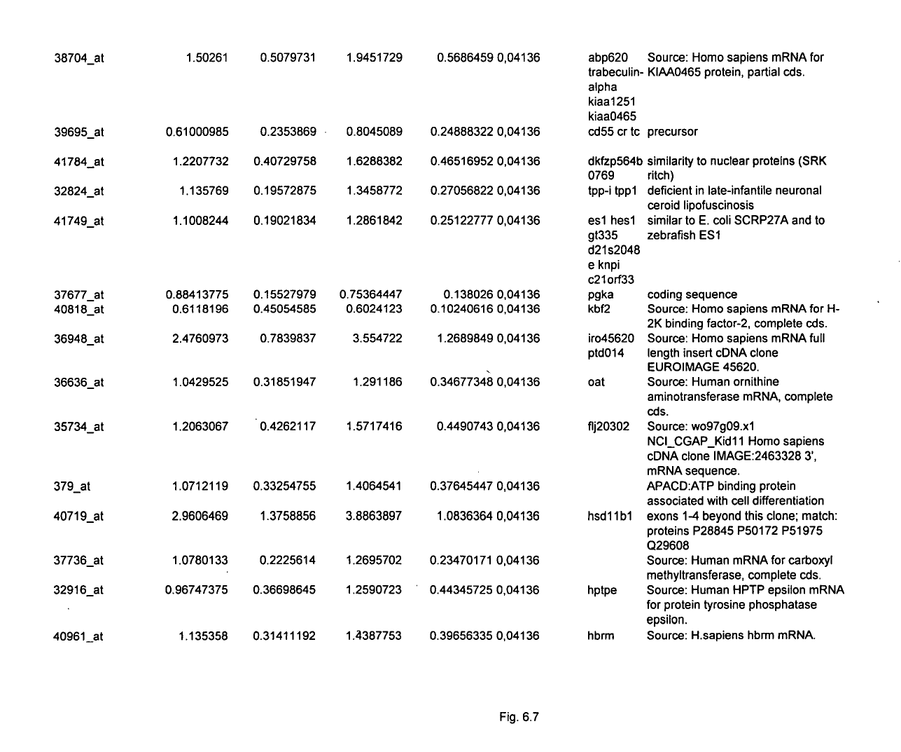

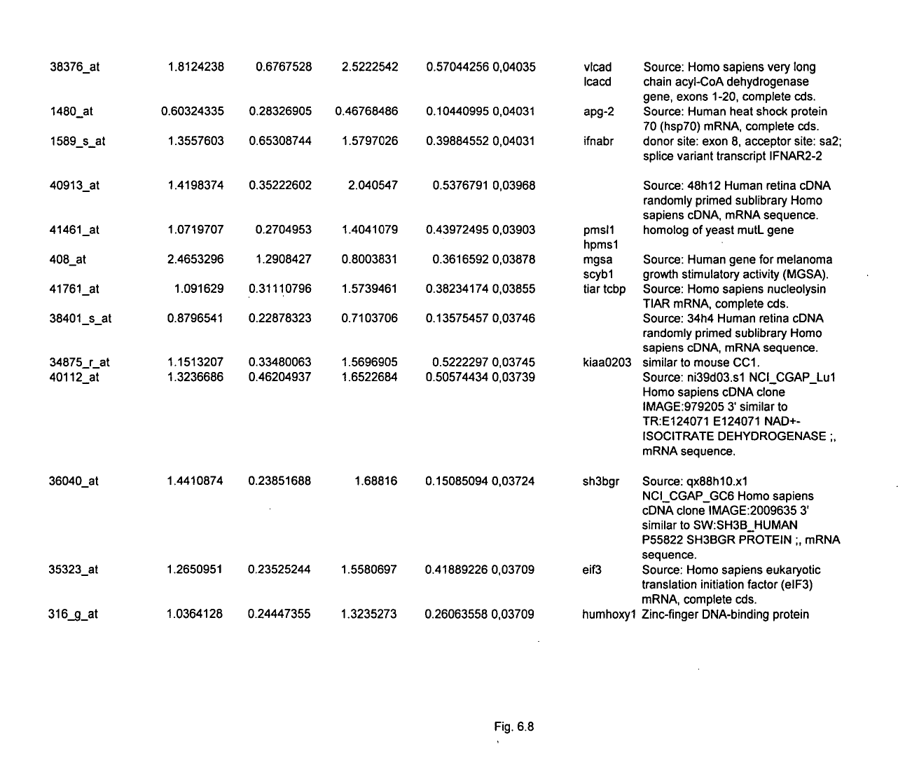

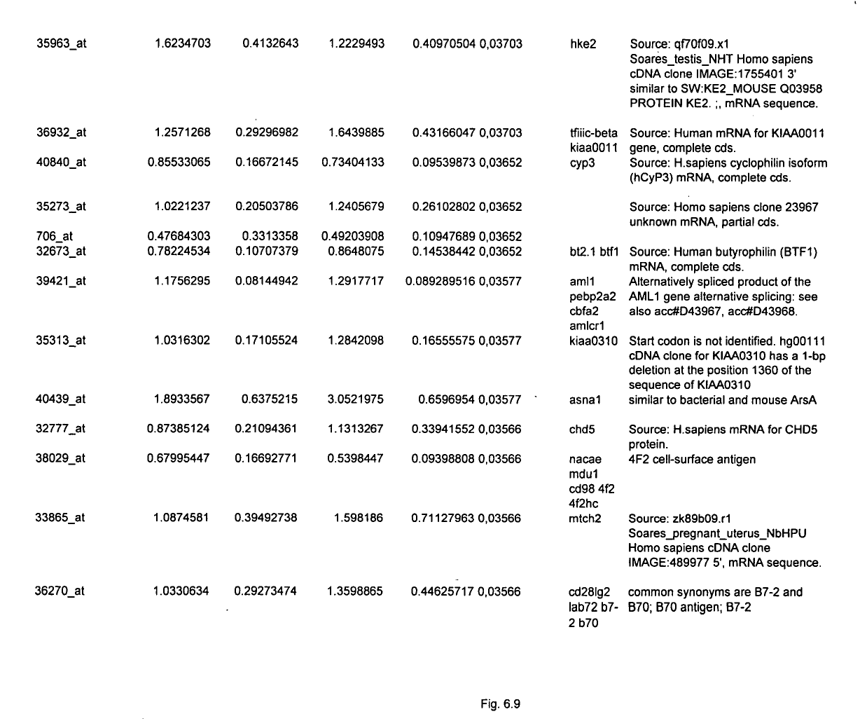

Actual results are described in FIGS. 5A and 5B and in Table 2.

### Example 2

### Characterization of gene expression profiles in Norvasc™ responsive vs. non-responsive patients with hypertension and/or angina using Affymetrix gene array.

Norvasc™ (amlodipine), a second-generation calcium channel blocker of the dihydropyridine class, has become the world's best-selling drug for the treatment of hypertension since its launch by Pfizer in 1990. Norvasc™ is a more selective calcium channel blocker in vascular smooth muscle cells than cardiac cells; hence, it has little or no negative inotropic effect. It provides reliable control of hypertension and angina with once-daily dosing due to its long plasma half-life of 35 to 50 hours.

Most calcium channel blockers are indicated for the treatment of hypertension, angina, or both, but Norvasc™, in addition to blocking calcium channels, has antiproliferative, membrane-modifying, and antioxidant activities that may be beneficial in other CVDs. In two clinical trials, Norvasc™ was shown to provide clinical benefit in patients with advanced congestive heart failure (CHF) and CHD. PRAISE (Prospective Randomized Amlodipine Survival Evaluation) showed that Norvasc™ significantly reduced the incidence of sudden deaths (21%) and pump failure deaths (6.6%) compared to placebo in patients with advanced CHF who were already taking ACE inhibitors, diuretics, and digitalis. PREVENT (Prospective Randomized Evaluation of the Vascular Effects of Norvasc Trial) showed that Norvasc™ reduced the incidence of fatal and nonfatal cardiovascular events by 31%, the need for angioplasty and coronary artery bypass grafting by 46%, and the hospitalization rate for severe angina by 35%.

At least five more clinical trials with Norvasc™ have begun. PRAISE-2 is a follow-on trial to PRAISE that will further evaluate Norvasc™ in the treatment of patients with non-ischemic CHF. The other trials will directly compare Norvasc™, alone or in combination with an ACE inhibitor, to other forms of therapy, including ACE inhibitors and diuretics, in reducing the incidence of fatal coronary artery disease and nonfatal MI in patients with hypertension or coronary artery disease. Another clinical trial will evaluate Norvasc™ in the treatment of renal disease. Pfizer and Warner-Lambert have initiated TNT (Treating to New Targets), a five-year trial to determine if lowering LDL cholesterol levels to even lower target levels with higher doses of Lipitor™ will provide additional benefits.

Angina Pectoris (“angina”) is another indication for Norvasc™. Angina refers to a recurring pain or discomfort in the chest that happens when some part of the heart does not receive enough blood. It is a common symptom of CHD, which occurs when vessels that carry blood to the heart become narrowed and blocked due to atherosclerosis. It is important to distinguish between the typical stable pattern of angina and “unstable” angina. Angina pectoris often recurs in a regular or characteristic pattern. Commonly a person recognizes that he or she is having angina only after several episodes have occurred, and a pattern has evolved. The level of activity or stress that provokes the angina is somewhat predictable, and the pattern changes only slowly. This is “stable” angina, the most common variety. However, instead of appearing gradually, angina can first appear as a very severe episode or as frequently recurring bouts of angina. Or, an established stable pattern of angina could change sharply; it may by provoked by far less exercise than in the past, or it may appear at rest. The term “unstable angina” is also used when symptoms suggest a heart attack, and hospital tests do not support that diagnosis. For example, a patient can have typical but prolonged chest pain and poor response to rest and medication, but there is no evidence of heart muscle damage either on the electrocardiogram or in blood enzyme tests.

Amlodipine is a dihydropyridine calcium antagonist (calcium ion antagonist or slow channel blocker) that inhibits the transmembrane influx of calcium ions into vascular smooth muscle and cardiac muscle. Experimental data suggest that Amlodipine binds to both dihydropyridine and nondihydropyridine binding sites. The contractile processes of cardiac muscle and vascular smooth muscle are dependent upon the movement of extracellular calcium ions into these cells through specific ion channels. Amlodipine inhibits calcium ion influx across cell membranes selectively, with a greater effect on vascular smooth muscle cells than on cardiac muscle cells. Negative inotropic effects can be detected in vitro, but such effects have not been seen in intact animals at therapeutic doses.

Following administration of therapeutic doses to patients with hypertension, Amlodipine produces vasodilation resulting in a reduction of supine and standing blood pressures. These decreases in blood pressure are not accompanied by a significant change in heart rate or plasma catecholamine levels with chronic dosing. Although the acute intravenous administration of amlodipine decreases arterial blood pressure and increases heart rate in hemodynamic studies of patients with chronic stable angina, chronic administration of oral amlodipine in clinical trials did not lead to clinically significant changes in heart rate or blood pressures in normotensive patients with angina.

With chronic once daily oral administration, anti-hypertensive effectiveness is maintained for at least 24 hours. Plasma concentrations correlate with effect in both young and elderly patients. The magnitude of reduction in blood pressure with Amlodipine is also correlated with the height of pretreatment elevation; thus, individuals with moderate hypertension (diastolic pressure 105-114 mm Hg) have about a 50% greater response than patients with mild hypertension (diastolic pressure 90-104 mm Hg). Normotensive subjects experienced no clinically significant change in blood pressure (+1/−2 mm Hg).

In the Circadian Anti-Ischemia Program in Europe (CAPE) trial, Norvasc™ significantly reduced angina attacks and nitroglycerin consumption. The CAPE trial was a randomized, double-blind, multi-country study of 315 male patients with chronic stable angina. A 2-week placebo run-in was followed by 8 weeks of treatment with Norvasc™ (5 mg/day titrated to 10 mg at week 4) or placebo. 271 patients were evaluated by diary for angina attack rate. 65% of patients in this study were already taking beta blockers before Norvasc™ was added. Once-daily Norvasc™ reduced the incidence of angina throughout the 24-hour dosing period. Significantly more Norvasc™ patients (75%) reported an improved ability to perform usual physical activities vs those taking placebo (59%, P=0.003). Only 2% of Norvasc patients discontinued therapy due to adverse events versus 4.4% taking placebo (P═NS).

In view of the above considerations, the following hypotheses were addressed:
A: that hypertensive patients who demonstrate clinical improvement when taking the drug, Norvasc™, can be identified by specific gene expression profiles in white blood cells, which are characteristic for patients who do respond to the drug and differ from the expression profiles in non-responders.
B: that these expression profiles can be used to develop an expression or DNA-based test that accurately predicts the responsiveness to Norvasc™.

To address these hypotheses, 65 patients were identified and recruited, 46 of whom demonstrate a clinical response (“responders”) to the calcium channel inhibitor, Norvasc™, and 19 of whom are “non-responders”. Norvasc™ response was be measured by a scoring function taking into account both clinical and laboratory parameters as described below. Gene expression profiles that accurately predict the responsiveness of these hypertensive patients to Norvasc™, using a gene expression array chip system and data mining algorithm(s), were obtained. The goal, confirmed below, was that the results from the expression studies would demonstrate distinctive gene expression profiles that allow for characterization between Norvasc™ responders and non-responders with predictive accuracy of 82%, and, based on these gene expression profiles, a diagnostic test to identify patients who are best suited for Norvasc™ therapy can be developed (FIG. 3).

The diagnostic test can be developed from such gene expression profile data to include one or more of the following aspects:
a) the genes whose expression patterns are highly predictive can be re-arrayed on a more cost-effective expression platform and can be used as a predictor of response in the clinic;
b) finding associated variants within the genes whose expression pattern is predictive and using the latter as a DNA-based predictor of response in the clinic.
   One of skill in the art would appreciate that such data can be used in a variety of ways to design a test that accurately separates responders from non-responders.
   Subject/Patient Criteria

Inclusion Criteria

The criteria the subject/patient had to satisfy to enter the study, includes the following:

a. Hypertension diagnosed by a Cardiologist. The method used to diagnose hypertension is based on the ICE-9 and ICD-10 classification system, and concurs with the diagnostic hypertension criteria outlined by the American Heart Association and includes the following measures: Blood pressure greater than 140 systolic and 90 diastolic (adult person). For this study, individuals will be recruited primarily with moderate to severe hypertension.
b. Patients with hypertension with a severity level that is judged to be appropriate to treatment with Norvasc™ were recruited to participate in the study. Study patients signed a consent and donate a blood sample for the study.
c. Phenotype was confirmed by measurement of blood pressure levels at different time points.
d. Age 12-80 years.
e. Both males and females were recruited.
f. White Caucasian (Icelandic hypertensive patient population).
g. Norvasc™ therapy for 8 weeks. Examination by a study cardiologist every 3 weeks who confirmed clinical response to Norvasc™ therapy.
h. Response to Norvasc™: Patients were categorized as either Norvasc™ responders or Norvasc™ non-responders. Norvasc™ response is defined by any of the following:
   i) improved control of hypertension symptoms (blood pressure reduced by 10 mm/Hg in systole or more and/or 6 mm/Hg in diastole or more) when taking Norvasc™,
   ii) improved quality of life/well being on Norvasc™ therapy as judged by the patient and his/her cardiologist.
   iii) Norvasc™ non-responders do not experience improvement in the above measures. The same cardiologists examined all 65 patients before and after Norvasc™ trial.

Exclusion Criteria

A precise list of criteria that would exclude the subject/patient from entering the study, included:
a. Therapies that could interfere with evaluation of efficacy or the incidence of adverse effects, including:
   i) Other investigational drugs (simultaneously)
   ii) Concurrent anti-hypertensive medication that is not taken regularly
b. Diseases or conditions that could interfere with the evaluation of efficacy or the incidence of adverse effects, including:
   i) Pregnancy or lactation
   ii) Hypersensitivity or serious adverse experiences to hypertension drugs in the past
c. Sensitivity to the study drug or its components
e. If compliance to medication is of question
   Summary of Study Design

Patients with hypertension diagnosed by the above criteria were invited to participate in the study and recruited to take the calcium channel inhibitor, Norvasc™. Sixty patients were recruited to participate; 46 of whom were identified as responders and 19 of whom were identified as non-responders according to medical records. A cardiologist, who was blinded to the expression array studies, confirmed the phenotype of all patients. All patients had a physical examination where their phenotype was confirmed. The patients donated a blood sample for the study. Fifty to sixty mL of EDTA blood was collected for the study in 7×8 mL tubes. PBMCs were isolated from the rest of the blood, their RNA extracted and transcribed to cDNA, which upon re-transcript to cRNA, was hybridized to Affymetrix GeneChip™ microarrays as described below.

Treatment Plan

All patients recruited were treated with therapeutic doses of Norvasc™ for minimum of 8 weeks and examined by the study cardiologist before and after starting the drug. Blood samples were drawn at a similar time (e.g., around 9 AM) in the morning from all patients. The dose was recorded for all patients. No diet restrictions were implemented.

Study Procedures

PBMCs were isolated by the standard Ficoll method.

The cells were then divided into 4 conditions each:
i) Immediate RNA isolation at baseline (0 hr or TO);
ii) Activation of calcium ion channels (using Ca²⁺), in the absence and presence of a one hour pre-treatment with Norvasc™ (0-6M), followed by immediate RNA isolation;
iii) Exposure to Ramace™ alone; and
iv) Exposure to the stimulant alone.

Upon RNA isolation, the samples were column cleaned, quantified by spectrophotometry, and equal amounts (5 μg) of total RNA will be used for cDNA synthesis for each condition. The cDNAs from the responders and non-responders are subsequently processed on to 12,600 Affymetrix GeneChip™ microarrays (Hu95) and the expression profiles compared between the different treatment conditions.

Data Analysis

Application of unique data mining techniques and preparing algorithm with respect to the in vitro modulation allowed for characterization of specific gene expression profiles in the context of patients phenotypes. The pool of genes identified was then examined to determine which genes are able to separate responders from non-responders with high accuracy (>80%). Using these algorithms, the predictive power of each gene is provided numerically and a p value is given for each gene (p<0.05 is considered significant). Based on data from a comparable project, which accurately predicts glucocorticoid responders from non-responders (p values for the 20 predictive genes range from 0.05-0.001), the following analysis procedures were anticipated:
1. 12,600 genes measured (Affymetrix Genechip)
2. ˜8,000 expressed in PBM cells
3. ˜400 genes are differentially expressed
4. ˜20-30 genes correlate with drug response
5. >80% predictive accuracy between groups (p<0.05-0.001)

Actual results are shown in FIG. 5C.

### Example 3

### Characterization of gene expression profiles in Cozaar™ responsive vs. non-responsive hypertensive patients using Affymetrix gene array.

Cozaar™ (Losartan potassium) and Hyzaar™ (Losartan potassium hydrochlorothiazide) continue to lead all competition in the angiotensin II antagonist (AIIA) class of anti-hypertensives by more than a two-to-one margin.

Angiotensin II (formed from angiotensin I in a reaction catalyzed by angiotensin converting enzyme (ACE, kininase II)), is a potent vasoconstrictor, the primary vasoactive hormone of the renin-angiotensin system and an important component in the pathophysiology of hypertension. It also stimulates aldosterone secretion by the adrenal cortex. The classical view has been that angiotensinogen, produced by the liver, is converted into angiotensin I (AI) by renin, which is produced by the kidney. Angiotensin I is converted into angiotensin II (AII) by angiotensin-converting enzyme (ACE). The final product, AII, acts on the AII receptors. Angiotensin II is a potent vasoconstrictor, the primary vasoactive hormone of the renin-angiotensin system and an important component in the pathophysiology of hypertension. However, other data suggest that there are non-ACE pathways for the production of AII. These pathways may exist in the walls of the blood vessels and in other tissues such as the heart, kidneys, and adrenals. For example, cathepsin G, chymostatin-sensitive angiotensin II generating enzyme (CAGE), and chymase are believed to generate AII from AI.

Irrespective of the route of formation, AII binds to the AT1 receptor, resulting in vasoconstriction and increases in sympathetic activation and aldosterone secretion. Each of these responses to AII is important in the regulation of blood pressure. All the known cardiovascular effects of AII are medicated through the AT1 receptor. There is also an AT2 receptor found in many tissues, but it has not been demonstrated to be associated with cardiovascular homeostasis.

Losartan and its principal active metabolite block the vasoconstrictor and aldosterone-secreting effects of angiotensin II by selectively blocking the binding of angiotensin II to the AT1 receptor found in many tissues. Both losartan and its principal active metabolite do not exhibit any partial agonist activity at the AT1 receptor and have much greater affinity (about 1000-fold) for the AT 1 receptor than for the AT2 receptor. In vitro binding studies indicate that losartan is a reversible, competitive inhibitor of the AT1 receptor. The active metabolite is 10 to 40 times more potent by weight than losartan and appears to be a reversible, non-competitive inhibitor of the AT1 receptor.

Neither losartan nor its active metabolite inhibits ACE, the enzyme that converts angiotensin I to angiotensin II nor do they bind to or block other hormone receptors or ion channels known to be important in cardiovascular regulation.

The active parent molecule reaches mean peak serum concentration within one hour. The long-acting metabolite is detectable in plasma up to 36 hours after a single dose. It is a noncompetitive inhibitor of the AT1 receptor and does not accumulate upon repeated once-daily dosing.

In view of the above considerations, the following hypotheses were addressed:

A: that hypertensive patients who demonstrate clinical improvement when taking the drug, Cozaar™, can be identified by specific gene expression profiles in white blood cells, which are characteristic for patients who do respond to the drug and differ from the expression profiles in non-responders.

B: that these expression profiles can be used to develop an expression or DNA-based test that accurately predicts the responsiveness to Cozaar™.

To address these hypotheses, 65 patients were identified and recruited, 47 of whom demonstrate a clinical response (“responders”) to the AIIA, Cozaar™, and 18 of whom are “non-responders”. Cozaar™ response was be measured by a scoring function taking into account both clinical and laboratory parameters as described below. Gene expression profiles that accurately predict the responsiveness of these hypertensive patients to Cozaar™, using a gene expression array chip system and data mining algorithm(s), were obtained. The goal, confirmed below, was that the results from the expression studies would demonstrate distinctive gene expression profiles that allow for characterization between Cozaar™ responders and non-responders with predictive accuracy>80%, and, based on these gene expression profiles, a diagnostic test to identify patients who are best suited for Cozaar™ therapy can be developed.

The diagnostic test can be developed from such gene expression profile data to include one or more of the following aspects:
a) the genes whose expression patterns are highly predictive can be re-arrayed on a more cost-effective expression platform and can be used as a predictor of response in the clinic;
b) finding associated variants within the genes whose expression pattern is predictive and using the latter as a DNA-based predictor of response in the clinic.
   One of skill in the art would appreciate that such data can be used in a variety of ways to design a test that accurately separates responders from non-responders.
   Subject/Patient Criteria

Inclusion Criteria

The criteria the subject/patient had to satisfy to enter the study, includes the following:

a. Hypertension diagnosed by a Cardiologist. The method used to diagnose hypertension is based on the ICE-9 and ICD-10 classification system, and concurs with the diagnostic hypertension criteria outlined by the American Heart Association and includes the following measures: Blood pressure greater than 140 systolic and 90 diastolic (adult person). For this study, individuals will be recruited primarily with moderate to severe hypertension.
b. Patients with hypertension with a severity level that is judged to be appropriate to treatment with Cozaar™ were recruited to participate in the study. Study patients signed a consent and donate a blood sample for the study.
c. Phenotype was confirmed by measurement of blood pressure levels at different time points.
d. Age 12-80 years.
e. Both males and females were recruited.
f. White Caucasian (Icelandic hypertensive patient population).
g. Cozaar™ therapy for 8 weeks. Examination by a study cardiologist every 3 weeks who confirmed clinical response to Cozaar™ therapy.
h. Response to Cozaar™: Patients were categorized as either Cozaar™ responders or Cozaar™ non-responders. Cozaar™ response is defined by any of the following:
   i) improved control of hypertension symptoms (blood pressure reduced by 10 mm/Hg in systole or more and/or 6 mm/Hg in diastole or more) when taking Cozaar™,
   ii) improved quality of life/well being on Cozaar™ therapy as judged by the patient and his/her cardiologist.
   iii) Cozaar™ non-responders do not experience improvement in the above measures. The same cardiologists examined all 65 patients before and after Cozaar™ trial.

Exclusion Criteria

A precise list of criteria that would exclude the subject/patient from entering the study, included:
a. Therapies that could interfere with evaluation of efficacy or the incidence of adverse effects, including:
   i) Other investigational drugs (simultaneously)
   ii) Concurrent anti-hypertensive medication that is not taken regularly
b. Diseases or conditions that could interfere with the evaluation of efficacy or the incidence of adverse effects, including:
   i) Pregnancy or lactation
   ii) Hypersensitivity or serious adverse experiences to hypertension drugs in the past
c. Sensitivity to the study drug or its components
e. If compliance to medication is of question
   Summary of Study Design

Patients with hypertension diagnosed by the above criteria were invited to participate in the study and recruited to take the AIIA, Cozaar™. Sixty patients were recruited to participate; 47 of whom were identified as responders and 18 of whom were identified as non-responders according to medical records. A cardiologist, who was blinded to the expression array studies, confirmed the phenotype of all patients. All patients had a physical examination where their phenotype was confirmed. The patients donated a blood sample for the study. Fifty to sixty mL of EDTA blood was collected for the study in 7×8 mL tubes. PBMCs were isolated from the rest of the blood, their RNA extracted and transcribed to cDNA, which upon re-transcript to cRNA, was hybridized to Affymetrix GeneChip™ microarrays as described below.

Treatment Plan

All patients recruited were treated with therapeutic doses of Cozaar™ for minimum of 8 weeks and examined by the study cardiologist before and after starting the drug. Blood samples were drawn at a similar time (e.g., around 9 AM) in the morning from all patients. The dose was recorded for all patients. No diet restrictions were implemented.

Study Procedures

PBMCs were isolated by the standard Ficoll method.

The cells were then divided into 4 conditions each:
i) Immediate RNA isolation at baseline (0 hr or TO);
ii) Exposure to a specific All receptor agonist (e.g., angiotensin II) for 4 hours, thereby activating the All receptor pathway, in the absence and presence of a one hour pre-treatment with Cozaar™ (10-6M), followed by immediate RNA isolation;
iii) Exposure to Ramace™ alone; and
iv) Exposure to the stimulant alone.

Upon RNA isolation, the samples were column cleaned, quantified by spectrophotometry, and equal amounts (5 μg) of total RNA will be used for cDNA synthesis for each condition. The cDNAs from the responders and non-responders are subsequently processed on to 12,600 Affymetrix GeneChip™ microarrays (Hu95) and the expression profiles compared between the different treatment conditions.

Data Analysis

Application of unique data mining techniques and preparing algorithm with respect to the in vitro modulation allowed for characterization of specific gene expression profiles in the context of patients phenotypes. The pool of genes identified was then examined to determine which genes are able to separate responders from non-responders with high accuracy (>80%). Using these algorithms, the predictive power of each gene is provided numerically and a p value is given for each gene (p<0.05 is considered significant). Based on data from a comparable project, which accurately predicts glucocorticoid responders from non-responders (p values for the 20 predictive genes range from 0.05-0.001), the following analysis procedures were anticipated:
1. 12,600 genes measured (Affymetrix Genechip)
2. ˜8,000 expressed in PBM cells
3. ˜400 genes are differentially expressed
4. ˜20-30 genes correlate with drug response
5. >80% predictive accuracy between groups (p<0.05-0.001)

Actual results are shown in FIG. 5D.

While this invention has been particularly shown and described with references to preferred embodiments thereof, it will be understood by those skilled in the art that various changes in form and details may be made therein without departing from the scope of the invention encompassed by the appended claims.

1. A method for predicting the efficacy of a drug for treating hypertension in a patient, comprising:
a) determining a gene expression profile from a sample obtained from cells of the patient in the absence and presence of in vitro modulation of the cells with specific mediators; and
b) comparing the gene expression profile of the sample with a reference gene expression profile, wherein similarity between the sample expression profile and the reference expression profile predicts the efficacy of the drug for treating hypertension in the patient.

2. The method of claim 1, wherein the expression profile comprises expression values for one or more genes listed in FIGS. 2-4.

3. The method of claim 1, further comprising exposing the sample to the drug for treating hypertension prior to obtaining the gene expression profile of the sample.

4. The method of claim 1, wherein the drug is selected from a class of drugs selected from the group consisting of: an angiotensin converting enzyme inhibitor, a calcium channel blocker and an angiotensin II receptor antagonist.

5. The method of claim 4, wherein the drug is selected from the group consisting of Ramace™, Norvasc™ and Cozaar™.

6. The method of claim 1, wherein the sample of cells is derived from blood.

7. The method of claim 6, wherein the sample is comprised of peripheral blood mononuclear cells.

8. The method of claim 1, wherein the gene expression profile of the sample is obtained using a hybridization assay to oligonucleotides contained in a microarray.

9. The method of claim 8, wherein the oligonucleotides are capable of hybridizing to polynucleotides corresponding to the informative genes and reagents for detecting hybridization.

10. The method of claim 1, wherein the gene expression profile of the sample is obtained by detecting the protein products of the informative genes.

11. The method of claim 10, wherein determining the gene expression profile comprises antibodies that are capable of specifically binding protein products of the informative genes and reagents for detecting antibody binding.

12. The method of claim 1, wherein the reference expression profile is that of cells derived from patients who do not have hypertension.

13. The method of claim 1, wherein the cells are treated with the drug candidate before the expression profile is obtained.

14. A microarray consisting of one or more genes listed in FIGS. 2-4.
